(19) United States
(12) Patent Application Publication
(10) Pub. No.: US 2015/0240301 A1
(43) Pub. Date: Aug. 27, 2015

(54) METHODS AND COMPOSITIONS RELATING TO NEXT GENERATION SEQUENCING FOR GENETIC TESTING IN ALK RELATED CANCERS

(71) Applicant: INSIGHT GENETICS, INC., Nashville, TN (US)

(72) Inventors: David Hout, Nashville, TN (US); Eric Dahlhauser, Goodlettsville, TN (US); Adam Platt, Nashville, TN (US)

(73) Assignee: Insight Genetics, Inc., Nashville, TN (US)

(21) Appl. No.: 14/431,430

(22) PCT Filed: Sep. 26, 2013
(86) PCT No.: PCT/US13/61950

§ 371 Date: Mar. 26, 2015

Related U.S. Application Data
(60) Provisional application No. 61/710,455, filed on Oct. 5, 2012.

(51) Int. Cl. C12Q 1/68

(52) U.S. Cl. CPC C12Q 1/6874

(57) ABSTRACT

Disclosed are methods, assays, and compositions for detecting the presence of a kinase inhibitor resistance. The disclosed method and primer panels work with any method for detecting nucleic acid variation in a sample including, but not limited to next generation sequencing.

### BACKGROUND

Mutations of anaplastic lymphoma kinase (ALK) gene are thought to be involved in the development of subsets of numerous cancers including i) non-small cell lung carcinoma (NSCLC); ii) diffuse large B-cell lymphoma; iii) esophageal squamous cell carcinoma; iv) anaplastic large-cell lymphoma (ALCL); v) neuroblastoma (a childhood cancer that arises from the developing peripheral nervous system); and vi) the sarcomas known as inflammatory myofibroblastic tumors (IMTs). Patient outcomes with many of these malignancies are poor, due in part to the late detection of the cancers because of the lack of efficient clinical diagnostic methods. Early detection and diagnosis of ALK-mediated cancers dramatically increases survival rates within the patient population; as an example, early detection of ALK-positive anaplastic large-cell lymphoma can result in survival rates of up to 83% whereas late detection is associated in some instances with survival of only 50% of the patient population.

The critical role of deregulated ALK signaling as a driver of subsets of NSCLC, ALCL, and other ALK-dependent cancer types has been validated in clinical trials, with dramatic anti-tumor efficacy observed in response to the ALK small-molecule inhibitor crizotinib (XALKORI®, Pfizer; approved by the US FDA in August 2011). Unfortunately, despite the marked anti-tumor responses to XALKORI® seen in patients with ALK-driven tumors, most patients eventually experience progression of their cancer as a consequence of treatment resistance. For example, the median duration of progression-free survival in patients with ALK-positive NSCLC treated with Xalkori is only about 10 months. What is needed are assays the can efficiently and reliably detect kinase inhibitor-resistance mutations and therefore predict which members of a patient population is likely to develop kinase inhibitor resistance. Additionally as new generations of small-molecule inhibitors are developed, also need is a clinically applicable diagnostic test to identify resistance mutations in the ALK kinase domain and therefore to guide the rational use of these small-molecule inhibitors for the treatment of ALK-driven cancers that have lost their responsiveness to 1^{st}-generation inhibitor therapy. Moreover, once several ALK small-molecule inhibitors are approved for clinical use, optimal management of patients with ALK-driven tumors will require screening for de novo inhibitor resistance mutations by healthcare providers treating newly diagnosed patients in order to assess their inhibitor sensitivity and choose the best ALK inhibitor drug(s) for personalized therapy.

### BRIEF SUMMARY

The methods, assays, and compositions disclosed herein relate to the field of detection or diagnosis of mutations that confer resistance to kinase inhibitors of a disease or condition such as cancer. In one aspect, the kinase inhibitors or ALK kinase inhibitors. Also disclosed herein are methods and assays for assessing the susceptibility or risk for developing resistance to an inhibitor, wherein the disease or condition is a cancer associated with expression of the ALK gene. It is understood and herein contemplated that the methods disclosed herein allow for rapid and sensitive detection of nucleic acid expression of mutations in ALK.

In another aspect, disclosed herein are kinase inhibitor resistance panels comprising one or more primer sets from each of the genes selected from group of genes comprising KRAS, BRAF, EGFR, ALK, and KIT.

In accordance with the purpose(s) of this invention, as embodied and broadly described herein, this invention, in one aspect, relates to an ALK kinase inhibitor resistance panel. In particular, the invention, in one aspect, relates to an ALK kinase inhibitor resistance panel comprising one or more primer sets for detecting the presence of a mutation in a gene that will confer resistance to the ALK kinase inhibitor.

Additional advantages of the disclosed methods and compositions will be set forth in part in the description which follows, and in part will be understood from the description, or may be learned by practice of the disclosed method and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

FIG. 1 shows XALKORI®-resistance mutations identified in patient specimens. The FIGURE depicts the XALKORI®-resistance mutations in the ALK kinase domain identified to date in patient cancer specimens.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a larger amount of a composition or compound, such as an amplification product relative to a control. Thus, for example, an increase in the amount in amplification products can include but is not limited to a 10%, 20%, 30%, 40%, 50%, 60%, 70%, 80%, 90%, 100%, 150%, 200%, 250%, 300%, 350%, 400%, 450%, 500%, 550%, 600%, 700%, 800%, 900%, 1000%, 1500%, 2000%, 2500%, 3000%, 3500%, 4000%, 4500%, or 5000% increase. It is further contemplated herein that the detection an increase in expression or abundance of a DNA, mRNA, or protein relative to a control necessarily includes detection of the presence of the DNA, mRNA, or protein in situations where the DNA, mRNA, or protein is not present in the control.

“Obtaining a tissue sample” or “obtain a tissue sample” means to collect a sample of tissue either from a party having previously harvested the tissue or harvesting directly from a subject. It is understood and herein contemplated that tissue samples obtained directly from the subject can be obtained by any means known in the art including invasive and non-invasive techniques. It is also understood that methods of measurement can be direct or indirect. Examples of methods of obtaining or measuring a tissue sample can include but are not limited to tissue biopsy, tissue lavage, blood collection, aspiration, tissue swab, spinal tap, magnetic resonance imaging (MRI), Computed Tomography (CT) scan, Positron Emission Tomography (PET) scan, and X-ray (with and without contrast media). It is further understood that a “tissue” can include, but is not limited to any grouping of one or more cells or analytes to be used in a an ex vivo or in vitro assays. Such tissues include but are not limited to blood, saliva, sputum, lymph, cellular mass, and tissue collected from a biopsy.

### Kinase Inhibitor Resistant Panels

In one aspect, disclosed herein are kinase inhibitor resistance panels such as, for example, an ALK kinase inhibitor panel. Kinase inhibitors are known in the art and have found use in the treatment of, amongst other things, the treatment of cancer. For example, cancers involving the overexpression or fusion of Analplastic Lymphoma Kinase can be treated through the use of a kinase inhibitor. Kinase inhibitors are known in the art and include, but are not limited to crizotinib, afatinib, Axitinib, bevacizumab, Bosutinib, Cetuximab, Dasatinib, Erlotinib, Fostamati nib, Gefitinib, Imatinib, Lapatinib, Lenvatinib, Nilotinib, Panitumumab, Pazopanib, Pegaptanib, Ranibizumab, Ruxolitinib, Sorafenib, Sunitinib, Trastuzumab, and Vemurafenib. Thus, in one aspect, disclosed herein are kinase inhibitor resistance panels for detecting susceptibility or resistance to treatment in a subject to a kinase inhibitor comprising crizotinib, afatinib, Axitinib, bevacizumab, Bosutinib, Cetuximab, Dasatinib, Erlotinib, Fostamati nib, Gefitinib, Imatinib, Lapatinib, Lenvatinib, Nilotinib, Panitumumab, Pazopanib, Pegaptanib, Ranibizumab, Ruxolitinib, Sorafenib, Sunitinib, Trastuzumab, or Vemurafenib.

Unfortunately, mutations in the ALK sequence and other genes, such as, BRAF, KIT, KRAS, and EGFR can lead to kinase inhibitor resistance. These mutations can comprise any of the mutations to ALK, KIT, BRAF, KRAS, or EGFR listed in Tables 2, 3, 4, 5, or 6. Accordingly, in a further aspect, disclosed herein are kinase inhibitor panels comprising one or more primer sets that selectively hybridize and can be used to amplify one of the genes selected from group of genes comprising KRAS (SEQ ID NO: 7718), BRAF (SEQ ID NO: 7717), EGFR (SEQ ID NO: 7716), ALK (SEQ ID NO: 7714 and SEQ ID NO: 7717 (cDNA)), and KIT. In one aspect, the kinase inhibitor resistance panel disclosed herein can comprise one or more primer set(s) that hybridizes and amplifies nucleic acid from exon 1 (SEQ ID NOs: 4601-4880 and 7181-7230) exon 2 (SEQ ID NOs: 4881-5200 and 7231-7326) or both exons 1 and 2 (SEQ ID NOs: 7327-7610) of KRAS; exon 18 (SEQ ID NOs: 1641-1760 and 5819-5934), exon 19 (SEQ ID NOs: 1761-1880), exon 20 (SEQ ID NOs: 1881-2000 and 5934-6042), exon 21 (SEQ ID NOs: 2001-2120 and 6043-6150), exon 22 (SEQ ID NOs: 2121-2240, 2321-2360, and 2401-2440), exons 18 and 19 (SEQ ID NOs: 2241-2280), exons 18, 19, and 20 (SEQ ID NOs: 6151-6274), exons 20 and 21 (SEQ ID NOs: 2281-2320 and 6275-6388), or exons 18, 19, 20, and 21 (SEQ ID NOs: 2361-2400 and 6389-6524) of EGFR; exon 8 (SEQ ID NOs: 2441-2800), exon 9 (SEQ ID NOs: 2841-3120), exon 10 (SEQ ID NOs: 3201-3360), exon 11 (SEQ ID NOs: 3361-3480), exon 12 (SEQ ID NOs: 3481-3640), exon 13 (SEQ ID NOs: 3641-3800), exon 17 (SEQ ID NOs: 4241-4600), exon 8 and 9 (SEQ ID NOs: 2801-2840), exons 9 and 10 (SEQ ID NOs: 3121-3160), exons 9, 10, and 11 (SEQ ID NOs: 3161-3200); exons 10 and 11 (SEQ ID NOs: 3801-3960), exons 12 and 13 (SEQ ID NOs: 3961-4120), or exons 10, 11, 12, and 13 (SEQ ID NOs: 4121-4240) of KIT; exons 10 and 11 (SEQ ID NOs: 6525-6832) or exons 13, 14, or 15 (SEQ ID NOs: 66833-7180) of BRAF, and/or exon 21 (SEQ ID NOs: 1-160), exon 22 (SEQ ID NOs: 401-560), exon 23 (SEQ ID NOs: 561-840 and 5311-5446), exon 24 (SEQ ID NOs: 921-1240), exon 25 (SEQ ID NOs: 1241-1600), exons 21 and 22 (SEQ ID NOs: 161-400 and 5201-5310), exons 21, 22, and 23 (SEQ ID NOs: 841-920), exons 24 and 25 (SEQ ID NOs: 1601-1640 and 5447-5576), or exons 21, 22, 23, 24, and 25 (SEQ ID NOs: 5577-5818) of ALK. As disclosed herein “primer set” refers to a forward and reverse primer pair (i.e., a left and right primer pair) that can be used together to amplify a given region of a nucleic acid (e.g., DNA, RNA, or cDNA) of interest. Thus, panels with multiple primer sets include multiple primer pairs. It is understood and herein contemplated that some primer sets may have a common forward or reverse primer and thus have an odd number of primers.

It is further understood and herein contemplated that the disclosed kinase inhibitor resistant panels can comprise a single primer sets that hybridizes to a single gene, region, or exon of a gene selected from the group of genes comprising KRAS, BRAF, EGFR, ALK, and KIT (i.e, a single primer sets for KRAS, BRAF, EGFR, ALK, or KIT); multiple primer sets that hybridize to a single gene, region, or exon of a gene selected from the group of genes comprising KRAS, BRAF, EGFR, ALK, and KIT (i.e, one or more primer sets for KRAS, BRAF, EGFR, ALK, or KIT); multiple primer sets comprising a single primer set that specifically hybridize to a single gene, region, or exon for each of the genes comprising KRAS, BRAF, EGFR, ALK, and KIT (i.e, a single primer set for each of KRAS, BRAF, EGFR, ALK, and/or KIT); or multiple primer sets comprising where in there is more than one primer set for each gene, region or exon for each of the genes selected from the group of genes comprising KRAS, BRAF, EGFR, ALK, and KIT (i.e, one or more primer sets for each of KRAS, BRAF, EGFR, ALK, and/or KIT). Thus, it is contemplated herein that the kinase inhibitor panel can comprise primer sets that recognize and specifically hybridize to a gene, region, or exon, of one or combination of the gene selected from the group consisting of KRAS, BRAF, EGFR, ALK, and KIT. For example, the panel can comprise primer sets that hybridize to a gene, region, or exon of KRAS, BRAF, EGFR, ALK, or KIT; KRAS and BRAF; KRAS and EGFR; KRAS and ALK; KRAS and KIT; BRAF and EGFR; BRAF and KIT; BRAF and ALK; EGFR and ALK; EGFR and KIT; ALK and KIT; KRAS, BRAF, and EGFR; KRAS, BRAF, and ALK; KRAS, BRAF, and KIT; KRAS, EGFR, and ALK; KRAS, EGFR, and KIT; KRAS, ALK, and KIT; BRAF, EGFR, and ALK, BRAF, EGFR, and KIT; BRAF, ALK, and KIT; EGFR, ALK, and KIT; KRAS, BRAF, EGFR, and ALK; KRAS, BRAF, EGFR, and KIT, BRAF, EGFR, ALK, and KIT; and KRAS, BRAF, EGFR, ALK, and KIT.

For example, the primer or primer sets in the kinase inhibitor resistance panel can detect any of the mutations in Tables 2-6. In another aspect, the primers or primer sets used in the inhibitor resistance panel can comprise one or more of the primers or primer sets listed in Tables 7-14 as disclosed herein and/or probes listed in Table 15 (i.e., SEQ ID NOs: 7611-7613).

### Methods of Detecting the Presence of a Kinase Inhibitor Resistant Cancer

The disclosed kinase inhibitor resistant panels, in one aspect, contain primers or primer sets for the detection of mutations that confer kinase inhibitor resistance. Thus, in another aspect disclosed herein are methods and assays for the detection of kinase inhibitor resistant forms of an ALK-related cancer. For example, disclosed herein are methods and assays for the detection of kinase inhibitor resistance, such as, for example ALK kinase inhibitor resistance, comprising obtaining a tissue sample from a subject with a cancer, such as a kinase related cancer (e.g., ALK-related cancers); conducting a high throughput sequencing (also known as next generation sequencing) reaction on the sample, wherein the presence of a mutation in the nucleic acid sequence of a gene associated with kinase inhibitor resistance indicates that that the cancer is resistant or will become resistant to a kinase inhibitor. In one aspect, the mutation can be a nucleic acid mutation in ALK, EGFR, KRAS, BRAF, or KIT. For example, the mutation can be any mutation listed in Tables 2-6. In a further aspect, the disclosed methods and assays for detection of kinase inhibitor resistance can comprise performing next generation sequencing using a kinase inhibitor resistant panel as disclosed herein which comprises a primer or primer set that hybridizes and amplifies nucleic acid from exon 1 or 2 of KRAS; exon 18, 19, 20, 21 or 22 of EGFR; exon 8, 9, 10, 11, 12, 13, or 17 of KIT; exon 10, 11, 13, 14, or 15 of BRAF, and/or exon 21, 22, 23, 24, or 25 of ALK. For example, the primer or primer set can comprise any of the primers or primer sets disclosed in Tables 7-14. Thus, disclosed herein are methods wherein the one or more primer set(s) that hybridizes and amplifies nucleic acid from exon 1 (SEQ ID NOs: 4601-4880 and 7181-7230) exon 2 (SEQ ID NOs: 4881-5200 and 7231-7326) or both exons 1 and 2 (SEQ ID NOs: 7327-7610) of KRAS; exon 18 (SEQ ID NOs: 1641-1760 and 5819-5934), exon 19 (SEQ ID NOs: 1761-1880), exon 20 (SEQ ID NOs: 1881-2000 and 5934-6042), exon 21 (SEQ ID NOs: 2001-2120 and 6043-6150), exon 22 (SEQ ID NOs: 2121-2240, 2321-2360, and 2401-2440), exons 18 and 19 (SEQ ID NOs: 2241-2280), exons 18, 19, and 20 (SEQ ID NOs: 6151-6274), exons 20 and 21 (SEQ ID NOs: 2281-2320 and 6275-6388), or exons 18, 19, 20, and 21 (SEQ ID NOs: 2361-2400 and 6389-6524) of EGFR; exon 8 (SEQ ID NOs: 2441-2800), exon 9 (SEQ ID NOs: 2841-3120), exon 10 (SEQ ID NOs: 3201-3360), exon 11 (SEQ ID NOs: 3361-3480), exon 12 (SEQ ID NOs: 3481-3640), exon 13 (SEQ ID NOs: 3641-3800), exon 17 (SEQ ID NOs: 4241-4600), exon 8 and 9 (SEQ ID NOs: 2801-2840), exons 9 and 10 (SEQ ID NOs: 3121-3160), exons 9, 10, and 11 (SEQ ID NOs: 3161-3200); exons 10 and 11 (SEQ ID NOs: 3801-3960), exons 12 and 13 (SEQ ID NOs: 3961-4120), or exons 10, 11, 12, and 13 (SEQ ID NOs: 4121-4240) of KIT; exons 10 and 11 (SEQ ID NOs: 6525-6832) or exons 13, 14, or 15 (SEQ ID NOs: 66833-7180) of BRAF, and/or exon 21 (SEQ ID NOs: 1-160), exon 22 (SEQ ID NOs: 401-560), exon 23 (SEQ ID NOs: 561-840 and 5311-5446), exon 24 (SEQ ID NOs: 921-1240), exon 25 (SEQ ID NOs: 1241-1600), exons 21 and 22 (SEQ ID NOs: 161-400 and 5201-5310), exons 21, 22, and 23 (SEQ ID NOs: 841-920), exons 24 and 25 (SEQ ID NOs: 1601-1640 and 5447-5576), or exons 21, 22, 23, 24, and 25 (SEQ ID NOs: 5577-5818) of ALK.

It is understood that the disclosed methods can further comprise synthesizing cDNA from the nucleic acid extracted from a tissue sample before detection of a mutation in ALK, EGFR, KRAS, BRAF, or KIT. Thus, in one aspect, disclosed herein are methods for detecting kinase inhibitor resistance in a cancer in a subject, for example ALK kinase inhibitor resistance, comprising obtaining a tissue sample from a subject with a cancer, such as a kinase related cancer (e.g., ALK-related cancers); synthesixing cDNA from the tissue sample, and conducting a high throughput sequencing (also known as next generation sequencing) reaction on the sample, wherein the presence of a mutation in the nucleic acid sequence of a gene associated with kinase inhibitor resistance indicates that that the cancer is resistant or will become resistant to a kinase inhibitor.

It is further understood and herein contemplated that the subject of the disclosed methods can be a subject that has been previously diagnosed with a cancer including but not limited to inflammatory breast cancer, non-small cell lung carcinoma, esophageal squamous cell carcinoma, colorectal carcinoma, Inflammatory myofibroblastic tumor, familial and sporadic neuroblastoma. In yet another aspect, the subject has been previously diagnosed with a cancer that results from ALK, ROS1, RET, DEPDC1 overexpression, dysregulation, or fusion. Examples of such fusions include but are not limited to nucleophosmin-ALK (NPM-ALK), 5-aminoimidazole-4-carboxamide ribonucleotide formyltransferase/IMP cyclohydrolase (ATIC-ALK), clathrin heavy chain-ALK (CLTC-ALK), kinesin-1 heavy chain gene-ALK (KIF5B-ALK); Ran-binding protein 2-ALK (RANBP2-ALK), SEC31L1-ALK, tropomyosin-3-ALK (TPM3-ALK), tropomyosin-4-ALK (TPM4-ALK), TRK-fused gene (Large)-ALK (TFG_{L}-ALK), TRK-fused gene (Small)-ALK (TFG_{S}-ALK), CARS-ALK, EML4-ALK, 5-aminoimidazole-4-carboxamide ribonucleotide formyltransferase/IMP cyclohydrolase-ALK (ATIC-ALK), ALO17-ALK, moesin-ALK (MSN-ALK), non-muscle myosin heavy chain gene-ALK (MYH9-ALK), and TRK-fused gene (Extra Large)-ALK (TFGxL-ALK). In a further aspect, the present methods could not only be used to diagnose a kinase inhibitor resistant cancer, but diagnose the cancer itself as the subject with a kinase inhibitor resistant cancer would necessarily not only have a cancer, but have a kinase related cancer such as those disclosed herein.

Therefore, in one aspect, disclosed herein are methods for the detection of kinase inhibitor resistance comprising obtaining a tissue sample from a subject with a cancer and conducting a high throughput sequencing (also known as next generation sequencing) reaction on the sample using one or more primer sets or primer panels with primer sets that specifically hybridizes to one or more of the genes selected from the group consisting of ALK, KRAS, EGFR, KIT, and BRAF, wherein the presence of a mutation in the nucleic acid sequence of a gene associated with kinase inhibitor resistance indicates that that the cancer is resistant or will become resistant to a kinase inhibitor.

Also disclosed are methods, wherein at least one primer sets hybridizes and amplifies nucleic acid from exon 1 or 2 of KRAS, wherein at least one primer sets hybridizes and amplifies nucleic acid from exon 18, 19, 20, 21 or 22 of EGFR, wherein at least one primer sets hybridizes and amplifies nucleic acid from exon 21, 22, 23, 24, or 25 of ALK, wherein at least one primer sets hybridizes and amplifies nucleic acid from exon 8, 9, 10, 11, 12, 13, or 17 of KIT, and/or wherein at least one primer sets hybridizes and amplifies nucleic acid from exon 10, 11, 13, 14, or 15 of BRAF.

In one aspect, disclosed are methods, wherein one or more KRAS hybridizing primers or primer sets comprise one or more of the primers of Tables 10 and/or 14 (SEQ ID NOs: 4601-5200 and 7181-7610); wherein one or more EGFR hybridizing primers or primer sets comprise one or more of the primers of Tables 8 and/or 12 (1641-2440 and 5819-6524); wherein one or more ALK hybridizing primers or primer sets comprise one or more of the primers of Tables 7 and/or 11 (SEQ ID NOs: 1-1640 and 5201-5818); wherein one or more KIT hybridizing primers or primer sets comprise one or more of the primers of Table 9 (SEQ ID NOs: 2441-4600); and/or wherein one or more BRAF hybridizing primers or primer sets comprise one or more of the primers of Table 13 (SEQ ID NOs: 6525-7180).

In one aspect are methods comprising the use of a kinase inhibitor resistance panel, wherein the panel comprises 2, 3, 4, 5, 6, 7, 8, 9, 10, or more primer sets for one or more of the genes selected from group of genes comprising KRAS, BRAF, EGFR, ALK, and KIT.

In another aspect, disclosed are methods wherein the panel comprises one or more primer sets for 2, 3, 4, of all 5 of the genes selected from group of genes comprising KRAS, BRAF, EGFR, ALK, and KIT.

Also disclosed are methods, wherein the kinase inhibitor is selected from the group consisting of crizotinib, afatinib, Axitinib, bevacizumab, Bosutinib, Cetuximab, Dasatinib, Erlotinib, Fostamati nib, Gefitinib, Imatinib, Lapatinib, Lenvatinib, Nilotinib, Panitumumab, Pazopanib, Pegaptanib, Ranibizumab, Ruxolitinib, Sorafenib, Sunitinib, Trastuzumab, and Vemurafenib.

### Methods, Assays, and Primer Panels for Assessing the Suitability of ALK Directed Treatments

Though not wishing to be bound by current theories, it is believed that inhibition of these over-expression or aberrant expressions of ALK with small-molecule drug candidates abrogates related abnormal cell proliferation and promotes apoptosis in ALK-related tumor cell lines. Furthermore, both preclinical animal models and the early clinical experience with these inhibitors indicate that ALK small-molecule inhibitors not only possess marked antitumor activity against ALK-related cancers but are also very well tolerated with no limiting target-associated toxicities. Therefore, such small molecules can be used to treat ALK-driven cancers.

However, the presence of a mutation in one of the genes associated with an ALK-related cancer can confer resistance to treatment with a kinase inhibitor, such as an ALK kinase inhibitor. Nevertheless, knowledge of the presence of said mutation can still be useful to the practicing physician in assessing the suitability of a treatment or prescribing a particular treatment regimen. For example, the presence of a mutation in a gene which confers kinase inhibitor resistance, such as, for example, ALK kinase inhibitor resistance, can inform the skilled artisan to choose a particular kinase inhibitor over another due to the presence of a mutation affecting one kinase inhibitor and not the other. Alternatively, the presence of a mutation can inform the physician to discontinue the course of treatment with one kinase inhibitor due to detection of kinase inhibitor resistance and select a different kinase inhibitor to which the patient is not yet resistant. Accordingly, disclosed herein are methods and assays for assessing the suitability of an ALK inhibitor treatment for a cancer, for example, NSCLC, in a subject comprising performing high throughput sequencing on nucleic acid from a tissue sample from the subject; wherein the presence of a mutation in ALK, EGFR, BRAF, KRAS, or KIT indicates a cancer that comprises resistance to an ALK kinase inhibitor. In one aspect, disclosed herein are methods and assays for assessing a subject's suitability for treatment with a kinase inhibitor comprising obtaining a tissue sample from a subject with a cancer, such as a kinase related cancer (e.g., ALK-related cancers); detecting the presence of a mutation through sequencing or other nucleic acid detection technique for the presence of a mutation in the nucleic acid sequence of a gene associated with kinase inhibitor resistance indicates that that the cancer is resistant or will become resistant to a kinase inhibitor and therefore continued use of an inhibitor to which the cancer has become resistant or to which the cancer is already resistant should be discontinued in favor of a cancer to which resistance has not developed.

It is understood and herein contemplated that any of the disclosed nucleic acid sequencing techniques disclosed herein can be used in these methods. Thus, disclosed herein are methods and assays assessing the suitability of an ALK kinase inhibitor treatment for an ALK related cancer in a subject comprising conducting high throughput sequencing (also known as next generation sequencing) on nucleic acid such as mRNA or DNA from a tissue sample from the subject; wherein the sequencing reaction reveals the nucleic acid sequence for one or more exons of KIT, BRAF, KRAS, EGFR, and ALK; and wherein the presence of one or more mutations in KIT, BRAF, KRAS, EGFR, and/or ALK indicates the presence of kinase inhibitor resistance. The mutations can occur in any exon of KIT, BRAF, KRAS, EGFR, and ALK. Thus, for example, the mutations can occur in and therefore the primers or primer sets can hybridize to exon 1 or 2 of KRAS; exon 18, 19, 20, 21 r 22 of EGFR; exon 8, 9, 10, 11, 12, 13, or 17 of KIT; exon 10, 11, 13, 14, or 15 of BRAF, and/or exon 21, 22, 23, 24, or 25 of ALK. In one aspect, the mutation can comprise any one or more of the mutations listed in Tables 2-6. It is further understood that the disclosed methods and assays can further comprise any of the primers disclosed herein in Tables 7-14 or probes listed in Table 15 and utilize the multiplexing PCR techniques disclosed.

In another aspect, two or more of the disclosed primers and primer sets can comprise a primer panel can be used in methods and assays for the assessment of the suitability of a kinase inhibitor for the treatment of a subjects' cancer. In one aspect, the primer panel comprises one or more primers that can detect a nucleic acid mutation in ALK, BRAF, EGFR, KRAS, or KIT. In a further aspect, the primers or primer sets that hybridizes and amplifies nucleic acid from exon 1 or 2 of KRAS; exon 18, 19, 20, 21 or 22 of EGFR; exon 8, 9, 10, 11, 12, 13, or 17 of KIT; exon 10, 11, 13, 14, or 15 of BRAF, and/or exon 21, 22, 23, 24, or 25 of ALK. In another aspect, the disclosed primer panel can comprise any primer or primer set which detects one or more of the mutations found in Tables 2-6. For example, the primer or primer set can comprise any of the primers or primer sets disclosed in Tables 7-14.

In another aspect, knowledge of kinase inhibitor resistant cancer can be used to screen for a drug that is not a kinase inhibitor. Thus, in one aspect, disclosed herein are methods of screening for a drug to treat a subject with a cancer comprising obtaining a tissue sample from a subject with a cancer, such as a kinase related cancer (e.g., ALK-related cancers); conducting a high throughput sequencing (also known as next generation sequencing) reaction on the sample, wherein the presence of a mutation in the nucleic acid sequence of a gene, region, or exon associated with kinase inhibitor resistance indicates that that the subject has a cancer is resistant or will become resistant to a kinase inhibitor, and contacting a tissue sample from subject with a cancer with an agent; wherein an agent that inhibits or reduces the growth or development of a kinase inhibitor resistant cancer is not a kinase inhibitor. The disclosed methods can further comprise the sue of the kinase inhibitor resistant panels disclosed herein or any of the primers, primer sets or probes disclosed herein. The methods can also further comprise the treatment of a subject with a kinase inhibitor resistant cancer with an agent that is identified in the method as not being a kinase inhibitor or discontinuing treatment in a subject with kinase inhibitor resistant cancer with an agent that has been found to be a kinase inhibitor.

### Methods of Identifying Subjects for Participation in Clinical Trials to Screen for New Cancer Treatments.

In one aspect, it is contemplated herein that the identification of individuals with a kinase inhibitor resistant cancer can be useful for establishing clinical trials to screen for drugs that can be used to treat individuals with kinase inhibitor resistant cancers. Thus, in one aspect, disclosed herein are methods for identifying a subject for screening for a drug that can treat a cancer in a subject with a kinase inhibitor resistant cancer, for example ALK kinase inhibitor resistance, comprising obtaining a tissue sample from a subject with a cancer, such as a kinase related cancer (e.g., ALK-related cancers); and conducting a high throughput sequencing (also known as next generation sequencing) reaction on the sample, wherein the presence of a mutation in the nucleic acid sequence of a gene, region, or exon associated with kinase inhibitor resistance indicates that that the subject has a cancer is resistant or will become resistant to a kinase inhibitor and the subject can be used in trials to screen for a drug to which a kinase inhibitor resistant subject will respond. In one aspect, the mutation can be a nucleic acid mutation in ALK, EGFR, KRAS, BRAF, or KIT. For example, the mutation can be any mutation listed in Tables 2-6. In one aspect, said methods can further comprise synthesizing cDNA from the tissue sample of the subject.

It is understood and herein contemplated that the disclosed methods can be used in conjunction with any of the kinase inhibitor resistant panels, primer sets, or probes disclosed herein. For example, the disclosed methods can be performed using a primer or primer set that hybridizes and amplifies nucleic acid from exon 1 or 2 of KRAS; exon 18, 19, 20, 21 or 22 of EGFR; exon 8, 9, 10, 11, 12, 13, or 17 of KIT; exon 10, 11, 13, 14, or 15 of BRAF, and/or exon 21, 22, 23, 24, or 25 of ALK. For example, the primer or primer set can comprise any of the primers or primer sets disclosed in Tables 7-14. Thus, disclosed herein are methods wherein the one or more primer set(s) that hybridizes and amplifies nucleic acid from exon 1 (SEQ ID NOs: 4601-4880 and 7181-7230) exon 2 (SEQ ID NOs: 4881-5200 and 7231-7326) or both exons 1 and 2 (SEQ ID NOs: 7327-7610) of KRAS; exon 18 (SEQ ID NOs: 1641-1760 and 5819-5934), exon 19 (SEQ ID NOs: 1761-1880), exon 20 (SEQ ID NOs: 1881-2000 and 5934-6042), exon 21 (SEQ ID NOs: 2001-2120 and 6043-6150), exon 22 (SEQ ID NOs: 2121-2240, 2321-2360, and 2401-2440), exons 18 and 19 (SEQ ID NOs: 2241-2280), exons 18, 19, and 20 (SEQ ID NOs: 6151-6274), exons 20 and 21 (SEQ ID NOs: 2281-2320 and 6275-6388), or exons 18, 19, 20, and 21 (SEQ ID NOs: 2361-2400 and 6389-6524) of EGFR; exon 8 (SEQ ID NOs: 2441-2800), exon 9 (SEQ ID NOs: 2841-3120), exon 10 (SEQ ID NOs: 3201-3360), exon 11 (SEQ ID NOs: 3361-3480), exon 12 (SEQ ID NOs: 3481-3640), exon 13 (SEQ ID NOs: 3641-3800), exon 17 (SEQ ID NOs: 4241-4600), exon 8 and 9 (SEQ ID NOs: 2801-2840), exons 9 and 10 (SEQ ID NOs: 3121-3160), exons 9, 10, and 11 (SEQ ID NOs: 3161-3200); exons 10 and 11 (SEQ ID NOs: 3801-3960), exons 12 and 13 (SEQ ID NOs: 3961-4120), or exons 10, 11, 12, and 13 (SEQ ID NOs: 4121-4240) of KIT; exons 10 and 11 (SEQ ID NOs: 6525-6832) or exons 13, 14, or 15 (SEQ ID NOs: 66833-7180) of BRAF, and/or exon 21 (SEQ ID NOs: 1-160), exon 22 (SEQ ID NOs: 401-560), exon 23 (SEQ ID NOs: 561-840 and 5311-5446), exon 24 (SEQ ID NOs: 921-1240), exon 25 (SEQ ID NOs: 1241-1600), exons 21 and 22 (SEQ ID NOs: 161-400 and 5201-5310), exons 21, 22, and 23 (SEQ ID NOs: 841-920), exons 24 and 25 (SEQ ID NOs: 1601-1640 and 5447-5576), or exons 21, 22, 23, 24, and 25 (SEQ ID NOs: 5577-5818) of ALK.

### Methods of Detecting a Kinase Inhibitor Resistance in an ALK-Related Cancer

In another aspect, the disclosed methods and assays relate to the detection or diagnosis of the presence of a kinase inhibitor resistance, such as, for example, ALK kinase inhibitor resistance, in a disease or condition such as a cancer and methods and assays for the determination of susceptibility or resistance to therapeutic treatment for a disease or condition such as a cancer in a subject comprising detecting the presence or measuring the expression level of nucleic acid (for example, DNA, mRNA, cDNA, RNA, etc) through the use of next generation sequencing (NGS) from a tissue sample from the subject; wherein the presence of a mutations in the nucleic acid code of the KIT, BRAF, KRAS, EGFR, or ALK gene or the ALK gene portion of an ALK fusion construct indicates the presence of a cancer that is resistant to a kinase inhibitor. In one aspect, the cancer is associated with amplification, overexpression, nucleic acid variation, truncation, or gene fusion of ALK. It is understood, that the kinase inhibitor resistance panels disclosed herein can be used to perform said methods and the detection of one or more of the mutations in Tables 2-6 indicates the presence of kinase inhibitor resistance. In one aspect, the disclosed methods can further comprise discontinuing use of a kinase inhibitor to treat a cancer in a subject that has been identified with a kinase inhibitor resistant cancer. In another embodiment, the disclosed methods can further comprise treating a subject with a kinase inhibitor resistant cancer with a chemotherapeutic that is not a kinase inhibitor. Thus, in one aspect, disclosed herein are methods of treating a subject with a kinase inhibitor resistant cancer (such as, for example, an ALK kinase inhibitor resistant cancer) comprising obtaining a tissue sample from a subject with a cancer, such as a kinase related cancer (e.g., ALK-related cancers); conducting a high throughput sequencing (also known as next generation sequencing) reaction on the sample, wherein the presence of a mutation in the nucleic acid sequence of a gene, region, or exon associated with kinase inhibitor resistance indicates that that the subject has a cancer is resistant or will become resistant to a kinase inhibitor; and treating the subject with a chemotherapeutic that is not a kinase inhibitor. Also disclosed are methods of treating a subject without a kinase inhibitor resistant cancer comprising obtaining a tissue sample from a subject with a cancer, such as a kinase related cancer (e.g., ALK-related cancers); conducting a high throughput sequencing (also known as next generation sequencing) reaction on the sample, wherein the absence of a mutation in the nucleic acid sequence of a gene, region, or exon associated with kinase inhibitor resistance indicates that that the subject does not have a cancer is resistant nor will become resistant to a kinase inhibitor; and treating the subject with a kinase inhibitor.

### Anaplastic Lymphoma Kinase (ALK)

ALK (SEQ ID NO: 7714 (Genbank Accession No. U62540 (human coding sequence)) is a receptor tyrosine kinase (RTK) of the insulin receptor superfamily encoded by the ALK gene and is normally expressed primarily in the central and peripheral nervous systems. The 1620aa ALK polypeptide comprises a 1030aa extracellular domain which includes a 26aa amino-terminal signal peptide sequence, and binding sites located between residues 391 and 401 for the ALK ligands pleiotrophin (PTN) and midkine (MK). Additionally, the ALK polypeptide comprises a kinase domain (residues 1116-1383) which includes three tyrosines responsible for autophosphorylation within the activation loop at residues 1278, 1282, and 1283. ALK amplification, overexpression, and mutations have been shown to constitutively activate the kinase catalytic function of the ALK protein, with the deregulated mutant ALK in turn activating downstream cellular signaling proteins in pathways that promote aberrant cell proliferation. In fact, the mutations that result in dysregulated ALK kinase activity are associated with several types of cancers.

ALK fusions represent the most common mutation of this tyrosine kinase. Such fusions include but are not limited to nucleophosmin-ALK (NPM-ALK), 5-aminoimidazole-4-carboxamide ribonucleotide formyltransferase/IMP cyclohydrolase (ATIC-ALK), clathrin heavy chain-ALK (CLTC-ALK), kinesin-1 heavy chain gene-ALK (KIF5B-ALK); Ran-binding protein 2-ALK (RANBP2-ALK), SEC31L1-ALK, tropomyosin-3-ALK (TPM3-ALK), tropomyosin-4-ALK (TPM4-ALK), TRK-fused gene (Large)-ALK (TFG_{L}-ALK), TRK-fused gene (Small)-ALK (TFGs-ALK), CARS-ALK, EML4-ALK, 5-aminoimidazole-4-carboxamide ribonucleotide formyltransferase/IMP cyclohydrolase-ALK (ATIC-ALK), ALO17-ALK, moesin-ALK (MSN-ALK), non-muscle myosin heavy chain gene-ALK (MYH9-ALK), and TRK-fused gene (Extra Large)-ALK (TFGxL-ALK). Six ALK fusions, CARS-ALK. CLTC-ALK, RANBP2-ALK, SEC31L1-ALK, TPM3-ALK, and TPM4-ALK have been identified in IMTs. TPM3-ALK, TPM4-ALK and CLTC-ALK fusions have been detected in both classical T- or null-cell lymphomas and IMT sarcomas, whereas CARS-ALK, RANBP2-ALK, and SEC31L1-ALK occur in IMT. CLTC-ALK and NPM-ALK also occur in B-cell plasmablastic/immunoblastic lymphomas. The TPM4-ALK fusion occurs in esophageal squamous cell carcinomas, and the ALK fusion EML4-ALK, TFG-ALK and KIF5B-ALK are found in non-small cell lung cancers. EML4-ALK has also recently been identified in both colorectal and breast carcinomas as well.

ALK fusions are associated with several known cancer types. It is understood that one or more ALK fusions can be associated with a particular cancer. It is further understood that there are several types of cancer associated with ALK fusions including but not limited to anaplastic large-cell lymphoma (ALCL), neuroblastoma, breast cancer, ovarian cancer, colorectal carcinoma, non-small cell lung carcinoma, diffuse large B-cell lymphoma, esophageal squamous cell carcinoma, anaplastic large-cell lymphoma, neuroblastoma, inflammatory myofibroblastic tumors, malignant histiocytosis, and glioblastomas.

ALCL. anaplastic large-cell lymphomas comprise ˜2.5% of all NHL; within the pediatric age group specifically, ˜13% of all NHL (30-40% of all childhood large-cell lymphomas) are of this type. Studies of ALCL patients now divide this NHL into ALK-positive and ALK-negative subsets; ˜60% of all ALCLs are caused by ALK fusions. For unclear reasons, ALK-positive ALCL patients fare significantly better following CHOP based multi-agent conventional chemotherapy than those with ALK-negative disease (with overall 5-year survivals of ˜75% vs. ˜35%, respectively). However, more than a third of patients suffer multiple relapses following chemotherapy, thus the 5-year disease-free survival of ALK-positive ALCL is only ˜40%.

ALK+ Diffuse large B-cell lymphoma. In 2003, ALK fusions were shown to occur in a non-ALCL form of NHL with the description of CLTC-ALK or NPM-ALK in diffuse large B-cell lymphomas (ALK+ DLBCLs). Consistent with their B-lineage, these NHLs express cytoplasmic IgA and plasma cell markers, and possess an immunoblastic morphology. Translational research studies revealed the t(2; 17) and CLTC-ALK mRNA in the majority of these lymphomas, while immunolabeling confirmed granular ALK staining identical to that observed in CLTC-ALK-positive ALCL. As for all other ALK fusion partner proteins, a self-association motif in the CLTC portion of CLTC-ALK mediates constitutive self-association and activation of the fusion kinase to drive lymphomagenesis. ALK+ DLBCLs occur predominately in adults; however, the t(2; 5) and NPM-ALK mRNA in pediatric lymphomas are phenotypically identical to CLTC-ALK-positive adult B-NHLs. Approximately 0.5-1% of all DLBCL is thought to be ALK-positive. The identification of DLBCLs caused by mutant ALK is important because patients with these lymphomas have outcomes that are much inferior to ALK-negative DLBCL patients following CHOP-based treatments; thus, ALK+ DLBCL patients should strongly be considered as candidates for ALK-targeted kinase inhibitor therapy.

ALK+ systemic histiocytosis. ALK fusions were described in 2008 in another hematopoietic neoplasm, systemic histiocytosis. Three cases of this previously uncharacterized form of histiocytosis, which presents in early infancy, exhibited ALK immunoreactivity and the one case analyzed molecularly expressed TPM3-ALK.

In addition to the aforementioned hematological malignancies in which constitutively activated ALK fusions have been shown to be a causative mechanism in many cases, the genesis of subsets of various solid tumors in some instances, very common human tumors such as non-small cell lung cancer, colorectal and breast cancers has recently been demonstrated to be due to aberrantly activated ALK.

Inflammatory myofibroblastic tumor. The first non-hematopoietic tumor discovered to express ALK fusions was the sarcoma known as inflammatory myofibroblastic tumor (IMT), a spindle cell proliferation in the soft tissue and viscera of children and young adults (mean age at diagnosis ˜10 years). Many IMTs are indolent and can be cured by resection. However, locally recurrent, invasive, and metastatic IMTs are not uncommon and current chemo- and radio-therapies are completely ineffective. Disclosed herein is the involvement of chromosome 2p23 (the location of the ALK gene) in IMTs, as well as ALK gene rearrangement. ALK immunoreactivity in 7 of 11 IMTs has been shown and TPM3-ALK and TPM4-ALK were identified in several cases. Additionally, two additional ALK fusions in IMT, CLTC- and RanBP2-ALK were identified. ALK fusions have also been examined by immunostaining in 73 IMTs, finding 60% (44 of the 73 cases) to be ALK-positive. Thus, ALK deregulation is of pathogenic importance in a majority of IMTs.

Non-small cell lung carcinoma. The role of ALK fusions in cancer expanded further with the description of the novel EML4-ALK chimeric protein in 5 of 75 (6.7%) Japanese non-small cell lung carcinoma patients. Shortly thereafter, the existence of ALK fusions in lung cancer was corroborated by a different group who found 6 of 137 (4.4%) Chinese lung cancer patients to express ALK fusions (EML4-ALK, 3 pts; TFG-ALK, 1 pt; X-ALK. Two common themes have emerged—1) ALK fusions occur predominately in patients with adenocarcinoma (although occasional ALK-positive NSCLCs of squamous or mixed histologies are observed), mostly in individuals with minimal/no smoking history, and 2) ALK abnormalities usually occur exclusive of other common genetic abnormalities (e.g., EGFR and KRAS mutations). The exact percentage of NSCLCs caused by ALK fusions is not yet clear but estimates based on reports in the biomedical literature suggest a range of ˜5-10%.

Esophageal squamous cell carcinoma. In 45 Iranian patients, a proteomics approach identified proteins under or over-represented in esophageal squamous cell carcinomas (ESCCs); TPM4-ALK was among those proteins over-represented. A second proteomics-based ESCC study—in this case, in Chinese patients—identified TPM4-ALK in these tumors as well.

Colorectal carcinoma, breast cancer. Three human tumor types—colorectal, breast, and non-small cell lung cancers were surveyed for the presence of the EML4-ALK fusion (other ALK mutations were not assessed in this study). In addition to confirming the expression of EML4-ALK in NSCLC (in 12 of 106 specimens studied, 11.3%), a subsets of breast (5 of 209 cases, 2.4%) and colorectal (2 of 83 cases, 2.4%) carcinomas were EML4-ALK-positive. In addition to known EML4-ALK variants 1 (E13; A20) and 2 (E20; A20), a novel variant (E21; A20) was found in colorectal carcinoma.

ALK in familial and sporadic neuroblastoma. Neuroblastoma is the most common extracranial solid tumor of childhood, and is derived from the developing neural crest. A small subset (˜1-2%) of neuroblastomas exhibit a familial predisposition with an autosomal dominant inheritance. Most neuroblastoma patients have aggressive disease associated with survival probabilities <40% despite intensive chemo- and radio-therapy, and the disease accounts for ˜15% of all childhood cancer mortality. ALK had previously been found to be constitutively activated also due to high-level over-expression as a result of gene amplification in a small number of neuroblastoma cell lines, in fact, ALK amplification occurs in ˜15% of neuroblastomas in addition to activating point mutations. These missense mutations in ALK have been confirmed as activating mutations that drive neuroblastoma growth; furthermore, incubation of neuroblastoma cell lines with ALK small-molecule inhibitors reveal those cells with ALK activation (but not cell lines with normal levels of expression of wild-type ALK) to exhibit robust cytotoxic responses.

The sensitive detection of a mutation at a known site in DNA is readily done with existing technologies. Allele specific primers can be designed to target a mutation at a known location such that its signal can be preferentially amplified over wild-type DNA.

### Next Generation Sequencing for Genetic Testing

From a technical perspective High-throughput or Next Generation Sequencing (NGS) represents an attractive option for detecting the somatic mutations within a gene. Unlike PCR, microarrays, high-resolution melting and mass spectrometry, which all indirectly infer sequence content, NGS directly ascertains the identity of each base and the order in which they fall within a gene. The newest platforms on the market have the capacity to cover an exonic region 10,000 times over, meaning the content of each base position in the sequence is measured thousands of different times. This high level of coverage ensures that the consensus sequence is extremely accurate and enables the detection of rare variants within a heterogeneous sample. For example, in a sample extracted from FFPE tissue, relevant mutations are only present at a frequency of 1% with the wild-type allele comprising the remainder. When this sample is sequenced at 10,000× coverage, then even the rare allele, comprising only 1% of the sample, is uniquely measured 100 times over. Thus, NGS can provide reliably accurate results with very high sensitivity, making it ideal for clinical diagnostic testing of FFPEs and other mixed samples.

In one aspect, disclosed herein are methods and assays for detecting kinase inhibitor resistance or determining the susceptibility to a particular kinase inhibitor treatment in an ALK-related cancer comprising performing next generation sequencing on a tissue sample obtained from a subject with an ALK-related cancer, wherein the presence of a nucleic acid variation in the ALK, BRAF, EGFR, KIT, or KRAS sequence of the tissue sample at a nucleic acid residue indicates that presence of kinase inhibitor resistance. For example, the methods and assays for detecting kinase inhibitor resistance or determining the susceptibility or developing kinase inhibitor resistance in an ALK-related cancer or determining the suitability of a particular kinase inhibitor for use in treating an ALK-related cancer in a subject can comprise the detection of any of the mutations in Tables 2-6. It is understood that the methods and assays can further comprise comparing the sequence to known kinase inhibitor resistance mutations list and determining what if any kinase inhibitors are affected by the mutation and altering or maintaining treatment as appropriate to utilize kinase inhibitors that are unaffected by the mutation. As the disclosed methods and assays employ the use of primers or primer sets to detect mutations that confer kinase inhibitor resistance, also disclosed herein are primer panels for use in next generation sequencing for the determination of kinase inhibitor resistance comprising one or more primer sets from each of KIT, BRAF, KRAS, EGFR, and ALK, for example, the disclosed primer panels, methods, and assays can comprise one or more of the primers or primer sets listed in Tables 7-14.

Examples of Next Generation Sequencing techniques include, but are not limited to Massively Parallel Signature Sequencing (MPSS), Polony sequencing, pyrosequencing, Reversible dye-terminator sequencing, SOLiD sequencing, Ion semiconductor sequencing, DNA nanoball sequencing, Helioscope single molecule sequencing, Single molecule real time (SMRT) sequencing, Single molecule real time (RNAP) sequencing, and Nanopore DNA sequencing.

MPSS was a bead-based method that used a complex approach of adapter ligation followed by adapter decoding, reading the sequence in increments of four nucleotides; this method made it susceptible to sequence-specific bias or loss of specific sequences.

Polony sequencing, combined an in vitro paired-tag library with emulsion PCR, an automated microscope, and ligation-based sequencing chemistry to sequence an E. coli genome at an accuracy of >99.9999% and a cost approximately 1/10 that of Sanger sequencing.

A parallelized version of pyrosequencing, the method amplifies DNA inside water droplets in an oil solution (emulsion PCR), with each droplet containing a single DNA template attached to a single primer-coated bead that then forms a clonal colony. The sequencing machine contains many picolitre-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

A sequencing technology based on reversible dye-terminators. DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. The result is sequences of quantities and lengths comparable to Illumina sequencing.

Ion semiconductor sequencing is based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

DNA nanoball sequencing is a type of high throughput sequencing technology used to determine the entire genomic sequence of an organism. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run.

Helicos's single-molecule sequencing uses DNA fragments with added polyA tail adapters, which are attached to the flow cell surface. The next steps involve extension-based sequencing with cyclic washes of the flow cell with fluorescently labeled nucleotides (one nucleotide type at a time, as with the Sanger method). The reads are performed by the Helioscope sequencer.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand.

Single molecule real time sequencing based on RNA polymerase (RNAP), which is attached to a polystyrene bead, with distal end of sequenced DNA is attached to another bead, with both beads being placed in optical traps. RNAP motion during transcription brings the beads in closer and their relative distance changes, which can then be recorded at a single nucleotide resolution. The sequence is deduced based on the four readouts with lowered concentrations of each of the four nucleotide types (similarly to Sangers method).

Nanopore sequencing is based on the readout of electrical signal occurring at nucleotides passing by alpha-hemolysin pores covalently bound with cyclodextrin. The DNA passing through the nanopore changes its ion current. This change is dependent on the shape, size and length of the DNA sequence. Each type of the nucleotide blocks the ion flow through the pore for a different period of time.

VisiGen Biotechnologies uses a specially engineered DNA polymerase. This polymerase acts as a sensor—having incorporated a donor fluorescent dye by its active centre. This donor dye acts by FRET (fluorescent resonant energy transfer), inducing fluorescence of differently labeled nucleotides. This approach allows reads performed at the speed at which polymerase incorporates nucleotides into the sequence (several hundred per second). The nucleotide fluorochrome is released after the incorporation into the DNA strand.

Sequencing by hybridization is a non-enzymatic method that uses a DNA microarray. A single pool of DNA whose sequence is to be determined is fluorescently labeled and hybridized to an array containing known sequences. Strong hybridization signals from a given spot on the array identify its sequence in the DNA being sequenced. Mass spectrometry may be used to determine mass differences between DNA fragments produced in chain-termination reactions.

Another NGS approach is sequencing by synthesis (SBS) technology which is capable of overcoming the limitations of existing pyrosequencing based NGS platforms. Such technologies rely on complex enzymatic cascades for read out, are unreliable for the accurate determination of the number of nucleotides in homopolymeric regions and require excessive amounts of time to run individual nucleotides across growing DNA strands. The SBS NGS platform uses a direct sequencing approach to produce a sequencing strategy with very a high precision, rapid pace and low cost.

SBS sequencing is initialized by fragmenting of the template DNA into fragments, amplification, annealing of DNA sequencing primers, and finally affixing as a high-density array of spots onto a glass chip. The array of DNA fragments are sequenced by extending each fragment with modified nucleotides containing cleavable chemical moieties linked to fluorescent dyes capable of discriminating all four possible nucleotides. The array is scanned continuously by a high-resolution electronic camera (Measure) to determine the fluorescent intensity of each base (A, C, G or T) that was newly incorporated into the extended DNA fragment. After the incorporation of each modified base the array is exposed to cleavage chemistry to break off the fluorescent dye and end cap allowing additional bases to be added. The process is then repeated until the fragment is completely sequenced or maximal read length has been achieved.

mRNA Detection and Quantification

A number of widely used procedures exist for detecting and determining the abundance of a particular mRNA in a total or poly(A) RNA sample. For example, specific mRNAs can be detected using Northern blot analysis, nuclease protection assays (NPA), in situ hybridization (e.g., fluorescence in situ hybridization (FISH)), or reverse transcription-polymerase chain reaction (RT-PCR), and microarray.

In theory, each of these techniques can be used to detect specific RNAs and to precisely determine their expression level. In general, Northern analysis is the only method that provides information about transcript size, whereas NPAs are the easiest way to simultaneously examine multiple messages. In situ hybridization is used to localize expression of a particular gene within a tissue or cell type, and RT-PCR is the most sensitive method for detecting and quantitating gene expression.

RT-PCR allows for the detection of the RNA transcript of any gene, regardless of the scarcity of the starting material or relative abundance of the specific mRNA. In RT-PCR, an RNA template is copied into a complementary DNA (cDNA) using a retroviral reverse transcriptase. The cDNA is then amplified exponentially by PCR using a DNA polymerase. The reverse transcription and PCR reactions can occur in the same or difference tubes. RT-PCR is somewhat tolerant of degraded RNA. As long as the RNA is intact within the region spanned by the primers, the target will be amplified.

Relative quantitative RT-PCR involves amplifying an internal control simultaneously with the gene of interest. The internal control is used to normalize the samples. Once normalized, direct comparisons of relative abundance of a specific mRNA can be made across the samples. It is crucial to choose an internal control with a constant level of expression across all experimental samples (i.e., not affected by experimental treatment). Commonly used internal controls (e.g., GAPDH, β-actin, cyclophilin) often vary in expression and, therefore, may not be appropriate internal controls. Additionally, most common internal controls are expressed at much higher levels than the mRNA being studied. For relative RT-PCR results to be meaningful, all products of the PCR reaction must be analyzed in the linear range of amplification. This becomes difficult for transcripts of widely different levels of abundance.

Competitive RT-PCR is used for absolute quantitation. This technique involves designing, synthesizing, and accurately quantitating a competitor RNA that can be distinguished from the endogenous target by a small difference in size or sequence. Known amounts of the competitor RNA are added to experimental samples and RT-PCR is performed. Signals from the endogenous target are compared with signals from the competitor to determine the amount of target present in the sample.

Northern analysis is the easiest method for determining transcript size, and for identifying alternatively spliced transcripts and multigene family members. It can also be used to directly compare the relative abundance of a given message between all the samples on a blot. The Northern blotting procedure is straightforward and provides opportunities to evaluate progress at various points (e.g., intactness of the RNA sample and how efficiently it has transferred to the membrane). RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes.

The Nuclease Protection Assay (NPA) (including both ribonuclease protection assays and Si nuclease assays) is a sensitive method for the detection and quantitation of specific mRNAs. The basis of the NPA is solution hybridization of an antisense probe (radiolabeled or nonisotopic) to an RNA sample. After hybridization, single-stranded, unhybridized probe and RNA are degraded by nucleases. The remaining protected fragments are separated on an acrylamide gel. Solution hybridization is typically more efficient than membrane-based hybridization, and it can accommodate up to 100 g of sample RNA, compared with the 20-30 μg maximum of blot hybridizations. NPAs are also less sensitive to RNA sample degradation than Northern analysis since cleavage is only detected in the region of overlap with the probe (probes are usually about 100-400 bases in length).

NPAs are the method of choice for the simultaneous detection of several RNA species. During solution hybridization and subsequent analysis, individual probe/target interactions are completely independent of one another. Thus, several RNA targets and appropriate controls can be assayed simultaneously (up to twelve have been used in the same reaction), provided that the individual probes are of different lengths. NPAs are also commonly used to precisely map mRNA termini and intron/exon junctions.

In situ hybridization (ISH) is a powerful and versatile tool for the localization of specific mRNAs in cells or tissues. Unlike Northern analysis and nuclease protection assays, ISH does not require the isolation or electrophoretic separation of RNA. Hybridization of the probe takes place within the cell or tissue. Since cellular structure is maintained throughout the procedure, ISH provides information about the location of mRNA within the tissue sample.

The procedure begins by fixing samples in neutral-buffered formalin, and embedding the tissue in paraffin. The samples are then sliced into thin sections and mounted onto microscope slides. (Alternatively, tissue can be sectioned frozen and post-fixed in paraformaldehyde.) After a series of washes to dewax and rehydrate the sections, a Proteinase K digestion is performed to increase probe accessibility, and a labeled probe is then hybridized to the sample sections. Radiolabeled probes are visualized with liquid film dried onto the slides, while non-isotopically labeled probes are conveniently detected with colorimetric or fluorescent reagents.

DNA Detection and Quantification

The methods, assays, and primer panels disclosed herein relate to the detection of nucleic acid variation that confer kinase inhibitor resistance in the form of, for example, point mutations and truncations of, KRAS, BRAF, KIT, EGFR, and/or ALK Thus, in one aspect, disclosed herein are methods, assays, and use of the disclosed primer panels for diagnosing an anaplastic lymphoma kinase (ALK) related cancer in a subject is resistant to a kinase inhibitor comprise performing NGS which sequences DNA from a tissue sample from the subject. It is understood that high throughput sequencing methods (also known as next generation sequencing methods) can comprise any known amplification and detection method for DNA known in the art.

A number of widely used procedures exist for detecting and determining the abundance of a particular DNA in a sample. For example, the technology of PCR permits amplification and subsequent detection of minute quantities of a target nucleic acid. Details of PCR are well described in the art, including, for example, U.S. Pat. No. 4,683,195 to Mullis et al., U.S. Pat. No. 4,683,202 to Mullis and U.S. Pat. No. 4,965,188 to Mullis et al. Generally, oligonucleotide primers are annealed to the denatured strands of a target nucleic acid, and primer extension products are formed by the polymerization of deoxynucleoside triphosphates by a polymerase. A typical PCR method involves repetitive cycles of template nucleic acid denaturation, primer annealing and extension of the annealed primers by the action of a thermostable polymerase. The process results in exponential amplification of the target nucleic acid, and thus allows the detection of targets existing in very low concentrations in a sample. It is understood and herein contemplated that there are variant PCR methods known in the art that may also be utilized in the disclosed methods, for example, Quantitative PCR (QPCR); microarrays, real-time PCR; hot start PCR; nested PCR; allele-specific PCR; and Touchdown PCR.

Microarrays

An array is an orderly arrangement of samples, providing a medium for matching known and unknown DNA samples based on base-pairing rules and automating the process of identifying the unknowns. An array experiment can make use of common assay systems such as microplates or standard blotting membranes, and can be created by hand or make use of robotics to deposit the sample. In general, arrays are described as macroarrays or microarrays, the difference being the size of the sample spots. Macroarrays contain sample spot sizes of about 300 microns or larger and can be easily imaged by existing gel and blot scanners. The sample spot sizes in microarray can be 300 microns or less, but typically less than 200 microns in diameter and these arrays usually contains thousands of spots. Microarrays require specialized robotics and/or imaging equipment that generally are not commercially available as a complete system. Terminologies that have been used in the literature to describe this technology include, but not limited to: biochip, DNA chip, DNA microarray, GENECHIP® (Affymetrix, Inc which refers to its high density, oligonucleotide-based DNA arrays), and gene array.

DNA microarrays, or DNA chips are fabricated by high-speed robotics, generally on glass or nylon substrates, for which probes with known identity are used to determine complementary binding, thus allowing massively parallel gene expression and gene discovery studies. An experiment with a single DNA chip can provide information on thousands of genes simultaneously. It is herein contemplated that the disclosed microarrays can be used to monitor gene expression, disease diagnosis, gene discovery, drug discovery (pharmacogenomics), and toxicological research or toxicogenomics.

There are two variants of the DNA microarray technology, in terms of the property of arrayed DNA sequence with known identity. Type I microarrays comprise a probe cDNA (500˜5,000 bases long) that is immobilized to a solid surface such as glass using robot spotting and exposed to a set of targets either separately or in a mixture. This method is traditionally referred to as DNA microarray. With Type I microarrays, localized multiple copies of one or more polynucleotide sequences, preferably copies of a single polynucleotide sequence are immobilized on a plurality of defined regions of the substrate's surface. A polynucleotide refers to a chain of nucleotides ranging from 5 to 10,000 nucleotides. These immobilized copies of a polynucleotide sequence are suitable for use as probes in hybridization experiments.

To prepare beads coated with immobilized probes, beads are immersed in a solution containing the desired probe sequence and then immobilized on the beads by covalent or non-covalent means. Alternatively, when the probes are immobilized on rods, a given probe can be spotted at defined regions of the rod. Typical dispensers include a micropipette delivering solution to the substrate with a robotic system to control the position of the micropipette with respect to the substrate. There can be a multiplicity of dispensers so that reagents can be delivered to the reaction regions simultaneously. In one embodiment, a microarray is formed by using ink-jet technology based on the piezoelectric effect, whereby a narrow tube containing a liquid of interest, such as oligonucleotide synthesis reagents, is encircled by an adapter. An electric charge sent across the adapter causes the adapter to expand at a different rate than the tube and forces a small drop of liquid onto a substrate.

Samples may be any sample containing polynucleotides (polynucleotide targets) of interest and obtained from any bodily fluid (blood, urine, saliva, phlegm, gastric juices, etc.), cultured cells, biopsies, or other tissue preparations. DNA or RNA can be isolated from the sample according to any of a number of methods well known to those of skill in the art. In one embodiment, total RNA is isolated using the TRIzol total RNA isolation reagent (Life Technologies, Inc., Rockville, Md.) and RNA is isolated using oligo d(T) column chromatography or glass beads. After hybridization and processing, the hybridization signals obtained should reflect accurately the amounts of control target polynucleotide added to the sample.

The plurality of defined regions on the substrate can be arranged in a variety of formats. For example, the regions may be arranged perpendicular or in parallel to the length of the casing. Furthermore, the targets do not have to be directly bound to the substrate, but rather can be bound to the substrate through a linker group. The linker groups may typically vary from about 6 to 50 atoms long. Linker groups include ethylene glycol oligomers, diamines, diacids and the like. Reactive groups on the substrate surface react with one of the terminal portions of the linker to bind the linker to the substrate. The other terminal portion of the linker is then functionalized for binding the probes.

Sample polynucleotides may be labeled with one or more labeling moieties to allow for detection of hybridized probe/target polynucleotide complexes. The labeling moieties can include compositions that can be detected by spectroscopic, photochemical, biochemical, bioelectronic, immunochemical, electrical, optical or chemical means. The labeling moieties include radioisotopes, such as ³²P, ³³P or ³⁵S, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, biotin, and the like.

Labeling can be carried out during an amplification reaction, such as polymerase chain reaction and in vitro or in vivo transcription reactions. Alternatively, the labeling moiety can be incorporated after hybridization once a probe-target complex his formed. In one embodiment, biotin is first incorporated during an amplification step as described above. After the hybridization reaction, unbound nucleic acids are rinsed away so that the only biotin remaining bound to the substrate is that attached to target polynucleotides that are hybridized to the polynucleotide probes. Then, an avidin-conjugated fluorophore, such as avidin-phycoerythrin, that binds with high affinity to biotin is added.

Hybridization causes a polynucleotide probe and a complementary target to form a stable duplex through base pairing. Hybridization methods are well known to those skilled in the art. Stringent conditions for hybridization can be defined by salt concentration, temperature, and other chemicals and conditions. Varying additional parameters, such as hybridization time, the concentration of detergent (sodium dodecyl sulfate, SDS) or solvent (formamide), and the inclusion or exclusion of carrier DNA, are well known to those skilled in the art. Additional variations on these conditions will be readily apparent to those skilled in the art.

Methods for detecting complex formation are well known to those skilled in the art. In one embodiment, the polynucleotide probes are labeled with a fluorescent label and measurement of levels and patterns of complex formation is accomplished by fluorescence microscopy, preferably confocal fluorescence microscopy. An argon ion laser excites the fluorescent label, emissions are directed to a photomultiplier and the amount of emitted light detected and quantitated. The detected signal should be proportional to the amount of probe/target polynucleotide complex at each position of the microarray. The fluorescence microscope can be associated with a computer-driven scanner device to generate a quantitative two-dimensional image of hybridization intensities. The scanned image is examined to determine the abundance/expression level of each hybridized target polynucleotide.

In a differential hybridization experiment, polynucleotide targets from two or more different biological samples are labeled with two or more different fluorescent labels with different emission wavelengths. Fluorescent signals are detected separately with different photomultipliers set to detect specific wavelengths. The relative abundances/expression levels of the target polynucleotides in two or more samples is obtained. Typically, microarray fluorescence intensities can be normalized to take into account variations in hybridization intensities when more than one microarray is used under similar test conditions. In one embodiment, individual polynucleotide probe/target complex hybridization intensities are normalized using the intensities derived from internal normalization controls contained on each microarray.

Type II microarrays comprise an array of oligonucleotides (20˜80-mer oligos) or peptide nucleic acid (PNA) probes that is synthesized either in situ (on-chip) or by conventional synthesis followed by on-chip immobilization. The array is exposed to labeled sample DNA, hybridized, and the identity/abundance of complementary sequences are determined. This method, “historically” called DNA chips, was developed at Affymetrix, Inc., which sells its photolithographically fabricated products under the GENECHIP® trademark.

The basic concept behind the use of Type II arrays for gene expression is simple: labeled cDNA or cRNA targets derived from the mRNA of an experimental sample are hybridized to nucleic acid probes attached to the solid support. By monitoring the amount of label associated with each DNA location, it is possible to infer the abundance of each mRNA species represented. Although hybridization has been used for decades to detect and quantify nucleic acids, the combination of the miniaturization of the technology and the large and growing amounts of sequence information, have enormously expanded the scale at which gene expression can be studied.

Microarray manufacturing can begin with a 5-inch square quartz wafer. Initially the quartz is washed to ensure uniform hydroxylation across its surface. Because quartz is naturally hydroxylated, it provides an excellent substrate for the attachment of chemicals, such as linker molecules, that are later used to position the probes on the arrays.

The wafer is placed in a bath of silane, which reacts with the hydroxyl groups of the quartz, and forms a matrix of covalently linked molecules. The distance between these silane molecules determines the probes' packing density, allowing arrays to hold over 500,000 probe locations, or features, within a mere 1.28 square centimeters. Each of these features harbors millions of identical DNA molecules. The silane film provides a uniform hydroxyl density to initiate probe assembly. Linker molecules, attached to the silane matrix, provide a surface that may be spatially activated by light.

Probe synthesis occurs in parallel, resulting in the addition of an A, C, T, or G nucleotide to multiple growing chains simultaneously. To define which oligonucleotide chains will receive a nucleotide in each step, photolithographic masks, carrying 18 to 20 square micron windows that correspond to the dimensions of individual features, are placed over the coated wafer. The windows are distributed over the mask based on the desired sequence of each probe. When ultraviolet light is shone over the mask in the first step of synthesis, the exposed linkers become deprotected and are available for nucleotide coupling.

Once the desired features have been activated, a solution containing a single type of deoxynucleotide with a removable protection group is flushed over the wafer's surface. The nucleotide attaches to the activated linkers, initiating the synthesis process.

Although each position in the sequence of an oligonucleotide can be occupied by 1 of 4 nucleotides, resulting in an apparent need for 25×4, or 100, different masks per wafer, the synthesis process can be designed to significantly reduce this requirement. Algorithms that help minimize mask usage calculate how to best coordinate probe growth by adjusting synthesis rates of individual probes and identifying situations when the same mask can be used multiple times.

Some of the key elements of selection and design are common to the production of all microarrays, regardless of their intended application. Strategies to optimize probe hybridization, for example, are invariably included in the process of probe selection. Hybridization under particular pH, salt, and temperature conditions can be optimized by taking into account melting temperatures and using empirical rules that correlate with desired hybridization behaviors.

To obtain a complete picture of a gene's activity, some probes are selected from regions shared by multiple splice or polyadenylation variants. In other cases, unique probes that distinguish between variants are favored. Inter-probe distance is also factored into the selection process.

A different set of strategies is used to select probes for genotyping arrays that rely on multiple probes to interrogate individual nucleotides in a sequence. The identity of a target base can be deduced using four identical probes that vary only in the target position, each containing one of the four possible bases.

Alternatively, the presence of a consensus sequence can be tested using one or two probes representing specific alleles. To genotype heterozygous or genetically mixed samples, arrays with many probes can be created to provide redundant information, resulting in unequivocal genotyping. In addition, generic probes can be used in some applications to maximize flexibility. Some probe arrays, for example, allow the separation and analysis of individual reaction products from complex mixtures, such as those used in some protocols to identify single nucleotide polymorphisms (SNPs).

Real-Time PCR

Real-time PCR monitors the fluorescence emitted during the reaction as an indicator of amplicon production during each PCR cycle (i.e., in real time) as opposed to the endpoint detection. The real-time progress of the reaction can be viewed in some systems. Real-time PCR does not detect the size of the amplicon and thus does not allow the differentiation between DNA and cDNA amplification, however, it is not influenced by non-specific amplification unless SYBR Green is used. Real-time PCR quantitation eliminates post-PCR processing of PCR products. This helps to increase throughput and reduce the chances of carryover contamination. Real-time PCR also offers a wide dynamic range of up to 10⁷-fold. Dynamic range of any assay determines how much target concentration can vary and still be quantified. A wide dynamic range means that a wide range of ratios of target and normaliser can be assayed with equal sensitivity and specificity. It follows that the broader the dynamic range, the more accurate the quantitation. When combined with RT-PCR, a real-time RT-PCR reaction reduces the time needed for measuring the amount of amplicon by providing for the visualization of the amplicon as the amplification process is progressing.

The real-time PCR system is based on the detection and quantitation of a fluorescent reporter. This signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed. A significant increase in fluorescence above the baseline value measured during the 3-15 cycles can indicate the detection of accumulated PCR product.

A fixed fluorescence threshold is set significantly above the baseline that can be altered by the operator. The parameter C_{T} (threshold cycle) is defined as the cycle number at which the fluorescence emission exceeds the fixed threshold.

There are three main fluorescence-monitoring systems for DNA amplification: (1) hydrolysis probes; (2) hybridising probes; and (3) DNA-binding agents. Hydrolysis probes include TaqMan probes, molecular beacons and scorpions. They use the fluorogenic 5′ exonuclease activity of Taq polymerase to measure the amount of target sequences in cDNA samples.

TaqMan probes are oligonucleotides longer than the primers (20-30 bases long with a Tm value of 10° C. higher) that contain a fluorescent dye usually on the 5′ base, and a quenching dye (usually TAMRA) typically on the 3′ base. When irradiated, the excited fluorescent dye transfers energy to the nearby quenching dye molecule rather than fluorescing (this is called FRET=Förster or fluorescence resonance energy transfer). Thus, the close proximity of the reporter and quencher prevents emission of any fluorescence while the probe is intact. TaqMan probes are designed to anneal to an internal region of a PCR product. When the polymerase replicates a template on which a TaqMan probe is bound, its 5′ exonuclease activity cleaves the probe. This ends the activity of quencher (no FRET) and the reporter dye starts to emit fluorescence which increases in each cycle proportional to the rate of probe cleavage. Accumulation of PCR products is detected by monitoring the increase in fluorescence of the reporter dye (note that primers are not labelled). TaqMan assay uses universal thermal cycling parameters and PCR reaction conditions. Because the cleavage occurs only if the probe hybridises to the target, the origin of the detected fluorescence is specific amplification. The process ofhybridisation and cleavage does not interfere with the exponential accumulation of the product. One specific requirement for fluorogenic probes is that there be no G at the 5′ end. A ‘G’ adjacent to the reporter dye can quench reporter fluorescence even after cleavage.

Molecular beacons also contain fluorescent (FAM, TAMRA, TET, ROX) and quenching dyes (typically DABCYL) at either end but they are designed to adopt a hairpin structure while free in solution to bring the fluorescent dye and the quencher in close proximity for FRET to occur. They have two arms with complementary sequences that form a very stable hybrid or stem. The close proximity of the reporter and the quencher in this hairpin configuration suppresses reporter fluorescence. When the beacon hybridises to the target during the annealing step, the reporter dye is separated from the quencher and the reporter fluoresces (FRET does not occur). Molecular beacons remain intact during PCR and must rebind to target every cycle for fluorescence emission. This will correlate to the amount of PCR product available. All real-time PCR chemistries allow detection of multiple DNA species (multiplexing) by designing each probe/beacon with a spectrally unique fluor/quench pair as long as the platform is suitable for melting curve analysis if SYBR green is used. By multiplexing, the target(s) and endogenous control can be amplified in single tube.

With Scorpion probes, sequence-specific priming and PCR product detection is achieved using a single oligonucleotide. The Scorpion probe maintains a stem-loop configuration in the unhybridised state. The fluorophore is attached to the 5′ end and is quenched by a moiety coupled to the 3′ end. The 3′ portion of the stem also contains sequence that is complementary to the extension product of the primer. This sequence is linked to the 5′ end of a specific primer via a non-amplifiable monomer. After extension of the Scorpion primer, the specific probe sequence is able to bind to its complement within the extended amplicon thus opening up the hairpin loop. This prevents the fluorescence from being quenched and a signal is observed.

Another alternative is the double-stranded DNA binding dye chemistry, which quantitates the amplicon production (including non-specific amplification and primer-dimer complex) by the use of a non-sequence specific fluorescent intercalating agent (SYBR-green I or ethidium bromide). It does not bind to ssDNA. SYBR green is a fluorogenic minor groove binding dye that exhibits little fluorescence when in solution but emits a strong fluorescent signal upon binding to double-stranded DNA. Disadvantages of SYBR green-based real-time PCR include the requirement for extensive optimisation. Furthermore, non-specific amplifications require follow-up assays (melting point curve or dissociation analysis) for amplicon identification. The method has been used in HFE-C282Y genotyping. Another controllable problem is that longer amplicons create a stronger signal (if combined with other factors, this may cause CDC camera saturation, see below). Normally SYBR green is used in singleplex reactions, however when coupled with melting point analysis, it can be used for multiplex reactions.

The threshold cycle or the C_{T} value is the cycle at which a significant increase in ΔRn is first detected (for definition of ΔRn, see below). The threshold cycle is when the system begins to detect the increase in the signal associated with an exponential growth of PCR product during the log-linear phase. This phase provides the most useful information about the reaction (certainly more important than the end-point). The slope of the log-linear phase is a reflection of the amplification efficiency. The efficiency (Eff) of the reaction can be calculated by the formula: Eff=10^{(-1/slope)}−1. The efficiency of the PCR should be 90-100% (3.6>slope>3.1). A number of variables can affect the efficiency of the PCR. These factors include length of the amplicon, secondary structure and primer quality. Although valid data can be obtained that fall outside of the efficiency range, the qRT-PCR should be further optimised or alternative amplicons designed. For the slope to be an indicator of real amplification (rather than signal drift), there has to be an inflection point. This is the point on the growth curve when the log-linear phase begins. It also represents the greatest rate of change along the growth curve. (Signal drift is characterised by gradual increase or decrease in fluorescence without amplification of the product.) The important parameter for quantitation is the C_{T}. The higher the initial amount of genomic DNA, the sooner accumulated product is detected in the PCR process, and the lower the C_{T} value. The threshold should be placed above any baseline activity and within the exponential increase phase (which looks linear in the log transformation). Some software allows determination of the cycle threshold (C_{T}) by a mathematical analysis of the growth curve. This provides better run-to-run reproducibility. A C_{T} value of 40 means no amplification and this value cannot be included in the calculations. Besides being used for quantitation, the C_{T} value can be used for qualitative analysis as a pass/fail measure.

Multiplex TaqMan assays can be performed using multiple dyes with distinct emission wavelengths. Available dyes for this purpose are FAM, TET, VIC and JOE (the most expensive). TAMRA is reserved as the quencher on the probe and ROX as the passive reference. For best results, the combination of FAM (target) and VIC (endogenous control) is recommended (they have the largest difference in emission maximum) whereas JOE and VIC should not be combined. It is important that if the dye layer has not been chosen correctly, the machine will still read the other dye's spectrum. For example, both VIC and FAM emit fluorescence in a similar range to each other and when doing a single dye, the wells should be labelled correctly. In the case of multiplexing, the spectral compensation for the post run analysis should be turned on (on ABI 7700: Instrument/Diagnostics/Advanced Options/Miscellaneous). Activating spectral compensation improves dye spectral resolution.

Nested PCR

The disclosed methods can further utilize nested PCR. Nested PCR increases the specificity of DNA amplification, by reducing background due to non-specific amplification of DNA. Two sets of primers are being used in two successive PCRs. In the first reaction, one pair of primers is used to generate DNA products, which besides the intended target, may still consist of non-specifically amplified DNA fragments. The product(s) are then used in a second PCR with a set of primers whose binding sites are completely or partially different from and located 3′ of each of the primers used in the first reaction. Nested PCR is often more successful in specifically amplifying long DNA fragments than conventional PCR, but it requires more detailed knowledge of the target sequences.

Primers and Probes

The disclosed methods and assays can use primers and probes. As used herein, “primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

As used herein, “probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Disclosed are assays and methods which include the use of primers and probes, as well as, the disclosed primer panels all of which are capable of interacting with the disclosed nucleic acids such as ALK (SEQ ID NO: 1), BRAF, EGFR, KIT, or KRAS or their complement. For example, any of the primers or primer sets from Table 7-14 can be used in the disclosed primer panels or any of the methods and assays disclosed herein. In certain embodiments the primers are used to support nucleic acid extension reactions, nucleic acid replication reactions, and/or nucleic acid amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are disclosed. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the disclosed nucleic acids or region of the nucleic acids or they hybridize with the complement of the nucleic acids or complement of a region of the nucleic acids. As an example of the use of primers, one or more primers can be used to create extension products from and templated by a first nucleic acid.

The size of the primers or probes for interaction with the nucleic acids can be any size that supports the desired enzymatic manipulation of the primer, such as DNA amplification or the simple hybridization of the probe or primer. A typical primer or probe would be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

The primers for the nucleic acid of interest typically will be used to produce extension products and/or other replicated or amplified products that contain a region of the nucleic acid of interest. The size of the product can be such that the size can be accurately determined to within 3, or 2 or 1 nucleotides.

In certain embodiments the product can be, for example, at least 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other embodiments the product can be, for example, less than or equal to 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

It is understood and herein contemplated that there are situations where it may be advantageous to utilize more than one primer pair to detect the presence of mutations conferring inhibitor resistance in EGFR, BRAF, KIT, KRAS, or ALK. Such RT-PCR, real-time PCT or other PCR reactions can be conducted separately, or in a single reaction. When multiple primer pairs are placed into a single reaction, this is referred to as “multiplex PCR.” It is understood and herein contemplated that any combination of two or more or three or more the forward and/or reverse primers disclosed herein can be used in the multiplex reaction.

Fluorescent Change Probes and Primers

Fluorescent change probes and fluorescent change primers refer to all probes and primers that involve a change in fluorescence intensity or wavelength based on a change in the form or conformation of the probe or primer and nucleic acid to be detected, assayed or replicated. Examples of fluorescent change probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes.

Fluorescent change probes and primers can be classified according to their structure and/or function. Fluorescent change probes include hairpin quenched probes, cleavage quenched probes, cleavage activated probes, and fluorescent activated probes. Fluorescent change primers include stem quenched primers and hairpin quenched primers.

Hairpin quenched probes are probes that when not bound to a target sequence form a hairpin structure (and, typically, a loop) that brings a fluorescent label and a quenching moiety into proximity such that fluorescence from the label is quenched. When the probe binds to a target sequence, the stem is disrupted, the quenching moiety is no longer in proximity to the fluorescent label and fluorescence increases. Examples of hairpin quenched probes are molecular beacons, fluorescent triplex oligos, triplex molecular beacons, triplex FRET probes, and QPNA probes.

Cleavage activated probes are probes where fluorescence is increased by cleavage of the probe. Cleavage activated probes can include a fluorescent label and a quenching moiety in proximity such that fluorescence from the label is quenched. When the probe is clipped or digested (typically by the 5′-3′ exonuclease activity of a polymerase during amplification), the quenching moiety is no longer in proximity to the fluorescent label and fluorescence increases. TaqMan probes are an example of cleavage activated probes.

Cleavage quenched probes are probes where fluorescence is decreased or altered by cleavage of the probe. Cleavage quenched probes can include an acceptor fluorescent label and a donor moiety such that, when the acceptor and donor are in proximity, fluorescence resonance energy transfer from the donor to the acceptor causes the acceptor to fluoresce. The probes are thus fluorescent, for example, when hybridized to a target sequence. When the probe is clipped or digested (typically by the 5′-3′ exonuclease activity of a polymerase during amplification), the donor moiety is no longer in proximity to the acceptor fluorescent label and fluorescence from the acceptor decreases. If the donor moiety is itself a fluorescent label, it can release energy as fluorescence (typically at a different wavelength than the fluorescence of the acceptor) when not in proximity to an acceptor. The overall effect would then be a reduction of acceptor fluorescence and an increase in donor fluorescence. Donor fluorescence in the case of cleavage quenched probes is equivalent to fluorescence generated by cleavage activated probes with the acceptor being the quenching moiety and the donor being the fluorescent label. Cleavable FRET (fluorescence resonance energy transfer) probes are an example of cleavage quenched probes.

Fluorescent activated probes are probes or pairs of probes where fluorescence is increased or altered by hybridization of the probe to a target sequence. Fluorescent activated probes can include an acceptor fluorescent label and a donor moiety such that, when the acceptor and donor are in proximity (when the probes are hybridized to a target sequence), fluorescence resonance energy transfer from the donor to the acceptor causes the acceptor to fluoresce. Fluorescent activated probes are typically pairs of probes designed to hybridize to adjacent sequences such that the acceptor and donor are brought into proximity. Fluorescent activated probes can also be single probes containing both a donor and acceptor where, when the probe is not hybridized to a target sequence, the donor and acceptor are not in proximity but where the donor and acceptor are brought into proximity when the probe hybridized to a target sequence. This can be accomplished, for example, by placing the donor and acceptor on opposite ends of the probe and placing target complement sequences at each end of the probe where the target complement sequences are complementary to adjacent sequences in a target sequence. If the donor moiety of a fluorescent activated probe is itself a fluorescent label, it can release energy as fluorescence (typically at a different wavelength than the fluorescence of the acceptor) when not in proximity to an acceptor (that is, when the probes are not hybridized to the target sequence). When the probes hybridize to a target sequence, the overall effect would then be a reduction of donor fluorescence and an increase in acceptor fluorescence. FRET probes are an example of fluorescent activated probes.

Stem quenched primers are primers that when not hybridized to a complementary sequence form a stem structure (either an intramolecular stem structure or an intermolecular stem structure) that brings a fluorescent label and a quenching moiety into proximity such that fluorescence from the label is quenched. When the primer binds to a complementary sequence, the stem is disrupted; the quenching moiety is no longer in proximity to the fluorescent label and fluorescence increases. In the disclosed method, stem quenched primers are used as primers for nucleic acid synthesis and thus become incorporated into the synthesized or amplified nucleic acid. Examples of stem quenched primers are peptide nucleic acid quenched primers and hairpin quenched primers.

Peptide nucleic acid quenched primers are primers associated with a peptide nucleic acid quencher or a peptide nucleic acid fluor to form a stem structure. The primer contains a fluorescent label or a quenching moiety and is associated with either a peptide nucleic acid quencher or a peptide nucleic acid fluor, respectively. This puts the fluorescent label in proximity to the quenching moiety. When the primer is replicated, the peptide nucleic acid is displaced, thus allowing the fluorescent label to produce a fluorescent signal.

Hairpin quenched primers are primers that when not hybridized to a complementary sequence form a hairpin structure (and, typically, a loop) that brings a fluorescent label and a quenching moiety into proximity such that fluorescence from the label is quenched. When the primer binds to a complementary sequence, the stem is disrupted; the quenching moiety is no longer in proximity to the fluorescent label and fluorescence increases. Hairpin quenched primers are typically used as primers for nucleic acid synthesis and thus become incorporated into the synthesized or amplified nucleic acid. Examples of hairpin quenched primers are Amplifluor primers and scorpion primers.

Cleavage activated primers are similar to cleavage activated probes except that they are primers that are incorporated into replicated strands and are then subsequently cleaved.

Labels

To aid in detection and quantitation of nucleic acids produced using the disclosed methods, labels can be directly incorporated into nucleotides and nucleic acids or can be coupled to detection molecules such as probes and primers. As used herein, a label is any molecule that can be associated with a nucleotide or nucleic acid, directly or indirectly, and which results in a measurable, detectable signal, either directly or indirectly. Many such labels for incorporation into nucleotides and nucleic acids or coupling to nucleic acid probes are known to those of skill in the art. Examples of labels suitable for use in the disclosed method are radioactive isotopes, fluorescent molecules, phosphorescent molecules, enzymes, antibodies, and ligands. Fluorescent labels, especially in the context of fluorescent change probes and primers, are useful for real-time detection of amplification.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, CASCADE BLUE®, OREGON GREEN®, pyrene, lissamine, xanthenes, acridines, oxazines, phycoerythrin, macrocyclic chelates of lanthanide ions such as quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, BerberineSulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (DiaminoNaphtylSulphonic Acid), Dansyl NH-CH3, Diamino Phenyl Oxydiazole (DAO), Dimethylamino-5-Sulphonic acid, DipyrrometheneboronDifluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, IntrawhiteCf Liquid, Leucophor PAF, Leucophor SF, Leucophor WS, LissamineRhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green PyronineStilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Phycoerythrin B, PolyazaindacenePontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbenelsothiosulphonic acid), Stilbene, Snarf 1, sulphoRhodamine B Can C, SulphoRhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

The absorption and emission maxima, respectively, for some of these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 Bi. Other labels of interest include those described in U.S. Pat. No. 5,563,037 which is incorporated herein by reference.

Labeled nucleotides are a form of label that can be directly incorporated into the amplification products during synthesis. Examples of labels that can be incorporated into amplified nucleic acids include nucleotide analogs such as BrdUrd, aminoallyldeoxyuridine, 5-methylcytosine, bromouridine, and nucleotides modified with biotin or with suitable haptens such as digoxygenin. Suitable fluorescence-labeled nucleotides are Fluorescein-isothiocyanate-dUTP, Cyanine-3-dUTP and Cyanine-5-dUTP. One example of a nucleotide analog label for DNA is BrdUrd (bromodeoxyuridine, BrdUrd, BrdU, BUdR, Sigma-Aldrich Co). Other examples of nucleotide analogs for incorporation of label into DNA are AA-dUTP (aminoallyl-deoxyuridine triphosphate, Sigma-Aldrich Co.), and 5-methyl-dCTP (Roche Molecular Biochemicals). One example of a nucleotide analog for incorporation of label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Labels that are incorporated into amplified nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo[3.3.1.1³′7]decane]-4-yl) phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these labels are also considered labels. Any of the known labels can be used with the disclosed probes, tags, and method to label and detect nucleic acid amplified using the disclosed method. Methods for detecting and measuring signals generated by labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary label coupled to the antibody. As used herein, detection molecules are molecules which interact with amplified nucleic acid and to which one or more labels are coupled.

The disclosed methods, assays, and primer panels can be used to diagnose any disease where uncontrolled cellular proliferation occurs herein referred to as “cancer”. A non-limiting list of different types of ALK related cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general. In particular, the disclosed methods, assays, and kits relate to the diagnosis, detection, or prognosis of inflammatory breast cancer

A representative but non-limiting list of cancers that the disclosed methods can be used to diagnose is the following: lymphoma, B cell lymphoma (including diffuse large B-cell lymphoma), B-cell plasmablastic/immunoblastic lymphomas, T cell lymphoma (including T- or null-cell lymphomas), mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, anaplastic large-cell lymphoma (ALCL), inflammatory myofibroblastic tumors, ovarian cancer, pancreatic cancer, prostate cancer, skin cancer, liver cancer, melanoma, malignant histiocytosis, squamous cell carcinomas of the mouth, throat, larynx, and lung, colon cancer, cervical cancer, cervical carcinoma, breast cancer (including inflammatory breast cancer), and epithelial cancer, renal cancer, genitourinary cancer, pulmonary cancer, esophageal squamous cell carcinoma, head and neck carcinoma, large bowel cancer, hematopoietic cancers; testicular cancer; colon and rectal cancers, prostatic cancer, or pancreatic cancer.

### Nucleic Acids

The disclosed method and compositions make use of various nucleic acids. Generally, any nucleic acid can be used in the disclosed method. For example, the disclosed nucleic acids of interest and the disclosed reference nucleic acids can be chosen based on the desired analysis and information that is to be obtained or assessed. The disclosed methods also produce new and altered nucleic acids. The nature and structure of such nucleic acids will be established by the manner in which they are produced and manipulated in the methods. Thus, for example, extension products and hybridizing nucleic acids are produced in the disclosed methods. As used herein, hybridizing nucleic acids are hybrids of extension products and the second nucleic acid.

It is understood and contemplated herein that a nucleic acid of interest can be any nucleic acid to which the determination of the presence or absence of nucleotide variation is desired. Thus, for example, the nucleic acid of interest can comprise a sequence that corresponds to the wild-type sequence of the reference nucleic acid. It is further disclosed herein that the disclosed methods can be performed where the first nucleic acid is a reference nucleic acid and the second nucleic acid is a nucleic acid of interest or where the first nucleic acid is the nucleic acid of interest and the second nucleic acid is the reference nucleic acid.

It is understood and herein contemplated that a reference nucleic acid can be any nucleic acid against which a nucleic acid of interest is to be compared. Typically, the reference nucleic acid has a known sequence (and/or is known to have a sequence of interest as a reference). Although not required, it is useful if the reference sequence has a known or suspected close relationship to the nucleic acid of interest. For example, if a single nucleotide variation is desired to be detected, the reference sequence can be usefully chosen to be a sequence that is a homolog or close match to the nucleic acid of interest, such as a nucleic acid derived from the same gene or genetic element from the same or a related organism or individual. Thus, for example, it is contemplated herein that the reference nucleic acid can comprise a wild-type sequence or alternatively can comprise a known mutation including, for example, a mutation the presence or absence of which is associated with a disease or resistance to therapeutic treatment. Thus, for example, it is contemplated that the disclosed methods can be used to detect or diagnose the presence of known mutations in a nucleic acid of interest by comparing the nucleic acid of interest to a reference nucleic acid that comprises a wild-type sequence (i.e., is known not to possess the mutation) and examining for the presence or absence of variation in the nucleic acid of interest, where the absence of variation would indicate the absence of a mutation. Alternatively, the reference nucleic acid can possess a known mutation. Thus, for example, it is contemplated that the disclosed methods can be used to detect susceptibility for a disease or condition by comparing the nucleic acid of interest to a reference nucleic acid comprising a known mutation that indicates susceptibility for a disease and examining for the presence or absence of the mutation, wherein the presence of the mutation indicates a disease.

Herein, the term “nucleotide variation” refers to any change or difference in the nucleotide sequence of a nucleic acid of interest relative to the nucleotide sequence of a reference nucleic acid. Thus, a nucleotide variation is said to occur when the sequences between the reference nucleic acid and the nucleic acid of interest (or its complement, as appropriate in context) differ. Thus, for example, a substitution of an adenine (A) to a guanine (G) at a particular position in a nucleic acid would be a nucleotide variation provided the reference nucleic acid comprised an A at the corresponding position. It is understood and herein contemplated that the determination of a variation is based upon the reference nucleic acid and does not indicate whether or not a sequence is wild-type. Thus, for example, when a nucleic acid with a known mutation is used as the reference nucleic acid, a nucleic acid not possessing the mutation (including a wild-type nucleic acid) would be considered to possess a nucleotide variation (relative to the reference nucleic acid).

### Nucleotides

The disclosed methods and compositions make use of various nucleotides. Throughout this application and the methods disclosed herein reference is made to the type of base for a nucleotide. It is understood and contemplated herein that where reference is made to a type of base, this refers a base that in a nucleotide in a nucleic acid strand is capable of hybridizing (binding) to a defined set of one or more of the canonical bases. Thus, for example, where reference is made to extension products extended in the presence of three types of nuclease resistant nucleotides and not in the presence of nucleotides that comprise the same type of base as the modified nucleotides, this means that if, for example, the base of the modified nucleotide was an adenine (A), the nuclease-resistant nucleotides can be, for example, guanine (G), thymine (T), and cytosine (C). Each of these bases (which represent the four canonical bases) is capable of hybridizing to a different one of the four canonical bases and thus each qualify as a different type of base as defined herein. As another example, inosine base pairs primarily with adenine and cytosine (in DNA) and thus can be considered a different type of base from adenine and from cytosine—which base pair with thymine and guanine, respectively—but not a different type of base from guanine or thymine-which base pair with cytosine and adenine, respectively-because the base pairing of guanine and thymine overlaps (that is, is not different from) the hybridization pattern of inosine.

Any type of modified or alternative base can be used in the disclosed methods and compositions, generally limited only by the capabilities of the enzymes used to use such bases. Many modified and alternative nucleotides and bases are known, some of which are described below and elsewhere herein. The type of base that such modified and alternative bases represent can be determined by the pattern of base pairing for that base as described herein. Thus for example, if the modified nucleotide was adenine, any analog, derivative, modified, or variant base that based pairs primarily with thymine would be considered the same type of base as adenine. In other words, so long as the analog, derivative, modified, or variant has the same pattern of base pairing as another base, it can be considered the same type of base. Modifications can made to the sugar or phosphate groups of a nucleotide. Generally such modifications will not change the base pairing pattern of the base. However, the base pairing pattern of a nucleotide in a nucleic acid strand determines the type of base of the base in the nucleotide.

Modified nucleotides to be incorporated into extension products and to be selectively removed by the disclosed agents in the disclosed methods can be any modified nucleotide that functions as needed in the disclosed method as is described elsewhere herein. Modified nucleotides can also be produced in existing nucleic acid strands, such as extension products, by, for example, chemical modification, enzymatic modification, or a combination.

Many types of nuclease-resistant nucleotides are known and can be used in the disclosed methods. For example, nucleotides have modified phosphate groups and/or modified sugar groups can be resistant to one or more nucleases. Nuclease-resistance is defined herein as resistance to removal from a nucleic acid by any one or more nucleases. Generally, nuclease resistance of a particular nucleotide can be defined in terms of a relevant nuclease. Thus, for example, if a particular nuclease is used in the disclosed method, the nuclease-resistant nucleotides need only be resistant to that particular nuclease. Examples of useful nuclease-resistant nucleotides include thio-modified nucleotides and borano-modified nucleotides.

There are a variety of molecules disclosed herein that are nucleic acid based. Non-limiting examples of these and other molecules are discussed herein. It is understood that for example, a nucleotide is a molecule that contains a base moiety, a sugar moiety and a phosphate moiety. Nucleotides can be linked together through their phosphate moieties and sugar moieties creating an intemucleoside linkage. The base moiety of a nucleotide can be adenine-9-yl (adenine, A), cytosine-1-yl (cytosine, C), guanine-9-yl (guanine, G), uracil-1-yl (uracil, U), and thymin-1-yl (thymine, T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (w), hypoxanthin-9-yl (inosine, I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, which is incorporated herein in its entirety for its teachings of base modifications. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine. 5-methylcytosine can increase the stability of duplex formation. Often time base modifications can be combined with for example a sugar modification, such as 2′-O-methoxyethyl, to achieve unique properties such as increased duplex stability.

Nucleotide analogs can also include modifications of the sugar moiety. Modifications to the sugar moiety would include natural modifications of the ribose and deoxy ribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH2)n O]m CH3, —O(CH2)n OCH3, —O(CH2)n NH2, —O(CH2)n CH3, —O(CH2)n—ONH2, and —O(CH2)nON[(CH2)n CH3)]2, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2 CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH2 and S. Nucleotide sugar analogs may also have sugar mimetics such as cyclobutyl moieties in place of the pentofuranosyl sugar.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included.

It is understood that nucleotide analogs need only contain a single modification, but may also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkylinternucleoside linkages, mixed heteroatom and alkyl or cycloalkylinternucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts.

It is also understood in a nucleotide substitute that both the sugar and the phosphate moieties of the nucleotide can be replaced, by for example an amide type linkage (aminoethylglycine) (PNA).

It is also possible to link other types of molecules (conjugates) to nucleotides or nucleotide analogs. Conjugates can be chemically linked to the nucleotide or nucleotide analogs. Such conjugates include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, Ni, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### Kits

Disclosed herein are kits that are drawn to reagents that can be used in practicing the methods disclosed herein. In particular, he kits can include any reagent or combination of reagents discussed herein or that would be understood to be required or beneficial in the practice of the disclosed methods. For example, the kits could include one or more primers from Tables 7-14 disclosed herein to perform the extension, replication and amplification reactions discussed in certain embodiments of the methods, as well as the buffers and enzymes required to use the primers as intended. The kit can also include other necessary reagents to perform any of the next generation sequencing techniques disclosed herein. In another aspect, the disclosed kits can include one or more of the probes listed in Table 15 in addition to or instead of one or more primers from Table 7-14.

It is understood and herein contemplated that the disclosed kits can comprise at least one primer set to detect the presence of nucleic acid variation in each of KIT, BRAF, KRAS, ALK, and EGFR. For example, the kits can comprise at least one primer or primer set for sequencing at least one of each of the KIT, BRAF, KRAS, ALK, and EGFR exons of Tables 1. In one aspect, the kits can comprise at least one primer or primer set from each of Tables 7-14. Alternatively, the kit can comprise a primer or primer set that will detect one or more of the specific mutations listed in Tables 2-6. Therefore, in one aspect disclosed herein are kits for performing a NGS sequencing reaction on a tissue sample to detect the presence of a mutation conferring kinase inhibitor resistance comprising at least one or more primer or primer set from each of Table 7-14. In another aspect, disclosed herein are kits for performing a NGS sequencing reaction on a tissue sample to detect the presence of a mutation conferring kinase inhibitor resistance comprising at least one or more primer or primer set capable of specifically hybridizing an amplifying any of the mutant sequences of KIT, BRAF, KRAS, ALK, and EGFR present in Tables 2-6.

Additionally, it is understood that the disclosed kits can include such other reagents and material for performing the disclosed methods such as enzymes (e.g., polymerases), buffers, sterile water, and/or reaction tubes. Additionally the kits can also include modified nucleotides, nuclease-resistant nucleotides, and or labeled nucleotides. Additionally, the disclosed kits can include instructions for performing the methods disclosed herein and software for enable the calculation of the presence of a kinase inhibitor mutation (i.e., a mutation in KIT, BRAF, KRAS, EGFR, and/or ALK).

The compositions disclosed herein and the compositions necessary to perform the disclosed methods can be made using any method known to those of skill in the art for that particular reagent or compound unless otherwise specifically noted.

### Nucleic Acid Synthesis

The disclosed nucleic acids, such as, the oligonucleotides to be used as primers can be made using standard chemical synthesis methods or can be produced using enzymatic methods or any other known method. Such methods can range from standard enzymatic digestion followed by nucleotide fragment isolation to purely synthetic methods, for example, by the cyanoethylphosphoramidite method using a Milligen or Beckman System 1Plus DNA synthesizer (for example, Model 8700 automated synthesizer of Milligen-Biosearch, Burlington, Mass. or ABI Model 380B).

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### ALK Inhibitor Resistance

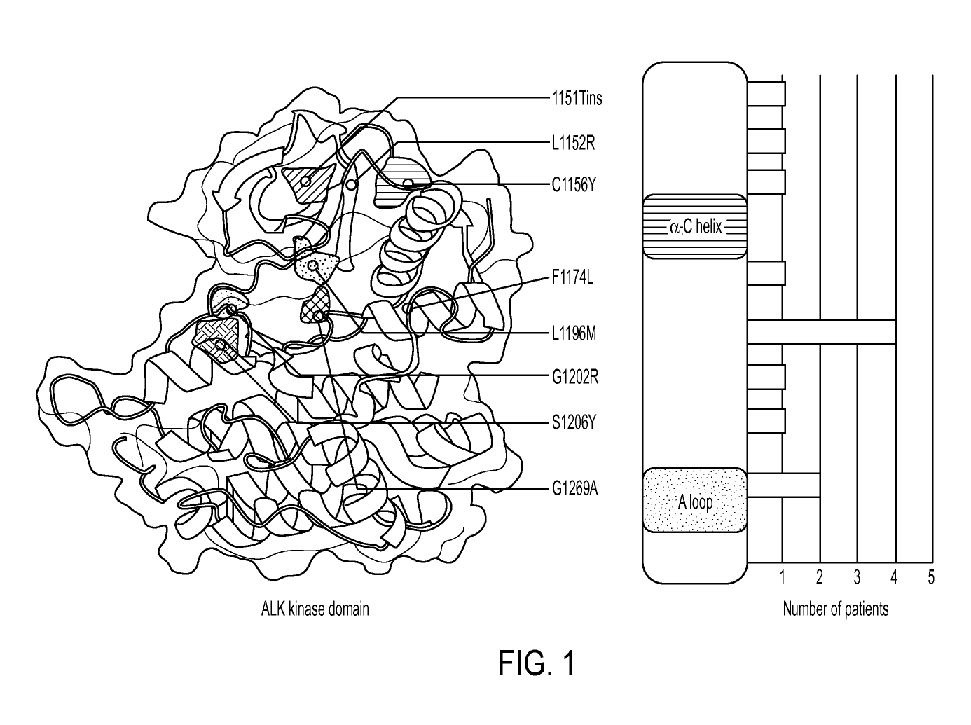

Using an in vitro assay known to predict clinically relevant kinase inhibitor-resistance mutations resistance selection studies were performed with XALKORI® and identified a large number ofALK kinase domain point mutations that confer high-level resistance to the Pfizer inhibitor (FIG. 1). In response to the issue of resistance, a number of pharma and biotech companies currently have 2^{nd}-generation ALK small-molecule inhibitors in development.

The need for more comprehensive oncogene profiling in patients with ALK inhibitor resistance was observed in an ALK positive crizotinib resistant cohort of patients that ALK specific kinase mutations accounted for only a third of crizotinib resistance. The larger subset of crizotinib resistant cases indicated that second (co-expression in conjunction with ALK) or separate (complete absence of ALK) oncogenic drivers such as EGFR, BRAF, KRAS or cKIT can relieve the sensitivity to crizotinib and drive oncogenesis. It was also observed in a single case that the complete loss of ALK expression did not correspond to the presence of an identifiable alternate driver indicating the genetic profiling of ALK inhibitor resistance cases should be extended past EGFR, BRAF, KRAS or cKIT expression using more versatile testing platforms. The presence of multiple oncogenes present in a single tumor sample is by no means a new phenomenon as EGFR driven tumors resistant to EGFR tyrosine kinase inhibitors can be driven by secondary MET gene amplification.

### Example 2

### A Diagnostic Cancer Panel that Employs NGS

Applicants have designed and developed a next generation sequencing panel to amplify and sequence one or more exons within ALK and other oncogenes implicated in driving tumorigenesis in the presence of crizotinib (i.e. ALK, BRAF, EGFR, KIT and KRAS. See Table 1 for an overarching description of the exons targeted for sequencing in the panel and Tables 2-6 for a more detailed list of each mutation detected by the Insight ALK resistance ID™ panel. Primer sequences used to amplify each gene segment are depicted in Tables 7-14.

TABLE 1
Exons That Are Covered

| Gene | Exon |
|---|---|
| KRAS | 1 |
| KRAS | 2 |
| EGFR | 18 |
| EGFR | 19 |
| EGFR | 20 |
| EGFR | 21 |
| EGFR | 22 |
| ALK | 21 |
| ALK | 22 |
| ALK | 23 |
| ALK | 24 |
| ALK | 25 |
| KIT | 8 |
| KIT | 9 |
| KIT | 10 |
| KIT | 11 |
| KIT | 12 |
| KIT | 13 |
| KIT | 17 |
| BRAF | 10 |
| BRAF | 11 |
| BRAF | 13 |
| BRAF | 14 |
| BRAF | 15 |

### Example 3

### Targeted Next Generation Sequencing Insight ALK Resistance ID™

Polymerase chain reaction is used to create amplicons that span the exonic regions mentioned above. The design described here is agnostic to the NGS platform used to perform the actual sequencing, and thus multiple PCR strategies can match the size of the PCR fragments to the read-length of the sequencing platform being employed. The PCR amplification can be done in a single-tube as a multiple reaction where all targets are covered at once. In the case of low coverage or ambiguous results, a single-plex PCR can be performed as a confirmatory step to ensure accurate mutation calling. This is also true in the case of highly-degraded samples where the template DNA has fragmented and large-amplicons cannot be extracted from the DNA that remains. See Tables 7-14 for a full list of the primers that have been designed and the general size of fragments each set produces. There are a large number of primers in the list to ensure that there is flexibility to run various multiplex PCR reactions where there is very little sequence overlap in the primers, which can lead to dimerization, and allow melting temperatures of all the oligos in a particular reaction to be matched. The amplification parameters of each PCR reaction consist of 95° C. 15-min heat denaturation phase followed by 40 cycles of denaturation at 95° C. for 15 sec and 55° C. annealing for 30 sec and 72° C. extension for 1 min and finally a 72° C. final extension step for 5 minutes. At the end of the PCR step a diverse set of fragments that cover the exons of interest can be synthesized. The fragments can then be adapted for sequencing on any commercially available NGS platform. Since there is a very wide range of read-lengths that the different NGS instruments produce, from as low as 35 bases to as high as 1500 and expectations of 100 kb read length in the near future, the Insight ALK resistance ID™ is designed to be able to produce fragments as short as 150 bases to as high as 5kb. This ensures for efficient sequencing where the size of each amplicon can be matched to the output of long-read and middle-read technologies (150-1000 bases) or have large enough fragments (5kb) that can be effectively sheared, either sonically or enzymatically, to be compatible with short-read sequencers (<150 bases).

The ALK resistance ID™ takes advantage of the very high-throughput offered by modern sequencers to cover the regions of interest at very high coverage (depth>5,000×) and thus enable the detection of rare variants only present in the sample at a frequency of 1% or less. The sequence reads that are generated can be compared to a reference sequence examined for the presence of any of the mutations listed in Tables 2-6.

TABLE 2
ALK Mutations That Are Covered

| Amino Acid Mutation | Nucleotide Mutation |
|---|---|
| p.V1471fs*45 | c.4409_4422delCCGTGGAAGGGGGA |
| p.Y1584Y | c.4752C > T |
| p.T1597T | c.4791T > A |
| p.L1062I | c.3185A > T |
| p.T1087I | c.3260C > T |
| p.D1091N | c.3271G > A |
| p.G1128A | c.3383G > C |
| p.M1166R | c.3497T > G |
| p.A1168P | c.3502C > G |
| p.I1171N | c.3512T > A |
| p.F1174I | c.3520T > A |
| p.F1174L | c.3522C > A |
| p.R1192P | c.3575G > C |
| p.F1245C | c.3734T > G |
| p.F1245V | c.3733T > G |
| p.F1245L | c.3735C > G |
| p.F1245I | c.3733T > A |
| p.I1250T | c.3749T > C |
| p.R1275Q | c.3824G > A |

TABLE 3
EGFR Mutations That Are Covered

| Amino Acid Mutation | Nucleotide Mutation |
|---|---|
| p.L747_T751 > S | c.2240_2251del12 |
| p.L861Q | c.2582T > A |
| p.L747_E749del | c.2239_2247del9 |
| p.E746_S752 > D | c.2238_2255del18 |
| p.E746_A750del | c.2235_2249del15 |
| p.L858R | c.2573T > G |
| p.E746_A750del | c.2236_2250del15 |
| p.R776C | c.2326C > T |
| p.H835L | c.2504A > T |
| p.G719A | c.2156G > C |
| p.T790M | c.2369C > T |
| p.S768I | c.2303G > T |
| p.V769L | c.2305G > T |
| p.G719S | c.2155G > A |
| p.G719C | c.2155G > T |
| p.L747_T751del | c.2239_2253del15 |
| p.L747_S752del | c.2239_2256del18 |
| p.S752_I759del | c.2254_2277del24 |
| p.P753S | c.2257C > T |
| p.L858M | c.2572C > A |
| p.E746_S752 > A | c.2237_2254del18 |
| p.L747_T751del | c.2240_2254del15 |
| p.L747_P753 > S | c.2240_2257del18 |
| p.E709V | c.2126A > T |
| p.I715S | c.2144T > G |
| p.S720F | c.2159C > T |
| p.L861R | c.2582T > G |
| p.V769_D770insASV | c.2307_2308ins9 |
| p.H773_V774insH | c.2319_2320insCAC |
| p.D770_N771insG | c.2310_2311insGGT |
| p.V769_D770insCV | c.2307_2308insTGCGTG |
| p.H773_V774insPH | c.2319_2320insCCCCAC |
| p.H773_V774insNPH | c.2319_2320ins9 |
| p.L747_A750 > P | c.2239_2248TTAAGAGAAG > C |
| p.L747_T751 > P | c.2239_2251 > C |
| p.E746_S752 > V | c.2237_2255 > T |
| p.E746_S752 > I | c.2235_2255 > AAT |
| p.E746_T751 > V | c.2237_2252 > T |
| p.L747_P753 > Q | c.2239_2258 > CA |
| p.H773 > NPY | c.2317_2317C > AACCCCT |
| p.V774_C775insHV | c.2322_2322G > CCACGTG |
| p.L747_S752 > Q | c.2239_2256 > CAA |
| p.E746_T751 > I | c.2229_2252 > AATTAAGA |
| p.T751_I759 > S | c.2252_2275 > G |
| p.E746_A750 > RP | c.2236_2248 > AGAC |
| p.E746_T751 > VA | c.2237_2253 > TTGCT |
| p.L747_T751 > Q | c.2238_2252 > GCA |
| p.L747_T751 > Q | c.2239_2252 > CA |
| p.L747_S752 > QH | c.2238_2255 > GCAACA |
| p.L747_A750 > P | c.2238_2248 > GC |
| p.I744_K745insKIPVAI | c.2231_2232ins18 |
| p.D761_E762insEAFQ | c.2283_2284ins12 |
| p.A767_S768insTLA | c.2302_2303ins9 |
| p.V769_D770insASV | c.2308_2309ins9 |
| p.D770 > GY | c.2308_2309insGTT |
| p.E709H | c.2125_2127GAA > CAT |
| p.L858R | c.2573_2574TG > GT |
| p.A859T | c.2575G > A |
| p.E746_T751 > A | c.2237_2251del15 |
| p.Y727C | c.2180A > G |
| p.V851I | c.2551G > A |
| p.E746_T751del | c.2236_2253del18 |
| p.D770_N771 > AGG | c.2309_2312ACAA > CTGGTGG |
| p.G857R | c.2569G > A |
| p.L858R | c.2573.T > G |
| p.E746_A750del | c.2235_2249del15 |
| p.E746_A750 > QP | c.2236_2248 > CAAC |
| p.G810D | c.2429G > A |
| p.E709K | c.2125G > A |
| p.D770_N771insN | c.2310_2311insAAC |
| p.D770_N771insG | c.2310_2311insGGC |
| p.H773L | c.2318A > T |
| p.V774M | c.2320G > A |
| p.G779F | c.2335_2336GG > TT |
| p.A871G | c.2612C > G |
| p.E709G | c.2126A > G |
| p.L861Q | c.2582T > A |
| p.L730F | c.2188C > T |
| p.P733L | c.2198C > T |
| p.G735S | c.2203G > A |
| p.V742A | c.2225T > C |
| p.E746K | c.2236G > A |
| p.T751I | c.2252C > T |
| p.S752Y | c.2255C > A |
| p.H850N | c.2548C > A |
| p.D761N | c.2281G > A |
| p.S784F | c.2351C > T |
| p.L792P | c.2375T > C |
| p.L798F | c.2392C > T |
| p.G810S | c.2428G > A |
| p.N826S | c.2477A > G |
| p.T847I | c.2540C > T |
| p.V851A | c.2552T > C |
| p.I853T | c.2558T > C |
| p.A864T | c.2590G > A |
| p.E866K | c.2596G > A |
| p.G873E | c.2618G > A |
| p.E746_P753 > LS | c.2236_2257 > CTCT |
| p.V819V | c.2457G > A |
| p.Y764Y | c.2292C > T |
| p.L833V | c.2497T > G |
| p.V769M | c.2305G > A |
| p.L838V | c.2512C > G |
| p.E709A | c.2126A > C |
| p.D770_N771insSVD | c.2311_2312ins9 |
| p.A839T | c.2515G > A |
| p.H773R | c.2318A > G |
| p.P772P | c.2316C > T |
| p.E746_T751 > A | c.2235_2251 > AG |
| p.E746_A750 > IP | c.2235_2248 > AATTC |
| p.E746_T751 > I | c.2235_2252 > AAT |
| p.E746_T751 > IP | c.2235_2251 > AATTC |
| p.L858R | c.2572_2573CT > AG |
| p.N771_P772 > SVDNR | c.2312_2315ACCC > GCGTGGACAACCG |
| p.D770_P772 > ASVDNR | c.2308_2315GACAACCC > CCAGCGTGGATAACCG |
| p.S752_I759del | c.2253_2276del24 |
| p.E746_A750 > QP | c.2236_2248 > CAAC |
| p.V769_D770insASV | c.2309_2310AC > CCAGCGTGGAT |
| p.M766_A767insAI | c.2298_2299insGCCATA |
| p.G724S | c.2170G > A |
| p.D770N | c.2308G > A |
| p.T783I | c.2348C > T |
| p.G863D | c.2588G > A |
| p.V897I | c.2689G > A |
| p.K745R | c.2234A > G |
| p.P741L | c.2222C > T |
| p.E734K | c.2200G > A |
| p.E746del | c.2234_2236delAGG |
| p.E746_T751 > VP | c.2237_2251 > TTC |
| p.Q787R | c.2360A > G |
| p.V834L | c.2500G > T |
| p.A755A | c.2265C > T |
| p.G719D | c.2156G > A |
| p.E746_S752 > V | c.2237_2256 > TC |
| p.E746_P753 > VS | c.2237_2257 > TCT |
| p.E746_A750 > DP | c.2238_2249 > TCC |
| p.V769_D770insGSV | c.2308_2309ins9 |
| p.V769_D770insGVV | c.2308_2309ins9 |
| p.N771 > GF | c.2311_2312AA > GGGTT |
| p.V774_C775insHV | c.2321_2322insCCACGT |
| p.G719C | c.2154_2155GG > TT |
| p.L747_R748 > FP | c.2241_2244AAGA > CCCG |
| p.E872 | c.2614G > T |
| p.G873G | c.2619A > T |
| p.P753P | c.2259G > A |
| p.G719fs*29 | c.2156delG |
| p.L747_K754 > ST | c.2240_2261 > CGAC |
| p.S768_V769insVAS | c.2303_2304ins9 |
| p.V769_D770insDNV | c.2307_2308ins9 |
| p.D770_N771insAPW | c.2310_2311ins9 |
| p.N771_P772insN | c.2313_2314insAAC |
| p.G796S | c.2386G > A |
| p.E804G | c.2411A > G |
| p.R841K | c.2522G > A |
| p.V834M | c.2500G > A |
| p.D761Y | c.2281G > T |
| p.R776H | c.2327G > A |
| p.L778L | c.2334G > T |
| p.G779C | c.2335G > T |
| p.P848L | c.2543C > T |
| p.L747_T751 > P | c.2238_2251 > GC |
| p.T751_I759 > S | c.2251_2277 > TCT |
| p.N771 > TH | c.2311_2312insCAC |
| p.H773_V774insPH | c.2318_2319insCCCCCA |
| p.V774_C775insHV | c.2322_2323insCACGTG |
| p.L862P | c.2585T > C |
| p.S784Y | c.2351C > A |
| p.F795S | c.2384T > C |
| p.F795S | c.2384T > C |
| p.Y813C | c.2438A > G |
| p.Y801H | c.2401T > C |
| p.C775Y | c.2324G > A |
| p.D770_N771insDG | c.2308_2309insACGGCG |
| p.T751_I759 > REA | c.2252_2277 > GAGAAGCG |
| p.L777Q | c.2330T > A |
| p.G721S | c.2161G > A |
| p.G721D | c.2162G > A |
| p.K754K | c.2262A > G |
| p.E746_T751 > Q | c.2236_2253 > CAA |
| p.L747_T751del | c.2238_2252del15 |
| p.L747_T751 > A | c.2239_2253 > GCT |
| p.C818Y | c.2453G > A |
| p.I759N | c.2276T > A |
| p.T751_E758del | c.2250_2273del24 |
| p.L747P | c.2239_2240TT > CC |
| p.L858K | c.2572_2573CT > AA |
| p.P753_I759del | c.2257_2277del21 |
| p.T751_I759 > N | c.2252_2277 > AT |
| p.G863G | c.2589T > G |
| p.N771 > SH | c.2311_2312insGTC |
| p.D770fs*61 | c.2309_2310ins14 |
| p.E829E | c.2487G > A |
| p.R831C | c.2491C > T |
| p.R831C | c.2491C > T |
| p.L861V | c.2581C > G |
| p.E746_T751del | c.2235_2252del18 |
| p.L747_K754del | c.2239_2262del24 |
| p.L838P | c.2513T > C |
| p.K757 > NK | c.2270_2271insCAA |
| p.G779S | c.2335G > A |
| p.V774L | c.2320G > T |
| p.L815L | c.2445C > T |
| p.E758D | c.2274A > C |
| p.K875R | c.2624A > G |
| p.A864E | c.2591C > A |
| p.Y869C | c.2606A > G |
| p.K745_E749del | c.2233_2247del15 |
| p.F723F | c.2169C > T |
| p.L858L | c.2572C > T |
| p.A859fs*38 | c.2575_2576insG |
| p.N756S | c.2267A > G |
| p.V845A | c.2534T > C |
| p.F856S | c.2567T > C |
| p.G874S | c.2620G > A |
| p.A750_E758del | c.2247_2273del27 |
| p.A750_E758 > P | c.2248_2273 > CC |
| p.L747_5752 > Q | c.2238_2256 > GCAA |
| p.A859_L883 > V | c.2576_2647del72 |
| p.I744_K745insKIPVAI | c.2232_2233ins18 |
| p.K745_E746insVPVAIK | c.2236_2237ins18 |
| p.A767V | c.2300C > T |
| p.N842D | c.2524A > G |
| p.A743T | c.2227G > A |
| p.L747S | c.2240T > C |
| p.K860I | c.2579A > T |
| p.A750_K754del | c.2246_2260del15 |
| p.D770_N771insMATP | c.2311_2312ins12 |
| p.A763_Y764insFQEA | c.2290_2291ins12 |
| p.D761G | c.2282A > G |
| p.V786M | c.2356G > A |
| p.G796A | c.2387G > C |
| p.K728 | c.2182A > T |
| p.R832C | c.2494C > T |
| p.G721A | c.2162G > C |
| p.I744V | c.2230A > G |
| p.S784P | c.2350T > C |
| p.R832L | c.2495G > T |
| p.V802F | c.2404G > T |
| p.E746_E749del | c.2235_2246del12 |
| p.T854A | c.2560A > G |
| p.E884K | c.2650G > A |
| p.F712S | c.2135T > C |
| p.I744M | c.2232C > G |
| p.V765M | c.2293G > A |
| p.R836C | c.2506C > T |
| p.A871T | c.2611G > A |
| p.D855G | c.2564A > G |
| p.E868G | c.2603A > G |
| p.L798H | c.2393T > A |
| p.K806E | c.2416A > G |
| p.L814P | c.2441T > C |
| p.E746_A750 > VP | c.2237_2250 > TCCCT |
| p.V769_D770insMASVD | c.2307_2308ins15 |
| p.F723S | c.2168T > C |
| p.T785N | c.2354C > A |
| p.V845M | c.2533G > A |
| p.M766T | c.2297T > C |
| p.S752P | c.2254T > C |
| p.T725T | c.2175G > A |
| p.D855N | c.2563G > A |
| p.L858Q | c.2573T > A |
| p.H870R | c.2609A > G |
| p.F712L | c.2134T > C |
| p.I821T | c.2462T > C |
| p.V834A | c.2501T > C |
| p.L718P | c.2153T > C |
| p.D770_N771insNPH | c.2310_2311insAACCCCCAC |
| p.D770_N771insGL | c.2310_2311insGGGTTA |
| p.D770_N771insSVD | c.2311_2312insGCGTGGACA |
| p.P772_H773insTHP | c.2315_2316insGACACACCC |
| p.S720T | c.2158T > A |
| p.E746V | c.2237_2238AA > TT |
| p.E746_P753 > VQ | c.2237_2258 > TTCA |
| p.E709_T710 > D | c.2127_2129delAAC |
| p.E746_T751 > IP | c.2236_2253 > ATTCCT |
| p.L747_T751 > Q | c.2239_2253 > CAA |
| p.H773_V774insGNPH | c.2320_2321ins12 |
| p.I732T | c.2195T > C |
| p.N756Y | c.2266A > T |
| p.L844P | c.2531T > C |
| p.I740T | c.2219T > C |
| p.E746_T751 > VP | c.2237_2253 > TTCCT |
| p.W731L | c.2192G > T |
| p.E734Q | c.2200G > C |
| p.T785A | c.2353A > G |
| p.C797Y | c.2390G > A |
| p.R831H | c.2492G > A |
| p.N771 > GY | c.2311_2311A > GGTT |
| p.P733S | c.2197C > T |
| p.R748I | c.2243G > T |
| p.Q849R | c.2546A > G |
| p.E746_T751 > VA | c.2237_2251 > TGG |
| p.E868D | c.2604A > T |
| p.S720S | c.2160C > A |
| p.T725A | c.2173A > G |
| p.R836S | c.2506C > A |
| p.I744I | c.2232C > A |
| p.E866G | c.2597A > G |
| p.I853I | c.2559C > T |
| p.K708E | c.2122A > G |
| p.G824G | c.2472C > A |
| p.F712F | c.2136C > T |
| p.Y827Y | c.2481C > T |
| p.T725M | c.2174C > T |
| p.T725M | c.2174C > T |
| p.K852N | c.2556G > T |
| p.A722V | c.2165C > T |
| p.E711K | c.2131G > A |
| p.T785I | c.2354C > T |
| p.D800N | c.2398G > A |
| p.E872G | c.2615A > G |
| p.E829K | c.2485G > A |
| p.E829K | c.2485G > A |
| p.H870Y | c.2608C > T |
| p.H870Y | c.2608C > T |
| p.D770_N771insSVD | c.2310_2311ins9 |
| p.S768_V769 > IL | c.2303_2305GCG > TCT |
| p.D770_N771insGD | c.2310_2311insGGGGAC |
| p.E709_T710 > A | c.2126_2128delAAA |
| p.E746_S752 > V | c.2235_2255 > GGT |
| p.I744_A750 > VK | c.2230_2249 > GTCAA |
| p.L747_K754 > N | c.2239_2264 > GCCAA |
| p.I740_P741insPVAIKI | c.2219_2220ins18 |
| p.R836R | c.2508C > T |
| p.V843I | c.2527G > A |
| p.K754R | c.2261A > G |
| p.A840T | c.2518G > A |
| p.K754E | c.2260A > G |
| p.A859D | c.2576C > A |
| p.Y801C | c.2402A > G |
| p.I744T | c.2231T > C |
| p.T854I | c.2561C > T |
| p.G863S | c.2587G > A |
| p.H850R | c.2549A > G |
| p.K754A | c.2260_2261AA > GC |
| p.D807N | c.2419G > A |
| p.S720P | c.2158T > C |
| p.K757M | c.2270A > T |
| p.L862Q | c.2585T > A |
| p.T751_I759 > N | c.2252_2276 > A |
| p.P772R | c.2315C > G |
| p.A839V | c.2516C > T |
| p.K716R | c.2147A > G |
| p.H773_V774insQ | c.2319_2320insCAG |
| p.E711V | c.2132_2133AA > TT |
| p.T710A | c.2128A > G |
| p.K714N | c.2142G > C |
| p.V717A | c.2150T > C |
| p.G729E | c.2186G > A |
| p.I744_E749 > LKR | c.2230_2247 > CTTAAGAGA |
| p.E746_T751 > L | c.2236_2253 > CTA |
| p.E746_S752 > I | c.2236_2255 > AT |
| p.E746_S752del | c.2236_2256del21 |
| p.E746_S752 > I | c.2236_2256 > ATC |
| p.E746_P753 > IS | c.2236_2259 > ATCTCG |
| p.E746_T751 > V | c.2237_2253 > TA |
| p.E746_T751 > V | c.2237_2253 > TC |
| p.L747_A750 > P | c.2239_2250 > CCA |
| p.L747_S752 > QH | c.2239_2256 > CAACAT |
| p.L747_P753 > S | c.2239_2257 > T |
| p.R748K | c.2243G > A |
| p.E749G | c.2246A > G |
| p.T751_I759 > S | c.2251_2277 > TCC |
| p.P772_H773insHV | c.2316_2317insCACGTG |
| p.G779D | c.2336G > A |
| p.V802A | c.2405T > C |
| p.L833W | c.2498T > G |
| p.D837G | c.2510A > G |
| p.L844V | c.2530C > G |
| p.T751_I759del | c.2252_2275del24 |
| p.V765G | c.2294T > G |
| p.G796D | c.2387G > A |
| p.R836H | c.2507G > A |
| p.K757R | c.2270A > G |
| p.E872K | c.2614G > A |
| p.L858L | c.2574G > A |
| p.I780S | c.2339T > G |
| p.T785P | c.2353A > C |
| p.Y801fs*1 | c.2402_2403insG |
| p.L858R | c.2573_2574TG > GA |
| p.N771_P772insRH | c.2311_2312insACCGGC |
| p.H850Y | c.2548C > T |
| p.E868K | c.2602G > A |
| p.I780T | c.2339T > C |
| p.E866D | c.2598G > T |
| p.L833F | c.2499G > T |
| p.A864V | c.2591C > T |
| p.K745_A750del | c.2232_2249del18 |
| p.P794H | c.2381C > A |
| p.E804K | c.2410G > A |
| p.G857E | c.2570G > A |

TABLE 4
KIT Mutations That Are Covered

| Amino Acid Mutation | Nucleotide Mutation |
|---|---|
| p.(550_592)ins7 | c.(1648_1774)ins21 |
| p.C443Y | c.1328G > A |
| p.P456S | c.1366C > T |
| p.L462L | c.1384C > T |
| p.P468P | c.1404G > A |
| p.F469L | c.1405T > C |
| p.L472L | c.1416A > G |
| p.S476I | c.1427G > T |
| p.D479fs*2 | c.1434_1462del29 |
| p.S480fs*47 | c.1439delC |
| p.N486D | c.1456A > G |
| p.V489I | c.1465G > A |
| p.V489A | c.1466T > C |
| p.E490G | c.1469A > G |
| p.E490_F504 > DHIVVSLTF | c.1470_1512 > CCACATCGTTGTAAGCCTTACATTC |
| p.N495I | c.1484A > T |
| p.N495I | c.1484A > T |
| p.D496V | c.1487A > T |
| p.V50M | c.148G > A |
| p.V497V | c.1491G > A |
| p.G498D | c.1493G > A |
| p.K499K | c.1497G > A |
| p.A502_Y503insSA | c.1504_1505insCTTCTG |
| p.A502_Y503insSA | c.1505_1506insTTCTGC |
| p.Y503_F504insSA | c.1507_1508insCTGCCT |
| p.A502_Y503insFA | c.1507_1508insTTGCCT |
| p.Y503_F504insAY | c.1509_1510insGCCTAT |
| p.F504L | c.1510T > C |
| p.N505H | c.1513A > C |
| p.F506L | c.1516T > C |
| p.F506_A507insAYFNF | c.1518_1519ins15 |
| p.F508_K509insNFAF | c.1524_1525ins12 |
| p.K509I | c.1526A > T |
| p.G510del | c.1528_1530delGGT |
| p.N512D | c.1534A > G |
| p.V530I | c.1588G > A |
| p.I531_V532insGF | c.1593_1594insGGGTTC |
| p.M541L | c.1621A > C |
| p.K546K | c.1638A > G |
| p.Q549_V555 > I | c.1645_1663 > A |
| p.K550_P551del | c.1648_1653delAAACCC |
| p.K550_E554del | c.1648_1662del15 |
| p.K550_V555del | c.1648_1665del18 |
| p.K550_V555del | c.1648_1665del18 |
| p.K550_Q556 > II | c.1648_1668 > ATTATT |
| p.K550_W557del | c.1648_1671del24 |
| p.K550fs*6 | c.1648_1672del25 |
| p.K550_K558del | c.1648_1674del27 |
| p.K550_V559del | c.1648_1677del30 |
| p.K550_V555 > I | c.1649_1663del15 |
| p.K550R | c.1649A > G |
| p.K550I | c.1649A > T |
| p.K550_V555 > KTL | c.1650_1663 > AACCC |
| p.P551_K558del | c.1650_1673del24 |
| p.K550N | c.1650A > C |
| p.P551del | c.1651_1653delCCC |
| p.P551_E554del | c.1651_1662del12 |
| p.P551_V555del | c.1651_1665del15 |
| p.P551_Q556del | c.1651_1668del18 |
| p.P551T | c.1651C > A |
| p.P551S | c.1651C > T |
| p.P551_M552 > L | c.1652_1654delCCA |
| p.P551_E554 > H | c.1652_1662 > AY |
| p.P551_V555 > L | c.1652_1663del12 |
| p.P551_V559del > L | c.1652_1678del27 |
| p.P551L | c.1652C > T |
| p.M552_Y553del | c.1653_1658delCATGTA |
| p.M552_Q556> | c.1653_1667 > TCT |
| p.M552_W557del | c.1653_1670del18 |
| p.M552_Y553del | c.1654_1659delATGTAT |
| p.M552_E554del | c.1654_1662del9 |
| p.M552_V555del | c.1654_1665del12 |
| p.M552_Q556del | c.1654_1668del15 |
| p.M552_W557del | c.1654_1671del18 |
| p.M552_K558del | c.1654_1674del21 |
| p.M552_D572del | c.1654_1716del63 |
| p.M552L | c.1654A > C |
| p.M552L | c.1654A > C |
| p.M552_Y553 > N | c.1655_1657delTGT |
| p.M552_E554 > K | c.1655_1660delTGTATG |
| p.M552_V555 > I | c.1655_1663del9 |
| p.M552_Q556 > K | c.1655_1666del12 |
| p.M552_W557 > R | c.1655_1669del15 |
| p.M552_W557del | c.1655_1672del18 |
| p.M552_K558 > T | c.1655_1674 > CN |
| p.M552_E561 > K | c.1655_1681del27 |
| p.M552_T574 > TESA | c.1655_1720 > CAGAATCAG |
| p.M552K | c.1655T > A |
| p.M552T | c.1655T > C |
| p.Y553_W557del | c.1656_1670del15 |
| p.Y553_K558> | c.1656_1673del18 |
| p.Y553V | c.1657_1658TA > GT |
| p.Y553_Q556del | c.1657_1668del12 |
| p.Y553_W557del | c.1657_1671del15 |
| p.Y553_K558del | c.1657_1674del18 |
| p.Y553_V559 > E | c.1657_1677 > GAA |
| p.Y553_V559del | c.1657_1677del21 |
| p.Y553N | c.1657T > A |
| p.Y553_T574 > S | c.1658_1720del63 |
| p.E554_K558del | c.1660_1674del15 |
| p.E554_E562del | c.1660_1686del27 |
| p.E554_N564del | c.1660_1692del33 |
| p.E554_I571del | c.1660_1713del54 |
| p.E554_D572del | c.1660_1716del57 |
| p.E554K | c.1660G > A |
| p.E554K | c.1660G > A |
| p.E554_K558del | c.1661_1675del15 |
| p.E554G | c.1661A > G |
| p.V555_E562del | c.1662_1685del24 |
| p.E554D | c.1662A > T |
| p.V555_Q556del | c.1663_1668delGTACAG |
| p.V555_K558del | c.1663_1674del12 |
| p.V555_V559del | c.1663_1677del15 |
| p.V555_V560del | c.1663_1680del18 |
| p.V555_I563del | c.1663_1689del27 |
| p.V555_G565del | c.1663_1695del33 |
| p.V555_Y570del | c.1663_1710del48 |
| p.V555_I571del | c.1663_1713del51 |
| p.V555_P573del | c.1663_1719del57 |
| p.V555I | c.1663G > A |
| p.V555_N566 > D | c.1664_1696del33 |
| p.Q556_V559del | c.1665_1676del12 |
| p.V555_V560 > V | c.1665_1679del15 |
| p.Q556_N566 > SNNLQLY | c.1665_1696 > TTCCAACAACCTTCCACTGT |
| p.Q556_D572del | c.1665_1716 > T |
| p.Q556_W557del | c.1666_1671delCAGTGG |
| p.Q556_V559del | c.1666_1677del12 |
| p.Q556_V560 > F | c.1666_1678 > T |
| p.Q556_V560 > TTF | c.1666_1680 > ACAACCTTC |
| p.Q556_V560del | c.1666_1680del15 |
| p.Q556_E561 > HH | c.1666_1683 > CATCAT |
| p.Q556_E561del | c.1666_1683del18 |
| p.Q556_D572 > PS | c.1666_1716 > CCATCC |
| p.Q556_P573del | c.1666_1719del54 |
| p.Q556_T574del | c.1666_1722del57 |
| p.Q556_L576del | c.1666_1728del63 |
| p.Q556_W557 > R | c.1667_1669delAGT |
| p.W557_K558del | c.1667_1672delAGTGGA |
| p.Q556_K558 > R | c.1667_1673AGTGGAA > G |
| p.W557_E561del | c.1667_1681del15 |
| p.Q556R | c.1667A > G |
| p.W557_K558del | c.1668_1673delGTGGAA |
| p.Q556_K558 > HPCR | c.1668_1673GTGGAA > CCCCTGCAG |
| p.Q556_K558 > H | c.1668_1674GTGGAAG > Y |
| p.Q556_V559 > H | c.1668_1676del9 |
| p.Q556_V559 > HT | c.1668_1677GTGGAAGGTT > TACT |
| p.Q556_V560 > HNLQLY | c.1668_1679 > CAACCTTCCACTGTA |
| p.Q556_V560 > H | c.1668_1679del12 |
| p.W557_I571del | c.1668_1712del45 |
| p.Q556_D572 > H | c.1668_1715del48 |
| p.W557_Q575del | c.1668_1724del57 |
| p.W557del | c.1669_1671delTGG |
| p.W557_K558 > E | c.1669_1672TGGA > G |
| p.W557_K558del | c.1669_1674delTGGAAG |
| p.W557_K558 > S | c.1669_1674TGGAAG > C |
| p.W557_V559 > I | c.1669_1675TGGAAGG > A |
| p.W557_V559del | c.1669_1677del9 |
| p.W557_V560del | c.1669_1680del12 |
| p.W557_E561del | c.1669_1683del15 |
| p.W557_E562del | c.1669_1686del18 |
| p.W557_Q575del | c.1669_1725del57 |
| p.W557R | c.1669T > A |
| p.W557R | c.1669T > C |
| p.W557G | c.1669T > G |
| p.W557_K558 > SS | c.1670_1673GGAA > CTTC |
| p.W557_K558 > FP | c.1670_1674GGAAG > TTCCT |
| p.W557_V559 > F | c.1670_1675delGGAAGG |
| p.W557_V560 > F | c.1670_1678del9 |
| p.W557_P573 > S | c.1670_1717del48 |
| p.W557S | c.1670G > C |
| p.W557_K558 > CT | c.1671_1673GAA > CAC |
| p.W557_K558 > CP | c.1671_1673GAA > TCC |
| p.W557_K558 > C | c.1671_1674GAAG > C |
| p.W557_V559 > C | c.1671_1676delGAAGGT |
| p.W557_V560 > C | c.1671_1679del9 |
| p.W557 | c.1671G > A |
| p.W557C | c.1671G > T |
| p.K558_V559 > SS | c.1672_1676AAGGT > TCTTC |
| p.K558_V559del | c.1672_1677delAAGGTT |
| p.K558_V560del | c.1672_1680del9 |
| p.K558_E562del | c.1672_1686del15 |
| p.K558_N564del | c.1672_1692del21 |
| p.K558_G565del | c.1672_1695del24 |
| p.K558_D572del | c.1672_1716del45 |
| p.K558_Q575del | c.1672_1725del54 |
| p.K558E | c.1672A > G |
| p.K558* | c.1672A > T |
| p.K558 > NP | c.1673_1674insTCC |
| p.K558_V560 > I | c.1673_1678delAGGTTG |
| p.K558_V560 > M | c.1673_1680AGGTTGTT > TG |
| p.K558_E562del | c.1673_1687del15 |
| p.K558_G565 > R | c.1673_1693del21 |
| p.K558R | c.1673A > G |
| p.K558 > NP | c.1674_1674G > TCCT |
| p.K558_V559 > N | c.1674_1676delGGT |
| p.K558_V560 > N | c.1674_1679delGGTTGT |
| p.K558_Y570 > N | c.1674_1709del36 |
| p.K558_L576 > NV | c.1674_1726 > CG |
| p.K558K | c.1674G > A |
| p.K558N | c.1674G > C |
| p.K558N | c.1674G > Y |
| p.V559del | c.1675_1677delGTT |
| p.V559K | c.1675_1677GTT > AAG |
| p.V559_V560del | c.1675_1680delGTTGTT |
| p.V559_E561del | c.1675_1683del9 |
| p.V559_G565del | c.1675_1695del21 |
| p.V559_I571del | c.1675_1713del39 |
| p.V559_L576del | c.1675_1728del54 |
| p.V559I | c.1675G > A |
| p.V559_E561del | c.1676_1684del9 |
| p.V559_E562del | c.1676_1687del12 |
| p.V559_P573 > A | c.1676_1717del42 |
| p.V559D | c.1676T > A |
| p.V559A | c.1676T > C |
| p.V559G | c.1676T > G |
| p.V560del | c.1678_1680delGTT |
| p.V560_L576del | c.1678_1728del51 |
| p.V560E | c.1679_1680TT > AG |
| p.V560E | c.1679_1680TT > AR |
| p.V560del | c.1679_1681delTTG |
| p.V560_I571del | c.1679_1714del36 |
| p.V560D | c.1679T > A |
| p.V560A | c.1679T > C |
| p.V560G | c.1679T > G |
| p.E561del | c.1680_1682delTGA |
| p.V560V | c.1680T > G |
| p.E561del | c.1681_1683delGAG |
| p.E561_P577del | c.1681_1731del51 |
| p.E561K | c.1681G > A |
| p.E561G | c.1682A > G |
| p.E561E | c.1683G > A |
| p.E562_P573del | c.1684_1719del36 |
| p.E562K | c.1684G > A |
| p.E562V | c.1685A > T |
| p.E562_V569 > D | c.1686_1706del21 |
| p.I563_D572del | c.1687_1716del30 |
| p.I563_L576del | c.1687_1728del42 |
| p.I563V | c.1687A > G |
| p.N564_T574del | c.1690_1722del33 |
| p.N564_L576del | c.1690_1728del39 |
| p.N564_P577del | c.1690_1731del42 |
| p.N564_Y578del | c.1690_1734del45 |
| p.N564H | c.1690A > C |
| p.N564_P573 > TS | c.1691_1717 > CCT |
| p.N564_P573 > T | c.1691_1717del27 |
| p.N564S | c.1691A > G |
| p.N564K | c.1692T > G |
| p.G565R | c.1693G > A |
| p.G565E | c.1694G > A |
| p.G565V | c.1694G > T |
| p.N566D | c.1696A > G |
| p.N566S | c.1697A > G |
| p.N567_L576 > E | c.1698_1728 > CGAA |
| p.N566N | c.1698C > T |
| p.N567_P573del | c.1699_1719del21 |
| p.N567H | c.1699A > C |
| p.N567K | c.1701T > A |
| p.Y568_T574del | c.1702_1722del21 |
| p.Y568D | c.1702T > G |
| p.Y568_L576 > CV | c.1703_1726 > GTG |
| p.Y568S | c.1703A > C |
| p.Y568C | c.1703A > G |
| p.Y568Y | c.1704T > C |
| p.V569_L576del | c.1705_?del? |
| p.V569_D572del | c.1705_1716del12 |
| p.V569_Q575del | c.1705_1725del21 |
| p.V569_L576del | c.1705_1728del24 |
| p.V569I | c.1705G > A |
| p.Y570_L576delYIDPTQL | c.1706_1726del21 |
| p.V569_L576 > G | c.1706_1727 > G |
| p.V569A | c.1706T > C |
| p.V569G | c.1706T > G |
| p.Y570_L576del | c.1708_1728del21 |
| p.Y570D | c.1708T > G |
| p.Y570* | c.1710C > A |
| p.I571_L576del | c.1711_1728del18 |
| p.I571_N587del | c.1712_1762del51 |
| p.I571R | c.1712T > G |
| p.I571M | c.1713A > G |
| p.571_572 > GE | c.1714_1715insGGGAAG |
| p.D572N | c.1714G > A |
| p.D572Y | c.1714G > T |
| p.D572A | c.1715A > C |
| p.D572D | c.1716C > T |
| p.P573L | c.1718C > T |
| p.P573_T574insYIDP | c.1719_1720ins12 |
| p.T574A | c.1720A > G |
| p.T574_Q575ins12 | c.1721_1722ins36 |
| p.T574I | c.1721C > T |
| p.Q575del | c.1723_1725delCAA |
| p.Q575_P577 > T | c.1723_1731CAACTTCCT > ACA |
| p.L576del | c.1726_1728delCTT |
| p.L576F | c.1726C > T |
| p.L576del | c.1727_1729delTTC |
| p.L576P | c.1727T > C |
| p.L576_P577insQL | c.1728_1729insCAACTT |
| p.P577_Y578del | c.1729_1734delCCTTAT |
| p.P577S | c.1729C > T |
| p.P577_D579del | c.1730_1738del9 |
| p.P577H | c.1730C > A |
| p.P577L | c.1730C > T |
| p.D579del | c.1735_1737delGAT |
| p.D579_H580insPTQLPYD | c.1737_1738ins21 |
| p.D579_H580insSYD | c.1737_1738ins9 |
| p.H580del | c.1737_1739delTCA |
| p.H580Y | c.1738C > T |
| p.H580_K581insHPYD | c.1739_1740ins12 |
| p.H580_K581insPYDH | c.1740_1741ins12 |
| p.H580_K581insPTQLPYDH | c.1740_1741ins24 |
| p.H580_K581insIDPTQLPYDH | c.1740_1741ins30 |
| p.H580_K581insYDH | c.1740_1741ins9 |
| p.K581R | c.1742A > G |
| p.W582* | c.1745G > A |
| p.W582* | c.1746G > A |
| p.E583_F584insPYDHKWE | c.1748_1749ins21 |
| p.E583G | c.1748A > G |
| p.F584L | c.1750T > C |
| p.F584S | c.1751T > C |
| p.F584_P585insLPYDHKWEF | c.1752_1753ins27 |
| p.F584_P585ins13 | c.1752_1753ins39 |
| p.F584_P585ins15 | c.1752_1753ins45 |
| p.P585_R586insYDHKWEFP | c.1754_1755ins24 |
| p.P585_R586ins12 | c.1754_1755ins36 |
| p.P585_R586insLPYDHKWEFP | c.1755_1756ins30 |
| p.P585_R586ins13 | c.1755_1756ins39 |
| p.P585_R586ins14 | c.1755_1756ins42 |
| p.P585_R586ins17 | c.1755_1756ins51 |
| p.P585P | c.1755C > T |
| p.N587_R588ins15 | c.1761_1762ins45 |
| p.N587N | c.1761C > T |
| p.R588_L589ins17 | c.1764_1765ins51 |
| p.S590N | c.1769G > A |
| p.F591L | c.1771T > C |
| p.F591_G592ins21 | c.1773_1774ins63 |
| p.G592_K593ins16 | c.1774_1775ins48 |
| P.? | c.1774 + 3C > T |
| p.G592_K593ins21 | c.1775_1776ins63 |
| p.T594I | c.1781C > T |
| p.A599T | c.1795G > A |
| p.P627L | c.1880C > T |
| p.T632I | c.1895C > T |
| p.E633G | c.1898A > G |
| p.R634R | c.1902G > A |
| p.E635G | c.1904A > G |
| p.A636V | c.1907C > T |
| p.L637F | c.1909C > T |
| p.S639P | c.1915T > C |
| p.K642Q | c.1924A > C |
| p.K642E | c.1924A > G |
| p.V643A | c.1928T > C |
| p.S645N | c.1934G > A |
| p.L647F | c.1939C > T |
| p.L647P | c.1940T > C |
| p.G648S | c.1942G > A |
| p.N649_H650insN | c.1947_1948insAAT |
| p.I653T | c.1958T > C |
| p.V654A | c.1961T > C |
| p.N655K | c.1965T > G |
| p.G663V | c.1988G > T |
| p.G664R | c.1990G > A |
| p.T670E | c.2008_2009AC > GA |
| p.T670I | c.2009C > T |
| p.L682fs*1 | c.2045delT |
| p.S692L | c.2075C > T |
| p.E695K | c.2083G > A |
| p.H697Y | c.2089C > T |
| p.H697fs*28 | c.2089delC |
| p.R815_D816insVI | c.2445_2446insGTCATA |
| p.D816I | c.2446_2447GA > AT |
| p.D816F | c.2446_2447GA > TT |
| p.D816N | c.2446G > A |
| p.D816H | c.2446G > C |
| p.D816Y | c.2446G > T |
| p.D816 > GP | c.2447_2448AC > GGCCA |
| p.D816 > VVA | c.2447_2448AC > TCGTTGCA |
| p.D816A | c.2447A > C |
| p.D816G | c.2447A > G |
| p.D816V | c.2447A > T |
| p.D816E | c.2448C > G |
| p.D816E | c.2448C > G |
| p.I817V | c.2449A > G |
| p.I817T | c.2450T > C |
| p.K818R | c.2453A > G |
| p.K818K | c.2454G > A |
| p.N819Y | c.2455A > T |
| p.D820N | c.2458G > A |
| p.D820H | c.2458G > C |
| p.D820H | c.2458G > C |
| p.D820Y | c.2458G > T |
| p.D820Y | c.2458G > T |
| p.D820A | c.2459A > C |
| p.D820G | c.2459A > G |
| p.D820V | c.2459A > T |
| p.D820E | c.2460T > A |
| p.D820E | c.2460T > G |
| p.N822H | c.2464A > C |
| p.N822Y | c.2464A > T |
| p.N822Y | c.2464A > T |
| p.N822S | c.2465A > G |
| p.N822K | c.2466T > A |
| p.N822N | c.2466T > C |
| p.N822K | c.2466T > G |
| p.N822K | c.2466T > R |
| p.Y823N | c.2467T > A |
| p.Y823D | c.2467T > G |
| p.Y823C | c.2468A > G |
| p.V825I | c.2473G > A |
| p.V825A | c.2474T > C |
| p.A829P | c.2485G > C |
| p.A829V | c.2486C > T |
| p.R830* | c.2488C > T |
| p.R830* | c.2488C > T |
| p.L831P | c.2492T > C |
| p.V833L | c.2497G > C |
| p.V833V | c.2499G > T |
| p.E839K | c.2515G > A |
| p.C844Y | c.2531G > A |
| p.Y846H | c.2536T > C |
| p.F848L | c.2542T > C |
| p.E849* | c.2545G > T |
| p.W853* | c.2558G > A |
| p.S854P | c.2560T > C |
| p.L859P | c.2576T > C |
| p.L859L | c.2577T > G |
| p.E861E | c.2583G > A |
| p.L862L | c.2586G > C |

TABLE 5
KRAS Mutations That Are Covered

| Amino Acid Mutation | Nucleotide Mutation |
|---|---|
| p.V9V | c.27T > C |
| p.A11P | c.31G > C |
| p.A11V | c.32C > T |
| p.G12F | c.34_35GG > TT |
| p.G12C | c.34_36GGT > TGC |
| p.G12L | c.34_35GG > CT |
| p.G12L | c.34_35GG > CT |
| p.G12V | c.35_36GT > TC |
| p.G12C | c.34G > T |
| p.G12S | c.34G > A |
| p.G12R | c.34G > C |
| p.G12E | c.35_36GT > AA |
| p.G12V | c.35G > T |
| p.G12D | c.35G > A |
| p.G12A | c.35G > C |
| p.G12G | c.36T > C |
| p.G13C | c.37G > T |
| p.G13S | c.37G > A |
| p.G13R | c.37G > C |
| p.G13D | c.38G > A |
| p.G13A | c.38G > C |
| p.G13V | c.38G > T |
| p.A18T | c.52G > A |
| p.A18D | c.53C > A |
| p.Q61K | c.181C > A |
| p.Q61E | c.181C > G |
| p.Q61P | c.182A > C |
| p.Q61R | c.182A > G |
| p.Q61L | c.182A > T |
| p.Q61H | c.183A > C |
| p.Q61H | c.183A > T |
| p.D69fs*4 | c.205delG |
| p.G12fs*3 | c.35delG |
| p.G13V | c.38_39GC > TT |
| p.V14I | c.40G > A |
| p.Q61K | c.180_181TC > CA |

TABLE 6
BRAF Mutations That Are Covered

| Amino Acid Mutation | cDNA Nucleotide Mutation |
|---|---|
| p.G30D | c.89G > A |
| p.M53T | c.158T > C |
| p.S102F | c.305C > T |
| p.S129L | c.386C > T |
| p.R146W | c.436C > T |
| p.I156I | c.468C > T |
| p.R178* | c.532C > T |
| p.A184T | c.550G > A |
| p.Y198H | c.592T > C |
| p.Q201H | c.603G > T |
| p.K205Q | c.613A > C |
| p.F247L | c.741T > G |
| p.Q257H | c.771G > T |
| p.G258V | c.773G > T |
| p.H298Y | c.892C > T |
| p.I300V | c.898A > G |
| p.A305V | c.914C > T |
| p.E309* | c.925G > T |
| p.T310I | c.929C > T |
| p.S323S | c.969G > A |
| p.I326V | c.976A > G |
| p.I326T | c.977T > C |
| p.F357S | c.1070T > C |
| p.G358G | c.1074G > C |
| p.S364L | c.1091C > T |
| p.S365L | c.1094C > T |
| p.P367R | c.1100C > G |
| p.S394* | c.1181C > G |
| p.T401I | c.1202C > T |
| p.P403fs*8 | c.1208delC |
| p.A404fs*9 | c.1208_1209insC |
| p.G421V | c.1262G > T |
| p.G421G | c.1263A > G |
| p.K439Q | c.1315A > C |
| p.K439T | c.1316A > C |
| p.T440P | c.1318A > C |
| p.T440A | c.1318A > G |
| p.T440T | c.1320A > G |
| p.G442S | c.1324G > A |
| p.R444W | c.1330C > T |
| p.R444Q | c.1331G > A |
| p.R444L | c.1331G > T |
| p.R444R | c.1332G > A |
| p.R444R | c.1332G > T |
| p.S447S | c.1341T > C |
| p.W450* | c.1349G > A |
| p.W450L | c.1349G > T |
| p.P453T | c.1357C > A |
| p.P453P | c.1359T > C |
| p.G455R | c.1363G > A |
| p.G455E | c.1364G > A |
| p.Q456* | c.1366C > T |
| p.Q456R | c.1367A > G |
| p.Q456Q | c.1368G > A |
| p.I457T | c.1370T > C |
| p.V459L | c.1375G > C |
| p.V459A | c.1376T > C |
| p.V459V | c.1377G > A |
| p.G460* | c.1378G > T |
| p.G460G | c.1380A > G |
| p.R462G | c.1384A > G |
| p.R462K | c.1385G > A |
| p.R462I | c.1385G > T |
| p.R462R | c.1386A > G |
| p.I463V | c.1387A > G |
| p.I463S | c.1388T > G |
| p.I463I | c.1389T > C |
| p.G464R | c.1390G > A |
| p.G464R | c.1390G > C |
| p.G464E | c.1391G > A |
| p.G464V | c.1391G > T |
| p.S465S | c.1395T > C |
| p.G466R | c.1396G > A |
| p.G466R | c.1396G > C |
| p.G466E | c.1397G > A |
| p.G466A | c.1397G > C |
| p.G466V | c.1397G > T |
| p.G466G | c.1398A > G |
| p.S467P | c.1399T > C |
| p.S467L | c.1400C > T |
| p.F468L | c.1402T > C |
| p.F468S | c.1403T > C |
| p.F468C | c.1403T > G |
| p.F468F | c.1404T > C |
| p.G469R | c.1405G > A |
| p.G469R | c.1405G > C |
| p.G469>? | c.1405_1406GG > CT |
| p.G469S | c.1405_1406GG > TC |
| p.G469L | c.1405_1406GG > TT |
| p.G469S | c.1405_1407GGA > AGC |
| p.G469S | c.1405_1407GGA > AGT |
| p.G469E | c.1406G > A |
| p.G469A | c.1406G > C |
| p.G469V | c.1406G > T |
| p.G469G | c.1407A > G |
| p.V471I | c.1411G > A |
| p.V471F | c.1411G > T |
| p.V471A | c.1412T > C |
| p.Y472S | c.1415A > C |
| p.Y472C | c.1415A > G |
| p.K475R | c.1424A > G |
| p.K475M | c.1424A > T |
| p.K475K | c.1425G > A |
| p.D479Y | c.1435G > T |
| p.L485L | c.1453T > C |
| p.L485S | c.1454T > C |
| p.L485_P490 > Y | c.1454_1469 > A |
| p.L485F | c.1455G > T |
| p.N486_P490del | c.1457_1471del15 |
| p.V487V | c.1461G > A |
| p.L505H | c.1514T > A |
| p.R509* | c.1525C > T |
| p.L514P | c.1541T > C |
| p.W531C | c.1593G > T |
| p.L537S | c.1610T > C |
| p.H539P | c.1616A > C |
| p.H542Y | c.1624C > T |
| p.K570K | c.1710G > A |
| p.H574N | c.1720C > A |
| p.H574Q | c.1722C > A |
| p.N581S | c.1742A > G |
| p.N581I | c.1742A > T |
| p.I582M | c.1746A > G |
| p.F583S | c.1748T > C |
| p.F583F | c.1749T > C |
| p.L584F | c.1750C > T |
| p.L584P | c.1751T > C |
| p.L584L | c.1752T > C |
| p.H585H | c.1755T > C |
| p.E586K | c.1756G > A |
| p.E586E | c.1758A > G |
| p.D587N | c.1759G > A |
| p.D587A | c.1760A > C |
| p.D587G | c.1760A > G |
| p.D587E | c.1761C > A |
| p.D587E | c.1761C > G |
| p.L588P | c.1763T > C |
| p.L588R | c.1763T > G |
| p.L588L | c.1764C > T |
| p.T589A | c.1765A > G |
| p.T589I | c.1766C > T |
| p.T589T | c.1767A > G |
| p.V590I | c.1768G > A |
| p.V590A | c.1769T > C |
| p.V590fs*3 | c.1769delT |
| p.V590V | c.1770A > G |
| p.K591E | c.1771A > G |
| p.K591R | c.1772A > G |
| p.I592V | c.1774A > G |
| p.I592M | c.1776A > G |
| p.I592I | c.1776A > T |
| p.G593S | c.1777G > A |
| p.G593C | c.1777G > T |
| p.G593D | c.1778G > A |
| p.D594N | c.1779_1780TG > GA |
| p.D594N | c.1780G > A |
| p.D594H | c.1780G > C |
| p.D594G | c.1781A > G |
| p.D594V | c.1781A > T |
| p.D594E | c.1782T > A |
| p.D594D | c.1782T > C |
| p.D594E | c.1782T > G |
| p.F595L | c.1783T > C |
| p.F595S | c.1784T > C |
| p.F595L | c.1785T > A |
| p.F595F | c.1785T > C |
| p.F595L | c.1785T > G |
| p.G596R | c.1786G > C |
| p.G596fs*2 | c.1786delG |
| p.G596D | c.1787G > A |
| p.G596G | c.1788T > C |
| p.L597V | c.1789C > G |
| p.L597S | c.1789_1790CT > TC |
| p.L597Q | c.1790T > A |
| p.L597P | c.1790T > C |
| p.L597R | c.1790T > G |
| p.L597L | c.1791A > G |
| p.A598T | c.1792G > A |
| p.A598V | c.1793C > T |
| p.A598A | c.1794T > A |
| p.A598_T599insV | c.1794_1795insGTT |
| p.T599del | c.1794_1796delTAC |
| p.T599I | c.1796C > T |
| p.T599_V600insT | c.1796_1797insTAC |
| p.T599_V600 > IAL | c.1796_1798CAG > TAGCTT |
| p.T599_R603 > I | c.1796 1809 > TC |
| p.T599T | c.1797A > B |
| p.T599T | c.1797A > G |
| p.T599T | c.1797A > T |
| p.T599_V600insTT | c.1797_1797A > TACTACG |
| p.T599_V600insTT | c.1797_1798ins? |
| p.T599_V600insT | c.1797_1798insACA |
| p.T599_V600insDFGLAT | c.1798_1799ins18 |
| p.V600R | c.1797_1799AGT > GAG |
| p.V600M | c.1798G > A |
| p.V600L | c.1798G > C |
| p.V600L | c.1798G > T |
| p.V600 > YM | c.1798_1798G > TACA |
| p.V600K | c.1798_1799GT > AA |
| p.V600R | c.1798_1799GT > AG |
| p.V600Q | c.1798_1799GT > CA |
| p.V600E | c.1799T > A |
| p.V600A | c.1799T > C |
| p.V600G | c.1799T > G |
| p.V600E | c.1799_1800TG > AA |
| p.V600D | c.1799_1800TG > AC |
| p.V600D | c.1799_1800TG > AT |
| p.V600fs*11 | c.1799_1800delTG |
| p.V600_K601 > E | c.1799_1801delTGA |
| p.V600_S602 > DT | c.1799_1804TGAAAT > ATA |
| p.V600_S605 > D | c.1799_1814 > A |
| p.V600_S605 > DV | c.1799_1814 > ATGT |
| p.V600_S605 > EK | c.1799_1815 > AAAAG |
| p.V600V | c.1800G > A |
| p.V600? | c.(1798-1800)? |
| p.K601E | c.1801A > G |
| p.K601del | c.1801_1803delAAA |
| p.K601R | c.1802A > G |
| p.K601I | c.1802A > T |
| p.K601N | c.1803A > C |
| p.K601K | c.1803A > G |
| p.K601N | c.1803A > T |
| p.S602S | c.1806T > G |
| p.R603R | c.1807C > A |
| p.R603* | c.1807C > T |
| p.R603L | c.1808G > T |
| p.R603R | c.1809A > G |
| p.W604del | c.1808_1810delGAT |
| p.W604R | c.1810T > A |
| p.W604G | c.1810T > G |
| p.W604* | c.1811G > A |
| p.W604* | c.1812G > A |
| p.S605G | c.1813A > G |
| p.S605F | c.1813_1814AG > TT |
| p.S605N | c.1814G > A |
| p.S605R | c.1815T > A |
| p.G606R | c.1816G > A |
| p.G606S | c.1816_1818GGG > AGT |
| p.G606E | c.1817G > A |
| p.G606A | c.1817G > C |
| p.G606V | c.1817G > T |
| p.G606G | c.1818G > A |
| p.S607P | c.1819T > C |
| p.H608R | c.1823A > G |
| p.H608H | c.1824T > C |
| p.Q609R | c.1826A > G |
| p.Q609Q | c.1827G > A |
| p.F610L | c.1828T > C |
| p.F610S | c.1829T > C |
| p.F610F | c.1830T > C |
| p.E611G | c.1832A > G |
| p.E611D | c.1833A > C |
| p.E611E | c.1833A > G |
| p.Q612E | c.1834C > G |
| p.Q612* | c.1834C > T |
| p.S614P | c.1840T > C |
| p.S614S | c.1842T > C |
| p.G615R | c.1843G > A |
| p.S616P | c.1846T > C |
| p.S616F | c.1847C > T |
| p.I617T | c.1850T > C |
| p.L618L | c.1852T > C |
| p.L618S | c.1853T > C |
| p.L618W | c.1853T > G |
| p.W619R | c.1855T > C |
| p.Q636E | c.1906C > G |
| p.Q636* | c.1906C > T |
| p.Q636R | c.1907A > G |
| p.S637P | c.1909T > C |
| p.S637* | c.1910C > G |
| p.S637L | c.1910C > T |
| p.S657S | c.1971A > G |
| p.R671Q | c.2012G > A |
| p.R682W | c.2044C > T |
| p.R682Q | c.2045G > A |
| p.K698R | c.2093A > G |
| p.A718V | c.2153C > T |
| p.P731S | c.2191C > T |
| p.P731P | c.2193C > T |

TABLE 7
ALK Capture Primers List for NGS Panel - Genomic DNA

| | | |
|---|---|---|
| Seq. | | |
| ID | Primer | Sequence |

ALK Exon21 130-150 bases

| | | |
|---|---|---|
| 1 | Left | CCTCTTGTCTTCTCCTTTGCAC |
| 2 | Right | GGGCAGGCTCAAGAGTGA |
| 3 | Left | CCTCTTGTCTTCTCCTTTGCAC |
| 4 | Right | AGGGCAGGCTCAAGAGTGA |
| 5 | Left | CCTCTTGTCTTCTCCTTTGCAC |
| 6 | Right | AAGGGCAGGCTCAAGAGTGA |
| 7 | Left | CCTCTTGTCTTCTCCTTTGCAC |
| 8 | Right | CAAGGGCAGGCTCAAGAGTGA |
| 9 | Left | CTCTTGTCTTCTCCTTTGCAC |
| 10 | Right | CAAGGGCAGGCTCAAGAGT |
| 11 | Left | CTCTTGTCTTCTCCTTTGCAC |
| 12 | Right | AAGGGCAGGCTCAAGAGTG |
| 13 | Left | CTCTTGTCTTCTCCTTTGCAC |
| 14 | Right | GGGCAGGCTCAAGAGTGA |
| 15 | Left | CTCTTGTCTTCTCCTTTGCAC |
| 16 | Right | AGGGCAGGCTCAAGAGTGA |
| 17 | Left | CCTCTTGTCTTCTCCTTTGCAC |
| 18 | Right | CCAAGGGCAGGCTCAAGAGTGA |
| 19 | Left | CTCTTGTCTTCTCCTTTGCAC |
| 20 | Right | AAGGGCAGGCTCAAGAGTGA |
| 21 | Left | CCTCTTGTCTTCTCCTTTGCAC |
| 22 | Right | AGCCAAGGGCAGGCTCAA |
| 23 | Left | CTCTTGTCTTCTCCTTTGCAC |
| 24 | Right | AGGGCAGGCTCAAGAGTG |
| 25 | Left | CCTCTTGTCTTCTCCTTTGC |
| 26 | Right | GGGCAGGCTCAAGAGTGA |
| 27 | Left | CCTCTTGTCTTCTCCTTTGC |
| 28 | Right | AGGGCAGGCTCAAGAGTGA |
| 29 | Left | CTCTTGTCTTCTCCTTTGCAC |
| 30 | Right | CAAGGGCAGGCTCAAGAGTG |
| 31 | Left | CCTCTTGTCTTCTCCTTTGC |
| 32 | Right | AAGGGCAGGCTCAAGAGTGA |
| 33 | Left | CCTCTTGTCTTCTCCTTTGCAC |
| 34 | Right | AGCCAAGGGCAGGCTCAAGAGTGA |
| 35 | Left | TCTTGTCTTCTCCTTTGCAC |
| 36 | Right | CAAGGGCAGGCTCAAGAGT |
| 37 | Left | TCTTGTCTTCTCCTTTGCAC |
| 38 | Right | AAGGGCAGGCTCAAGAGTG |
| 39 | Left | CTCTTGTCTTCTCCTTTGCAC |
| 40 | Right | CCAAGGGCAGGCTCAAGAGT |

ALK Exon21 151-200 bases

| | | |
|---|---|---|
| 41 | Left | ACTCTGTCTCCTCTTGTCTTCTCCT |
| 42 | Right | CTGAGAACTGCAGCCTACAGAGT |
| 43 | Left | CTCTGTCTCCTCTTGTCTTCTCCTT |
| 44 | Right | CTGAGAACTGCAGCCTACAGAGT |
| 45 | Left | TCTGTCTCCTCTTGTCTTCTCCTT |
| 46 | Right | CTGAGAACTGCAGCCTACAGAGT |
| 47 | Left | TCTGTCTCCTCTTGTCTTCTCCTTT |
| 48 | Right | CTGAGAACTGCAGCCTACAGAGT |
| 49 | Left | ACTCTGTCTCCTCTTGTCTTCTCCT |
| 50 | Right | AGAACTGCAGCCTACAGAGTCC |
| 51 | Left | CTCTGTCTCCTCTTGTCTTCTCCT |
| 52 | Right | CTGAGAACTGCAGCCTACAGAGT |
| 53 | Left | TTGACTCTGTCTCCTCTTGTCTTCT |
| 54 | Right | CTGAGAACTGCAGCCTACAGAG |
| 55 | Left | CTGTCTCCTCTTGTCTTCTCCTTT |
| 56 | Right | CTGAGAACTGCAGCCTACAGAGT |
| 57 | Left | CTCTGTCTCCTCTTGTCTTCTCCTT |
| 58 | Right | AGAACTGCAGCCTACAGAGTCC |
| 59 | Left | ACTCTGTCTCCTCTTGTCTTCTCCT |
| 60 | Right | CTGAGAACTGCAGCCTACAGAG |
| 61 | Left | GTTTGACTCTGTCTCCTCTTGTCTT |
| 62 | Right | CTGAGAACTGCAGCCTACAGAG |
| 63 | Left | TCTGTCTCCTCTTGTCTTCTCCTT |
| 64 | Right | AGAACTGCAGCCTACAGAGTCC |
| 65 | Left | ACTCTGTCTCCTCTTGTCTTCTCCT |
| 66 | Right | GAGAACTGCAGCCTACAGAGTCC |
| 67 | Left | TCTGTCTCCTCTTGTCTTCTCCTTT |
| 68 | Right | AGAACTGCAGCCTACAGAGTCC |
| 69 | Left | CTGTCTCCTCTTGTCTTCTCCTTTG |
| 70 | Right | CTGAGAACTGCAGCCTACAGAGT |
| 71 | Left | CTCTGTCTCCTCTTGTCTTCTCCT |
| 72 | Right | AGAACTGCAGCCTACAGAGTCC |
| 73 | Left | CTGTTTGACTCTGTCTCCTCTTGTC |
| 74 | Right | CTGAGAACTGCAGCCTACAGAG |
| 75 | Left | CTCTGTCTCCTCTTGTCTTCTCCTT |
| 76 | Right | CTGAGAACTGCAGCCTACAGAG |
| 77 | Left | CTGTCTCCTCTTGTCTTCTCCTTT |
| 78 | Right | AGAACTGCAGCCTACAGAGTCC |
| 79 | Left | ACTCTGTCTCCTCTTGTCTTCTCC |
| 80 | Right | AGAACTGCAGCCTACAGAGTCC |

ALK Exon21 201-300 bases

| | | |
|---|---|---|
| 81 | Left | TGTTGAGGGTATTACTCCTGAGTGT |
| 82 | Right | CTGAGAACTGCAGCCTACAGAGT |
| 83 | Left | TTGAGGGTATTACTCCTGAGTGTGT |
| 84 | Right | CTGAGAACTGCAGCCTACAGAGT |
| 85 | Left | GTTGAGGGTATTACTCCTGAGTGTG |
| 86 | Right | AGAACTGCAGCCTACAGAGTCC |
| 87 | Left | TGTTGAGGGTATTACTCCTGAGTGT |
| 88 | Right | AGAACTGCAGCCTACAGAGTCC |
| 89 | Left | TTGAGGGTATTACTCCTGAGTGTGT |
| 90 | Right | AGAACTGCAGCCTACAGAGTCC |
| 91 | Left | CTCTCGTGTTTGTCCACTAAATGT |
| 92 | Right | CTGAGAACTGCAGCCTACAGAGT |
| 93 | Left | TGAGGGTATTACTCCTGAGTGTGTAT |
| 94 | Right | CTGAGAACTGCAGCCTACAGAGT |
| 95 | Left | GTTGAGGGTATTACTCCTGAGTGTG |
| 96 | Right | CTGAGAACTGCAGCCTACAGAG |
| 97 | Left | TGTTGAGGGTATTACTCCTGAGTGT |
| 98 | Right | CTGAGAACTGCAGCCTACAGAG |
| 99 | Left | TTGAGGGTATTACTCCTGAGTGTGT |
| 100 | Right | CTGAGAACTGCAGCCTACAGAG |
| 101 | Left | TGTTGAGGGTATTACTCCTGAGTGT |
| 102 | Right | TGAGAACTGCAGCCTACAGAGT |
| 103 | Left | TTGAGGGTATTACTCCTGAGTGTGT |
| 104 | Right | TGAGAACTGCAGCCTACAGAGT |
| 105 | Left | TGAGGGTATTACTCCTGAGTGTGT |
| 106 | Right | CTGAGAACTGCAGCCTACAGAGT |
| 107 | Left | GTTGAGGGTATTACTCCTGAGTGTG |
| 108 | Right | GAGAACTGCAGCCTACAGAGTCC |
| 109 | Left | TGTTGAGGGTATTACTCCTGAGTGT |
| 110 | Right | GAGAACTGCAGCCTACAGAGTCC |
| 111 | Left | TTGAGGGTATTACTCCTGAGTGTGT |
| 112 | Right | GAGAACTGCAGCCTACAGAGTCC |
| 113 | Left | GTTGAGGGTATTACTCCTGAGTGTGT |
| 114 | Right | CTGAGAACTGCAGCCTACAGAGT |
| 115 | Left | CTCTCGTGTTTGTCCACTAAATGTG |
| 116 | Right | CTGAGAACTGCAGCCTACAGAGT |
| 117 | Left | TTGACTCTGTCTCCTCTTGTCTTCT |
| 118 | Right | GAGGCTGTGAGCTGAGAACTG |
| 119 | Left | CTCTCGTGTTTGTCCACTAAATGT |
| 120 | Right | AGAACTGCAGCCTACAGAGTCC |

ALK Exon21 301-400 bases

| | | |
|---|---|---|
| 121 | Left | GAATCCTTCTTACCAGTTTTCAGGT |
| 122 | Right | CTGAGAACTGCAGCCTACAGAGT |
| 123 | Left | GTTGGAATCCTTCTTACCAGTTTTC |
| 124 | Right | CTGAGAACTGCAGCCTACAGAGT |
| 125 | Left | AATCCTTCTTACCAGTTTTCAGGTG |
| 126 | Right | CTGAGAACTGCAGCCTACAGAGT |
| 127 | Left | ATCCTTCTTACCAGTTTTCAGGTG |
| 128 | Right | CTGAGAACTGCAGCCTACAGAGT |
| 129 | Left | GAATCCTTCTTACCAGTTTTCAGGT |
| 130 | Right | AGAACTGCAGCCTACAGAGTCC |
| 131 | Left | TTGGAATCCTTCTTACCAGTTTTC |
| 132 | Right | CTGAGAACTGCAGCCTACAGAGT |
| 133 | Left | GTTGGAATCCTTCTTACCAGTTTTC |
| 134 | Right | AGAACTGCAGCCTACAGAGTCC |
| 135 | Left | GAATCCTTCTTACCAGTTTTCAGG |
| 136 | Right | CTGAGAACTGCAGCCTACAGAGT |
| 137 | Left | GGAATCCTTCTTACCAGTTTTCAG |
| 138 | Right | CTGAGAACTGCAGCCTACAGAGT |
| 139 | Left | ATGTTGAGGGTATTACTCCTGAGTGT |
| 140 | Right | CTGAGAACTGCAGCCTACAGAGT |
| 141 | Left | GAATCCTTCTTACCAGTTTTCAGGT |
| 142 | Right | CTGAGAACTGCAGCCTACAGAG |
| 143 | Left | CAAAGCCATGTTGAGGGTATTACT |
| 144 | Right | CTGAGAACTGCAGCCTACAGAGT |
| 145 | Left | GAATCCTTCTTACCAGTTTTCAGGT |
| 146 | Right | TGAGAACTGCAGCCTACAGAGT |
| 147 | Left | GAATCCTTCTTACCAGTTTTCAGGT |
| 148 | Right | GAGAACTGCAGCCTACAGAGTCC |
| 149 | Left | GTTGGAATCCTTCTTACCAGTTTTC |
| 150 | Right | CTGAGAACTGCAGCCTACAGAG |
| 151 | Left | AATCCTTCTTACCAGTTTTCAGGTG |
| 152 | Right | AGAACTGCAGCCTACAGAGTCC |
| 153 | Left | ATCCTTCTTACCAGTTTTCAGGTG |
| 154 | Right | AGAACTGCAGCCTACAGAGTCC |
| 155 | Left | GGTTGGAATCCTTCTTACCAGTTT |
| 156 | Right | CTGAGAACTGCAGCCTACAGAGT |
| 157 | Left | TGGAATCCTTCTTACCAGTTTTCAG |
| 158 | Right | CTGAGAACTGCAGCCTACAGAGT |
| 159 | Left | TTGGAATCCTTCTTACCAGTTTTC |
| 160 | Right | AGAACTGCAGCCTACAGAGTCC |

ALK Exon21-22 301-400 bases

| | | |
|---|---|---|
| 161 | Left | TTGACTCTGTCTCCTCTTGTCTTCT |
| 162 | Right | TGGAGATATCGATCTGTTAGAAACC |
| 163 | Left | TTTGACTCTGTCTCCTCTTGTCTTC |
| 164 | Right | CCTTGGAGATATCGATCTGTTAGAA |
| 165 | Left | ACTCTGTCTCCTCTTGTCTTCTCCT |
| 166 | Right | CCTTGGAGATATCGATCTGTTAGAA |
| 167 | Left | GTTTGACTCTGTCTCCTCTTGTCTT |
| 168 | Right | CCTTGGAGATATCGATCTGTTAGAA |
| 169 | Left | TTTGACTCTGTCTCCTCTTGTCTTC |
| 170 | Right | TGGAGATATCGATCTGTTAGAAACC |
| 171 | Left | ACTCTGTCTCCTCTTGTCTTCTCCT |
| 172 | Right | TGGAGATATCGATCTGTTAGAAACC |
| 173 | Left | TTGACTCTGTCTCCTCTTGTCTTCT |
| 174 | Right | TATCGATCTGTTAGAAACCTCTCCA |
| 175 | Left | TGACTCTGTCTCCTCTTGTCTTCTC |
| 176 | Right | CCTTGGAGATATCGATCTGTTAGAA |
| 177 | Left | GTTTGACTCTGTCTCCTCTTGTCTT |
| 178 | Right | TGGAGATATCGATCTGTTAGAAACC |
| 179 | Left | TGACTCTGTCTCCTCTTGTCTTCTC |
| 180 | Right | TGGAGATATCGATCTGTTAGAAACC |
| 181 | Left | TGTTTGACTCTGTCTCCTCTTGTCT |
| 182 | Right | CCTTGGAGATATCGATCTGTTAGAA |
| 183 | Left | CTGTTTGACTCTGTCTCCTCTTGTC |
| 184 | Right | CCTTGGAGATATCGATCTGTTAGAA |
| 185 | Left | CTCTGTCTCCTCTTGTCTTCTCCTT |
| 186 | Right | CCTTGGAGATATCGATCTGTTAGAA |
| 187 | Left | TTTGACTCTGTCTCCTCTTGTCTTC |
| 188 | Right | TATCGATCTGTTAGAAACCTCTCCA |
| 189 | Left | ACTCTGTCTCCTCTTGTCTTCTCCT |
| 190 | Right | TATCGATCTGTTAGAAACCTCTCCA |
| 191 | Left | TTGACTCTGTCTCCTCTTGTCTTCT |
| 192 | Right | GTTAGAAACCTCTCCAGGTTCTTTG |
| 193 | Left | GTTTGACTCTGTCTCCTCTTGTCTT |
| 194 | Right | TATCGATCTGTTAGAAACCTCTCCA |
| 195 | Left | TGTTTGACTCTGTCTCCTCTTGTCT |
| 196 | Right | TGGAGATATCGATCTGTTAGAAACC |
| 197 | Left | CTGTTTGACTCTGTCTCCTCTTGTC |
| 198 | Right | TGGAGATATCGATCTGTTAGAAACC |
| 199 | Left | CTCTGTCTCCTCTTGTCTTCTCCTT |
| 200 | Right | TGGAGATATCGATCTGTTAGAAACC |

ALK Exon21-22 401-500 bases

| | | |
|---|---|---|
| 201 | Left | TTGACTCTGTCTCCTCTTGTCTTCT |
| 202 | Right | TAGAATGTTTGGGAGTCTCCTACTG |
| 203 | Left | TTGACTCTGTCTCCTCTTGTCTTCT |
| 204 | Right | GTTGTTCCATTCTGGTAAGAAGTGT |
| 205 | Left | GTTGAGGGTATTACTCCTGAGTGTG |
| 206 | Right | CCTTGGAGATATCGATCTGTTAGAA |
| 207 | Left | TGTTGAGGGTATTACTCCTGAGTGT |
| 208 | Right | CCTTGGAGATATCGATCTGTTAGAA |
| 209 | Left | TTGAGGGTATTACTCCTGAGTGTGT |
| 210 | Right | CCTTGGAGATATCGATCTGTTAGAA |
| 211 | Left | GTTGAGGGTATTACTCCTGAGTGTG |
| 212 | Right | TGGAGATATCGATCTGTTAGAAACC |
| 213 | Left | TGTTGAGGGTATTACTCCTGAGTGT |
| 214 | Right | TGGAGATATCGATCTGTTAGAAACC |
| 215 | Left | TTGAGGGTATTACTCCTGAGTGTGT |
| 216 | Right | TGGAGATATCGATCTGTTAGAAACC |
| 217 | Left | TTGACTCTGTCTCCTCTTGTCTTCT |
| 218 | Right | GAAGTGTCTAGAATGTTTGGGAGTC |
| 219 | Left | TTTGACTCTGTCTCCTCTTGTCTTC |
| 220 | Right | TAGAATGTTTGGGAGTCTCCTACTG |
| 221 | Left | ACTCTGTCTCCTCTTGTCTTCTCCT |
| 222 | Right | TAGAATGTTTGGGAGTCTCCTACTG |
| 223 | Left | TTGACTCTGTCTCCTCTTGTCTTCT |
| 224 | Right | TGTTCCATTCTGGTAAGAAGTGTCT |
| 225 | Left | GTTTGACTCTGTCTCCTCTTGTCTT |
| 226 | Right | TAGAATGTTTGGGAGTCTCCTACTG |
| 227 | Left | TTTGACTCTGTCTCCTCTTGTCTTC |
| 228 | Right | GTTGTTCCATTCTGGTAAGAAGTGT |
| 229 | Left | ACTCTGTCTCCTCTTGTCTTCTCCT |
| 230 | Right | GTTGTTCCATTCTGGTAAGAAGTGT |
| 231 | Left | GTTTGACTCTGTCTCCTCTTGTCTT |
| 232 | Right | GTTGTTCCATTCTGGTAAGAAGTGT |
| 233 | Left | TGACTCTGTCTCCTCTTGTCTTCTC |
| 234 | Right | TAGAATGTTTGGGAGTCTCCTACTG |
| 235 | Left | TGACTCTGTCTCCTCTTGTCTTCTC |
| 236 | Right | GTTGTTCCATTCTGGTAAGAAGTGT |
| 237 | Left | GTTGAGGGTATTACTCCTGAGTGTG |
| 238 | Right | TATCGATCTGTTAGAAACCTCTCCA |
| 239 | Left | TGTTGAGGGTATTACTCCTGAGTGT |
| 240 | Right | TATCGATCTGTTAGAAACCTCTCCA |

ALK Exon21-22 501-600 bases

| | | |
|---|---|---|
| 241 | Left | GAATCCTTCTTACCAGTTTTCAGGT |
| 242 | Right | CCTTGGAGATATCGATCTGTTAGAA |
| 243 | Left | GAATCCTTCTTACCAGTTTTCAGGT |
| 244 | Right | TGGAGATATCGATCTGTTAGAAACC |
| 245 | Left | GTTGAGGGTATTACTCCTGAGTGTG |
| 246 | Right | TAGAATGTTTGGGAGTCTCCTACTG |
| 247 | Left | TGTTGAGGGTATTACTCCTGAGTGT |
| 248 | Right | TAGAATGTTTGGGAGTCTCCTACTG |
| 249 | Left | TTGAGGGTATTACTCCTGAGTGTGT |
| 250 | Right | TAGAATGTTTGGGAGTCTCCTACTG |
| 251 | Left | GTTGAGGGTATTACTCCTGAGTGTG |
| 252 | Right | GTTGTTCCATTCTGGTAAGAAGTGT |
| 253 | Left | TGTTGAGGGTATTACTCCTGAGTGT |
| 254 | Right | GTTGTTCCATTCTGGTAAGAAGTGT |
| 255 | Left | TTGAGGGTATTACTCCTGAGTGTGT |
| 256 | Right | GTTGTTCCATTCTGGTAAGAAGTGT |
| 257 | Left | GTTGGAATCCTTCTTACCAGTTTTC |
| 258 | Right | CCTTGGAGATATCGATCTGTTAGAA |
| 259 | Left | GTTGAGGGTATTACTCCTGAGTGTG |
| 260 | Right | GAAGTGTCTAGAATGTTTGGGAGTC |
| 261 | Left | TTGAGGGTATTACTCCTGAGTGTGT |
| 262 | Right | GAAGTGTCTAGAATGTTTGGGAGTC |
| 263 | Left | TGTTGAGGGTATTACTCCTGAGTGT |
| 264 | Right | GAAGTGTCTAGAATGTTTGGGAGTC |
| 265 | Left | GAATCCTTCTTACCAGTTTTCAGGT |
| 266 | Right | TATCGATCTGTTAGAAACCTCTCCA |
| 267 | Left | GTTGAGGGTATTACTCCTGAGTGTG |
| 268 | Right | TGTTCCATTCTGGTAAGAAGTGTCT |
| 269 | Left | TGTTGAGGGTATTACTCCTGAGTGT |
| 270 | Right | TGTTCCATTCTGGTAAGAAGTGTCT |
| 271 | Left | TTGAGGGTATTACTCCTGAGTGTGT |
| 272 | Right | TGTTCCATTCTGGTAAGAAGTGTCT |
| 273 | Left | AATCCTTCTTACCAGTTTTCAGGTG |
| 274 | Right | CCTTGGAGATATCGATCTGTTAGAA |
| 275 | Left | GTTGGAATCCTTCTTACCAGTTTTC |
| 276 | Right | TATCGATCTGTTAGAAACCTCTCCA |
| 277 | Left | ATCCTTCTTACCAGTTTTCAGGTG |
| 278 | Right | CCTTGGAGATATCGATCTGTTAGAA |
| 279 | Left | GTTGAGGGTATTACTCCTGAGTGTG |
| 280 | Right | TTGTTCCATTCTGGTAAGAAGTGTC |

ALK Exon21-22 601-800 bases

| | | |
|---|---|---|
| 281 | Left | TTGACTCTGTCTCCTCTTGTCTTCT |
| 282 | Right | AAAGTCTAGCATGCTCCATTTCTTA |
| 283 | Left | GAATCCTTCTTACCAGTTTTCAGGT |
| 284 | Right | TAGAATGTTTGGGAGTCTCCTACTG |
| 285 | Left | GAATCCTTCTTACCAGTTTTCAGGT |
| 286 | Right | AAAGTCTAGCATGCTCCATTTCTTA |
| 287 | Left | GTTGAGGGTATTACTCCTGAGTGTG |
| 288 | Right | AAAGTCTAGCATGCTCCATTTCTTA |
| 289 | Left | TGTTGAGGGTATTACTCCTGAGTGT |
| 290 | Right | AAAGTCTAGCATGCTCCATTTCTTA |
| 291 | Left | TTGAGGGTATTACTCCTGAGTGTGT |
| 292 | Right | AAAGTCTAGCATGCTCCATTTCTTA |
| 293 | Left | CCTCTGTCACTCACTGGAAATACTC |
| 294 | Right | CCTTGGAGATATCGATCTGTTAGAA |
| 295 | Left | TCCTCTGTCACTCACTGGAAATACT |
| 296 | Right | CCTTGGAGATATCGATCTGTTAGAA |
| 297 | Left | CTCTGTCACTCACTGGAAATACTCC |
| 298 | Right | CCTTGGAGATATCGATCTGTTAGAA |
| 299 | Left | TTTGACTCTGTCTCCTCTTGTCTTC |
| 300 | Right | AAAGTCTAGCATGCTCCATTTCTTA |
| 301 | Left | ACTCTGTCTCCTCTTGTCTTCTCCT |
| 302 | Right | AAAGTCTAGCATGCTCCATTTCTTA |
| 303 | Left | TTGACTCTGTCTCCTCTTGTCTTCT |
| 304 | Right | GGTCTTGGAGGGAGATTATATCTTG |
| 305 | Left | GTTGGAATCCTTCTTACCAGTTTTC |
| 306 | Right | TAGAATGTTTGGGAGTCTCCTACTG |
| 307 | Left | GTTTGACTCTGTCTCCTCTTGTCTT |
| 308 | Right | AAAGTCTAGCATGCTCCATTTCTTA |
| 309 | Left | GAATCCTTCTTACCAGTTTTCAGGT |
| 310 | Right | GAAGTGTCTAGAATGTTTGGGAGTC |
| 311 | Left | CCTCTGTCACTCACTGGAAATACTC |
| 312 | Right | TGGAGATATCGATCTGTTAGAAACC |
| 313 | Left | TCCTCTGTCACTCACTGGAAATACT |
| 314 | Right | TGGAGATATCGATCTGTTAGAAACC |
| 315 | Left | CTCTGTCACTCACTGGAAATACTCC |
| 316 | Right | TGGAGATATCGATCTGTTAGAAACC |
| 317 | Left | GTTGGAATCCTTCTTACCAGTTTTC |
| 318 | Right | AAAGTCTAGCATGCTCCATTTCTTA |
| 319 | Left | TGACTCTGTCTCCTCTTGTCTTCTC |
| 320 | Right | AAAGTCTAGCATGCTCCATTTCTTA |

ALK Exon21-22 801-1000 bases

| | | |
|---|---|---|
| 321 | Left | CTCTCCTCAAAATTCATTCAGATGT |
| 322 | Right | CCTTGGAGATATCGATCTGTTAGAA |
| 323 | Left | ATGTTGGCTTACATTAACTCCCATA |
| 324 | Right | CCTTGGAGATATCGATCTGTTAGAA |
| 325 | Left | CTCTCCTCAAAATTCATTCAGATGT |
| 326 | Right | TAGAATGTTTGGGAGTCTCCTACTG |
| 327 | Left | ATGTTGGCTTACATTAACTCCCATA |
| 328 | Right | TAGAATGTTTGGGAGTCTCCTACTG |
| 329 | Left | CTCTCCTCAAAATTCATTCAGATGT |
| 330 | Right | TGGAGATATCGATCTGTTAGAAACC |
| 331 | Left | CTCTCCTCAAAATTCATTCAGATGT |
| 332 | Right | GTTGTTCCATTCTGGTAAGAAGTGT |
| 333 | Left | ATGTTGGCTTACATTAACTCCCATA |
| 334 | Right | TGGAGATATCGATCTGTTAGAAACC |
| 335 | Left | AAAATTCATTCAGATGTGCTCTCTC |
| 336 | Right | TAGAATGTTTGGGAGTCTCCTACTG |
| 337 | Left | AAAATTCATTCAGATGTGCTCTCTC |
| 338 | Right | TGGAGATATCGATCTGTTAGAAACC |
| 339 | Left | AAAATTCATTCAGATGTGCTCTCTC |
| 340 | Right | GTTGTTCCATTCTGGTAAGAAGTGT |
| 341 | Left | CTCTCCTCAAAATTCATTCAGATGT |
| 342 | Right | GAAGTGTCTAGAATGTTTGGGAGTC |
| 343 | Left | CTCTCCTCAAAATTCATTCAGATGT |
| 344 | Right | TGTTCCATTCTGGTAAGAAGTGTCT |
| 345 | Left | CTCTCCTCAAAATTCATTCAGATGT |
| 346 | Right | TATCGATCTGTTAGAAACCTCTCCA |
| 347 | Left | ATGTTGGCTTACATTAACTCCCATA |
| 348 | Right | TATCGATCTGTTAGAAACCTCTCCA |
| 349 | Left | AAAATTCATTCAGATGTGCTCTCTC |
| 350 | Right | GAAGTGTCTAGAATGTTTGGGAGTC |
| 351 | Left | AAAATTCATTCAGATGTGCTCTCTC |
| 352 | Right | TGTTCCATTCTGGTAAGAAGTGTCT |
| 353 | Left | TGGCTTACATTAACTCCCATAGTTT |
| 354 | Right | CCTTGGAGATATCGATCTGTTAGAA |
| 355 | Left | TTGGCTTACATTAACTCCCATAGTT |
| 356 | Right | CCTTGGAGATATCGATCTGTTAGAA |
| 357 | Left | TGTTGGCTTACATTAACTCCCATAG |
| 358 | Right | CCTTGGAGATATCGATCTGTTAGAA |
| 359 | Left | AAAATTCATTCAGATGTGCTCTCTC |
| 360 | Right | TATCGATCTGTTAGAAACCTCTCCA |

ALK Exon21-22 2 kb

| | | |
|---|---|---|
| 361 | Left | CTCTCCTCAAAATTCATTCAGATGT |
| 362 | Right | GCAGGAGAGTGTCTTTCTCAGATAC |
| 363 | Left | ATGTTGGCTTACATTAACTCCCATA |
| 364 | Right | GCAGGAGAGTGTCTTTCTCAGATAC |
| 365 | Left | AAAATTCATTCAGATGTGCTCTCTC |
| 366 | Right | GCAGGAGAGTGTCTTTCTCAGATAC |
| 367 | Left | CTCTCCTCAAAATTCATTCAGATGT |
| 368 | Right | GGAGAGTGTCTTTCTCAGATACTGG |
| 369 | Left | ATGTTGGCTTACATTAACTCCCATA |
| 370 | Right | GGAGAGTGTCTTTCTCAGATACTGG |
| 371 | Left | AAAATTCATTCAGATGTGCTCTCTC |
| 372 | Right | CAAAGTTACATTTTCAGCAGCTACA |
| 373 | Left | AAAATTCATTCAGATGTGCTCTCTC |
| 374 | Right | GGAGAGTGTCTTTCTCAGATACTGG |
| 375 | Left | GAATCCTTCTTACCAGTTTTCAGGT |
| 376 | Right | CAAAGTTACATTTTCAGCAGCTACA |
| 377 | Left | CTCTCCTCAAAATTCATTCAGATGT |
| 378 | Right | GCAGCTACAATGTATAAAGGCATTC |
| 379 | Left | GTTGAGGGTATTACTCCTGAGTGTG |
| 380 | Right | CAAAGTTACATTTTCAGCAGCTACA |
| 381 | Left | TGTTGAGGGTATTACTCCTGAGTGT |
| 382 | Right | CAAAGTTACATTTTCAGCAGCTACA |
| 383 | Left | ATGTTGGCTTACATTAACTCCCATA |
| 384 | Right | TTAACATGATCCCTTTAGGACACAC |
| 385 | Left | ATGTTGGCTTACATTAACTCCCATA |
| 386 | Right | TGTTAACATGATCCCTTTAGGACAC |
| 387 | Left | ATGTTGGCTTACATTAACTCCCATA |
| 388 | Right | GTTAACATGATCCCTTTAGGACACA |
| 389 | Left | AAAATTCATTCAGATGTGCTCTCTC |
| 390 | Right | GCAGCTACAATGTATAAAGGCATTC |
| 391 | Left | CTCTGTCACTCACTGGAAATACTCC |
| 392 | Right | GCAGGAGAGTGTCTTTCTCAGATAC |
| 393 | Left | TCCTCTGTCACTCACTGGAAATACT |
| 394 | Right | GCAGGAGAGTGTCTTTCTCAGATAC |
| 395 | Left | TGGCTTACATTAACTCCCATAGTTT |
| 396 | Right | GCAGGAGAGTGTCTTTCTCAGATAC |
| 397 | Left | TTGGCTTACATTAACTCCCATAGTT |
| 398 | Right | GCAGGAGAGTGTCTTTCTCAGATAC |
| 399 | Left | TGTTGGCTTACATTAACTCCCATAG |
| 400 | Right | GCAGGAGAGTGTCTTTCTCAGATAC |

ALK Exon22 90-150 bases

| | | |
|---|---|---|
| 401 | Left | AGTTCTCAGCTCACAGCCTCCT |
| 402 | Right | AGGGTGTCTCTCTGTGGCTTTAC |
| 403 | Left | AGTTCTCAGCTCACAGCCTCCT |
| 404 | Right | GGGTGTCTCTCTGTGGCTTTAC |
| 405 | Left | GTTCTCAGCTCACAGCCTCCT |
| 406 | Right | AGGGTGTCTCTCTGTGGCTTTAC |
| 407 | Left | TAGGCTGCAGTTCTCAGCTCAC |
| 408 | Right | AGGGTGTCTCTCTGTGGCTTTAC |
| 409 | Left | GTTCTCAGCTCACAGCCTCCT |
| 410 | Right | GGGTGTCTCTCTGTGGCTTTAC |
| 411 | Left | AGTTCTCAGCTCACAGCCTCCT |
| 412 | Right | AGGGTGTCTCTCTGTGGCTTTA |
| 413 | Left | TAGGCTGCAGTTCTCAGCTCAC |
| 414 | Right | GGGTGTCTCTCTGTGGCTTTAC |
| 415 | Left | TTCTCAGCTCACAGCCTCCT |
| 416 | Right | AGGGTGTCTCTCTGTGGCTTTAC |
| 417 | Left | GTTCTCAGCTCACAGCCTCCT |
| 418 | Right | AGGGTGTCTCTCTGTGGCTTTA |
| 419 | Left | TTCTCAGCTCACAGCCTCCT |
| 420 | Right | GGGTGTCTCTCTGTGGCTTTAC |
| 421 | Left | TAGGCTGCAGTTCTCAGCTCAC |
| 422 | Right | AGGGTGTCTCTCTGTGGCTTTA |
| 423 | Left | GCTGCAGTTCTCAGCTCACAG |
| 424 | Right | AGGGTGTCTCTCTGTGGCTTTAC |
| 425 | Left | TTCTCAGCTCACAGCCTCCT |
| 426 | Right | AGGGTGTCTCTCTGTGGCTTTA |
| 427 | Left | GCTGCAGTTCTCAGCTCACAG |
| 428 | Right | GGGTGTCTCTCTGTGGCTTTAC |
| 429 | Left | AGTTCTCAGCTCACAGCCTCCT |
| 430 | Right | GAGGGTGTCTCTCTGTGGCTTTAC |
| 431 | Left | AGTTCTCAGCTCACAGCCTCCTC |
| 432 | Right | AGGGTGTCTCTCTGTGGCTTTAC |
| 433 | Left | GTTCTCAGCTCACAGCCTCCT |
| 434 | Right | GAGGGTGTCTCTCTGTGGCTTTAC |
| 435 | Left | TAGGCTGCAGTTCTCAGCTCAC |
| 436 | Right | GAGGGTGTCTCTCTGTGGCTTTAC |
| 437 | Left | AGTTCTCAGCTCACAGCCTCCTC |
| 438 | Right | GGGTGTCTCTCTGTGGCTTTAC |
| 439 | Left | TCTCAGCTCACAGCCTCCTC |
| 440 | Right | AGGGTGTCTCTCTGTGGCTTTAC |

ALK Exon22 151-200 bases

| | | |
|---|---|---|
| 441 | Left | AGTTCTCAGCTCACAGCCTCCT |
| 442 | Right | TATCGATCTGTTAGAAACCTCTCCA |
| 443 | Left | GTTCTCAGCTCACAGCCTCCT |
| 444 | Right | TATCGATCTGTTAGAAACCTCTCCA |
| 445 | Left | TTCTCAGCTCACAGCCTCCT |
| 446 | Right | TATCGATCTGTTAGAAACCTCTCCA |
| 447 | Left | AGTTCTCAGCTCACAGCCTCCT |
| 448 | Right | GTTAGAAACCTCTCCAGGTTCTTTG |
| 449 | Left | GTTCTCAGCTCACAGCCTCCT |
| 450 | Right | GTTAGAAACCTCTCCAGGTTCTTTG |
| 451 | Left | TCTCAGCTCACAGCCTCCTC |
| 452 | Right | TGGAGATATCGATCTGTTAGAAACC |
| 453 | Left | TAGGCTGCAGTTCTCAGCTCAC |
| 454 | Right | GTTAGAAACCTCTCCAGGTTCTTTG |
| 455 | Left | AGTTCTCAGCTCACAGCCTCCT |
| 456 | Right | GATATCGATCTGTTAGAAACCTCTCC |
| 457 | Left | TTCTCAGCTCACAGCCTCCT |
| 458 | Right | GTTAGAAACCTCTCCAGGTTCTTTG |
| 459 | Left | AGTTCTCAGCTCACAGCCTCCT |
| 460 | Right | TTAGAAACCTCTCCAGGTTCTTTG |
| 461 | Left | AGTTCTCAGCTCACAGCCTCCTC |
| 462 | Right | TATCGATCTGTTAGAAACCTCTCCA |
| 463 | Left | GTTCTCAGCTCACAGCCTCCT |
| 464 | Right | GATATCGATCTGTTAGAAACCTCTCC |
| 465 | Left | GTTCTCAGCTCACAGCCTCCT |
| 466 | Right | TTAGAAACCTCTCCAGGTTCTTTG |
| 467 | Left | AGTTCTCAGCTCACAGCCTCCT |
| 468 | Right | ATCGATCTGTTAGAAACCTCTCCAG |
| 469 | Left | TAGGCTGCAGTTCTCAGCTCAC |
| 470 | Right | TTAGAAACCTCTCCAGGTTCTTTG |
| 471 | Left | AGCTCACAGCCTCCTCCTC |
| 472 | Right | CCTTGGAGATATCGATCTGTTAGAA |
| 473 | Left | GCTGCAGTTCTCAGCTCACAG |
| 474 | Right | GTTAGAAACCTCTCCAGGTTCTTTG |
| 475 | Left | TCTCAGCTCACAGCCTCCTC |
| 476 | Right | TATCGATCTGTTAGAAACCTCTCCA |
| 477 | Left | TTCTCAGCTCACAGCCTCCT |
| 478 | Right | GATATCGATCTGTTAGAAACCTCTCC |
| 479 | Left | GTTCTCAGCTCACAGCCTCCTC |
| 480 | Right | TATCGATCTGTTAGAAACCTCTCCA |

ALK Exon22 201-300 bases

| | | |
|---|---|---|
| 481 | Left | GGACTCTGTAGGCTGCAGTTCTC |
| 482 | Right | CCTTGGAGATATCGATCTGTTAGAA |
| 483 | Left | GGACTCTGTAGGCTGCAGTTCTC |
| 484 | Right | TAGAATGTTTGGGAGTCTCCTACTG |
| 485 | Left | GGACTCTGTAGGCTGCAGTTCTC |
| 486 | Right | TGGAGATATCGATCTGTTAGAAACC |
| 487 | Left | GGACTCTGTAGGCTGCAGTTCTC |
| 488 | Right | GAAGTGTCTAGAATGTTTGGGAGTC |
| 489 | Left | GGACTCTGTAGGCTGCAGTTCTC |
| 490 | Right | TATCGATCTGTTAGAAACCTCTCCA |
| 491 | Left | GGACTCTGTAGGCTGCAGTTCTC |
| 492 | Right | GTTAGAAACCTCTCCAGGTTCTTTG |
| 493 | Left | GGACTCTGTAGGCTGCAGTTCTC |
| 494 | Right | AAGAAGTGTCTAGAATGTTTGGGAGT |
| 495 | Left | GGACTCTGTAGGCTGCAGTTCTC |
| 496 | Right | TGGTAAGAAGTGTCTAGAATGTTTGG |
| 497 | Left | GGACTCTGTAGGCTGCAGTTCTC |
| 498 | Right | AGAATGTTTGGGAGTCTCCTACTG |
| 499 | Left | GGACTCTGTAGGCTGCAGTTCTC |
| 500 | Right | TCTAGAATGTTTGGGAGTCTCCTACT |
| 501 | Left | GGACTCTGTAGGCTGCAGTTCTC |
| 502 | Right | GATATCGATCTGTTAGAAACCTCTCC |
| 503 | Left | GGACTCTGTAGGCTGCAGTTCTC |
| 504 | Right | TTAGAAACCTCTCCAGGTTCTTTG |
| 505 | Left | GGACTCTGTAGGCTGCAGTTCTC |
| 506 | Right | AGAAGTGTCTAGAATGTTTGGGAGT |
| 507 | Left | GGACTCTGTAGGCTGCAGTTCTC |
| 508 | Right | AAGTGTCTAGAATGTTTGGGAGTCT |
| 509 | Left | AGTTCTCAGCTCACAGCCTCCT |
| 510 | Right | CCTTGGAGATATCGATCTGTTAGAA |
| 511 | Left | AGTTCTCAGCTCACAGCCTCCT |
| 512 | Right | TAGAATGTTTGGGAGTCTCCTACTG |
| 513 | Left | AGTTCTCAGCTCACAGCCTCCT |
| 514 | Right | TGGAGATATCGATCTGTTAGAAACC |
| 515 | Left | GGACTCTGTAGGCTGCAGTTCTC |
| 516 | Right | ATCGATCTGTTAGAAACCTCTCCAG |
| 517 | Left | GTTCTCAGCTCACAGCCTCCT |
| 518 | Right | CCTTGGAGATATCGATCTGTTAGAA |
| 519 | Left | AGTTCTCAGCTCACAGCCTCCT |
| 520 | Right | GTTGTTCCATTCTGGTAAGAAGTGT |

ALK Exon22 301-400 bases

| | | |
|---|---|---|
| 521 | Left | GGACTCTGTAGGCTGCAGTTCTC |
| 522 | Right | GTTGTTCCATTCTGGTAAGAAGTGT |
| 523 | Left | GGACTCTGTAGGCTGCAGTTCTC |
| 524 | Right | TGTTCCATTCTGGTAAGAAGTGTCT |
| 525 | Left | GGACTCTGTAGGCTGCAGTTCTC |
| 526 | Right | TTGTTCCATTCTGGTAAGAAGTGTC |
| 527 | Left | GGACTCTGTAGGCTGCAGTTCTC |
| 528 | Right | GGTTGTTCCATTCTGGTAAGAAGT |
| 529 | Left | GGACTCTGTAGGCTGCAGTTCTC |
| 530 | Right | GGATTATTAGGCCACACAGACTTT |
| 531 | Left | GGACTCTGTAGGCTGCAGTTCTC |
| 532 | Right | TGTTCCATTCTGGTAAGAAGTGTCTA |
| 533 | Left | GGACTCTGTAGGCTGCAGTTCTC |
| 534 | Right | TGTTCCATTCTGGTAAGAAGTGTC |
| 535 | Left | GGACTCTGTAGGCTGCAGTTCTC |
| 536 | Right | ATACTGGTTGCAGACAGTGACATC |
| 537 | Left | GGACTCTGTAGGCTGCAGTTCTC |
| 538 | Right | GATACTGGTTGCAGACAGTGACAT |
| 539 | Left | GGACTCTGTAGGCTGCAGTTCTC |
| 540 | Right | ATTAGGCCACACAGACTTTGTTTCT |
| 541 | Left | GGACTCTGTAGGCTGCAGTTCTC |
| 542 | Right | TTGTTCCATTCTGGTAAGAAGTGT |
| 543 | Left | GGACTCTGTAGGCTGCAGTTCTC |
| 544 | Right | GTTCCATTCTGGTAAGAAGTGTCTA |
| 545 | Left | GGACTCTGTAGGCTGCAGTTCTC |
| 546 | Right | GATACTGGTTGCAGACAGTGACATC |
| 547 | Left | CGGACTCTGTAGGCTGCAGTT |
| 548 | Right | GTTGTTCCATTCTGGTAAGAAGTGT |
| 549 | Left | GGACTCTGTAGGCTGCAGTTCTC |
| 550 | Right | TAGGCCACACAGACTTTGTTTCT |
| 551 | Left | GGACTCTGTAGGCTGCAGTTCTC |
| 552 | Right | TACTGGTTGCAGACAGTGACATC |
| 553 | Left | GTAGGCTGCAGTTCTCAGCTCACAG |
| 554 | Right | GTTGTTCCATTCTGGTAAGAAGTGT |
| 555 | Left | AGTTCTCAGCTCACAGCCTCCT |
| 556 | Right | GGATTATTAGGCCACACAGACTTT |
| 557 | Left | GGACTCTGTAGGCTGCAGTTCTC |
| 558 | Right | TTAGGCCACACAGACTTTGTTTCT |
| 559 | Left | GGACTCTGTAGGCTGCAGTTCTC |
| 560 | Right | ACAGTGACATCGGTGGGATTATTAG |

ALK Exon23 151-200 bases

| | | |
|---|---|---|
| 561 | Left | TTAATTTTGGTTACATCCCTCTCTG |
| 562 | Right | AGCAAAGACTGGTTCTCACTCAC |
| 563 | Left | CAGACTCAGCTCAGTTAATTTTGGT |
| 564 | Right | AGCAAAGACTGGTTCTCACTCAC |
| 565 | Left | AGCTCAGTTAATTTTGGTTACATCC |
| 566 | Right | AGCAAAGACTGGTTCTCACTCAC |
| 567 | Left | AGACTCAGCTCAGTTAATTTTGGTT |
| 568 | Right | AGCAAAGACTGGTTCTCACTCAC |
| 569 | Left | CTCAGCTCAGTTAATTTTGGTTACA |
| 570 | Right | AGCAAAGACTGGTTCTCACTCAC |
| 571 | Left | TCAGCTCAGTTAATTTTGGTTACATC |
| 572 | Right | AGCAAAGACTGGTTCTCACTCAC |
| 573 | Left | CAGTTAATTTTGGTTACATCCCTCT |
| 574 | Right | AGCAAAGACTGGTTCTCACTCAC |
| 575 | Left | ACTCAGCTCAGTTAATTTTGGTTACA |
| 576 | Right | AGCAAAGACTGGTTCTCACTCAC |
| 577 | Left | CAGTTAATTTTGGTTACATCCCTCTC |
| 578 | Right | AGCAAAGACTGGTTCTCACTCAC |
| 579 | Left | TCAGTTAATTTTGGTTACATCCCTCT |
| 580 | Right | AGCAAAGACTGGTTCTCACTCAC |
| 581 | Left | GTTAATTTTGGTTACATCCCTCTCTG |
| 582 | Right | AGCAAAGACTGGTTCTCACTCAC |
| 583 | Left | CAGACTCAGCTCAGTTAATTTTGG |
| 584 | Right | AGCAAAGACTGGTTCTCACTCAC |
| 585 | Left | CAGCTCAGTTAATTTTGGTTACATC |
| 586 | Right | AGCAAAGACTGGTTCTCACTCAC |
| 587 | Left | TCAGCTCAGTTAATTTTGGTTACAT |
| 588 | Right | AGCAAAGACTGGTTCTCACTCAC |
| 589 | Left | TTAATTTTGGTTACATCCCTCTCTG |
| 590 | Right | AACTGCAGCAAAGACTGGTTCT |
| 591 | Left | TTAATTTTGGTTACATCCCTCTCTG |
| 592 | Right | ACAACAACTGCAGCAAAGACTG |
| 593 | Left | TTAATTTTGGTTACATCCCTCTCTG |
| 594 | Right | CACAACAACTGCAGCAAAGACT |
| 595 | Left | CTCAGCTCAGTTAATTTTGGTTACAT |
| 596 | Right | AGCAAAGACTGGTTCTCACTCAC |
| 597 | Left | TTAATTTTGGTTACATCCCTCTCTG |
| 598 | Right | CAGCAAAGACTGGTTCTCACTCAC |
| 599 | Left | AGTTAATTTTGGTTACATCCCTCTC |
| 600 | Right | AGCAAAGACTGGTTCTCACTCAC |

ALK Exon23 201-300 bases

| | | |
|---|---|---|
| 601 | Left | TGTAGCTGCTGAAAATGTAACTTTG |
| 602 | Right | AGCAAAGACTGGTTCTCACTCAC |
| 603 | Left | TTAATTTTGGTTACATCCCTCTCTG |
| 604 | Right | CTGTCCAAGCCTAAAGTTGACAC |
| 605 | Left | TATCCTGTTCCTCCCAGTTTAAGAT |
| 606 | Right | AGCAAAGACTGGTTCTCACTCAC |
| 607 | Left | ATGCCTTTATACATTGTAGCTGCTG |
| 608 | Right | AGCAAAGACTGGTTCTCACTCAC |
| 609 | Left | GCCTTTATACATTGTAGCTGCTGAA |
| 610 | Right | AGCAAAGACTGGTTCTCACTCAC |
| 611 | Left | GTATCCTGTTCCTCCCAGTTTAAGA |
| 612 | Right | AGCAAAGACTGGTTCTCACTCAC |
| 613 | Left | GCCTTTATACATTGTAGCTGCTGA |
| 614 | Right | AGCAAAGACTGGTTCTCACTCAC |
| 615 | Left | AGTTTAAGATTTGCCCAGACTCAG |
| 616 | Right | AGCAAAGACTGGTTCTCACTCAC |
| 617 | Left | CCCAGACTCAGCTCAGTTAATTTT |
| 618 | Right | AGCAAAGACTGGTTCTCACTCAC |
| 619 | Left | TTAATTTTGGTTACATCCCTCTCTG |
| 620 | Right | GTCCAAGCCTAAAGTTGACACC |
| 621 | Left | CAGTTAATTTTGGTTACATCCCTCT |
| 622 | Right | CTGTCCAAGCCTAAAGTTGACAC |
| 623 | Left | TTAATTTTGGTTACATCCCTCTCTG |
| 624 | Right | CCTGTCCAAGCCTAAAGTTGAC |
| 625 | Left | TATCCTGTTCCTCCCAGTTTAAGA |
| 626 | Right | AGCAAAGACTGGTTCTCACTCAC |
| 627 | Left | CTGTTCCTCCCAGTTTAAGATTTG |
| 628 | Right | AGCAAAGACTGGTTCTCACTCAC |
| 629 | Left | CAGAATGCCTTTATACATTGTAGCTG |
| 630 | Right | AGCAAAGACTGGTTCTCACTCAC |
| 631 | Left | CCCATGTTTACAGAATGCCTTTAT |
| 632 | Right | AGCAAAGACTGGTTCTCACTCAC |
| 633 | Left | GCTGCTGAAAATGTAACTTTGTATC |
| 634 | Right | AGCAAAGACTGGTTCTCACTCAC |
| 635 | Left | GTATCCTGTTCCTCCCAGTTTAAG |
| 636 | Right | AGCAAAGACTGGTTCTCACTCAC |
| 637 | Left | TGTAGCTGCTGAAAATGTAACTTTG |
| 638 | Right | AACTGCAGCAAAGACTGGTTCT |
| 639 | Left | ATCCTGTTCCTCCCAGTTTAAGAT |
| 640 | Right | AGCAAAGACTGGTTCTCACTCAC |
| 641 | Left | TTAATTTTGGTTACATCCCTCTCTG |
| 642 | Right | TCAGCCATCATCTACCTCTATCTTC |
| 643 | Left | CAGACTCAGCTCAGTTAATTTTGGT |
| 644 | Right | TCAGCCATCATCTACCTCTATCTTC |
| 645 | Left | TTAATTTTGGTTACATCCCTCTCTG |
| 646 | Right | CTATCTTCTGTCCATTCTCTTCCAG |
| 647 | Left | CAGACTCAGCTCAGTTAATTTTGGT |
| 648 | Right | CTATCTTCTGTCCATTCTCTTCCAG |
| 649 | Left | TATCCTGTTCCTCCCAGTTTAAGAT |
| 650 | Right | CTATCTTCTGTCCATTCTCTTCCAG |
| 651 | Left | TTAATTTTGGTTACATCCCTCTCTG |
| 652 | Right | TCTATCTTCTGTCCATTCTCTTCCA |
| 653 | Left | CAGACTCAGCTCAGTTAATTTTGGT |
| 654 | Right | TCTATCTTCTGTCCATTCTCTTCCA |
| 655 | Left | TTAATTTTGGTTACATCCCTCTCTG |
| 656 | Right | CAGCCATCATCTACCTCTATCTTCT |
| 657 | Left | TTAATTTTGGTTACATCCCTCTCTG |
| 658 | Right | CTCAGCCATCATCTACCTCTATCTT |
| 659 | Left | TTAATTTTGGTTACATCCCTCTCTG |
| 660 | Right | AGCCATCATCTACCTCTATCTTCTG |
| 661 | Left | CAGACTCAGCTCAGTTAATTTTGGT |
| 662 | Right | CAGCCATCATCTACCTCTATCTTCT |
| 663 | Left | CAGACTCAGCTCAGTTAATTTTGGT |
| 664 | Right | CTCAGCCATCATCTACCTCTATCTT |
| 665 | Left | CAGACTCAGCTCAGTTAATTTTGGT |
| 666 | Right | AGCCATCATCTACCTCTATCTTCTG |
| 667 | Left | TTAATTTTGGTTACATCCCTCTCTG |
| 668 | Right | GCCATCATCTACCTCTATCTTCTGT |
| 669 | Left | CAGACTCAGCTCAGTTAATTTTGGT |
| 670 | Right | GCCATCATCTACCTCTATCTTCTGT |
| 671 | Left | AGCTCAGTTAATTTTGGTTACATCC |
| 672 | Right | TCAGCCATCATCTACCTCTATCTTC |
| 673 | Left | AGCTCAGTTAATTTTGGTTACATCC |
| 674 | Right | CTATCTTCTGTCCATTCTCTTCCAG |
| 675 | Left | AGACTCAGCTCAGTTAATTTTGGTT |
| 676 | Right | TCAGCCATCATCTACCTCTATCTTC |
| 677 | Left | CTCAGCTCAGTTAATTTTGGTTACA |
| 678 | Right | TCAGCCATCATCTACCTCTATCTTC |
| 679 | Left | TATCCTGTTCCTCCCAGTTTAAGAT |
| 680 | Right | TCTATCTTCTGTCCATTCTCTTCCA |

ALK Exon23 401-600 bases

| | | |
|---|---|---|
| 681 | Left | TGTAGCTGCTGAAAATGTAACTTTG |
| 682 | Right | TCAGCCATCATCTACCTCTATCTTC |
| 683 | Left | TTAATTTTGGTTACATCCCTCTCTG |
| 684 | Right | ACCTTCTGCAATGATTGTAAGTTTC |
| 685 | Left | CAGACTCAGCTCAGTTAATTTTGGT |
| 686 | Right | ACCTTCTGCAATGATTGTAAGTTTC |
| 687 | Left | TTAATTTTGGTTACATCCCTCTCTG |
| 688 | Right | CTTCTGCAATGATTGTAAGTTTCCT |
| 689 | Left | CAGACTCAGCTCAGTTAATTTTGGT |
| 690 | Right | CTTCTGCAATGATTGTAAGTTTCCT |
| 691 | Left | TGTAGCTGCTGAAAATGTAACTTTG |
| 692 | Right | CTATCTTCTGTCCATTCTCTTCCAG |
| 693 | Left | TATCCTGTTCCTCCCAGTTTAAGAT |
| 694 | Right | ACCTTCTGCAATGATTGTAAGTTTC |
| 695 | Left | TATCCTGTTCCTCCCAGTTTAAGAT |
| 696 | Right | CTTCTGCAATGATTGTAAGTTTCCT |
| 697 | Left | TGTAGCTGCTGAAAATGTAACTTTG |
| 698 | Right | TCTATCTTCTGTCCATTCTCTTCCA |
| 699 | Left | TGTAGCTGCTGAAAATGTAACTTTG |
| 700 | Right | CAGCCATCATCTACCTCTATCTTCT |
| 701 | Left | TGTAGCTGCTGAAAATGTAACTTTG |
| 702 | Right | AGCCATCATCTACCTCTATCTTCTG |
| 703 | Left | TGTAGCTGCTGAAAATGTAACTTTG |
| 704 | Right | CTCAGCCATCATCTACCTCTATCTT |
| 705 | Left | TGTAGCTGCTGAAAATGTAACTTTG |
| 706 | Right | GCCATCATCTACCTCTATCTTCTGT |
| 707 | Left | TTAATTTTGGTTACATCCCTCTCTG |
| 708 | Right | CACCTTCTGCAATGATTGTAAGTTT |
| 709 | Left | AGCTCAGTTAATTTTGGTTACATCC |
| 710 | Right | ACCTTCTGCAATGATTGTAAGTTTC |
| 711 | Left | CAGACTCAGCTCAGTTAATTTTGGT |
| 712 | Right | CACCTTCTGCAATGATTGTAAGTTT |
| 713 | Left | AGCTCAGTTAATTTTGGTTACATCC |
| 714 | Right | CTTCTGCAATGATTGTAAGTTTCCT |
| 715 | Left | ATGCCTTTATACATTGTAGCTGCTG |
| 716 | Right | TCAGCCATCATCTACCTCTATCTTC |
| 717 | Left | TGTAGCTGCTGAAAATGTAACTTTG |
| 718 | Right | GAGCCACTTAAATCTCTTTTCTTTG |
| 719 | Left | TGTAGCTGCTGAAAATGTAACTTTG |
| 720 | Right | TGAGCCACTTAAATCTCTTTTCTTT |

ALK Exon23 601-800 bases

| | | |
|---|---|---|
| 721 | Left | TGTAGCTGCTGAAAATGTAACTTTG |
| 722 | Right | ACCTTCTGCAATGATTGTAAGTTTC |
| 723 | Left | TGTAGCTGCTGAAAATGTAACTTTG |
| 724 | Right | GTTGAATTGTAATCCCTAGTGTTGG |
| 725 | Left | TGTAGCTGCTGAAAATGTAACTTTG |
| 726 | Right | CTTCTGCAATGATTGTAAGTTTCCT |
| 727 | Left | TTAATTTTGGTTACATCCCTCTCTG |
| 728 | Right | GCTATAGAATGTGGATATGGTTTGG |
| 729 | Left | TTAATTTTGGTTACATCCCTCTCTG |
| 730 | Right | GGCTATAGAATGTGGATATGGTTTG |
| 731 | Left | CAGACTCAGCTCAGTTAATTTTGGT |
| 732 | Right | GCTATAGAATGTGGATATGGTTTGG |
| 733 | Left | CAGACTCAGCTCAGTTAATTTTGGT |
| 734 | Right | GGCTATAGAATGTGGATATGGTTTG |
| 735 | Left | TTAATTTTGGTTACATCCCTCTCTG |
| 736 | Right | GTTGAATTGTAATCCCTAGTGTTGG |
| 737 | Left | CAGACTCAGCTCAGTTAATTTTGGT |
| 738 | Right | GTTGAATTGTAATCCCTAGTGTTGG |
| 739 | Left | CCAGTATCTGAGAAAGACACTCTCC |
| 740 | Right | TCAGCCATCATCTACCTCTATCTTC |
| 741 | Left | TTAATTTTGGTTACATCCCTCTCTG |
| 742 | Right | TAGAATGTGGATATGGTTTGGATTT |
| 743 | Left | CAGACTCAGCTCAGTTAATTTTGGT |
| 744 | Right | TAGAATGTGGATATGGTTTGGATTT |
| 745 | Left | CCAGTATCTGAGAAAGACACTCTCC |
| 746 | Right | CTATCTTCTGTCCATTCTCTTCCAG |
| 747 | Left | TTAATTTTGGTTACATCCCTCTCTG |
| 748 | Right | TCATGAGACCTGGTTGTTTAAAAGT |
| 749 | Left | TTAATTTTGGTTACATCCCTCTCTG |
| 750 | Right | CAGGCTATAGAATGTGGATATGGTT |
| 751 | Left | TGTAGCTGCTGAAAATGTAACTTTG |
| 752 | Right | CTCATGAGACCTGGTTGTTTAAAAG |
| 753 | Left | CAGACTCAGCTCAGTTAATTTTGGT |
| 754 | Right | TCATGAGACCTGGTTGTTTAAAAGT |
| 755 | Left | CAGACTCAGCTCAGTTAATTTTGGT |
| 756 | Right | CAGGCTATAGAATGTGGATATGGTT |
| 757 | Left | TTAATTTTGGTTACATCCCTCTCTG |
| 758 | Right | ATCCAGGCTATAGAATGTGGATATG |
| 759 | Left | CAGACTCAGCTCAGTTAATTTTGGT |
| 760 | Right | ATCCAGGCTATAGAATGTGGATATG |

ALK Exon23 801-1000 bases

| | | |
|---|---|---|
| 761 | Left | TGTAGCTGCTGAAAATGTAACTTTG |
| 762 | Right | GCTATAGAATGTGGATATGGTTTGG |
| 763 | Left | TGTAGCTGCTGAAAATGTAACTTTG |
| 764 | Right | GGCTATAGAATGTGGATATGGTTTG |
| 765 | Left | TGTAGCTGCTGAAAATGTAACTTTG |
| 766 | Right | GTCATGAAAGTTCTCCTCTGTGTTT |
| 767 | Left | TGTAGCTGCTGAAAATGTAACTTTG |
| 768 | Right | ATGAAAGTTCTCCTCTGTGTTTGTC |
| 769 | Left | CCAGTATCTGAGAAAGACACTCTCC |
| 770 | Right | ACCTTCTGCAATGATTGTAAGTTTC |
| 771 | Left | TGTAGCTGCTGAAAATGTAACTTTG |
| 772 | Right | TAGAATGTGGATATGGTTTGGATTT |
| 773 | Left | TGTAGCTGCTGAAAATGTAACTTTG |
| 774 | Right | CTCTAGTTTGGTTTTCCAGAGTCAG |
| 775 | Left | TGTAGCTGCTGAAAATGTAACTTTG |
| 776 | Right | CCTCTGTGTTTGTCTCTAGTTTGGT |
| 777 | Left | CCAGTATCTGAGAAAGACACTCTCC |
| 778 | Right | CTTCTGCAATGATTGTAAGTTTCCT |
| 779 | Left | TGTAGCTGCTGAAAATGTAACTTTG |
| 780 | Right | GAAAGTTCTCCTCTGTGTTTGTCTC |
| 781 | Left | CATGTTAACAAGAAAACCCAAGTCT |
| 782 | Right | TCAGCCATCATCTACCTCTATCTTC |
| 783 | Left | TTAATTTTGGTTACATCCCTCTCTG |
| 784 | Right | TATTATCCCTACTTGAGACGTGAGG |
| 785 | Left | TTAATTTTGGTTACATCCCTCTCTG |
| 786 | Right | CAGGTCAGTTGCTTGAGTAGTTACA |
| 787 | Left | TTAATTTTGGTTACATCCCTCTCTG |
| 788 | Right | GTCATGAAAGTTCTCCTCTGTGTTT |
| 789 | Left | TTAATTTTGGTTACATCCCTCTCTG |
| 790 | Right | ATGAAAGTTCTCCTCTGTGTTTGTC |
| 791 | Left | CAGACTCAGCTCAGTTAATTTTGGT |
| 792 | Right | TATTATCCCTACTTGAGACGTGAGG |
| 793 | Left | CAGACTCAGCTCAGTTAATTTTGGT |
| 794 | Right | CAGGTCAGTTGCTTGAGTAGTTACA |
| 795 | Left | CAGACTCAGCTCAGTTAATTTTGGT |
| 796 | Right | GTCATGAAAGTTCTCCTCTGTGTTT |
| 797 | Left | CAGACTCAGCTCAGTTAATTTTGGT |
| 798 | Right | ATGAAAGTTCTCCTCTGTGTTTGTC |
| 799 | Left | TTAATTTTGGTTACATCCCTCTCTG |
| 800 | Right | CTCTAGTTTGGTTTTCCAGAGTCAG |

ALK Exon23 2 kb

| | | |
|---|---|---|
| 801 | Left | TTCTAACAGATCGATATCTCCAAGG |
| 802 | Right | GCTATAGAATGTGGATATGGTTTGG |
| 803 | Left | GAGCATGCTAGACTTTGACAGTACA |
| 804 | Right | GCTATAGAATGTGGATATGGTTTGG |
| 805 | Left | GAGCATGCTAGACTTTGACAGTACA |
| 806 | Right | GGCTATAGAATGTGGATATGGTTTG |
| 807 | Left | TTCTAACAGATCGATATCTCCAAGG |
| 808 | Right | ACCTTCTGCAATGATTGTAAGTTTC |
| 809 | Left | GAGCATGCTAGACTTTGACAGTACA |
| 810 | Right | CCAAGCTCTGTTAACCATAAGATGT |
| 811 | Left | TTCTAACAGATCGATATCTCCAAGG |
| 812 | Right | GTTGAATTGTAATCCCTAGTGTTGG |
| 813 | Left | TTCTAACAGATCGATATCTCCAAGG |
| 814 | Right | CTTCTGCAATGATTGTAAGTTTCCT |
| 815 | Left | GAGCATGCTAGACTTTGACAGTACA |
| 816 | Right | GTTGAATTGTAATCCCTAGTGTTGG |
| 817 | Left | ACAGGAGGACACACAAAATAACATT |
| 818 | Right | ATAGTACAGTGGTTCGTTGAGGAAG |
| 819 | Left | GAGCATGCTAGACTTTGACAGTACA |
| 820 | Right | TATTATCCCTACTTGAGACGTGAGG |
| 821 | Left | GAGCATGCTAGACTTTGACAGTACA |
| 822 | Right | CAGGTCAGTTGCTTGAGTAGTTACA |
| 823 | Left | GAGCATGCTAGACTTTGACAGTACA |
| 824 | Right | GTCATGAAAGTTCTCCTCTGTGTTT |
| 825 | Left | AGATATTTGACCTCAAGATCAGGTG |
| 826 | Right | AGGTTAAAGGTTTAAGACTGCCCTA |
| 827 | Left | CAGTAGGAGACTCCCAAACATTCTA |
| 828 | Right | GTTGAATTGTAATCCCTAGTGTTGG |
| 829 | Left | ACAGGAGGACACACAAAATAACATT |
| 830 | Right | AGGTTAAAGGTTTAAGACTGCCCTA |
| 831 | Left | AGATATTTGACCTCAAGATCAGGTG |
| 832 | Right | CCAAGCTCTGTTAACCATAAGATGT |
| 833 | Left | TTCTAACAGATCGATATCTCCAAGG |
| 834 | Right | TAGAATGTGGATATGGTTTGGATTT |
| 835 | Left | CATGTTAACAAGAAAACCCAAGTCT |
| 836 | Right | ATAGTACAGTGGTTCGTTGAGGAAG |
| 837 | Left | AGATATTTGACCTCAAGATCAGGTG |
| 838 | Right | CACCTTTGAGATGTTCTAGTCCAAT |
| 839 | Left | ACAGGAGGACACACAAAATAACATT |
| 840 | Right | CACCTTTGAGATGTTCTAGTCCAAT |

ALK Exon21-23 2 kb

| | | |
|---|---|---|
| 841 | Left | TTGACTCTGTCTCCTCTTGTCTTCT |
| 842 | Right | TCAGCCATCATCTACCTCTATCTTC |
| 843 | Left | TTGACTCTGTCTCCTCTTGTCTTCT |
| 844 | Right | CTATCTTCTGTCCATTCTCTTCCAG |
| 845 | Left | TTTGACTCTGTCTCCTCTTGTCTTC |
| 846 | Right | TCAGCCATCATCTACCTCTATCTTC |
| 847 | Left | ACTCTGTCTCCTCTTGTCTTCTCCT |
| 848 | Right | TCAGCCATCATCTACCTCTATCTTC |
| 849 | Left | GTTTGACTCTGTCTCCTCTTGTCTT |
| 850 | Right | TCAGCCATCATCTACCTCTATCTTC |
| 851 | Left | TTTGACTCTGTCTCCTCTTGTCTTC |
| 852 | Right | CTATCTTCTGTCCATTCTCTTCCAG |
| 853 | Left | ACTCTGTCTCCTCTTGTCTTCTCCT |
| 854 | Right | CTATCTTCTGTCCATTCTCTTCCAG |
| 855 | Left | GTTTGACTCTGTCTCCTCTTGTCTT |
| 856 | Right | CTATCTTCTGTCCATTCTCTTCCAG |
| 857 | Left | TTGACTCTGTCTCCTCTTGTCTTCT |
| 858 | Right | TCTATCTTCTGTCCATTCTCTTCCA |
| 859 | Left | TGACTCTGTCTCCTCTTGTCTTCTC |
| 860 | Right | TCAGCCATCATCTACCTCTATCTTC |
| 861 | Left | TTGACTCTGTCTCCTCTTGTCTTCT |
| 862 | Right | CAGCCATCATCTACCTCTATCTTCT |
| 863 | Left | TTGACTCTGTCTCCTCTTGTCTTCT |
| 864 | Right | CTCAGCCATCATCTACCTCTATCTT |
| 865 | Left | TTGACTCTGTCTCCTCTTGTCTTCT |
| 866 | Right | AGCCATCATCTACCTCTATCTTCTG |
| 867 | Left | TGACTCTGTCTCCTCTTGTCTTCTC |
| 868 | Right | CTATCTTCTGTCCATTCTCTTCCAG |
| 869 | Left | TTGACTCTGTCTCCTCTTGTCTTCT |
| 870 | Right | GCCATCATCTACCTCTATCTTCTGT |
| 871 | Left | TGTTTGACTCTGTCTCCTCTTGTCT |
| 872 | Right | TCAGCCATCATCTACCTCTATCTTC |
| 873 | Left | CTGTTTGACTCTGTCTCCTCTTGTC |
| 874 | Right | TCAGCCATCATCTACCTCTATCTTC |
| 875 | Left | CTCTGTCTCCTCTTGTCTTCTCCTT |
| 876 | Right | TCAGCCATCATCTACCTCTATCTTC |
| 877 | Left | TTTGACTCTGTCTCCTCTTGTCTTC |
| 878 | Right | TCTATCTTCTGTCCATTCTCTTCCA |
| 879 | Left | TGTTTGACTCTGTCTCCTCTTGTCT |
| 880 | Right | CTATCTTCTGTCCATTCTCTTCCAG |

ALK Exon21-23 5 kb

| | | |
|---|---|---|
| 881 | Left | CAGTGTAGGGGCTGAATGTTATC |
| 882 | Right | TACAACTTTCTCTCCTTAAGCCTCA |
| 883 | Left | CAGTGTAGGGGCTGAATGTTATC |
| 884 | Right | ACAACTTTCTCTCCTTAAGCCTCA |
| 885 | Left | CAGTGTAGGGGCTGAATGTTATC |
| 886 | Right | ATACAACTTTCTCTCCTTAAGCCTCA |
| 887 | Left | GCAGTGTAGGGGCTGAATGTTAT |
| 888 | Right | TACAACTTTCTCTCCTTAAGCCTCA |
| 889 | Left | CAGACTCCTCTAGCCACAAAAGG |
| 890 | Right | TACAACTTTCTCTCCTTAAGCCTCA |
| 891 | Left | GCAGACTCCTCTAGCCACAAAAG |
| 892 | Right | TACAACTTTCTCTCCTTAAGCCTCA |
| 893 | Left | GCAGACTCCTCTAGCCACAAAA |
| 894 | Right | TACAACTTTCTCTCCTTAAGCCTCA |
| 895 | Left | AGACTCCTCTAGCCACAAAAGG |
| 896 | Right | TACAACTTTCTCTCCTTAAGCCTCA |
| 897 | Left | CAGTGTAGGGGCTGAATGTTATC |
| 898 | Right | GCATGCATACAACTTTCTCTCCTT |
| 899 | Left | GCAGTGTAGGGGCTGAATGTTATC |
| 900 | Right | TACAACTTTCTCTCCTTAAGCCTCA |
| 901 | Left | GTAGGGGCTGAATGTTATCACAGC |
| 902 | Right | TACAACTTTCTCTCCTTAAGCCTCA |
| 903 | Left | GAGGACAAGCCTTGACATTCAG |
| 904 | Right | TACAACTTTCTCTCCTTAAGCCTCA |
| 905 | Left | ATGTTGGCTTACATTAACTCCCATA |
| 906 | Right | TTTGCAAAGTCCCTCTCCTTT |
| 907 | Left | GTTATCACAGCACCGCAGACT |
| 908 | Right | TACAACTTTCTCTCCTTAAGCCTCA |
| 909 | Left | GCAGACTCCTCTAGCCACAAA |
| 910 | Right | TACAACTTTCTCTCCTTAAGCCTCA |
| 911 | Left | GCAGTGTAGGGGCTGAATGTTA |
| 912 | Right | TACAACTTTCTCTCCTTAAGCCTCA |
| 913 | Left | TAGGGGCTGAATGTTATCACAGC |
| 914 | Right | TACAACTTTCTCTCCTTAAGCCTCA |
| 915 | Left | CAGACTCCTCTAGCCACAAAAGG |
| 916 | Right | GCATGCATACAACTTTCTCTCCTTA |
| 917 | Left | GCAGTGTAGGGGCTGAATGTTAT |
| 918 | Right | ACAACTTTCTCTCCTTAAGCCTCA |
| 919 | Left | ATGTTGGCTTACATTAACTCCCATA |
| 920 | Right | CAAAGTCCCTCTCCTTTGCAT |

ALK Exon24 130-150 bases

| | | |
|---|---|---|
| 921 | Left | AGTGGCCCGCTTCTGTCT |
| 922 | Right | GATGACAGGAAGAGCACAGTCAC |
| 923 | Left | CGCTTCTGTCTCCCCACAG |
| 924 | Right | GATGACAGGAAGAGCACAGTCAC |
| 925 | Left | CGCTTCTGTCTCCCCACA |
| 926 | Right | GATGACAGGAAGAGCACAGTCAC |
| 927 | Left | AGTGGCCCGCTTCTGTCT |
| 928 | Right | AGGATGACAGGAAGAGCACAGT |
| 929 | Left | CGCTTCTGTCTCCCCACAG |
| 930 | Right | AGGATGACAGGAAGAGCACAGT |
| 931 | Left | CGCTTCTGTCTCCCCACA |
| 932 | Right | AGGATGACAGGAAGAGCACAGT |
| 933 | Left | AGTGGCCCGCTTCTGTCT |
| 934 | Right | GATGACAGGAAGAGCACAGTCA |
| 935 | Left | CGCTTCTGTCTCCCCACAG |
| 936 | Right | GATGACAGGAAGAGCACAGTCA |
| 937 | Left | CGCTTCTGTCTCCCCACA |
| 938 | Right | GATGACAGGAAGAGCACAGTCA |
| 939 | Left | AGTGGCCCGCTTCTGTCT |
| 940 | Right | ATGACAGGAAGAGCACAGTCAC |
| 941 | Left | CGCTTCTGTCTCCCCACAG |
| 942 | Right | ATGACAGGAAGAGCACAGTCAC |
| 943 | Left | CGCTTCTGTCTCCCCACA |
| 944 | Right | ATGACAGGAAGAGCACAGTCAC |
| 945 | Left | AGTGGCCCGCTTCTGTCT |
| 946 | Right | AGGATGACAGGAAGAGCACAGTC |
| 947 | Left | CGCTTCTGTCTCCCCACAG |
| 948 | Right | AGGATGACAGGAAGAGCACAGTC |
| 949 | Left | CGCTTCTGTCTCCCCACA |
| 950 | Right | AGGATGACAGGAAGAGCACAGTC |
| 951 | Left | AGTGGCCCGCTTCTGTCT |
| 952 | Right | GGATGACAGGAAGAGCACAGTC |
| 953 | Left | CGCTTCTGTCTCCCCACAG |
| 954 | Right | GGATGACAGGAAGAGCACAGTC |
| 955 | Left | CGCTTCTGTCTCCCCACAG |
| 956 | Right | GACAGGATGACAGGAAGAGCAC |
| 957 | Left | CGCTTCTGTCTCCCCACA |
| 958 | Right | GGATGACAGGAAGAGCACAGTC |
| 959 | Left | CGCTTCTGTCTCCCCACA |
| 960 | Right | GACAGGATGACAGGAAGAGCAC |

ALK Exon24 161-200 bases

| | | |
|---|---|---|
| 961 | Left | ATTTCAGATTTCCCTCCTCTCACT |
| 962 | Right | GATGACAGGAAGAGCACAGTCAC |
| 963 | Left | ATTTCAGATTTCCCTCCTCTCACT |
| 964 | Right | AGGATGACAGGAAGAGCACAGT |
| 965 | Left | ATTTCAGATTTCCCTCCTCTCACT |
| 966 | Right | GATGACAGGAAGAGCACAGTCA |
| 967 | Left | ATTTCAGATTTCCCTCCTCTCAC |
| 968 | Right | GATGACAGGAAGAGCACAGTCAC |
| 969 | Left | ATTTCAGATTTCCCTCCTCTCAC |
| 970 | Right | AGGATGACAGGAAGAGCACAGT |
| 971 | Left | ATTTCAGATTTCCCTCCTCTCACT |
| 972 | Right | ATGACAGGAAGAGCACAGTCAC |
| 973 | Left | ATTTCAGATTTCCCTCCTCTCAC |
| 974 | Right | GATGACAGGAAGAGCACAGTCA |
| 975 | Left | ATTTCAGATTTCCCTCCTCTCAC |
| 976 | Right | ATGACAGGAAGAGCACAGTCAC |
| 977 | Left | TTTCAGATTTCCCTCCTCTCACT |
| 978 | Right | GATGACAGGAAGAGCACAGTCAC |
| 979 | Left | TTTCAGATTTCCCTCCTCTCACT |
| 980 | Right | AGGATGACAGGAAGAGCACAGT |
| 981 | Left | ATTTCAGATTTCCCTCCTCTCACT |
| 982 | Right | AGGATGACAGGAAGAGCACAGTC |
| 983 | Left | ATTTCAGATTTCCCTCCTCTCACT |
| 984 | Right | GGATGACAGGAAGAGCACAGTC |
| 985 | Left | TTTCAGATTTCCCTCCTCTCACT |
| 986 | Right | GATGACAGGAAGAGCACAGTCA |
| 987 | Left | ATTTCAGATTTCCCTCCTCTCAC |
| 988 | Right | AGGATGACAGGAAGAGCACAGTC |
| 989 | Left | TTTCAGATTTCCCTCCTCTCACT |
| 990 | Right | ATGACAGGAAGAGCACAGTCAC |
| 991 | Left | ATTTCAGATTTCCCTCCTCTCAC |
| 992 | Right | GGATGACAGGAAGAGCACAGTC |
| 993 | Left | ATTTCAGATTTCCCTCCTCTCACT |
| 994 | Right | AGGATGACAGGAAGAGCACAG |
| 995 | Left | ATTTCCCTCCTCTCACTGACAA |
| 996 | Right | GATGACAGGAAGAGCACAGTCAC |
| 997 | Left | ATTTCCCTCCTCTCACTGACAA |
| 998 | Right | AGGATGACAGGAAGAGCACAGT |
| 999 | Left | CATTTCAGATTTCCCTCCTCTCACT |
| 1000 | Right | GATGACAGGAAGAGCACAGTCAC |

ALK Exon24 201-300 bases

| | | |
|---|---|---|
| 1001 | Left | ATTTCAGATTTCCCTCCTCTCACT |
| 1002 | Right | GAGACCTAGTATTCTGCTCTGAAGG |
| 1003 | Left | ATTTCAGATTTCCCTCCTCTCACT |
| 1004 | Right | GGAGACCTAGTATTCTGCTCTGAAG |
| 1005 | Left | ATTTCAGATTTCCCTCCTCTCACT |
| 1006 | Right | CTCTGGAGGGAGACCTAGTATTCTG |
| 1007 | Left | ATTTCAGATTTCCCTCCTCTCAC |
| 1008 | Right | GAGACCTAGTATTCTGCTCTGAAGG |
| 1009 | Left | ATTTCAGATTTCCCTCCTCTCAC |
| 1010 | Right | GGAGACCTAGTATTCTGCTCTGAAG |
| 1011 | Left | ATTTCAGATTTCCCTCCTCTCACT |
| 1012 | Right | AGGGAGACCTAGTATTCTGCTCTGA |
| 1013 | Left | ATTTCAGATTTCCCTCCTCTCACT |
| 1014 | Right | TCTGGAGGGAGACCTAGTATTCTG |
| 1015 | Left | ATTTCAGATTTCCCTCCTCTCAC |
| 1016 | Right | CTCTGGAGGGAGACCTAGTATTCTG |
| 1017 | Left | ATTTCAGATTTCCCTCCTCTCACT |
| 1018 | Right | GGGAGACCTAGTATTCTGCTCTGA |
| 1019 | Left | ATTTCAGATTTCCCTCCTCTCAC |
| 1020 | Right | AGGGAGACCTAGTATTCTGCTCTGA |
| 1021 | Left | ATTTCAGATTTCCCTCCTCTCAC |
| 1022 | Right | TCTGGAGGGAGACCTAGTATTCTG |
| 1023 | Left | CTCCTCTCACTGACAAGCTCCT |
| 1024 | Right | AAACAAAGCTGAATCATCCTACATC |
| 1025 | Left | ATTTCAGATTTCCCTCCTCTCAC |
| 1026 | Right | GGGAGACCTAGTATTCTGCTCTGA |
| 1027 | Left | TTTCAGATTTCCCTCCTCTCACT |
| 1028 | Right | GAGACCTAGTATTCTGCTCTGAAGG |
| 1029 | Left | TTTCAGATTTCCCTCCTCTCACT |
| 1030 | Right | GGAGACCTAGTATTCTGCTCTGAAG |
| 1031 | Left | TTTCAGATTTCCCTCCTCTCACT |
| 1032 | Right | CTCTGGAGGGAGACCTAGTATTCTG |
| 1033 | Left | TTTCAGATTTCCCTCCTCTCACT |
| 1034 | Right | AGGGAGACCTAGTATTCTGCTCTGA |
| 1035 | Left | TTTCAGATTTCCCTCCTCTCACT |
| 1036 | Right | TCTGGAGGGAGACCTAGTATTCTG |
| 1037 | Left | ATTTCAGATTTCCCTCCTCTCACT |
| 1038 | Right | GCTCTGGAGGGAGACCTAGTATTC |
| 1039 | Left | TTTCAGATTTCCCTCCTCTCACT |
| 1040 | Right | GGGAGACCTAGTATTCTGCTCTGA |

ALK Exon24 301-400 bases

| | | |
|---|---|---|
| 1041 | Left | ATTTCAGATTTCCCTCCTCTCACT |
| 1042 | Right | AAACAAAGCTGAATCATCCTACATC |
| 1043 | Left | ATTTCAGATTTCCCTCCTCTCACT |
| 1044 | Right | ACAATAAAACAAAGCTGAATCATCC |
| 1045 | Left | ATTTCAGATTTCCCTCCTCTCAC |
| 1046 | Right | AAACAAAGCTGAATCATCCTACATC |
| 1047 | Left | ATTTCAGATTTCCCTCCTCTCAC |
| 1048 | Right | ACAATAAAACAAAGCTGAATCATCC |
| 1049 | Left | ATTTCAGATTTCCCTCCTCTCACT |
| 1050 | Right | AAAACAAAGCTGAATCATCCTACAT |
| 1051 | Left | ATTTCAGATTTCCCTCCTCTCAC |
| 1052 | Right | AAAACAAAGCTGAATCATCCTACAT |
| 1053 | Left | ATTTCAGATTTCCCTCCTCTCACT |
| 1054 | Right | TAAAACAAAGCTGAATCATCCTACA |
| 1055 | Left | TTTCAGATTTCCCTCCTCTCACT |
| 1056 | Right | AAACAAAGCTGAATCATCCTACATC |
| 1057 | Left | ATTTCAGATTTCCCTCCTCTCAC |
| 1058 | Right | TAAAACAAAGCTGAATCATCCTACA |
| 1059 | Left | TTTCAGATTTCCCTCCTCTCACT |
| 1060 | Right | ACAATAAAACAAAGCTGAATCATCC |
| 1061 | Left | ATTTCAGATTTCCCTCCTCTCACT |
| 1062 | Right | CAATAAAACAAAGCTGAATCATCCTA |
| 1063 | Left | ATTTCAGATTTCCCTCCTCTCACT |
| 1064 | Right | AGCTGAATCATCCTACATCCAAAT |
| 1065 | Left | AATGCCTCCAGGTGATTTCTAAT |
| 1066 | Right | GAGACCTAGTATTCTGCTCTGAAGG |
| 1067 | Left | AATGCCTCCAGGTGATTTCTAAT |
| 1068 | Right | GGAGACCTAGTATTCTGCTCTGAAG |
| 1069 | Left | TTTCAGATTTCCCTCCTCTCACT |
| 1070 | Right | AAAACAAAGCTGAATCATCCTACAT |
| 1071 | Left | ATTTCAGATTTCCCTCCTCTCAC |
| 1072 | Right | CAATAAAACAAAGCTGAATCATCCTA |
| 1073 | Left | ATTTCAGATTTCCCTCCTCTCACT |
| 1074 | Right | AAGCTGAATCATCCTACATCCAAAT |
| 1075 | Left | AAATGCCTCCAGGTGATTTCTAAT |
| 1076 | Right | GAGACCTAGTATTCTGCTCTGAAGG |
| 1077 | Left | AAATGCCTCCAGGTGATTTCTAAT |
| 1078 | Right | GGAGACCTAGTATTCTGCTCTGAAG |
| 1079 | Left | AATGCCTCCAGGTGATTTCTAAT |
| 1080 | Right | CTCTGGAGGGAGACCTAGTATTCTG |

ALK Exon24 401-600 bases

| | | |
|---|---|---|
| 1081 | Left | TGCACAATAAATTAAAAGGGAAAGA |
| 1082 | Right | AAACAAAGCTGAATCATCCTACATC |
| 1083 | Left | TGCACAATAAATTAAAAGGGAAAGA |
| 1084 | Right | ACAATAAAACAAAGCTGAATCATCC |
| 1085 | Left | GTGCACAATAAATTAAAAGGGAAAG |
| 1086 | Right | AAACAAAGCTGAATCATCCTACATC |
| 1087 | Left | TGCACAATAAATTAAAAGGGAAAGA |
| 1088 | Right | AAAACAAAGCTGAATCATCCTACAT |
| 1089 | Left | ATCATATTACCTGGGAAGACTTCAA |
| 1090 | Right | AAACAAAGCTGAATCATCCTACATC |
| 1091 | Left | GTGCACAATAAATTAAAAGGGAAAG |
| 1092 | Right | ACAATAAAACAAAGCTGAATCATCC |
| 1093 | Left | TGTGCACAATAAATTAAAAGGGAAA |
| 1094 | Right | AAACAAAGCTGAATCATCCTACATC |
| 1095 | Left | TGCACAATAAATTAAAAGGGAAAGA |
| 1096 | Right | TAAAACAAAGCTGAATCATCCTACA |
| 1097 | Left | TAAATTAAAAGGGAAAGAACACCTG |
| 1098 | Right | AAACAAAGCTGAATCATCCTACATC |
| 1099 | Left | GCACAATAAATTAAAAGGGAAAGAA |
| 1100 | Right | AAACAAAGCTGAATCATCCTACATC |
| 1101 | Left | TGGGAAGACTTCAAATGTACAAATA |
| 1102 | Right | AAACAAAGCTGAATCATCCTACATC |
| 1103 | Left | TGCACAATAAATTAAAAGGGAAAGA |
| 1104 | Right | GAGACCTAGTATTCTGCTCTGAAGG |
| 1105 | Left | TGCACAATAAATTAAAAGGGAAAGA |
| 1106 | Right | GGAGACCTAGTATTCTGCTCTGAAG |
| 1107 | Left | TGTGCACAATAAATTAAAAGGGAAA |
| 1108 | Right | ACAATAAAACAAAGCTGAATCATCC |
| 1109 | Left | TGTTTATAAATTGGGGGTATTCAAA |
| 1110 | Right | GAGACCTAGTATTCTGCTCTGAAGG |
| 1111 | Left | TTGTTTATAAATTGGGGGTATTCAA |
| 1112 | Right | GAGACCTAGTATTCTGCTCTGAAGG |
| 1113 | Left | TGTTTATAAATTGGGGGTATTCAAA |
| 1114 | Right | GGAGACCTAGTATTCTGCTCTGAAG |
| 1115 | Left | TTGTTTATAAATTGGGGGTATTCAA |
| 1116 | Right | GGAGACCTAGTATTCTGCTCTGAAG |
| 1117 | Left | GTGCACAATAAATTAAAAGGGAAAG |
| 1118 | Right | AAAACAAAGCTGAATCATCCTACAT |
| 1119 | Left | TAAATTAAAAGGGAAAGAACACCTG |
| 1120 | Right | ACAATAAAACAAAGCTGAATCATCC |

ALK Exon24 601-800 bases

| | | |
|---|---|---|
| 1121 | Left | TGTTTATAAATTGGGGGTATTCAAA |
| 1122 | Right | AAACAAAGCTGAATCATCCTACATC |
| 1123 | Left | TTGTTTATAAATTGGGGGTATTCAA |
| 1124 | Right | AAACAAAGCTGAATCATCCTACATC |
| 1125 | Left | TGTTTATAAATTGGGGGTATTCAAA |
| 1126 | Right | ACAATAAAACAAAGCTGAATCATCC |
| 1127 | Left | TGCACAATAAATTAAAAGGGAAAGA |
| 1128 | Right | AGTTACCATCTCAAAGACAAAGCTG |
| 1129 | Left | TGTTTATAAATTGGGGGTATTCAAA |
| 1130 | Right | AGTTACCATCTCAAAGACAAAGCTG |
| 1131 | Left | TTGTTTATAAATTGGGGGTATTCAA |
| 1132 | Right | AGTTACCATCTCAAAGACAAAGCTG |
| 1133 | Left | CAAACTTGTTTATAAATTGGGGGTA |
| 1134 | Right | AAACAAAGCTGAATCATCCTACATC |
| 1135 | Left | CTTGTTTATAAATTGGGGGTATTCA |
| 1136 | Right | AAACAAAGCTGAATCATCCTACATC |
| 1137 | Left | TGCACAATAAATTAAAAGGGAAAGA |
| 1138 | Right | GCAAGTGAATCCCTGATAGAATAAG |
| 1139 | Left | CTGGATCTGCTTGAAGAAAATTAGT |
| 1140 | Right | AAACAAAGCTGAATCATCCTACATC |
| 1141 | Left | CAAACTTGTTTATAAATTGGGGGTA |
| 1142 | Right | ACAATAAAACAAAGCTGAATCATCC |
| 1143 | Left | TTTATAAATTGGGGGTATTCAAATG |
| 1144 | Right | AAACAAAGCTGAATCATCCTACATC |
| 1145 | Left | TGTTTATAAATTGGGGGTATTCAAA |
| 1146 | Right | AAAACAAAGCTGAATCATCCTACAT |
| 1147 | Left | TTGTTTATAAATTGGGGGTATTCAA |
| 1148 | Right | AAAACAAAGCTGAATCATCCTACAT |
| 1149 | Left | CTGGATCTGCTTGAAGAAAATTAGT |
| 1150 | Right | ACAATAAAACAAAGCTGAATCATCC |
| 1151 | Left | GTGCACAATAAATTAAAAGGGAAAG |
| 1152 | Right | AGTTACCATCTCAAAGACAAAGCTG |
| 1153 | Left | ATCATATTACCTGGGAAGACTTCAA |
| 1154 | Right | GCGAGGATATTTTATGACACTTGTT |
| 1155 | Left | TTTATAAATTGGGGGTATTCAAATG |
| 1156 | Right | ACAATAAAACAAAGCTGAATCATCC |
| 1157 | Left | ATCTCCTTTTGAATGAAAGAGACCT |
| 1158 | Right | GAGACCTAGTATTCTGCTCTGAAGG |
| 1159 | Left | ATCTCCTTTTGAATGAAAGAGACCT |
| 1160 | Right | GGAGACCTAGTATTCTGCTCTGAAG |

ALK Exon24 801-1000 bases

| | | |
|---|---|---|
| 1161 | Left | TAGGAATTAAAAGAGAGGCCAAGAT |
| 1162 | Right | AAACAAAGCTGAATCATCCTACATC |
| 1163 | Left | ATCTCCTTTTGAATGAAAGAGACCT |
| 1164 | Right | AAACAAAGCTGAATCATCCTACATC |
| 1165 | Left | TAGGAATTAAAAGAGAGGCCAAGAT |
| 1166 | Right | ACAATAAAACAAAGCTGAATCATCC |
| 1167 | Left | ATCTCCTTTTGAATGAAAGAGACCT |
| 1168 | Right | ACAATAAAACAAAGCTGAATCATCC |
| 1169 | Left | TGCACAATAAATTAAAAGGGAAAGA |
| 1170 | Right | AAAGATGACTAAAACAGCATCCTTG |
| 1171 | Left | ACGTCAGGGATTTAGGAATTAAAAG |
| 1172 | Right | ACAATAAAACAAAGCTGAATCATCC |
| 1173 | Left | TGCACAATAAATTAAAAGGGAAAGA |
| 1174 | Right | GCGAGGATATTTTATGACACTTGTT |
| 1175 | Left | TGTTTATAAATTGGGGGTATTCAAA |
| 1176 | Right | GCGAGGATATTTTATGACACTTGTT |
| 1177 | Left | TTGTTTATAAATTGGGGGTATTCAA |
| 1178 | Right | GCGAGGATATTTTATGACACTTGTT |
| 1179 | Left | TGCACAATAAATTAAAAGGGAAAGA |
| 1180 | Right | TTGTCAAAAATGCAATTCCTTAACT |
| 1181 | Left | TTTAGGAATTAAAAGAGAGGCCAAG |
| 1182 | Right | AAACAAAGCTGAATCATCCTACATC |
| 1183 | Left | TAGGAATTAAAAGAGAGGCCAAGAT |
| 1184 | Right | AAAACAAAGCTGAATCATCCTACAT |
| 1185 | Left | ATCTCCTTTTGAATGAAAGAGACCT |
| 1186 | Right | AAAACAAAGCTGAATCATCCTACAT |
| 1187 | Left | AAAGCTTGAGATAGCTCATAATTGC |
| 1188 | Right | AAACAAAGCTGAATCATCCTACATC |
| 1189 | Left | TTTAGGAATTAAAAGAGAGGCCAAG |
| 1190 | Right | ACAATAAAACAAAGCTGAATCATCC |
| 1191 | Left | ACGTCAGGGATTTAGGAATTAAAAG |
| 1192 | Right | AAAACAAAGCTGAATCATCCTACAT |
| 1193 | Left | TGTTTATAAATTGGGGGTATTCAAA |
| 1194 | Right | GCAAGTGAATCCCTGATAGAATAAG |
| 1195 | Left | TTGTTTATAAATTGGGGGTATTCAA |
| 1196 | Right | GCAAGTGAATCCCTGATAGAATAAG |
| 1197 | Left | GTGCACAATAAATTAAAAGGGAAAG |
| 1198 | Right | AAAGATGACTAAAACAGCATCCTTG |
| 1199 | Left | CAAACTTGTTTATAAATTGGGGGTA |
| 1200 | Right | GCGAGGATATTTTATGACACTTGTT |

ALK Exon24 2 kb

| | | |
|---|---|---|
| 1201 | Left | ATCTCCTTTTGAATGAAAGAGACCT |
| 1202 | Right | CATGTTAGGAGTGACTTTGGAACTT |
| 1203 | Left | ACTGTAGTCACATACATACGCTCCA |
| 1204 | Right | AAACAAAGCTGAATCATCCTACATC |
| 1205 | Left | ATCTCCTTTTGAATGAAAGAGACCT |
| 1206 | Right | CTAAAAGGATGAAGTGACAGGAAGA |
| 1207 | Left | ATCTCCTTTTGAATGAAAGAGACCT |
| 1208 | Right | TAAAAGGATGAAGTGACAGGAAGAG |
| 1209 | Left | ACTGTAGTCACATACATACGCTCCA |
| 1210 | Right | GCGAGGATATTTTATGACACTTGTT |
| 1211 | Left | ACTGTAGTCACATACATACGCTCCA |
| 1212 | Right | ACAATAAAACAAAGCTGAATCATCC |
| 1213 | Left | ACGTCAGGGATTTAGGAATTAAAAG |
| 1214 | Right | CAAGTGTACTTCCTGACCTCTCATT |
| 1215 | Left | TGCACAATAAATTAAAAGGGAAAGA |
| 1216 | Right | CATGTTAGGAGTGACTTTGGAACTT |
| 1217 | Left | ACTGTAGTCACATACATACGCTCCA |
| 1218 | Right | AGTTACCATCTCAAAGACAAAGCTG |
| 1219 | Left | TGTTTATAAATTGGGGGTATTCAAA |
| 1220 | Right | CATGTTAGGAGTGACTTTGGAACTT |
| 1221 | Left | TTGTTTATAAATTGGGGGTATTCAA |
| 1222 | Right | CATGTTAGGAGTGACTTTGGAACTT |
| 1223 | Left | ATCTCCTTTTGAATGAAAGAGACCT |
| 1224 | Right | ATAAGCTCTTCTGAGAGTTGACTGC |
| 1225 | Left | AGGCACTCTCTCTTCCATTTTAACT |
| 1226 | Right | AAACAAAGCTGAATCATCCTACATC |
| 1227 | Left | TGCACAATAAATTAAAAGGGAAAGA |
| 1228 | Right | AAGGGCTGAAAAACTACCTTAAAAA |
| 1229 | Left | TGCACAATAAATTAAAAGGGAAAGA |
| 1230 | Right | AAAGGGCTGAAAAACTACCTTAAAA |
| 1231 | Left | TGCACAATAAATTAAAAGGGAAAGA |
| 1232 | Right | TTAGAAGGAGCAGATGGTAAAGCTA |
| 1233 | Left | TGTTTATAAATTGGGGGTATTCAAA |
| 1234 | Right | AAGGGCTGAAAAACTACCTTAAAAA |
| 1235 | Left | TGTTTATAAATTGGGGGTATTCAAA |
| 1236 | Right | AAAGGGCTGAAAAACTACCTTAAAA |
| 1237 | Left | TTGTTTATAAATTGGGGGTATTCAA |
| 1238 | Right | AAAGGGCTGAAAAACTACCTTAAAA |
| 1239 | Left | TTGTTTATAAATTGGGGGTATTCAA |
| 1240 | Right | AAGGGCTGAAAAACTACCTTAAAAA |

ALK Exon25 130-160 bases

| | | |
|---|---|---|
| 1241 | Left | TCCTAGGGATAAAATTAGGAAATGC |
| 1242 | Right | AGGGGTGAGGCAGTCTTTACTC |
| 1243 | Left | TCCTAGGGATAAAATTAGGAAATGC |
| 1244 | Right | GAGGGGTGAGGCAGTCTTTACT |
| 1245 | Left | TCCTAGGGATAAAATTAGGAAATGC |
| 1246 | Right | GGGGTGAGGCAGTCTTTACTC |
| 1247 | Left | TCCTAGGGATAAAATTAGGAAATGC |
| 1248 | Right | GAGGGGTGAGGCAGTCTTTAC |
| 1249 | Left | TCCTAGGGATAAAATTAGGAAATGC |
| 1250 | Right | GGGTGAGGCAGTCTTTACTCA |
| 1251 | Left | TCCTAGGGATAAAATTAGGAAATGC |
| 1252 | Right | GAGGGGTGAGGCAGTCTTTACTC |
| 1253 | Left | TCCTAGGGATAAAATTAGGAAATGC |
| 1254 | Right | AGGGGTGAGGCAGTCTTTACT |
| 1255 | Left | TCCTAGGGATAAAATTAGGAAATGC |
| 1256 | Right | AGGGGTGAGGCAGTCTTTACTCA |
| 1257 | Left | TCCTAGGGATAAAATTAGGAAATGC |
| 1258 | Right | GAGGGGTGAGGCAGTCTTTA |
| 1259 | Left | TTCCTAGGGATAAAATTAGGAAATG |
| 1260 | Right | AGGGGTGAGGCAGTCTTTACTC |
| 1261 | Left | TCCTAGGGATAAAATTAGGAAATGC |
| 1262 | Right | GGGGTGAGGCAGTCTTTACTCA |
| 1263 | Left | TTCCTAGGGATAAAATTAGGAAATG |
| 1264 | Right | GGGGTGAGGCAGTCTTTACTC |
| 1265 | Left | TTCCTAGGGATAAAATTAGGAAATGC |
| 1266 | Right | AGGGGTGAGGCAGTCTTTACTC |
| 1267 | Left | CTTCCTAGGGATAAAATTAGGAAATG |
| 1268 | Right | GGGGTGAGGCAGTCTTTACTC |
| 1269 | Left | TCCTAGGGATAAAATTAGGAAATGC |

ALK Exon25 161-200 bases

| | | |
|---|---|---|
| 1270 | Right | GAGGGGTGAGGCAGTCTTTACTCA |
| 1271 | Left | TTCCTAGGGATAAAATTAGGAAATGC |
| 1272 | Right | GGGGTGAGGCAGTCTTTACTC |
| 1273 | Left | CCTAGGGATAAAATTAGGAAATGC |
| 1274 | Right | AGGGGTGAGGCAGTCTTTACTC |
| 1275 | Left | CCTAGGGATAAAATTAGGAAATGC |
| 1276 | Right | GAGGGGTGAGGCAGTCTTTACT |
| 1277 | Left | TTCCTAGGGATAAAATTAGGAAATG |
| 1278 | Right | GGGTGAGGCAGTCTTTACTCA |
| 1279 | Left | TTCCTAGGGATAAAATTAGGAAATG |
| 1280 | Right | AGGGGTGAGGCAGTCTTTACT |
| 1281 | Left | CTTGGAGATAAAATCCTAGTGATGG |
| 1282 | Right | GGGGTGAGGCAGTCTTTACTC |
| 1283 | Left | TCCTAGGGATAAAATTAGGAAATGC |
| 1284 | Right | GCTGAGGTGGAAGAGACAGG |
| 1285 | Left | TCCTAGGGATAAAATTAGGAAATGC |
| 1286 | Right | GGCTGAGGTGGAAGAGACAG |
| 1287 | Left | CTTGGAGATAAAATCCTAGTGATGG |
| 1288 | Right | GGGTGAGGCAGTCTTTACTCA |
| 1289 | Left | TGTACACTCATCTTCCTAGGGATAAA |
| 1290 | Right | GAGGGGTGAGGCAGTCTTTACT |
| 1291 | Left | TGTACACTCATCTTCCTAGGGATAAA |
| 1292 | Right | GAGGGGTGAGGCAGTCTTTAC |
| 1293 | Left | TCATCTTCCTAGGGATAAAATTAGG |
| 1294 | Right | AGGGGTGAGGCAGTCTTTACTC |
| 1295 | Left | TCATCTTCCTAGGGATAAAATTAGG |
| 1296 | Right | GAGGGGTGAGGCAGTCTTTACT |
| 1297 | Left | TTCCTAGGGATAAAATTAGGAAATG |
| 1298 | Right | GAGGGGTGAGGCAGTCTTTACT |
| 1299 | Left | TCATCTTCCTAGGGATAAAATTAGG |
| 1300 | Right | GGGGTGAGGCAGTCTTTACTC |
| 1301 | Left | TCATCTTCCTAGGGATAAAATTAGG |
| 1302 | Right | GAGGGGTGAGGCAGTCTTTAC |
| 1303 | Left | TTCCTAGGGATAAAATTAGGAAATG |
| 1304 | Right | GAGGGGTGAGGCAGTCTTTAC |
| 1305 | Left | CTCATCTTCCTAGGGATAAAATTAGG |
| 1306 | Right | AGGGGTGAGGCAGTCTTTACTC |
| 1307 | Left | CTCATCTTCCTAGGGATAAAATTAGG |
| 1308 | Right | GAGGGGTGAGGCAGTCTTTACT |
| 1309 | Left | CTTCCTAGGGATAAAATTAGGAAATG |
| 1310 | Right | AGGGGTGAGGCAGTCTTTACTC |
| 1311 | Left | CTTCCTAGGGATAAAATTAGGAAATG |
| 1312 | Right | GAGGGGTGAGGCAGTCTTTACT |
| 1313 | Left | CTCATCTTCCTAGGGATAAAATTAGG |
| 1314 | Right | GGGGTGAGGCAGTCTTTACTC |
| 1315 | Left | CTCATCTTCCTAGGGATAAAATTAGG |
| 1316 | Right | GAGGGGTGAGGCAGTCTTTAC |
| 1317 | Left | TTCCTAGGGATAAAATTAGGAAATGC |
| 1318 | Right | GAGGGGTGAGGCAGTCTTTACT |
| 1319 | Left | CTTCCTAGGGATAAAATTAGGAAATG |
| 1320 | Right | GAGGGGTGAGGCAGTCTTTAC |

ALK Exon25 201-300 bases

| | | |
|---|---|---|
| 1321 | Left | TCCTAGGGATAAAATTAGGAAATGC |
| 1322 | Right | AGCCTGAAAAGGAACTTAGTGAAAT |
| 1323 | Left | TCCTAGGGATAAAATTAGGAAATGC |
| 1324 | Right | TAGCCTGAAAAGGAACTTAGTGAAA |
| 1325 | Left | TCCTAGGGATAAAATTAGGAAATGC |
| 1326 | Right | CATAGCCTGAAAAGGAACTTAGTGA |
| 1327 | Left | TCCTAGGGATAAAATTAGGAAATGC |
| 1328 | Right | CTAATTAAGGTTTCCCATAGCCTGA |
| 1329 | Left | TCCTAGGGATAAAATTAGGAAATGC |
| 1330 | Right | ATAGCCTGAAAAGGAACTTAGTGAAA |
| 1331 | Left | TCCTAGGGATAAAATTAGGAAATGC |
| 1332 | Right | TAGCCTGAAAAGGAACTTAGTGAAAT |
| 1333 | Left | TCCTAGGGATAAAATTAGGAAATGC |
| 1334 | Right | AGGTAGAAAGTTGACAGGGTACCAG |
| 1335 | Left | TCCTAGGGATAAAATTAGGAAATGC |
| 1336 | Right | AGCCTGAAAAGGAACTTAGTGAAA |
| 1337 | Left | TCCTAGGGATAAAATTAGGAAATGC |
| 1338 | Right | TAATTAAGGTTTCCCATAGCCTGA |
| 1339 | Left | TCCTAGGGATAAAATTAGGAAATGC |
| 1340 | Right | TAATTAAGGTTTCCCATAGCCTGAA |
| 1341 | Left | TCCTAGGGATAAAATTAGGAAATGC |
| 1342 | Right | GGTAGAAAGTTGACAGGGTACCAG |
| 1343 | Left | TGTACACTCATCTTCCTAGGGATAAA |
| 1344 | Right | AGCCTGAAAAGGAACTTAGTGAAAT |
| 1345 | Left | TGTACACTCATCTTCCTAGGGATAAA |
| 1346 | Right | TAGCCTGAAAAGGAACTTAGTGAAA |
| 1347 | Left | TCCTAGGGATAAAATTAGGAAATGC |
| 1348 | Right | CCATAGCCTGAAAAGGAACTTAGTG |
| 1349 | Left | CTTGGAGATAAAATCCTAGTGATGG |
| 1350 | Right | AGGTAGAAAGTTGACAGGGTACCAG |
| 1351 | Left | TGTACACTCATCTTCCTAGGGATAAA |
| 1352 | Right | CATAGCCTGAAAAGGAACTTAGTGA |
| 1353 | Left | TCCTAGGGATAAAATTAGGAAATGC |
| 1354 | Right | CACTAATTAAGGTTTCCCATAGCC |
| 1355 | Left | TCATCTTCCTAGGGATAAAATTAGG |
| 1356 | Right | AGCCTGAAAAGGAACTTAGTGAAAT |
| 1357 | Left | TCCTAGGGATAAAATTAGGAAATGC |
| 1358 | Right | GCCTGAAAAGGAACTTAGTGAAAT |
| 1359 | Left | AACTTCAGCTTGGAGATAAAATCCT |
| 1360 | Right | ACAGGGTACCAGGAGATGATGTAAG |

ALK Exon25 301-400 bases

| | | |
|---|---|---|
| 1361 | Left | AACTTCAGCTTGGAGATAAAATCCT |
| 1362 | Right | TGGTCACTAATTAAGGTTTCCCATA |
| 1363 | Left | GGGAAGGAACTATTTAAACTTCAGC |
| 1364 | Right | TGGTCACTAATTAAGGTTTCCCATA |
| 1365 | Left | TCCTAGGGATAAAATTAGGAAATGC |
| 1366 | Right | TGGTCACTAATTAAGGTTTCCCATA |
| 1367 | Left | AACTTCAGCTTGGAGATAAAATCCT |
| 1368 | Right | AGCCTGAAAAGGAACTTAGTGAAAT |
| 1369 | Left | GGGAAGGAACTATTTAAACTTCAGC |
| 1370 | Right | AGCCTGAAAAGGAACTTAGTGAAAT |
| 1371 | Left | AACTTCAGCTTGGAGATAAAATCCT |
| 1372 | Right | TAGCCTGAAAAGGAACTTAGTGAAA |
| 1373 | Left | GGGAAGGAACTATTTAAACTTCAGC |
| 1374 | Right | TAGCCTGAAAAGGAACTTAGTGAAA |
| 1375 | Left | AACTTCAGCTTGGAGATAAAATCCT |
| 1376 | Right | CATAGCCTGAAAAGGAACTTAGTGA |
| 1377 | Left | GGGAAGGAACTATTTAAACTTCAGC |
| 1378 | Right | CATAGCCTGAAAAGGAACTTAGTGA |
| 1379 | Left | AAACTTCAGCTTGGAGATAAAATCC |
| 1380 | Right | TGGTCACTAATTAAGGTTTCCCATA |
| 1381 | Left | AAACTTCAGCTTGGAGATAAAATCC |
| 1382 | Right | AGCCTGAAAAGGAACTTAGTGAAAT |
| 1383 | Left | AAACTTCAGCTTGGAGATAAAATCC |
| 1384 | Right | TAGCCTGAAAAGGAACTTAGTGAAA |
| 1385 | Left | AAACTTCAGCTTGGAGATAAAATCC |
| 1386 | Right | CATAGCCTGAAAAGGAACTTAGTGA |
| 1387 | Left | CTTGGAGATAAAATCCTAGTGATGG |
| 1388 | Right | TGGTCACTAATTAAGGTTTCCCATA |
| 1389 | Left | CTTGGAGATAAAATCCTAGTGATGG |
| 1390 | Right | AGCCTGAAAAGGAACTTAGTGAAAT |
| 1391 | Left | CTTGGAGATAAAATCCTAGTGATGG |
| 1392 | Right | TAGCCTGAAAAGGAACTTAGTGAAA |
| 1393 | Left | GGAACTATTTAAACTTCAGCTTGGA |
| 1394 | Right | TGGTCACTAATTAAGGTTTCCCATA |
| 1395 | Left | CTTGGAGATAAAATCCTAGTGATGG |
| 1396 | Right | CATAGCCTGAAAAGGAACTTAGTGA |
| 1397 | Left | GGAACTATTTAAACTTCAGCTTGGA |
| 1398 | Right | AGCCTGAAAAGGAACTTAGTGAAAT |
| 1399 | Left | GGAACTATTTAAACTTCAGCTTGGA |
| 1400 | Right | TAGCCTGAAAAGGAACTTAGTGAAA |

ALK Exon25 401-600 bases

| | | |
|---|---|---|
| 1401 | Left | TTAATCATTTCCCCTAATCCTTTTC |
| 1402 | Right | TGGTCACTAATTAAGGTTTCCCATA |
| 1403 | Left | GAAGCACTTACAACAACACTTAGCA |
| 1404 | Right | TGGTCACTAATTAAGGTTTCCCATA |
| 1405 | Left | TGAAGCACTTACAACAACACTTAGC |
| 1406 | Right | TGGTCACTAATTAAGGTTTCCCATA |
| 1407 | Left | ATTGTTAAGGCTGTTTCTCTCACAC |
| 1408 | Right | TGGTCACTAATTAAGGTTTCCCATA |
| 1409 | Left | GAAGCACTTACAACAACACTTAGCA |
| 1410 | Right | AGCCTGAAAAGGAACTTAGTGAAAT |
| 1411 | Left | TGAAGCACTTACAACAACACTTAGC |
| 1412 | Right | AGCCTGAAAAGGAACTTAGTGAAAT |
| 1413 | Left | CATTTTTAATCATTTCCCCTAATCC |
| 1414 | Right | TGGTCACTAATTAAGGTTTCCCATA |
| 1415 | Left | AGAATTGTTAAGGCTGTTTCTCTCA |
| 1416 | Right | TGGTCACTAATTAAGGTTTCCCATA |
| 1417 | Left | ATTGTTAAGGCTGTTTCTCTCACAC |
| 1418 | Right | AGCCTGAAAAGGAACTTAGTGAAAT |
| 1419 | Left | GCACCTTGTGTCTTATAAGGTTGTT |
| 1420 | Right | AGCCTGAAAAGGAACTTAGTGAAAT |
| 1421 | Left | TTCCATTTCTCTCTTAGTTGTGAGG |
| 1422 | Right | TGGTCACTAATTAAGGTTTCCCATA |
| 1423 | Left | GAAGCACTTACAACAACACTTAGCA |
| 1424 | Right | TAGCCTGAAAAGGAACTTAGTGAAA |
| 1425 | Left | TGAAGCACTTACAACAACACTTAGC |
| 1426 | Right | TAGCCTGAAAAGGAACTTAGTGAAA |
| 1427 | Left | GAAGCACTTACAACAACACTTAGCA |
| 1428 | Right | CATAGCCTGAAAAGGAACTTAGTGA |
| 1429 | Left | TGAAGCACTTACAACAACACTTAGC |
| 1430 | Right | CATAGCCTGAAAAGGAACTTAGTGA |
| 1431 | Left | ATTGTTAAGGCTGTTTCTCTCACAC |
| 1432 | Right | TAGCCTGAAAAGGAACTTAGTGAAA |
| 1433 | Left | GCACCTTGTGTCTTATAAGGTTGTT |
| 1434 | Right | TAGCCTGAAAAGGAACTTAGTGAAA |
| 1435 | Left | GAATTGTTAAGGCTGTTTCTCTCAC |
| 1436 | Right | TGGTCACTAATTAAGGTTTCCCATA |
| 1437 | Left | ATTGTTAAGGCTGTTTCTCTCACAC |
| 1438 | Right | CATAGCCTGAAAAGGAACTTAGTGA |
| 1439 | Left | GCACCTTGTGTCTTATAAGGTTGTT |
| 1440 | Right | CATAGCCTGAAAAGGAACTTAGTGA |

ALK Exon25 401-600 bases

| | | |
|---|---|---|
| 1441 | Left | TTAATCATTTCCCCTAATCCTTTTC |
| 1442 | Right | TGGTCACTAATTAAGGTTTCCCATA |
| 1443 | Left | GAAGCACTTACAACAACACTTAGCA |
| 1444 | Right | TGGTCACTAATTAAGGTTTCCCATA |
| 1445 | Left | TGAAGCACTTACAACAACACTTAGC |
| 1446 | Right | TGGTCACTAATTAAGGTTTCCCATA |
| 1447 | Left | ATTGTTAAGGCTGTTTCTCTCACAC |
| 1448 | Right | TGGTCACTAATTAAGGTTTCCCATA |
| 1449 | Left | GAAGCACTTACAACAACACTTAGCA |
| 1450 | Right | AGCCTGAAAAGGAACTTAGTGAAAT |
| 1451 | Left | TGAAGCACTTACAACAACACTTAGC |
| 1452 | Right | AGCCTGAAAAGGAACTTAGTGAAAT |
| 1453 | Left | CATTTTTAATCATTTCCCCTAATCC |
| 1454 | Right | TGGTCACTAATTAAGGTTTCCCATA |
| 1455 | Left | AGAATTGTTAAGGCTGTTTCTCTCA |
| 1456 | Right | TGGTCACTAATTAAGGTTTCCCATA |
| 1457 | Left | ATTGTTAAGGCTGTTTCTCTCACAC |
| 1458 | Right | AGCCTGAAAAGGAACTTAGTGAAAT |
| 1459 | Left | GCACCTTGTGTCTTATAAGGTTGTT |
| 1460 | Right | AGCCTGAAAAGGAACTTAGTGAAAT |
| 1461 | Left | TTCCATTTCTCTCTTAGTTGTGAGG |
| 1462 | Right | TGGTCACTAATTAAGGTTTCCCATA |
| 1463 | Left | GAAGCACTTACAACAACACTTAGCA |
| 1464 | Right | TAGCCTGAAAAGGAACTTAGTGAAA |
| 1465 | Left | TGAAGCACTTACAACAACACTTAGC |
| 1466 | Right | TAGCCTGAAAAGGAACTTAGTGAAA |
| 1467 | Left | GAAGCACTTACAACAACACTTAGCA |
| 1468 | Right | CATAGCCTGAAAAGGAACTTAGTGA |
| 1469 | Left | TGAAGCACTTACAACAACACTTAGC |
| 1470 | Right | CATAGCCTGAAAAGGAACTTAGTGA |
| 1471 | Left | ATTGTTAAGGCTGTTTCTCTCACAC |
| 1472 | Right | TAGCCTGAAAAGGAACTTAGTGAAA |
| 1473 | Left | GCACCTTGTGTCTTATAAGGTTGTT |
| 1474 | Right | TAGCCTGAAAAGGAACTTAGTGAAA |
| 1475 | Left | GAATTGTTAAGGCTGTTTCTCTCAC |
| 1476 | Right | TGGTCACTAATTAAGGTTTCCCATA |
| 1477 | Left | ATTGTTAAGGCTGTTTCTCTCACAC |
| 1478 | Right | CATAGCCTGAAAAGGAACTTAGTGA |
| 1479 | Left | GCACCTTGTGTCTTATAAGGTTGTT |
| 1480 | Right | CATAGCCTGAAAAGGAACTTAGTGA |

ALK Exon25 601-800 bases

| | | |
|---|---|---|
| 1481 | Left | GGACAGTAATAGCACCTTGTGTCTT |
| 1482 | Right | TGGTCACTAATTAAGGTTTCCCATA |
| 1483 | Left | AAATGAGGACAGTAATAGCACCTTG |
| 1484 | Right | TGGTCACTAATTAAGGTTTCCCATA |
| 1485 | Left | GGACAGTAATAGCACCTTGTGTCTT |
| 1486 | Right | AGCCTGAAAAGGAACTTAGTGAAAT |
| 1487 | Left | AAATGAGGACAGTAATAGCACCTTG |
| 1488 | Right | AGCCTGAAAAGGAACTTAGTGAAAT |
| 1489 | Left | GCACCTTGTGTCTTATAAGGTTGTT |
| 1490 | Right | TGGTCACTAATTAAGGTTTCCCATA |
| 1491 | Left | GGACAGTAATAGCACCTTGTGTCTT |
| 1492 | Right | TAGCCTGAAAAGGAACTTAGTGAAA |
| 1493 | Left | AAATGAGGACAGTAATAGCACCTTG |
| 1494 | Right | TAGCCTGAAAAGGAACTTAGTGAAA |
| 1495 | Left | AACTTCAGCTTGGAGATAAAATCCT |
| 1496 | Right | AGTATCCAAGTTATCCCATGTCTCA |
| 1497 | Left | AACTTCAGCTTGGAGATAAAATCCT |
| 1498 | Right | GTATCCAAGTTATCCCATGTCTCAG |
| 1499 | Left | GGACAGTAATAGCACCTTGTGTCTT |
| 1500 | Right | CATAGCCTGAAAAGGAACTTAGTGA |
| 1501 | Left | AAATGAGGACAGTAATAGCACCTTG |
| 1502 | Right | CATAGCCTGAAAAGGAACTTAGTGA |
| 1503 | Left | TCCTAGGGATAAAATTAGGAAATGC |
| 1504 | Right | AGTATCCAAGTTATCCCATGTCTCA |
| 1505 | Left | TCCTAGGGATAAAATTAGGAAATGC |
| 1506 | Right | GTATCCAAGTTATCCCATGTCTCAG |
| 1507 | Left | TCCTAGGGATAAAATTAGGAAATGC |
| 1508 | Right | TCAATAAAGCTCACTTTGAAGGTCT |
| 1509 | Left | TCCTAGGGATAAAATTAGGAAATGC |
| 1510 | Right | AGTTGAGGAAGTTCAATAAAGCTCA |
| 1511 | Left | TCCTAGGGATAAAATTAGGAAATGC |
| 1512 | Right | TTGAGGAAGTTCAATAAAGCTCACT |
| 1513 | Left | TCCTAGGGATAAAATTAGGAAATGC |
| 1514 | Right | TGAGGAAGTTCAATAAAGCTCACTT |
| 1515 | Left | TCCTAGGGATAAAATTAGGAAATGC |
| 1516 | Right | CTGAGGAGTTGAGGAAGTTCAATAA |
| 1517 | Left | TCCTAGGGATAAAATTAGGAAATGC |
| 1518 | Right | GGAAGTTCAATAAAGCTCACTTTGA |
| 1519 | Left | TCCTAGGGATAAAATTAGGAAATGC |
| 1520 | Right | TTCAATAAAGCTCACTTTGAAGGTC |

ALK Exon25 801-1000 bases

| | | |
|---|---|---|
| 1521 | Left | AACTTCAGCTTGGAGATAAAATCCT |
| 1522 | Right | AGTACTGAGGAGTTGAGGAAGTTCA |
| 1523 | Left | TTAATCATTTCCCCTAATCCTTTTC |
| 1524 | Right | AGTACTGAGGAGTTGAGGAAGTTCA |
| 1525 | Left | TTAATCATTTCCCCTAATCCTTTTC |
| 1526 | Right | TGAGTACTGAGGAGTTGAGGAAGTT |
| 1527 | Left | AACTTCAGCTTGGAGATAAAATCCT |
| 1528 | Right | TCAATAAAGCTCACTTTGAAGGTCT |
| 1529 | Left | AACTTCAGCTTGGAGATAAAATCCT |
| 1530 | Right | TTGAGGAAGTTCAATAAAGCTCACT |
| 1531 | Left | AACTTCAGCTTGGAGATAAAATCCT |
| 1532 | Right | TGAGGAAGTTCAATAAAGCTCACTT |
| 1533 | Left | TTAATCATTTCCCCTAATCCTTTTC |
| 1534 | Right | AGTATCCAAGTTATCCCATGTCTCA |
| 1535 | Left | TTAATCATTTCCCCTAATCCTTTTC |
| 1536 | Right | GTATCCAAGTTATCCCATGTCTCAG |
| 1537 | Left | TCCTAGGGATAAAATTAGGAAATGC |
| 1538 | Right | AGTACTGAGGAGTTGAGGAAGTTCA |
| 1539 | Left | TCCTAGGGATAAAATTAGGAAATGC |
| 1540 | Right | TGAGTACTGAGGAGTTGAGGAAGTT |
| 1541 | Left | TTAATCATTTCCCCTAATCCTTTTC |
| 1542 | Right | TCAATAAAGCTCACTTTGAAGGTCT |
| 1543 | Left | TTAATCATTTCCCCTAATCCTTTTC |
| 1544 | Right | AGTTGAGGAAGTTCAATAAAGCTCA |
| 1545 | Left | TTAATCATTTCCCCTAATCCTTTTC |
| 1546 | Right | TGAGGAAGTTCAATAAAGCTCACTT |
| 1547 | Left | TTAATCATTTCCCCTAATCCTTTTC |
| 1548 | Right | TTGAGGAAGTTCAATAAAGCTCACT |
| 1549 | Left | AACTTCAGCTTGGAGATAAAATCCT |
| 1550 | Right | CTGAGGAGTTGAGGAAGTTCAATAA |
| 1551 | Left | GGGAAGGAACTATTTAAACTTCAGC |
| 1552 | Right | AGTATCCAAGTTATCCCATGTCTCA |
| 1553 | Left | GGGAAGGAACTATTTAAACTTCAGC |
| 1554 | Right | GTATCCAAGTTATCCCATGTCTCAG |
| 1555 | Left | AACTTCAGCTTGGAGATAAAATCCT |
| 1556 | Right | TTCAATAAAGCTCACTTTGAAGGTC |
| 1557 | Left | AACTTCAGCTTGGAGATAAAATCCT |
| 1558 | Right | GGAAGTTCAATAAAGCTCACTTTGA |
| 1559 | Left | AACTTCAGCTTGGAGATAAAATCCT |
| 1560 | Right | GTCTTTCCACATCAAGTATCCAAGT |

ALK Exon25 2 kb

| | | |
|---|---|---|
| 1561 | Left | GGACAGTAATAGCACCTTGTGTCTT |
| 1562 | Right | GTGCTGAGGACATAAATAGGTCAGT |
| 1563 | Left | AAAATCATGGACAAAAGAACCATAA |
| 1564 | Right | TTAAAGCTCCATATAACGATTGCTC |
| 1565 | Left | GGACAGTAATAGCACCTTGTGTCTT |
| 1566 | Right | AGTCTCTCTCTCCCAAGGATATTGT |
| 1567 | Left | AAAATCATGGACAAAAGAACCATAA |
| 1568 | Right | AGTCTCACTTATTCCCCAAAGAGTT |
| 1569 | Left | ATCACTTTTTAAAACAACCATTCCA |
| 1570 | Right | TGGTCACTAATTAAGGTTTCCCATA |
| 1571 | Left | TCACTTTTTAAAACAACCATTCCAT |
| 1572 | Right | TGGTCACTAATTAAGGTTTCCCATA |
| 1573 | Left | TGTAGCTTAGCAAGGGCTTTAGATA |
| 1574 | Right | TTAAAGCTCCATATAACGATTGCTC |
| 1575 | Left | AAATGAGGACAGTAATAGCACCTTG |
| 1576 | Right | TGCATTGCAATATAGAAAACACAGT |
| 1577 | Left | AAAATCATGGACAAAAGAACCATAA |
| 1578 | Right | GGTGTCTGGATCAGTCTCACTTATT |
| 1579 | Left | AAAATCATGGACAAAAGAACCATAA |
| 1580 | Right | GGAACTAGAGGCTAGGAAGAGAAGA |
| 1581 | Left | AAATGAGGACAGTAATAGCACCTTG |
| 1582 | Right | GTGCTGAGGACATAAATAGGTCAGT |
| 1583 | Left | AACTTCAGCTTGGAGATAAAATCCT |
| 1584 | Right | TGCATTGCAATATAGAAAACACAGT |
| 1585 | Left | AAATGAGGACAGTAATAGCACCTTG |
| 1586 | Right | AGTCTCTCTCTCCCAAGGATATTGT |
| 1587 | Left | GTCACTCTCCCAACTCTTGATGTAT |
| 1588 | Right | TGGTCACTAATTAAGGTTTCCCATA |
| 1589 | Left | AACTTCAGCTTGGAGATAAAATCCT |
| 1590 | Right | GTGCTGAGGACATAAATAGGTCAGT |
| 1591 | Left | TGTAGCTTAGCAAGGGCTTTAGATA |
| 1592 | Right | AGTCTCACTTATTCCCCAAAGAGTT |
| 1593 | Left | TGTAGCTTAGCAAGGGCTTTAGATA |
| 1594 | Right | GGTGTCTGGATCAGTCTCACTTATT |
| 1595 | Left | TGTAGCTTAGCAAGGGCTTTAGATA |
| 1596 | Right | GGAACTAGAGGCTAGGAAGAGAAGA |
| 1597 | Left | GGACAGTAATAGCACCTTGTGTCTT |
| 1598 | Right | TCAGTGACACAAATGAAGAATTGAT |
| 1599 | Left | TTAATCATTTCCCCTAATCCTTTTC |
| 1600 | Right | TGCATTGCAATATAGAAAACACAGT |

ALK Exon24-25 5 kb

| | | |
|---|---|---|
| 1601 | Left | ATCTCCTTTTGAATGAAAGAGACCT |
| 1602 | Right | TGGTCACTAATTAAGGTTTCCCATA |
| 1603 | Left | ATCTCCTTTTGAATGAAAGAGACCT |
| 1604 | Right | AGCCTGAAAAGGAACTTAGTGAAAT |
| 1605 | Left | ACGTCAGGGATTTAGGAATTAAAAG |
| 1606 | Right | TGGTCACTAATTAAGGTTTCCCATA |
| 1607 | Left | ATGTGAATCATACTCCTCCAGGTAA |
| 1608 | Right | TGGTCACTAATTAAGGTTTCCCATA |
| 1609 | Left | ATCTCCTTTTGAATGAAAGAGACCT |
| 1610 | Right | GTATCCAAGTTATCCCATGTCTCAG |
| 1611 | Left | ATCTCCTTTTGAATGAAAGAGACCT |
| 1612 | Right | TAGCCTGAAAAGGAACTTAGTGAAA |
| 1613 | Left | ACGTCAGGGATTTAGGAATTAAAAG |
| 1614 | Right | AGCCTGAAAAGGAACTTAGTGAAAT |
| 1615 | Left | ATCTCCTTTTGAATGAAAGAGACCT |
| 1616 | Right | CATAGCCTGAAAAGGAACTTAGTGA |
| 1617 | Left | AGGTATGTGAATCATACTCCTCCAG |
| 1618 | Right | TGGTCACTAATTAAGGTTTCCCATA |
| 1619 | Left | AAGAGTCACCAGCTTAAACAAACAC |
| 1620 | Right | AGCCTGAAAAGGAACTTAGTGAAAT |
| 1621 | Left | ATGTGAATCATACTCCTCCAGGTAA |
| 1622 | Right | AGCCTGAAAAGGAACTTAGTGAAAT |
| 1623 | Left | TGCACAATAAATTAAAAGGGAAAGA |
| 1624 | Right | TGGTCACTAATTAAGGTTTCCCATA |
| 1625 | Left | TGTTTATAAATTGGGGGTATTCAAA |
| 1626 | Right | TGGTCACTAATTAAGGTTTCCCATA |
| 1627 | Left | TTGTTTATAAATTGGGGGTATTCAA |
| 1628 | Right | TGGTCACTAATTAAGGTTTCCCATA |
| 1629 | Left | TGAATCATACTCCTCCAGGTAAATC |
| 1630 | Right | TGGTCACTAATTAAGGTTTCCCATA |
| 1631 | Left | AGGTATGTGAATCATACTCCTCCAG |
| 1632 | Right | AGCCTGAAAAGGAACTTAGTGAAAT |
| 1633 | Left | ACGTCAGGGATTTAGGAATTAAAAG |
| 1634 | Right | TAGCCTGAAAAGGAACTTAGTGAAA |
| 1635 | Left | AAGAGTCACCAGCTTAAACAAACAC |
| 1636 | Right | TAGCCTGAAAAGGAACTTAGTGAAA |
| 1637 | Left | ATGTGAATCATACTCCTCCAGGTAA |
| 1638 | Right | TAGCCTGAAAAGGAACTTAGTGAAA |
| 1639 | Left | TGCACAATAAATTAAAAGGGAAAGA |
| 1640 | Right | AGCCTGAAAAGGAACTTAGTGAAAT |

TABLE 8
EGFR Capture Primer List for NGS Panel

| | | |
|---|---|---|
| Seq. | | |
| ID | Primer | Sequence |

EGFR Exon18 100-200 bases

| | | |
|---|---|---|
| 1641 | Left | TGCCAAAGAAGTAGAATGAG |
| 1642 | Right | AAAGCATCTTCACCCACAGC |
| 1643 | Left | TGCCAAAGAAGTAGAATGAG |
| 1644 | Right | TTCTTGACGAGGTCCATGTG |
| 1645 | Left | TGCCAAAGAAGTAGAATGAG |
| 1646 | Right | GTCAGAAATGCAGGAAAGCA |
| 1647 | Left | TGCCAAAGAAGTAGAATGAG |
| 1648 | Right | AGTCAGAAATGCAGGAAAGCA |
| 1649 | Left | TGCCAAAGAAGTAGAATGAG |
| 1650 | Right | CAGTCAGAAATGCAGGAAAGC |
| 1651 | Left | TGCCAAAGAAGTAGAATGAG |
| 1652 | Right | ATTCTTGACGAGGTCCATGTG |
| 1653 | Left | TGCCAAAGAAGTAGAATGAG |
| 1654 | Right | CATTCTTGACGAGGTCCATGT |
| 1655 | Left | TGCCAAAGAAGTAGAATGAG |
| 1656 | Right | GGAAAGCATCTTCACCCACA |
| 1657 | Left | TGCCAAAGAAGTAGAATGAG |
| 1658 | Right | CAGCAGTGTGGTCATTCTTGA |
| 1659 | Left | TGCCAAAGAAGTAGAATGAG |
| 1660 | Right | AGGACAGTCAGAAATGCAGGA |
| 1661 | Left | TGCCAAAGAAGTAGAATGAG |
| 1662 | Right | GGACAGTCAGAAATGCAGGA |
| 1663 | Left | GCCAAAGAAGTAGAATGAGA |
| 1664 | Right | AAAGCATCTTCACCCACAGC |
| 1665 | Left | TGCCAAAGAAGTAGAATGAG |
| 1666 | Right | GGACAGTCAGAAATGCAGGAA |
| 1667 | Left | GCCAAAGAAGTAGAATGAGA |
| 1668 | Right | TTCTTGACGAGGTCCATGTG |
| 1669 | Left | TGCCAAAGAAGTAGAATGAG |
| 1670 | Right | TGCAGGAAAGCATCTTCACC |
| 1671 | Left | TGCCAAAGAAGTAGAATGAG |
| 1672 | Right | TCAGAAATGCAGGAAAGCATC |
| 1673 | Left | TGCCAAAGAAGTAGAATGAG |
| 1674 | Right | GTCATTCTTGACGAGGTCCA |
| 1675 | Left | TGCCAAAGAAGTAGAATGAG |
| 1676 | Right | TGGTCATTCTTGACGAGGTC |
| 1677 | Left | TGCCAAAGAAGTAGAATGAG |
| 1678 | Right | AGCATCTTCACCCACAGCA |
| 1679 | Left | TGCCAAAGAAGTAGAATGAG |
| 1680 | Right | GCATCTTCACCCACAGCAG |

EGFR Exon18 200-400 bases

| | | |
|---|---|---|
| 1681 | Left | TGCCAAAGAAGTAGAATGAG |
| 1682 | Right | CCAGCACTGTGTGTCCAACT |
| 1683 | Left | TGCCAAAGAAGTAGAATGAG |
| 1684 | Right | TCCCTCCACTGAGGACAAAG |
| 1685 | Left | TGCCAAAGAAGTAGAATGAG |
| 1686 | Right | CTTTCCCTCCACTGAGGACA |
| 1687 | Left | TGCCAAAGAAGTAGAATGAG |
| 1688 | Right | CCAACTTTCCCTCCACTGAG |
| 1689 | Left | TGCCAAAGAAGTAGAATGAG |
| 1690 | Right | CAAAACCAGTGGAACCAAGG |
| 1691 | Left | TGCCAAAGAAGTAGAATGAG |
| 1692 | Right | TGTCCAACTTTCCCTCCACT |
| 1693 | Left | TGCCAAAGAAGTAGAATGAG |
| 1694 | Right | GTCCAACTTTCCCTCCACTG |
| 1695 | Left | TGCCAAAGAAGTAGAATGAG |
| 1696 | Right | GCAAAACCAGTGGAACCAAG |
| 1697 | Left | TGCCAAAGAAGTAGAATGAG |
| 1698 | Right | AGCAAAACCAGTGGAACCAA |
| 1699 | Left | TGCCAAAGAAGTAGAATGAG |
| 1700 | Right | AAACCAGTGGAACCAAGGAA |
| 1701 | Left | TGCCAAAGAAGTAGAATGAG |
| 1702 | Right | AAAACCAGTGGAACCAAGGA |
| 1703 | Left | TGCCAAAGAAGTAGAATGAG |
| 1704 | Right | GTGTCCAACTTTCCCTCCAC |
| 1705 | Left | TGCCAAAGAAGTAGAATGAG |
| 1706 | Right | GGCCCAGAGCCATAGAAACT |
| 1707 | Left | TGCCAAAGAAGTAGAATGAG |
| 1708 | Right | TTCCCTCCACTGAGGACAAA |
| 1709 | Left | TGCCAAAGAAGTAGAATGAG |
| 1710 | Right | TCCAACTTTCCCTCCACTGA |
| 1711 | Left | TGCCAAAGAAGTAGAATGAG |
| 1712 | Right | CCCTCCACTGAGGACAAAGT |
| 1713 | Left | TGCCAAAGAAGTAGAATGAG |
| 1714 | Right | AACCAGCTGGGCAGTCTCT |
| 1715 | Left | TGCCAAAGAAGTAGAATGAG |
| 1716 | Right | GAAACCCTGGCTGAGGGTAG |
| 1717 | Left | GCCAAAGAAGTAGAATGAGA |
| 1718 | Right | CCAGCACTGTGTGTCCAACT |
| 1719 | Left | GCCAAAGAAGTAGAATGAGA |
| 1720 | Right | TCCCTCCACTGAGGACAAAG |

EGFR Exon18 400-1000 bases

| | | |
|---|---|---|
| 1721 | Left | TGCCAAAGAAGTAGAATGAG |
| 1722 | Right | CAGTGTGGAGTGGGGAAGTT |
| 1723 | Left | TGCCAAAGAAGTAGAATGAG |
| 1724 | Right | ACTCCCCTATGCTGGAGGTT |
| 1725 | Left | TGCCAAAGAAGTAGAATGAG |
| 1726 | Right | TGGGAAAGAAAGCAAGGAGA |
| 1727 | Left | TGCCAAAGAAGTAGAATGAG |
| 1728 | Right | TCTGGGAAAGAAAGCAAGGA |
| 1729 | Left | TGCCAAAGAAGTAGAATGAG |
| 1730 | Right | ACCAATGGGGTAAGTGGACA |
| 1731 | Left | TGCCAAAGAAGTAGAATGAG |
| 1732 | Right | CCTCGATCATGTGACACTGG |
| 1733 | Left | TGCCAAAGAAGTAGAATGAG |
| 1734 | Right | AAAATGGCAAACAGGTGCTC |
| 1735 | Left | TGCCAAAGAAGTAGAATGAG |
| 1736 | Right | AACTGGCCAGAGCTGATGTT |
| 1737 | Left | TGCCAAAGAAGTAGAATGAG |
| 1738 | Right | AAACTGGCCAGAGCTGATGT |
| 1739 | Left | TGCCAAAGAAGTAGAATGAG |
| 1740 | Right | ACGCCATCGAGAGTAACACC |
| 1741 | Left | TGCCAAAGAAGTAGAATGAG |
| 1742 | Right | AGGAGCATGCCAAAATGAAG |
| 1743 | Left | TGCCAAAGAAGTAGAATGAG |
| 1744 | Right | TGTTGAAGGAAGCCCTTTTG |
| 1745 | Left | TGCCAAAGAAGTAGAATGAG |
| 1746 | Right | CCAATGGGGTAAGTGGACAG |
| 1747 | Left | TGCCAAAGAAGTAGAATGAG |
| 1748 | Right | TTGCCTTCTTCCTCGATCAT |
| 1749 | Left | TGCCAAAGAAGTAGAATGAG |
| 1750 | Right | CATCGAACAGAAAGGCCACT |
| 1751 | Left | TGCCAAAGAAGTAGAATGAG |
| 1752 | Right | GGTGGCAGGAGAGAGAGCTA |
| 1753 | Left | TGCCAAAGAAGTAGAATGAG |
| 1754 | Right | ATGGGACCAATGGGGTAAGT |
| 1755 | Left | TGCCAAAGAAGTAGAATGAG |
| 1756 | Right | TGGAGGTTGTCATCGAACAG |
| 1757 | Left | TGCCAAAGAAGTAGAATGAG |
| 1758 | Right | CTGGAGGTTGTCATCGAACA |
| 1759 | Left | TGCCAAAGAAGTAGAATGAG |
| 1760 | Right | TATGCTGGAGGTTGTCATCG |

EGFR Exon19 100-200 bases

| | | |
|---|---|---|
| 1761 | Left | CTTCCTTGTTCCTCCACCTCAT |
| 1762 | Right | ACCCAGGACTGGCACTCAC |
| 1763 | Left | CTTCCTTGTTCCTCCACCTCAT |
| 1764 | Right | CCCAGGACTGGCACTCAC |
| 1765 | Left | CTTCCTTGTTCCTCCACCTCAT |
| 1766 | Right | ACCCAGGACTGGCACTCA |
| 1767 | Left | CTTGTTCCTCCACCTCATTCC |
| 1768 | Right | ACCCAGGACTGGCACTCAC |
| 1769 | Left | CCTTGTTCCTCCACCTCATTC |
| 1770 | Right | ACCCAGGACTGGCACTCAC |
| 1771 | Left | TCCTTGTTCCTCCACCTCATT |
| 1772 | Right | ACCCAGGACTGGCACTCAC |
| 1773 | Left | TTCCTTGTTCCTCCACCTCAT |
| 1774 | Right | ACCCAGGACTGGCACTCAC |
| 1775 | Left | CTTCCTTGTTCCTCCACCTCATT |
| 1776 | Right | ACCCAGGACTGGCACTCAC |
| 1777 | Left | CAACCTCACCCTTCCTTGTTC |
| 1778 | Right | ACCCAGGACTGGCACTCAC |
| 1779 | Left | CTTGTTCCTCCACCTCATTCC |
| 1780 | Right | CCCAGGACTGGCACTCAC |
| 1781 | Left | CCTTGTTCCTCCACCTCATTC |
| 1782 | Right | CCCAGGACTGGCACTCAC |
| 1783 | Left | CCTTGTTCCTCCACCTCATTC |
| 1784 | Right | ACCCAGGACTGGCACTCA |
| 1785 | Left | CTTGTTCCTCCACCTCATTCC |
| 1786 | Right | ACCCAGGACTGGCACTCA |
| 1787 | Left | TCCTTGTTCCTCCACCTCATT |
| 1788 | Right | CCCAGGACTGGCACTCAC |
| 1789 | Left | TCCTTGTTCCTCCACCTCATT |
| 1790 | Right | ACCCAGGACTGGCACTCA |
| 1791 | Left | TTCCTTGTTCCTCCACCTCAT |
| 1792 | Right | CCCAGGACTGGCACTCAC |
| 1793 | Left | TTCCTTGTTCCTCCACCTCAT |
| 1794 | Right | ACCCAGGACTGGCACTCA |
| 1795 | Left | CTTCCTTGTTCCTCCACCTCATT |
| 1796 | Right | CCCAGGACTGGCACTCAC |
| 1797 | Left | CTTCCTTGTTCCTCCACCTCATT |
| 1798 | Right | ACCCAGGACTGGCACTCA |
| 1799 | Left | CAACCTCACCCTTCCTTGTTC |
| 1800 | Right | CCCAGGACTGGCACTCAC |

EGFR Exon19 200-400 bases

| | | |
|---|---|---|
| 1801 | Left | AAGATCATTCTACAAGATGTCAGTGG |
| 1802 | Right | AACTGCACATTCAGAGATTCTTTCT |
| 1803 | Left | AGATCATTCTACAAGATGTCAGTGC |
| 1804 | Right | AACTGCACATTCAGAGATTCTTTCT |
| 1805 | Left | TCCAAGATCATTCTACAAGATGTCA |
| 1806 | Right | ACATTCAGAGATTCTTTCTGCATCA |
| 1807 | Left | TCCAAGATCATTCTACAAGATGTCA |
| 1808 | Right | ATTCAGAGATTCTTTCTGCATCATA |
| 1809 | Left | TCCAAGATCATTCTACAAGATGTCA |
| 1810 | Right | CATTCAGAGATTCTTTCTGCATCA |
| 1811 | Left | CAAGATCATTCTACAAGATGTCAGTG |
| 1812 | Right | AACTGCACATTCAGAGATTCTTTCT |
| 1813 | Left | TCCAAGATCATTCTACAAGATGTCA |
| 1814 | Right | CATTCAGAGATTCTTTCTGCATCATA |
| 1815 | Left | AAGATCATTCTACAAGATGTCAGTGG |
| 1816 | Right | CATTCAGAGATTCTTTCTGCATCAT |
| 1817 | Left | AAGATCATTCTACAAGATGTCAGTGG |
| 1818 | Right | TAACTGCACATTCAGAGATTCTTTC |
| 1819 | Left | AGATCATTCTACAAGATGTCAGTGC |
| 1820 | Right | CATTCAGAGATTCTTTCTGCATCAT |
| 1821 | Left | AGATCATTCTACAAGATGTCAGTGC |
| 1822 | Right | TAACTGCACATTCAGAGATTCTTTC |
| 1823 | Left | AAGATCATTCTACAAGATGTCAGTGC |
| 1824 | Right | TAACTGCACATTCAGAGATTCTTTCT |
| 1825 | Left | AGATCATTCTACAAGATGTCAGTGC |
| 1826 | Right | TAACTGCACATTCAGAGATTCTTTCT |
| 1827 | Left | TCCAAGATCATTCTACAAGATGTCA |
| 1828 | Right | TTCAGAGATTCTTTCTGCATCATAATA |
| 1829 | Left | TCCAAGATCATTCTACAAGATGTCA |
| 1830 | Right | ACTGCACATTCAGAGATTCTTTCT |
| 1831 | Left | TCCAAGATCATTCTACAAGATGTCA |
| 1832 | Right | GCACATTCAGAGATTCTTTCTGC |
| 1833 | Left | TCCAAGATCATTCTACAAGATGTCA |
| 1834 | Right | TCAGAGATTCTTTCTGCATCATAATA |
| 1835 | Left | CCAAGATCATTCTACAAGATGTCAGT |
| 1836 | Right | ACATTCAGAGATTCTTTCTGCATCA |
| 1837 | Left | CCAAGATCATTCTACAAGATGTCAGT |
| 1838 | Right | ATTCAGAGATTCTTTCTGCATCATA |
| 1839 | Left | AAGATCATTCTACAAGATGTCAGTGC |
| 1840 | Right | ACATTCAGAGATTCTTTCTGCATCA |

EGFR Exon19 400-1000 bases

| | | |
|---|---|---|
| 1841 | Left | AATACCAATCCATGAAAAAGCATTA |
| 1842 | Right | AACATGTCACCAACTGGGTATAACT |
| 1843 | Left | CCTATTCCTTTATAACCCCTTTCAA |
| 1844 | Right | AACATGTCACCAACTGGGTATAACT |
| 1845 | Left | TCCAAGATCATTCTACAAGATGTCA |
| 1846 | Right | AACATGTCACCAACTGGGTATAACT |
| 1847 | Left | TTTCAAGCTCGTTCAGAGAGTATTT |
| 1848 | Right | AACATGTCACCAACTGGGTATAACT |
| 1849 | Left | TTCAGAGAGTATTTCACACAATCCA |
| 1850 | Right | AACATGTCACCAACTGGGTATAACT |
| 1851 | Left | GTGTCTCACTTTCCAAGATCATTCT |
| 1852 | Right | AACATGTCACCAACTGGGTATAACT |
| 1853 | Left | AGTGTCTCACTTTCCAAGATCATTC |
| 1854 | Right | AACATGTCACCAACTGGGTATAACT |
| 1855 | Left | CCATGAAAAAGCATTATTGAAGTCT |
| 1856 | Right | AACATGTCACCAACTGGGTATAACT |
| 1857 | Left | TATTCCTTTATAACCCCTTTCAAGC |
| 1858 | Right | AACATGTCACCAACTGGGTATAACT |
| 1859 | Left | ATGGAAATACTCTTGGAATGAACAA |
| 1860 | Right | AACATGTCACCAACTGGGTATAACT |
| 1861 | Left | CTCGTTCAGAGAGTATTTCACACAA |
| 1862 | Right | AACATGTCACCAACTGGGTATAACT |
| 1863 | Left | TCCAAGATCATTCTACAAGATGTCA |
| 1864 | Right | ACTGAACAGCTACCTTTCAACAAAC |
| 1865 | Left | CCTATTCCTTTATAACCCCTTTCAA |
| 1866 | Right | AACTGCACATTCAGAGATTCTTTCT |
| 1867 | Left | TCCAAGATCATTCTACAAGATGTCA |
| 1868 | Right | AACTGCACATTCAGAGATTCTTTCT |
| 1869 | Left | ACTCTTGGAATGAACAAAATACCAA |
| 1870 | Right | AACATGTCACCAACTGGGTATAACT |
| 1871 | Left | CCTATTCCTTTATAACCCCTTTCAA |
| 1872 | Right | GGGTATAACTGCACATTCAGAGATT |
| 1873 | Left | TCCAAGATCATTCTACAAGATGTCA |
| 1874 | Right | GGGTATAACTGCACATTCAGAGATT |
| 1875 | Left | AGTGTCTCACTTTCCAAGATCATTC |
| 1876 | Right | ACTGAACAGCTACCTTTCAACAAAC |
| 1877 | Left | GTGTCTCACTTTCCAAGATCATTCT |
| 1878 | Right | ACTGAACAGCTACCTTTCAACAAAC |
| 1879 | Left | AGTGTCTCACTTTCCAAGATCATTC |
| 1880 | Right | AACTGCACATTCAGAGATTCTTTCT |

EGFR Exon20 100-200 bases

| | | |
|---|---|---|
| 1881 | Left | GTGACCCTTGTCTCTGTGTTCTT |
| 1882 | Right | CCTGTGCCAGGGACCTTAC |
| 1883 | Left | ACCCTTGTCTCTGTGTTCTTGTC |
| 1884 | Right | CCTGTGCCAGGGACCTTAC |
| 1885 | Left | GACCCTTGTCTCTGTGTTCTTGT |
| 1886 | Right | CCTGTGCCAGGGACCTTAC |
| 1887 | Left | GACCCTTGTCTCTGTGTTCTTGTC |
| 1888 | Right | CCTGTGCCAGGGACCTTAC |
| 1889 | Left | GTGACCCTTGTCTCTGTGTTCTTGT |
| 1890 | Right | CCTGTGCCAGGGACCTTAC |
| 1891 | Left | AGGTGACCCTTGTCTCTGTGTT |
| 1892 | Right | CCTGTGCCAGGGACCTTAC |
| 1893 | Left | GACCCTTGTCTCTGTGTTCTTGT |
| 1894 | Right | CCTGTGCCAGGGACCTTA |
| 1895 | Left | GTGACCCTTGTCTCTGTGTTCTT |
| 1896 | Right | CCTGTGCCAGGGACCTTA |
| 1897 | Left | ACCCTTGTCTCTGTGTTCTTGTC |
| 1898 | Right | CCTGTGCCAGGGACCTTA |
| 1899 | Left | GACCCTTGTCTCTGTGTTCTTGTC |
| 1900 | Right | CCTGTGCCAGGGACCTTA |
| 1901 | Left | GTGACCCTTGTCTCTGTGTTCTTG |
| 1902 | Right | CCTGTGCCAGGGACCTTAC |
| 1903 | Left | TGACCCTTGTCTCTGTGTTCTTGT |
| 1904 | Right | CCTGTGCCAGGGACCTTAC |
| 1905 | Left | GAGGTGACCCTTGTCTCTGTGT |
| 1906 | Right | CCTGTGCCAGGGACCTTAC |
| 1907 | Left | TGACCCTTGTCTCTGTGTTCTTG |
| 1908 | Right | CCTGTGCCAGGGACCTTAC |
| 1909 | Left | AGGTGACCCTTGTCTCTGTGTTCT |
| 1910 | Right | CCTGTGCCAGGGACCTTAC |
| 1911 | Left | CTGAGGTGACCCTTGTCTCTGT |
| 1912 | Right | CCTGTGCCAGGGACCTTAC |
| 1913 | Left | AGGTGACCCTTGTCTCTGTGTTCTT |
| 1914 | Right | CCTGTGCCAGGGACCTTAC |
| 1915 | Left | GGTGACCCTTGTCTCTGTGTTCT |
| 1916 | Right | CCTGTGCCAGGGACCTTAC |
| 1917 | Left | AGGTGACCCTTGTCTCTGTGTTC |
| 1918 | Right | CCTGTGCCAGGGACCTTAC |
| 1919 | Left | GAGGTGACCCTTGTCTCTGTGTT |
| 1920 | Right | CCTGTGCCAGGGACCTTAC |

EGFR Exon20 200-400 bases

| | | |
|---|---|---|
| 1921 | Left | AAGCTCTGTAGAGAAGGCGTACAT |
| 1922 | Right | AAATATACAGCTTGCAAGGACTCTG |
| 1923 | Left | AGCTCTGTAGAGAAGGCGTACATT |
| 1924 | Right | AAATATACAGCTTGCAAGGACTCTG |
| 1925 | Left | TCTGTAGAGAAGGCGTACATTTGTC |
| 1926 | Right | AAATATACAGCTTGCAAGGACTCTG |
| 1927 | Left | GTAGAGAAGGCGTACATTTGTCCT |
| 1928 | Right | AAATATACAGCTTGCAAGGACTCTG |
| 1929 | Left | AAGCTCTGTAGAGAAGGCGTACATT |
| 1930 | Right | AAATATACAGCTTGCAAGGACTCTG |
| 1931 | Left | GCTCTGTAGAGAAGGCGTACATTT |
| 1932 | Right | AAATATACAGCTTGCAAGGACTCTG |
| 1933 | Left | CTCTGTAGAGAAGGCGTACATTTG |
| 1934 | Right | AAATATACAGCTTGCAAGGACTCTG |
| 1935 | Left | CTGTAGAGAAGGCGTACATTTGTC |
| 1936 | Right | AAATATACAGCTTGCAAGGACTCTG |
| 1937 | Left | TTCTGTCAAGCTCTGTAGAGAAGG |
| 1938 | Right | AAATATACAGCTTGCAAGGACTCTG |
| 1939 | Left | AAGCTCTGTAGAGAAGGCGTACA |
| 1940 | Right | AAATATACAGCTTGCAAGGACTCTG |
| 1941 | Left | TACATTTGTCCTTCCAAATGAGC |
| 1942 | Right | AAATATACAGCTTGCAAGGACTCTG |
| 1943 | Left | CAAGCTCTGTAGAGAAGGCGTACAT |
| 1944 | Right | AAATATACAGCTTGCAAGGACTCTG |
| 1945 | Left | AGCTCTGTAGAGAAGGCGTACATT |
| 1946 | Right | GGAAATATACAGCTTGCAAGGACTC |
| 1947 | Left | AAGCTCTGTAGAGAAGGCGTACAT |
| 1948 | Right | GGAAATATACAGCTTGCAAGGACTC |
| 1949 | Left | GTCAAGCTCTGTAGAGAAGGCGTA |
| 1950 | Right | AAATATACAGCTTGCAAGGACTCTG |
| 1951 | Left | TCTGTAGAGAAGGCGTACATTTGTC |
| 1952 | Right | GGAAATATACAGCTTGCAAGGACTC |
| 1953 | Left | TGTAGAGAAGGCGTACATTTGTCCT |
| 1954 | Right | AAATATACAGCTTGCAAGGACTCTG |
| 1955 | Left | GTAGAGAAGGCGTACATTTGTCCT |
| 1956 | Right | GGAAATATACAGCTTGCAAGGACTC |
| 1957 | Left | AAGCTCTGTAGAGAAGGCGTACATT |
| 1958 | Right | GGAAATATACAGCTTGCAAGGACTC |
| 1959 | Left | AGCTCTGTAGAGAAGGCGTACATT |
| 1960 | Right | ATGGAAATATACAGCTTGCAAGGAC |

EGFR Exon20 400-1000 bases

| | | |
|---|---|---|
| 1961 | Left | TTTCTACCAACTTCTGTCAAGCTCT |
| 1962 | Right | ATCTAGAAGAAGCAAACGAAGATGA |
| 1963 | Left | GTTTCTACCAACTTCTGTCAAGCTC |
| 1964 | Right | ATCTAGAAGAAGCAAACGAAGATGA |
| 1965 | Left | GTTTCTACCAACTTCTGTCAAGCTC |
| 1966 | Right | GATCTAGAAGAAGCAAACGAAGATG |
| 1967 | Left | GTTTCTACCAACTTCTGTCAAGCTC |
| 1968 | Right | CTATGACAGAGAGAGAAGGAAGACG |
| 1969 | Left | CTGTGTTTCTACCAACTTCTGTCAA |
| 1970 | Right | GATCTAGAAGAAGCAAACGAAGATG |
| 1971 | Left | TGTGTTTCTACCAACTTCTGTCAAG |
| 1972 | Right | ATCTAGAAGAAGCAAACGAAGATGA |
| 1973 | Left | TGTGTTTCTACCAACTTCTGTCAAG |
| 1974 | Right | GATCTAGAAGAAGCAAACGAAGATG |
| 1975 | Left | CTGTGTTTCTACCAACTTCTGTCAA |
| 1976 | Right | ATCTAGAAGAAGCAAACGAAGATGA |
| 1977 | Left | AGAAAGAATCTCTGAATGTGCAGTT |
| 1978 | Right | AAATATACAGCTTGCAAGGACTCTG |
| 1979 | Left | GAAATTGTGTTTGTTGAAAGGTAGC |
| 1980 | Right | ATCTAGAAGAAGCAAACGAAGATGA |
| 1981 | Left | GAAATTGTGTTTGTTGAAAGGTAGC |
| 1982 | Right | GATCTAGAAGAAGCAAACGAAGATG |
| 1983 | Left | AATCTCTGAATGTGCAGTTATACCC |
| 1984 | Right | AAATATACAGCTTGCAAGGACTCTG |
| 1985 | Left | TTTCTACCAACTTCTGTCAAGCTCT |
| 1986 | Right | GTTATAAAGTCCGTGTGGATCATTT |
| 1987 | Left | CTGTGTTTCTACCAACTTCTGTCAA |
| 1988 | Right | AAATATACAGCTTGCAAGGACTCTG |
| 1989 | Left | TTTCTACCAACTTCTGTCAAGCTCT |
| 1990 | Right | TTATAAAGTCCGTGTGGATCATTTC |
| 1991 | Left | GAAATTGTGTTTGTTGAAAGGTAGC |
| 1992 | Right | AAATATACAGCTTGCAAGGACTCTG |
| 1993 | Left | TTTCTACCAACTTCTGTCAAGCTCT |
| 1994 | Right | CTGTTATAAAGTCCGTGTGGATCAT |
| 1995 | Left | GTTTCTACCAACTTCTGTCAAGCTC |
| 1996 | Right | GTTATAAAGTCCGTGTGGATCATTT |
| 1997 | Left | TTTCTACCAACTTCTGTCAAGCTCT |
| 1998 | Right | TCTAGAAGAAGCAAACGAAGATGAG |
| 1999 | Left | TTTCTACCAACTTCTGTCAAGCTCT |
| 2000 | Right | CTCCACGAATCACACTGATTATTTA |

EGFR Exon21 100-200 bases

| | | |
|---|---|---|
| 2001 | Left | ACGTCTTCCTTCTCTCTCTGTCATA |
| 2002 | Right | ACACAGCAAAGCAGAAACTCAC |
| 2003 | Left | TTAACGTCTTCCTTCTCTCTCTGTC |
| 2004 | Right | ACACAGCAAAGCAGAAACTCAC |
| 2005 | Left | TAACGTCTTCCTTCTCTCTCTGTCA |
| 2006 | Right | ACACAGCAAAGCAGAAACTCAC |
| 2007 | Left | ACGTCTTCCTTCTCTCTCTGTCATA |
| 2008 | Right | CACACAGCAAAGCAGAAACTCAC |
| 2009 | Left | ACGTCTTCCTTCTCTCTCTGTCAT |
| 2010 | Right | ACACAGCAAAGCAGAAACTCAC |
| 2011 | Left | CCAGTTAACGTCTTCCTTCTCTCTC |
| 2012 | Right | ACACAGCAAAGCAGAAACTCAC |
| 2013 | Left | ACGTCTTCCTTCTCTCTCTGTCATA |
| 2014 | Right | CCACACAGCAAAGCAGAAACT |
| 2015 | Left | AACGTCTTCCTTCTCTCTCTGTCAT |
| 2016 | Right | ACACAGCAAAGCAGAAACTCAC |
| 2017 | Left | CAGTTAACGTCTTCCTTCTCTCTCT |
| 2018 | Right | ACACAGCAAAGCAGAAACTCAC |
| 2019 | Left | AGTTAACGTCTTCCTTCTCTCTCTG |
| 2020 | Right | ACACAGCAAAGCAGAAACTCAC |
| 2021 | Left | ACGTCTTCCTTCTCTCTCTGTCATA |
| 2022 | Right | CACACAGCAAAGCAGAAACTCA |
| 2023 | Left | GTTAACGTCTTCCTTCTCTCTCTGT |
| 2024 | Right | ACACAGCAAAGCAGAAACTCAC |
| 2025 | Left | TTAACGTCTTCCTTCTCTCTCTGTC |
| 2026 | Right | CACACAGCAAAGCAGAAACTCAC |
| 2027 | Left | GTTAACGTCTTCCTTCTCTCTCTGTC |
| 2028 | Right | ACACAGCAAAGCAGAAACTCAC |
| 2029 | Left | AGTTAACGTCTTCCTTCTCTCTCTGT |
| 2030 | Right | ACACAGCAAAGCAGAAACTCAC |
| 2031 | Left | TTAACGTCTTCCTTCTCTCTCTGTC |
| 2032 | Right | CCACACAGCAAAGCAGAAACT |
| 2033 | Left | CCAGTTAACGTCTTCCTTCTCTCT |
| 2034 | Right | ACACAGCAAAGCAGAAACTCAC |
| 2035 | Left | TTAACGTCTTCCTTCTCTCTCTGTC |
| 2036 | Right | CACACAGCAAAGCAGAAACTCA |
| 2037 | Left | TAACGTCTTCCTTCTCTCTCTGTCA |
| 2038 | Right | CACACAGCAAAGCAGAAACTCAC |
| 2039 | Left | CGTCTTCCTTCTCTCTCTGTCATA |
| 2040 | Right | ACACAGCAAAGCAGAAACTCAC |

EGFR Exon21 200-400 bases

| | | |
|---|---|---|
| 2041 | Left | ACGTCTTCCTTCTCTCTCTGTCATA |
| 2042 | Right | TGTCTCTAAGGGGAGGGAGTTATAC |
| 2043 | Left | ACGTCTTCCTTCTCTCTCTGTCATA |
| 2044 | Right | GAAAGTGAACATTTAGGATGTGGAG |
| 2045 | Left | ACGTCTTCCTTCTCTCTCTGTCATA |
| 2046 | Right | AAAGTGAACATTTAGGATGTGGAGA |
| 2047 | Left | ACGTCTTCCTTCTCTCTCTGTCATA |
| 2048 | Right | AGAAAGTGAACATTTAGGATGTGGA |
| 2049 | Left | TTAACGTCTTCCTTCTCTCTCTGTC |
| 2050 | Right | TGTCTCTAAGGGGAGGGAGTTATAC |
| 2051 | Left | ACGTCTTCCTTCTCTCTCTGTCATA |
| 2052 | Right | GTGTCAAGAAACTAGTGCTGGGTAG |
| 2053 | Left | TTAACGTCTTCCTTCTCTCTCTGTC |
| 2054 | Right | AAAGTGAACATTTAGGATGTGGAGA |
| 2055 | Left | TTAACGTCTTCCTTCTCTCTCTGTC |
| 2056 | Right | GAAAGTGAACATTTAGGATGTGGAG |
| 2057 | Left | TTAACGTCTTCCTTCTCTCTCTGTC |
| 2058 | Right | AGAAAGTGAACATTTAGGATGTGGA |
| 2059 | Left | ACGTCTTCCTTCTCTCTCTGTCATA |
| 2060 | Right | AGTGAACATTTAGGATGTGGAGATG |
| 2061 | Left | ACGTCTTCCTTCTCTCTCTGTCATA |
| 2062 | Right | AAGTGAACATTTAGGATGTGGAGAT |
| 2063 | Left | ACGTCTTCCTTCTCTCTCTGTCATA |
| 2064 | Right | GTCAAGAAACTAGTGCTGGGTAGAT |
| 2065 | Left | TTAACGTCTTCCTTCTCTCTCTGTC |
| 2066 | Right | GTGTCAAGAAACTAGTGCTGGGTAG |
| 2067 | Left | TTAACGTCTTCCTTCTCTCTCTGTC |
| 2068 | Right | AGTGAACATTTAGGATGTGGAGATG |
| 2069 | Left | TTAACGTCTTCCTTCTCTCTCTGTC |
| 2070 | Right | AAGTGAACATTTAGGATGTGGAGAT |
| 2071 | Left | ACGTCTTCCTTCTCTCTCTGTCATA |
| 2072 | Right | TCAAGAAACTAGTGCTGGGTAGATG |
| 2073 | Left | TTAACGTCTTCCTTCTCTCTCTGTC |
| 2074 | Right | GTCAAGAAACTAGTGCTGGGTAGAT |
| 2075 | Left | ACGTCTTCCTTCTCTCTCTGTCATA |
| 2076 | Right | GAAAGGGAAAGACATAGAAAGTGAAC |
| 2077 | Left | ACGTCTTCCTTCTCTCTCTGTCATA |
| 2078 | Right | TGTCAAGAAACTAGTGCTGGGTAG |
| 2079 | Left | TAACGTCTTCCTTCTCTCTCTGTCA |
| 2080 | Right | TGTCTCTAAGGGGAGGGAGTTATAC |

EGFR Exon21 400-1000 bases

| | | |
|---|---|---|
| 2081 | Left | ACGTCTTCCTTCTCTCTCTGTCATA |
| 2082 | Right | CAAAGTAACAATCAACAGACACTGG |
| 2083 | Left | ACGTCTTCCTTCTCTCTCTGTCATA |
| 2084 | Right | AAAGATGAGATAACTTGGTGGAGTG |
| 2085 | Left | ACGTCTTCCTTCTCTCTCTGTCATA |
| 2086 | Right | CAAAGATGAGATAACTTGGTGGAGT |
| 2087 | Left | ACGTCTTCCTTCTCTCTCTGTCATA |
| 2088 | Right | TGAGGTAATAAGTCAGCCATTTTTC |
| 2089 | Left | ACGTCTTCCTTCTCTCTCTGTCATA |
| 2090 | Right | CCACAAAGTAACAATCAACAGACAC |
| 2091 | Left | TTCTAGATCCTCTTTGCATGAAATC |
| 2092 | Right | AAAGATGAGATAACTTGGTGGAGTG |
| 2093 | Left | TTCTAGATCCTCTTTGCATGAAATC |
| 2094 | Right | CAAAGATGAGATAACTTGGTGGAGT |
| 2095 | Left | ACGTCTTCCTTCTCTCTCTGTCATA |
| 2096 | Right | GGTAATAAGTCAGCCATTTTTCCTT |
| 2097 | Left | ACGTCTTCCTTCTCTCTCTGTCATA |
| 2098 | Right | AATTTCTTTATGCCTCCATTTCTTC |
| 2099 | Left | ACGTCTTCCTTCTCTCTCTGTCATA |
| 2100 | Right | GATGAGATAACTTGGTGGAGTGAAT |
| 2101 | Left | ACGTCTTCCTTCTCTCTCTGTCATA |
| 2102 | Right | GAATTTTCCAAGAACTTATTCCACA |
| 2103 | Left | ACGTCTTCCTTCTCTCTCTGTCATA |
| 2104 | Right | TGTGGAATTTTCCAAGAACTTATTC |
| 2105 | Left | ACGTCTTCCTTCTCTCTCTGTCATA |
| 2106 | Right | AATAAGTCAGCCATTTTTCCTTTTC |
| 2107 | Left | ACGTCTTCCTTCTCTCTCTGTCATA |
| 2108 | Right | CCATTTCAAAGATGAGATAACTTGG |
| 2109 | Left | AGGCTTTACAAGCTTGAGATTCTTT |
| 2110 | Right | CAAAGTAACAATCAACAGACACTGG |
| 2111 | Left | TTCTAGATCCTCTTTGCATGAAATC |
| 2112 | Right | AATTTCTTTATGCCTCCATTTCTTC |
| 2113 | Left | ACGTCTTCCTTCTCTCTCTGTCATA |
| 2114 | Right | AGATAACTTGGTGGAGTGAATTGAA |
| 2115 | Left | TTCTAGATCCTCTTTGCATGAAATC |
| 2116 | Right | GATGAGATAACTTGGTGGAGTGAAT |
| 2117 | Left | TTCTAGATCCTCTTTGCATGAAATC |
| 2118 | Right | CCATTTCAAAGATGAGATAACTTGG |
| 2119 | Left | AGGCTTTACAAGCTTGAGATTCTTT |
| 2120 | Right | AAAGATGAGATAACTTGGTGGAGTG |

EGFR Exon22 100-200 bases

| | | |
|---|---|---|
| 2121 | Left | CACACTGACGTGCCTCTCC |
| 2122 | Right | CGTATCTCCCTTCCCTGATTAC |
| 2123 | Left | CCACACTGACGTGCCTCTC |
| 2124 | Right | CGTATCTCCCTTCCCTGATTAC |
| 2125 | Left | CACACTGACGTGCCTCTCC |
| 2126 | Right | CCGTATCTCCCTTCCCTGATTAC |
| 2127 | Left | CCACACTGACGTGCCTCTC |
| 2128 | Right | CCGTATCTCCCTTCCCTGATTAC |
| 2129 | Left | CACACTGACGTGCCTCTCC |
| 2130 | Right | CCGTATCTCCCTTCCCTGATTA |
| 2131 | Left | CCACACTGACGTGCCTCTC |
| 2132 | Right | CCGTATCTCCCTTCCCTGATTA |
| 2133 | Left | CGAAGCCACACTGACGTG |
| 2134 | Right | CGTATCTCCCTTCCCTGATTAC |
| 2135 | Left | CGAAGCCACACTGACGTG |
| 2136 | Right | CCGTATCTCCCTTCCCTGATTAC |
| 2137 | Left | ACCATGCGAAGCCACACT |
| 2138 | Right | CGTATCTCCCTTCCCTGATTAC |
| 2139 | Left | ACCATGCGAAGCCACACT |
| 2140 | Right | CCGTATCTCCCTTCCCTGATTAC |
| 2141 | Left | CGAAGCCACACTGACGTG |
| 2142 | Right | CCGTATCTCCCTTCCCTGATTA |
| 2143 | Left | CATGCGAAGCCACACTGAC |
| 2144 | Right | CGTATCTCCCTTCCCTGATTAC |
| 2145 | Left | CATGCGAAGCCACACTGA |
| 2146 | Right | CGTATCTCCCTTCCCTGATTAC |
| 2147 | Left | CATGCGAAGCCACACTGAC |
| 2148 | Right | CCGTATCTCCCTTCCCTGATTAC |
| 2149 | Left | CACACTGACGTGCCTCTCC |
| 2150 | Right | TATCTCCCCTCCCCGTATCT |
| 2151 | Left | CATGCGAAGCCACACTGA |
| 2152 | Right | CCGTATCTCCCTTCCCTGATTAC |
| 2153 | Left | CACACTGACGTGCCTCTCC |
| 2154 | Right | CCGTATCTCCCTTCCCTGAT |
| 2155 | Left | CCACACTGACGTGCCTCTC |
| 2156 | Right | CCGTATCTCCCTTCCCTGAT |
| 2157 | Left | ACCATGCGAAGCCACACT |
| 2158 | Right | CCGTATCTCCCTTCCCTGATTA |
| 2159 | Left | CACACTGACGTGCCTCTCC |
| 2160 | Right | TATCTCCCCTCCCCGTATCTC |

EGFR Exon22 200-400 bases

| | | |
|---|---|---|
| 2161 | Left | GTATTTTGAAACTCAAGATCGCATT |
| 2162 | Right | CGTATCTCCCTTCCCTGATTAC |
| 2163 | Left | CCTCCATGAGTACGTATTTTGAAAC |
| 2164 | Right | CGTATCTCCCTTCCCTGATTAC |
| 2165 | Left | GTATTTTGAAACTCAAGATCGCATT |
| 2166 | Right | CCGTATCTCCCTTCCCTGATTAC |
| 2167 | Left | CCTCCATGAGTACGTATTTTGAAAC |
| 2168 | Right | CCGTATCTCCCTTCCCTGATTAC |
| 2169 | Left | GTATTTTGAAACTCAAGATCGCATT |
| 2170 | Right | CATGGCAAACTCTTGCTATCC |
| 2171 | Left | GTATTTTGAAACTCAAGATCGCATT |
| 2172 | Right | ATATCCCCATGGCAAACTCTT |
| 2173 | Left | CCATGAGTACGTATTTTGAAACTCA |
| 2174 | Right | CGTATCTCCCTTCCCTGATTAC |
| 2175 | Left | GTATTTTGAAACTCAAGATCGCATT |
| 2176 | Right | CCGTATCTCCCTTCCCTGATTA |
| 2177 | Left | CCTCCATGAGTACGTATTTTGAAAC |
| 2178 | Right | CCGTATCTCCCTTCCCTGATTA |
| 2179 | Left | CCATGAGTACGTATTTTGAAACTCA |
| 2180 | Right | CCGTATCTCCCTTCCCTGATTAC |
| 2181 | Left | CCATGAGTACGTATTTTGAAACTCA |
| 2182 | Right | CATGGCAAACTCTTGCTATCC |
| 2183 | Left | TATTTTGAAACTCAAGATCGCATTC |
| 2184 | Right | CGTATCTCCCTTCCCTGATTAC |
| 2185 | Left | GTATTTTGAAACTCAAGATCGCATT |
| 2186 | Right | CTTATCTCCCCTCCCCGTATCT |
| 2187 | Left | GTATTTTGAAACTCAAGATCGCATT |
| 2188 | Right | ACATATCCCCATGGCAAACTCT |
| 2189 | Left | CTTTTCCTCCATGAGTACGTATTTT |
| 2190 | Right | CGTATCTCCCTTCCCTGATTAC |
| 2191 | Left | TATTTTGAAACTCAAGATCGCATTC |
| 2192 | Right | CCGTATCTCCCTTCCCTGATTAC |
| 2193 | Left | GTATTTTGAAACTCAAGATCGCATT |
| 2194 | Right | ACATATCCCCATGGCAAACTCTT |
| 2195 | Left | CCTCCATGAGTACGTATTTTGAAAC |
| 2196 | Right | CTTATCTCCCCTCCCCGTATCT |
| 2197 | Left | CCATGAGTACGTATTTTGAAACTCA |
| 2198 | Right | CCGTATCTCCCTTCCCTGATTA |
| 2199 | Left | GTATTTTGAAACTCAAGATCGCATT |
| 2200 | Right | ACATATCCCCATGGCAAACTC |

EGFR Exon22 400-1000 bases

| | | |
|---|---|---|
| 2201 | Left | GTATTTTGAAACTCAAGATCGCATT |
| 2202 | Right | TTGAATCCAAAATAAAGGAATGTGT |
| 2203 | Left | GTATTTTGAAACTCAAGATCGCATT |
| 2204 | Right | CACACTGAGCACTCAATAAAGAGAA |
| 2205 | Left | GTATTTTGAAACTCAAGATCGCATT |
| 2206 | Right | TTCTCCACTACAAATCACCACAGTA |
| 2207 | Left | GTATTTTGAAACTCAAGATCGCATT |
| 2208 | Right | ATTCTTCAAAGGTAGCTGATTGATG |
| 2209 | Left | GTATTTTGAAACTCAAGATCGCATT |
| 2210 | Right | ATCCAAAATAAAGGAATGTGTGTGT |
| 2211 | Left | GTATTTTGAAACTCAAGATCGCATT |
| 2212 | Right | GCTTACCTTGTTATCAAGTCCTGAA |
| 2213 | Left | TGGTCTATTGAAAGAGCTTATCCAG |
| 2214 | Right | TTGAATCCAAAATAAAGGAATGTGT |
| 2215 | Left | CCTCCATGAGTACGTATTTTGAAAC |
| 2216 | Right | TTGAATCCAAAATAAAGGAATGTGT |
| 2217 | Left | CCTCCATGAGTACGTATTTTGAAAC |
| 2218 | Right | CACACTGAGCACTCAATAAAGAGAA |
| 2219 | Left | CCTCCATGAGTACGTATTTTGAAAC |
| 2220 | Right | TTCTCCACTACAAATCACCACAGTA |
| 2221 | Left | TGGTCTATTGAAAGAGCTTATCCAG |
| 2222 | Right | ATCCAAAATAAAGGAATGTGTGTGT |
| 2223 | Left | CCTCCATGAGTACGTATTTTGAAAC |
| 2224 | Right | ATCCAAAATAAAGGAATGTGTGTGT |
| 2225 | Left | CCTCCATGAGTACGTATTTTGAAAC |
| 2226 | Right | GCTTACCTTGTTATCAAGTCCTGAA |
| 2227 | Left | GTATTTTGAAACTCAAGATCGCATT |
| 2228 | Right | CAAAGGTAGCTGATTGATGAGAGTT |
| 2229 | Left | GTATTTTGAAACTCAAGATCGCATT |
| 2230 | Right | TATTCCTTCTCCACTACAAATCACC |
| 2231 | Left | GTATTTTGAAACTCAAGATCGCATT |
| 2232 | Right | CCACTACAAATCACCACAGTATTCA |
| 2233 | Left | GTATTTTGAAACTCAAGATCGCATT |
| 2234 | Right | CTTGATTGAATCCAAAATAAAGGAA |
| 2235 | Left | GTATTTTGAAACTCAAGATCGCATT |
| 2236 | Right | TAAGAACAGAGACATCAGACCACAC |
| 2237 | Left | CCTCCATGAGTACGTATTTTGAAAC |
| 2238 | Right | CAAAGGTAGCTGATTGATGAGAGTT |
| 2239 | Left | GTATTTTGAAACTCAAGATCGCATT |
| 2240 | Right | AAATTCTTCAAAGGTAGCTGATTGA |

EGFR Exon18-19 2 kb

| | | |
|---|---|---|
| 2241 | Left | ATGCCAAAGAAGTAGAATGAGAAAA |
| 2242 | Right | AACATGTCACCAACTGGGTATAACT |
| 2243 | Left | ATGCCAAAGAAGTAGAATGAGAAAA |
| 2244 | Right | GGGTATAACTGCACATTCAGAGATT |
| 2245 | Left | TCTCCAAAATATATGCCAAAGAAGT |
| 2246 | Right | AACATGTCACCAACTGGGTATAACT |
| 2247 | Left | TGCCAAAGAAGTAGAATGAGAAAAA |
| 2248 | Right | AACATGTCACCAACTGGGTATAACT |
| 2249 | Left | AATCTCCAAAATATATGCCAAAGAA |
| 2250 | Right | AACATGTCACCAACTGGGTATAACT |
| 2251 | Left | AAATCTCCAAAATATATGCCAAAGA |
| 2252 | Right | AACATGTCACCAACTGGGTATAACT |
| 2253 | Left | TCTCCAAAATATATGCCAAAGAAGT |
| 2254 | Right | AACTGCACATTCAGAGATTCTTTCT |
| 2255 | Left | TCTCCAAAATATATGCCAAAGAAGT |
| 2256 | Right | GGGTATAACTGCACATTCAGAGATT |
| 2257 | Left | TGCCAAAGAAGTAGAATGAGAAAAA |
| 2258 | Right | GGGTATAACTGCACATTCAGAGATT |
| 2259 | Left | AAATCTCCAAAATATATGCCAAAGA |
| 2260 | Right | AACTGCACATTCAGAGATTCTTTCT |
| 2261 | Left | AATCTCCAAAATATATGCCAAAGAA |
| 2262 | Right | GGGTATAACTGCACATTCAGAGATT |
| 2263 | Left | AAATCTCCAAAATATATGCCAAAGA |
| 2264 | Right | GGGTATAACTGCACATTCAGAGATT |
| 2265 | Left | ATCTCCAAAATATATGCCAAAGAAG |
| 2266 | Right | AACATGTCACCAACTGGGTATAACT |
| 2267 | Left | AAAAATCTCCAAAATATATGCCAAAG |
| 2268 | Right | AACATGTCACCAACTGGGTATAACT |
| 2269 | Left | AAAATCTCCAAAATATATGCCAAAG |
| 2270 | Right | AACATGTCACCAACTGGGTATAACT |
| 2271 | Left | ATGCCAAAGAAGTAGAATGAGAAAA |
| 2272 | Right | ACTGGGTATAACTGCACATTCAGAG |
| 2273 | Left | ATCTCCAAAATATATGCCAAAGAAG |
| 2274 | Right | AACTGCACATTCAGAGATTCTTTCT |
| 2275 | Left | AATCTCCAAAATATATGCCAAAGAAG |
| 2276 | Right | AACATGTCACCAACTGGGTATAACT |
| 2277 | Left | ATCTCCAAAATATATGCCAAAGAAG |
| 2278 | Right | GGGTATAACTGCACATTCAGAGATT |
| 2279 | Left | AAAAATCTCCAAAATATATGCCAAAG |
| 2280 | Right | AACTGCACATTCAGAGATTCTTTCT |

EGFR Exon20-21 2 kb

| | | |
|---|---|---|
| 2281 | Left | TTTCTACCAACTTCTGTCAAGCTCT |
| 2282 | Right | TGCTATGTATTCTGTGGGTTAGACA |
| 2283 | Left | TTTCTACCAACTTCTGTCAAGCTCT |
| 2284 | Right | TGAGGTAATAAGTCAGCCATTTTTC |
| 2285 | Left | TTTCTACCAACTTCTGTCAAGCTCT |
| 2286 | Right | GGTAATAAGTCAGCCATTTTTCCTT |
| 2287 | Left | AGAAAGAATCTCTGAATGTGCAGTT |
| 2288 | Right | CAAAGTAACAATCAACAGACACTGG |
| 2289 | Left | TTTCTACCAACTTCTGTCAAGCTCT |
| 2290 | Right | ATAAAGGCCCATGTTCTCTTTACTT |
| 2291 | Left | TTTCTACCAACTTCTGTCAAGCTCT |
| 2292 | Right | CCTTCTTGGCTGTAAGATCAACTAA |
| 2293 | Left | AATCTCTGAATGTGCAGTTATACCC |
| 2294 | Right | CAAAGTAACAATCAACAGACACTGG |
| 2295 | Left | TTTCTACCAACTTCTGTCAAGCTCT |
| 2296 | Right | ATTACTCTCTGGCTTTTGTCCTTCT |
| 2297 | Left | GTTTCTACCAACTTCTGTCAAGCTC |
| 2298 | Right | TGAGGTAATAAGTCAGCCATTTTTC |
| 2299 | Left | AGAAAGAATCTCTGAATGTGCAGTT |
| 2300 | Right | AAAGATGAGATAACTTGGTGGAGTG |
| 2301 | Left | AGAAAGAATCTCTGAATGTGCAGTT |
| 2302 | Right | CAAAGATGAGATAACTTGGTGGAGT |
| 2303 | Left | AGAAAGAATCTCTGAATGTGCAGTT |
| 2304 | Right | TTTTCCAAGAACTTATTCCACAAAG |
| 2305 | Left | AATCTCTGAATGTGCAGTTATACCC |
| 2306 | Right | AAAGATGAGATAACTTGGTGGAGTG |
| 2307 | Left | AATCTCTGAATGTGCAGTTATACCC |
| 2308 | Right | CAAAGATGAGATAACTTGGTGGAGT |
| 2309 | Left | TTTCTACCAACTTCTGTCAAGCTCT |
| 2310 | Right | AATAAGTCAGCCATTTTTCCTTTTC |
| 2311 | Left | TTTCTACCAACTTCTGTCAAGCTCT |
| 2312 | Right | CTGCCCAGAGAAAATTAAACTGTAG |
| 2313 | Left | AATCTCTGAATGTGCAGTTATACCC |
| 2314 | Right | TTTTCCAAGAACTTATTCCACAAAG |
| 2315 | Left | GTTTCTACCAACTTCTGTCAAGCTC |
| 2316 | Right | GGTAATAAGTCAGCCATTTTTCCTT |
| 2317 | Left | TGTGTTTCTACCAACTTCTGTCAAG |
| 2318 | Right | TGCTATGTATTCTGTGGGTTAGACA |
| 2319 | Left | GTTTCTACCAACTTCTGTCAAGCTC |
| 2320 | Right | ATAAAGGCCCATGTTCTCTTTACTT |

EGFR Exon22 2 kb

| | | |
|---|---|---|
| 2321 | Left | CACATAGCATTTGCACTGTATTAGG |
| 2322 | Right | TTGAATCCAAAATAAAGGAATGTGT |
| 2323 | Left | ATTTTGATATTTAAGGGAGGTCCTG |
| 2324 | Right | TTCTCCACTACAAATCACCACAGTA |
| 2325 | Left | CACATAGCATTTGCACTGTATTAGG |
| 2326 | Right | TTCTCCACTACAAATCACCACAGTA |
| 2327 | Left | GTCTGTAGGTTACACACAAATGCTG |
| 2328 | Right | CACACTGAGCACTCAATAAAGAGAA |
| 2329 | Left | CACATAGCATTTGCACTGTATTAGG |
| 2330 | Right | ATTCTTCAAAGGTAGCTGATTGATG |
| 2331 | Left | GTCTGTAGGTTACACACAAATGCTG |
| 2332 | Right | TTCTCCACTACAAATCACCACAGTA |
| 2333 | Left | AGGTAATCAGGAGATGCTGTAGATG |
| 2334 | Right | CACACTGAGCACTCAATAAAGAGAA |
| 2335 | Left | GTCTGTAGGTTACACACAAATGCTG |
| 2336 | Right | ATTCTTCAAAGGTAGCTGATTGATG |
| 2337 | Left | ATTTTGATATTTAAGGGAGGTCCTG |
| 2338 | Right | GCTTACCTTGTTATCAAGTCCTGAA |
| 2339 | Left | CACATAGCATTTGCACTGTATTAGG |
| 2340 | Right | GCTTACCTTGTTATCAAGTCCTGAA |
| 2341 | Left | AGGTAATCAGGAGATGCTGTAGATG |
| 2342 | Right | ATTCTTCAAAGGTAGCTGATTGATG |
| 2343 | Left | GTCTGTAGGTTACACACAAATGCTG |
| 2344 | Right | GCTTACCTTGTTATCAAGTCCTGAA |
| 2345 | Left | AGGTAATCAGGAGATGCTGTAGATG |
| 2346 | Right | GCTTACCTTGTTATCAAGTCCTGAA |
| 2347 | Left | GTATTTTGAAACTCAAGATCGCATT |
| 2348 | Right | TTATACACATAGCGGAGTGATCAAA |
| 2349 | Left | TCTGAGAAAGAGTCTGCTAAGGAAG |
| 2350 | Right | TTGAATCCAAAATAAAGGAATGTGT |
| 2351 | Left | CTCTGAGAAAGAGTCTGCTAAGGAA |
| 2352 | Right | TTGAATCCAAAATAAAGGAATGTGT |
| 2353 | Left | TCGGTACTGAACATATACGGACTTT |
| 2354 | Right | TTGAATCCAAAATAAAGGAATGTGT |
| 2355 | Left | TCGGTACTGAACATATACGGACTTT |
| 2356 | Right | CACACTGAGCACTCAATAAAGAGAA |
| 2357 | Left | CACATAGCATTTGCACTGTATTAGG |
| 2358 | Right | CAAAGGTAGCTGATTGATGAGAGTT |
| 2359 | Left | TCTGAGAAAGAGTCTGCTAAGGAAG |
| 2360 | Right | ATCCAAAATAAAGGAATGTGTGTGT |

EGFR Exon18-21 5 kb

| | | |
|---|---|---|
| 2361 | Left | ATGCCAAAGAAGTAGAATGAGAAAA |
| 2362 | Right | TGCTATGTATTCTGTGGGTTAGACA |
| 2363 | Left | ATGCCAAAGAAGTAGAATGAGAAAA |
| 2364 | Right | CCTTCTTGGCTGTAAGATCAACTAA |
| 2365 | Left | ATGCCAAAGAAGTAGAATGAGAAAA |
| 2366 | Right | GTGCACTTAACTTTTAAGCCTTGAC |
| 2367 | Left | ATGCCAAAGAAGTAGAATGAGAAAA |
| 2368 | Right | AGATTGTAAGTGAAAGGCTTCACAG |
| 2369 | Left | TCTCCAAAATATATGCCAAAGAAGT |
| 2370 | Right | ATCTATCTTCTACCCCATTTCCAAC |
| 2371 | Left | ATGCCAAAGAAGTAGAATGAGAAAA |
| 2372 | Right | CTGCCCAGAGAAAATTAAACTGTAG |
| 2373 | Left | TCTCCAAAATATATGCCAAAGAAGT |
| 2374 | Right | TGCTATGTATTCTGTGGGTTAGACA |
| 2375 | Left | TGCCAAAGAAGTAGAATGAGAAAAA |
| 2376 | Right | ATCTATCTTCTACCCCATTTCCAAC |
| 2377 | Left | TCTCCAAAATATATGCCAAAGAAGT |
| 2378 | Right | TGAGGTAATAAGTCAGCCATTTTTC |
| 2379 | Left | TGCCAAAGAAGTAGAATGAGAAAAA |
| 2380 | Right | TGCTATGTATTCTGTGGGTTAGACA |
| 2381 | Left | ATGCCAAAGAAGTAGAATGAGAAAA |
| 2382 | Right | AGACATTTTTATAAAGGCCCATGTT |
| 2383 | Left | ATGCCAAAGAAGTAGAATGAGAAAA |
| 2384 | Right | ATTGTAAGTGAAAGGCTTCACAGAT |
| 2385 | Left | TCTCCAAAATATATGCCAAAGAAGT |
| 2386 | Right | GGTAATAAGTCAGCCATTTTTCCTT |
| 2387 | Left | AATCTCCAAAATATATGCCAAAGAA |
| 2388 | Right | ATCTATCTTCTACCCCATTTCCAAC |
| 2389 | Left | AAATCTCCAAAATATATGCCAAAGA |
| 2390 | Right | ATCTATCTTCTACCCCATTTCCAAC |
| 2391 | Left | AAATCTCCAAAATATATGCCAAAGA |
| 2392 | Right | TGCTATGTATTCTGTGGGTTAGACA |
| 2393 | Left | TCTCCAAAATATATGCCAAAGAAGT |
| 2394 | Right | CCTTCTTGGCTGTAAGATCAACTAA |
| 2395 | Left | TCTCCAAAATATATGCCAAAGAAGT |
| 2396 | Right | GTGCACTTAACTTTTAAGCCTTGAC |
| 2397 | Left | AATCTCCAAAATATATGCCAAAGAA |
| 2398 | Right | TGAGGTAATAAGTCAGCCATTTTTC |
| 2399 | Left | AAATCTCCAAAATATATGCCAAAGA |
| 2400 | Right | TGAGGTAATAAGTCAGCCATTTTTC |

EGFR Exon22 5 kb

| | | |
|---|---|---|
| 2401 | Left | GTTGGAAATGGGGTAGAAGATAGAT |
| 2402 | Right | TTGAATCCAAAATAAAGGAATGTGT |
| 2403 | Left | GTTGGAAATGGGGTAGAAGATAGAT |
| 2404 | Right | CACACTGAGCACTCAATAAAGAGAA |
| 2405 | Left | ATTTTGATATTTAAGGGAGGTCCTG |
| 2406 | Right | CTCTCCCATCAACATTTAGAAGAAA |
| 2407 | Left | CACATAGCATTTGCACTGTATTAGG |
| 2408 | Right | CTCTCCCATCAACATTTAGAAGAAA |
| 2409 | Left | ATTTTGATATTTAAGGGAGGTCCTG |
| 2410 | Right | TACAACAAACACAAGAATGGCTTTA |
| 2411 | Left | CACATAGCATTTGCACTGTATTAGG |
| 2412 | Right | TACAACAAACACAAGAATGGCTTTA |
| 2413 | Left | GTTGGAAATGGGGTAGAAGATAGAT |
| 2414 | Right | TTCTCCACTACAAATCACCACAGTA |
| 2415 | Left | ATATCTGAATAAAAGGTCACCACCA |
| 2416 | Right | CTCTCCCATCAACATTTAGAAGAAA |
| 2417 | Left | CCATATCTGAATAAAAGGTCACCAC |
| 2418 | Right | CTCTCCCATCAACATTTAGAAGAAA |
| 2419 | Left | TATCTGAATAAAAGGTCACCACCAT |
| 2420 | Right | CTCTCCCATCAACATTTAGAAGAAA |
| 2421 | Left | CACATAGCATTTGCACTGTATTAGG |
| 2422 | Right | GGGTCAAATAAACCTCCACTTATCT |
| 2423 | Left | GTTGGAAATGGGGTAGAAGATAGAT |
| 2424 | Right | ATTCTTCAAAGGTAGCTGATTGATG |
| 2425 | Left | ATTTTGATATTTAAGGGAGGTCCTG |
| 2426 | Right | TATAAGCCAATAAATCCCATTTTGA |
| 2427 | Left | CACATAGCATTTGCACTGTATTAGG |
| 2428 | Right | TATAAGCCAATAAATCCCATTTTGA |
| 2429 | Left | GTCTGTAGGTTACACACAAATGCTG |
| 2430 | Right | TACAACAAACACAAGAATGGCTTTA |
| 2431 | Left | TACAGATTATGATGACTGCCTCAAA |
| 2432 | Right | TACAACAAACACAAGAATGGCTTTA |
| 2433 | Left | AGGAAAATAACACACACTCTCCTTG |
| 2434 | Right | TTACTGGGAGATGATTAAGAACAGC |
| 2435 | Left | GTCTGTAGGTTACACACAAATGCTG |
| 2436 | Right | GGGTCAAATAAACCTCCACTTATCT |
| 2437 | Left | ATATCTGAATAAAAGGTCACCACCA |
| 2438 | Right | TATAAGCCAATAAATCCCATTTTGA |
| 2439 | Left | CCATATCTGAATAAAAGGTCACCAC |
| 2440 | Right | TATAAGCCAATAAATCCCATTTTGA |

TABLE 9
KIT Capture Primer List for NGS Panel

| | | |
|---|---|---|
| Seq. | | |
| ID | Primer | Sequence |

KIT Exon8 150-175 bases

| | | |
|---|---|---|
| 2441 | Left | ATATGGCCATTTCTGTTTTCCTGTA |
| 2442 | Right | ATAAGCAGTGCCAAAAATAATCATC |
| 2443 | Left | CTGACATATGGCCATTTCTGTTT |
| 2444 | Right | ATAAGCAGTGCCAAAAATAATCATC |
| 2445 | Left | GACATATGGCCATTTCTGTTTTC |
| 2446 | Right | ATAAGCAGTGCCAAAAATAATCATC |
| 2447 | Left | CTGACATATGGCCATTTCTGTTTT |
| 2448 | Right | ATAAGCAGTGCCAAAAATAATCATC |
| 2449 | Left | ATATGGCCATTTCTGTTTTCCTGTA |
| 2450 | Right | GCATTATAAGCAGTGCCAAAAATAA |
| 2451 | Left | TATGGCCATTTCTGTTTTCCTGTAG |
| 2452 | Right | ATAAGCAGTGCCAAAAATAATCATC |
| 2453 | Left | TGACATATGGCCATTTCTGTTTT |
| 2454 | Right | ATAAGCAGTGCCAAAAATAATCATC |
| 2455 | Left | ATATGGCCATTTCTGTTTTCCTGTA |
| 2456 | Right | TTATAAGCAGTGCCAAAAATAATCA |
| 2457 | Left | TATGGCCATTTCTGTTTTCCTGTA |
| 2458 | Right | ATAAGCAGTGCCAAAAATAATCATC |
| 2459 | Left | GACATATGGCCATTTCTGTTTTC |
| 2460 | Right | TTATAAGCAGTGCCAAAAATAATCA |
| 2461 | Left | CTGACATATGGCCATTTCTGTTTTC |
| 2462 | Right | ATAAGCAGTGCCAAAAATAATCATC |
| 2463 | Left | ATATGGCCATTTCTGTTTTCCTGTA |
| 2464 | Right | TATAAGCAGTGCCAAAAATAATCATC |
| 2465 | Left | GGCCATTTCTGTTTTCCTGTAG |
| 2466 | Right | ATAAGCAGTGCCAAAAATAATCATC |
| 2467 | Left | TATGGCCATTTCTGTTTTCCTGTAG |
| 2468 | Right | GCATTATAAGCAGTGCCAAAAATAA |
| 2469 | Left | ATATGGCCATTTCTGTTTTCCTGT |
| 2470 | Right | ATAAGCAGTGCCAAAAATAATCATC |
| 2471 | Left | ACATATGGCCATTTCTGTTTTCCT |
| 2472 | Right | ATAAGCAGTGCCAAAAATAATCATC |
| 2473 | Left | TATGGCCATTTCTGTTTTCCTGTAG |
| 2474 | Right | TTATAAGCAGTGCCAAAAATAATCA |
| 2475 | Left | TGACATATGGCCATTTCTGTTTTC |
| 2476 | Right | ATAAGCAGTGCCAAAAATAATCATC |
| 2477 | Left | ATATGGCCATTTCTGTTTTCCTGTA |
| 2478 | Right | TAAGCAGTGCCAAAAATAATCATC |
| 2479 | Left | GACATATGGCCATTTCTGTTTTC |
| 2480 | Right | TATAAGCAGTGCCAAAAATAATCATC |

KIT Exon8 176-200 bases

| | | |
|---|---|---|
| 2481 | Left | AGGTTTTCCAGCACTCTGACATA |
| 2482 | Right | ATAAGCAGTGCCAAAAATAATCATC |
| 2483 | Left | ACTCTGACATATGGCCATTTCTGTT |
| 2484 | Right | ATAAGCAGTGCCAAAAATAATCATC |
| 2485 | Left | CTGACATATGGCCATTTCTGTTT |
| 2486 | Right | ATAAGCAGTGCCAAAAATAATCATC |
| 2487 | Left | CTGACATATGGCCATTTCTGTTTT |
| 2488 | Right | ATAAGCAGTGCCAAAAATAATCATC |
| 2489 | Left | ATATGGCCATTTCTGTTTTCCTGTA |
| 2490 | Right | GCATTATAAGCAGTGCCAAAAATAA |
| 2491 | Left | CTCTGACATATGGCCATTTCTGTT |
| 2492 | Right | ATAAGCAGTGCCAAAAATAATCATC |
| 2493 | Left | CTCTGACATATGGCCATTTCTGTTT |
| 2494 | Right | ATAAGCAGTGCCAAAAATAATCATC |
| 2495 | Left | AGGTTTTCCAGCACTCTGACATA |
| 2496 | Right | GCATTATAAGCAGTGCCAAAAATAA |
| 2497 | Left | ACTCTGACATATGGCCATTTCTGTT |
| 2498 | Right | GCATTATAAGCAGTGCCAAAAATAA |
| 2499 | Left | TCTGACATATGGCCATTTCTGTT |
| 2500 | Right | ATAAGCAGTGCCAAAAATAATCATC |
| 2501 | Left | GACATATGGCCATTTCTGTTTTC |
| 2502 | Right | GCATTATAAGCAGTGCCAAAAATAA |
| 2503 | Left | AGGTTTTCCAGCACTCTGACATA |
| 2504 | Right | TTATAAGCAGTGCCAAAAATAATCA |
| 2505 | Left | ACTCTGACATATGGCCATTTCTGTT |
| 2506 | Right | TTATAAGCAGTGCCAAAAATAATCA |
| 2507 | Left | GAGGTTTTCCAGCACTCTGACATA |
| 2508 | Right | ATAAGCAGTGCCAAAAATAATCATC |
| 2509 | Left | CTGACATATGGCCATTTCTGTTT |
| 2510 | Right | TTATAAGCAGTGCCAAAAATAATCA |
| 2511 | Left | TCTGACATATGGCCATTTCTGTTT |
| 2512 | Right | ATAAGCAGTGCCAAAAATAATCATC |
| 2513 | Left | GACATATGGCCATTTCTGTTTTC |
| 2514 | Right | TTATAAGCAGTGCCAAAAATAATCA |
| 2515 | Left | CTGACATATGGCCATTTCTGTTTTC |
| 2516 | Right | ATAAGCAGTGCCAAAAATAATCATC |
| 2517 | Left | TCTGACATATGGCCATTTCTGTTTT |
| 2518 | Right | ATAAGCAGTGCCAAAAATAATCATC |
| 2519 | Left | CTGACATATGGCCATTTCTGTTTT |
| 2520 | Right | TTATAAGCAGTGCCAAAAATAATCA |

KIT Exon8 201-300 bases

| | | |
|---|---|---|
| 2521 | Left | TTAGAGAGGGAGTGAAGTGAATGTT |
| 2522 | Right | ATAAGCAGTGCCAAAAATAATCATC |
| 2523 | Left | GATTAGAGAGGGAGTGAAGTGAATG |
| 2524 | Right | ATAAGCAGTGCCAAAAATAATCATC |
| 2525 | Left | GGATTAGAGAGGGAGTGAAGTGAAT |
| 2526 | Right | ATAAGCAGTGCCAAAAATAATCATC |
| 2527 | Left | GTAGGGATTAGAGAGGGAGTGAAGT |
| 2528 | Right | ATAAGCAGTGCCAAAAATAATCATC |
| 2529 | Left | TTAGAGAGGGAGTGAAGTGAATGTT |
| 2530 | Right | GCATTATAAGCAGTGCCAAAAATAA |
| 2531 | Left | GATTAGAGAGGGAGTGAAGTGAATG |
| 2532 | Right | GCATTATAAGCAGTGCCAAAAATAA |
| 2533 | Left | CAGGAAGGTTGTAGGGATTAGAGAG |
| 2534 | Right | ATAAGCAGTGCCAAAAATAATCATC |
| 2535 | Left | CTCAGGAAGGTTGTAGGGATTAGAG |
| 2536 | Right | ATAAGCAGTGCCAAAAATAATCATC |
| 2537 | Left | GGATTAGAGAGGGAGTGAAGTGAAT |
| 2538 | Right | GCATTATAAGCAGTGCCAAAAATAA |
| 2539 | Left | GTAGGGATTAGAGAGGGAGTGAAGT |
| 2540 | Right | GCATTATAAGCAGTGCCAAAAATAA |
| 2541 | Left | GATTAGAGAGGGAGTGAAGTGAATG |
| 2542 | Right | TTATAAGCAGTGCCAAAAATAATCA |
| 2543 | Left | GGATTAGAGAGGGAGTGAAGTGAAT |
| 2544 | Right | TTATAAGCAGTGCCAAAAATAATCA |
| 2545 | Left | TCAGGAAGGTTGTAGGGATTAGAG |
| 2546 | Right | ATAAGCAGTGCCAAAAATAATCATC |
| 2547 | Left | CAGGAAGGTTGTAGGGATTAGAGA |
| 2548 | Right | ATAAGCAGTGCCAAAAATAATCATC |
| 2549 | Left | CTCAGGAAGGTTGTAGGGATTAGA |
| 2550 | Right | ATAAGCAGTGCCAAAAATAATCATC |
| 2551 | Left | TAGAGAGGGAGTGAAGTGAATGTTG |
| 2552 | Right | ATAAGCAGTGCCAAAAATAATCATC |
| 2553 | Left | GTAGGGATTAGAGAGGGAGTGAAGT |
| 2554 | Right | TTATAAGCAGTGCCAAAAATAATCA |
| 2555 | Left | AGGTTGTAGGGATTAGAGAGGGAGT |
| 2556 | Right | ATAAGCAGTGCCAAAAATAATCATC |
| 2557 | Left | ATTAGAGAGGGAGTGAAGTGAATGTT |
| 2558 | Right | ATAAGCAGTGCCAAAAATAATCATC |
| 2559 | Left | GGATTAGAGAGGGAGTGAAGTGAA |
| 2560 | Right | ATAAGCAGTGCCAAAAATAATCATC |

KIT Exon8 301-400 bases

| | | |
|---|---|---|
| 2561 | Left | TTAGAGAGGGAGTGAAGTGAATGTT |
| 2562 | Right | TCATTCAGTAATGATTTTTCAGCAA |
| 2563 | Left | TTAGAGAGGGAGTGAAGTGAATGTT |
| 2564 | Right | TCAGTAATGATTTTTCAGCAAACAA |
| 2565 | Left | GATTAGAGAGGGAGTGAAGTGAATG |
| 2566 | Right | TCAGTAATGATTTTTCAGCAAACAA |
| 2567 | Left | GATTAGAGAGGGAGTGAAGTGAATG |
| 2568 | Right | TTCAGTAATGATTTTTCAGCAAACA |
| 2569 | Left | GGATTAGAGAGGGAGTGAAGTGAAT |
| 2570 | Right | TCAGTAATGATTTTTCAGCAAACAA |
| 2571 | Left | GGATTAGAGAGGGAGTGAAGTGAAT |
| 2572 | Right | TTCAGTAATGATTTTTCAGCAAACA |
| 2573 | Left | TTAGAGAGGGAGTGAAGTGAATGTT |
| 2574 | Right | AATTATCCCTTCTAAAAAGCCACAT |
| 2575 | Left | GATTAGAGAGGGAGTGAAGTGAATG |
| 2576 | Right | AATTATCCCTTCTAAAAAGCCACAT |
| 2577 | Left | GGATTAGAGAGGGAGTGAAGTGAAT |
| 2578 | Right | AATTATCCCTTCTAAAAAGCCACAT |
| 2579 | Left | TTAGAGAGGGAGTGAAGTGAATGTT |
| 2580 | Right | GTCATTCAGTAATGATTTTTCAGCA |
| 2581 | Left | TTAGAGAGGGAGTGAAGTGAATGTT |
| 2582 | Right | CAGCAAACAAAATTAATGTCTACCA |
| 2583 | Left | GATTAGAGAGGGAGTGAAGTGAATG |
| 2584 | Right | CAGCAAACAAAATTAATGTCTACCA |
| 2585 | Left | GGATTAGAGAGGGAGTGAAGTGAAT |
| 2586 | Right | CAGCAAACAAAATTAATGTCTACCA |
| 2587 | Left | GTAGGGATTAGAGAGGGAGTGAAGT |
| 2588 | Right | CAGCAAACAAAATTAATGTCTACCA |
| 2589 | Left | TAGAGAGGGAGTGAAGTGAATGTTG |
| 2590 | Right | TCATTCAGTAATGATTTTTCAGCAA |
| 2591 | Left | ATTAGAGAGGGAGTGAAGTGAATGTT |
| 2592 | Right | TCATTCAGTAATGATTTTTCAGCAA |
| 2593 | Left | TAGAGAGGGAGTGAAGTGAATGTTG |
| 2594 | Right | TCAGTAATGATTTTTCAGCAAACAA |
| 2595 | Left | TAGAGAGGGAGTGAAGTGAATGTTG |
| 2596 | Right | TTCAGTAATGATTTTTCAGCAAACA |
| 2597 | Left | ATTAGAGAGGGAGTGAAGTGAATGTT |
| 2598 | Right | TCAGTAATGATTTTTCAGCAAACAA |
| 2599 | Left | CAGGAAGGTTGTAGGGATTAGAGAG |
| 2600 | Right | AATTATCCCTTCTAAAAAGCCACAT |

KIT Exon8 401-500 bases

| | | |
|---|---|---|
| 2601 | Left | GATTAGAGAGGGAGTGAAGTGAATG |
| 2602 | Right | TCATTCAGTAATGATTTTTCAGCAA |
| 2603 | Left | GGATTAGAGAGGGAGTGAAGTGAAT |
| 2604 | Right | TCATTCAGTAATGATTTTTCAGCAA |
| 2605 | Left | GTAGGGATTAGAGAGGGAGTGAAGT |
| 2606 | Right | TCATTCAGTAATGATTTTTCAGCAA |
| 2607 | Left | GTAGGGATTAGAGAGGGAGTGAAGT |
| 2608 | Right | TCAGTAATGATTTTTCAGCAAACAA |
| 2609 | Left | GTAGGGATTAGAGAGGGAGTGAAGT |
| 2610 | Right | TTCAGTAATGATTTTTCAGCAAACA |
| 2611 | Left | TTAGAGAGGGAGTGAAGTGAATGTT |
| 2612 | Right | AAATTGCATGATAAATCCAGAAAGA |
| 2613 | Left | TTAGAGAGGGAGTGAAGTGAATGTT |
| 2614 | Right | GAAATTGCATGATAAATCCAGAAAG |
| 2615 | Left | GATTAGAGAGGGAGTGAAGTGAATG |
| 2616 | Right | AAATTGCATGATAAATCCAGAAAGA |
| 2617 | Left | GATTAGAGAGGGAGTGAAGTGAATG |
| 2618 | Right | GAAATTGCATGATAAATCCAGAAAG |
| 2619 | Left | GGATTAGAGAGGGAGTGAAGTGAAT |
| 2620 | Right | AAATTGCATGATAAATCCAGAAAGA |
| 2621 | Left | GGATTAGAGAGGGAGTGAAGTGAAT |
| 2622 | Right | GAAATTGCATGATAAATCCAGAAAG |
| 2623 | Left | GTAGGGATTAGAGAGGGAGTGAAGT |
| 2624 | Right | AAATTGCATGATAAATCCAGAAAGA |
| 2625 | Left | GTAGGGATTAGAGAGGGAGTGAAGT |
| 2626 | Right | GAAATTGCATGATAAATCCAGAAAG |
| 2627 | Left | TTAGAAGCAGTCTTCAGATCCCTAC |
| 2628 | Right | ATAAGCAGTGCCAAAAATAATCATC |
| 2629 | Left | GATTAGAGAGGGAGTGAAGTGAATG |
| 2630 | Right | GTCATTCAGTAATGATTTTTCAGCA |
| 2631 | Left | GGATTAGAGAGGGAGTGAAGTGAAT |
| 2632 | Right | GTCATTCAGTAATGATTTTTCAGCA |
| 2633 | Left | AGATTTTTACCTGTGGAACACTTTG |
| 2634 | Right | GCATTATAAGCAGTGCCAAAAATAA |
| 2635 | Left | GTAGGGATTAGAGAGGGAGTGAAGT |
| 2636 | Right | GTCATTCAGTAATGATTTTTCAGCA |
| 2637 | Left | CAGGAAGGTTGTAGGGATTAGAGAG |
| 2638 | Right | TCATTCAGTAATGATTTTTCAGCAA |
| 2639 | Left | CTCAGGAAGGTTGTAGGGATTAGAG |
| 2640 | Right | TCATTCAGTAATGATTTTTCAGCAA |

KIT Exon8 501-600 bases

| | | |
|---|---|---|
| 2641 | Left | TTAGAGAGGGAGTGAAGTGAATGTT |
| 2642 | Right | CGATCATTACTTTTTGGTAACTTGG |
| 2643 | Left | GATTAGAGAGGGAGTGAAGTGAATG |
| 2644 | Right | CGATCATTACTTTTTGGTAACTTGG |
| 2645 | Left | GGATTAGAGAGGGAGTGAAGTGAAT |
| 2646 | Right | CGATCATTACTTTTTGGTAACTTGG |
| 2647 | Left | TTAGAGAGGGAGTGAAGTGAATGTT |
| 2648 | Right | CATTACTTTTTGGTAACTTGGCAAT |
| 2649 | Left | TTAGAGAGGGAGTGAAGTGAATGTT |
| 2650 | Right | GCGATCATTACTTTTTGGTAACTTG |
| 2651 | Left | TTAGAGAGGGAGTGAAGTGAATGTT |
| 2652 | Right | TGCGATCATTACTTTTTGGTAACTT |
| 2653 | Left | GATTAGAGAGGGAGTGAAGTGAATG |
| 2654 | Right | CATTACTTTTTGGTAACTTGGCAAT |
| 2655 | Left | GATTAGAGAGGGAGTGAAGTGAATG |
| 2656 | Right | GCGATCATTACTTTTTGGTAACTTG |
| 2657 | Left | GGATTAGAGAGGGAGTGAAGTGAAT |
| 2658 | Right | CATTACTTTTTGGTAACTTGGCAAT |
| 2659 | Left | ACTCCTAATTTCATCCATTCCAGTT |
| 2660 | Right | ATAAGCAGTGCCAAAAATAATCATC |
| 2661 | Left | AACTCCTAATTTCATCCATTCCAGT |
| 2662 | Right | ATAAGCAGTGCCAAAAATAATCATC |
| 2663 | Left | GTAGGGATTAGAGAGGGAGTGAAGT |
| 2664 | Right | CATTACTTTTTGGTAACTTGGCAAT |
| 2665 | Left | GGCAGGAATCCTTTAAAGTAGATTT |
| 2666 | Right | ATAAGCAGTGCCAAAAATAATCATC |
| 2667 | Left | TTAGAAGCAGTCTTCAGATCCCTAC |
| 2668 | Right | AATTATCCCTTCTAAAAAGCCACAT |
| 2669 | Left | TTAGAGAGGGAGTGAAGTGAATGTT |
| 2670 | Right | GATAACTACAGTCACATTTCCCACA |
| 2671 | Left | AACAACTCCTAATTTCATCCATTCC |
| 2672 | Right | ATAAGCAGTGCCAAAAATAATCATC |
| 2673 | Left | GATTAGAGAGGGAGTGAAGTGAATG |
| 2674 | Right | GATAACTACAGTCACATTTCCCACA |
| 2675 | Left | TTAGAGAGGGAGTGAAGTGAATGTT |
| 2676 | Right | TAACTACAGTCACATTTCCCACACA |
| 2677 | Left | GGATTAGAGAGGGAGTGAAGTGAAT |
| 2678 | Right | GATAACTACAGTCACATTTCCCACA |
| 2679 | Left | CACTTTGGAGTCCTAGAGTTTGATT |
| 2680 | Right | AATTATCCCTTCTAAAAAGCCACAT |

KIT Exon8 601-800 bases

| | | |
|---|---|---|
| 2681 | Left | AGATTTTTACCTGTGGAACACTTTG |
| 2682 | Right | TCATTCAGTAATGATTTTTCAGCAA |
| 2683 | Left | AGATTTTTACCTGTGGAACACTTTG |
| 2684 | Right | TTCAGTAATGATTTTTCAGCAAACA |
| 2685 | Left | AGATTTTTACCTGTGGAACACTTTG |
| 2686 | Right | TCAGTAATGATTTTTCAGCAAACAA |
| 2687 | Left | GGAGAAAATTCATGTAAGAGCAAAA |
| 2688 | Right | ATAAGCAGTGCCAAAAATAATCATC |
| 2689 | Left | AATTCATGTAAGAGCAAAAGAGTGG |
| 2690 | Right | ATAAGCAGTGCCAAAAATAATCATC |
| 2691 | Left | GGAGAAAATTCATGTAAGAGCAAAA |
| 2692 | Right | AATTATCCCTTCTAAAAAGCCACAT |
| 2693 | Left | AGATTTTTACCTGTGGAACACTTTG |
| 2694 | Right | AAATTGCATGATAAATCCAGAAAGA |
| 2695 | Left | AGATTTTTACCTGTGGAACACTTTG |
| 2696 | Right | GAAATTGCATGATAAATCCAGAAAG |
| 2697 | Left | AATTCATGTAAGAGCAAAAGAGTGG |
| 2698 | Right | AATTATCCCTTCTAAAAAGCCACAT |
| 2699 | Left | GGGCTTATCTTTTCCTCTAACAACT |
| 2700 | Right | TCATTCAGTAATGATTTTTCAGCAA |
| 2701 | Left | GGGCTTATCTTTTCCTCTAACAACT |
| 2702 | Right | TCAGTAATGATTTTTCAGCAAACAA |
| 2703 | Left | GGGCTTATCTTTTCCTCTAACAACT |
| 2704 | Right | TTCAGTAATGATTTTTCAGCAAACA |
| 2705 | Left | AGATTTTTACCTGTGGAACACTTTG |
| 2706 | Right | AATTATCCCTTCTAAAAAGCCACAT |
| 2707 | Left | AGGCTGGTTTTCTTTTCTAGTTTTC |
| 2708 | Right | ATAAGCAGTGCCAAAAATAATCATC |
| 2709 | Left | GGCTGGTTTTCTTTTCTAGTTTTCT |
| 2710 | Right | ATAAGCAGTGCCAAAAATAATCATC |
| 2711 | Left | ACTCCTAATTTCATCCATTCCAGTT |
| 2712 | Right | TCATTCAGTAATGATTTTTCAGCAA |
| 2713 | Left | AACTCCTAATTTCATCCATTCCAGT |
| 2714 | Right | TCATTCAGTAATGATTTTTCAGCAA |
| 2715 | Left | AGGAGAAAATTCATGTAAGAGCAAA |
| 2716 | Right | ATAAGCAGTGCCAAAAATAATCATC |
| 2717 | Left | AAGGAGAAAATTCATGTAAGAGCAA |
| 2718 | Right | ATAAGCAGTGCCAAAAATAATCATC |
| 2719 | Left | AAAGGAGAAAATTCATGTAAGAGCA |
| 2720 | Right | ATAAGCAGTGCCAAAAATAATCATC |

KIT Exon8 801-1000 bases

| | | |
|---|---|---|
| 2721 | Left | GGAGAAAATTCATGTAAGAGCAAAA |
| 2722 | Right | TCATTCAGTAATGATTTTTCAGCAA |
| 2723 | Left | GGAGAAAATTCATGTAAGAGCAAAA |
| 2724 | Right | TCAGTAATGATTTTTCAGCAAACAA |
| 2725 | Left | GGAGAAAATTCATGTAAGAGCAAAA |
| 2726 | Right | TTCAGTAATGATTTTTCAGCAAACA |
| 2727 | Left | AATTCATGTAAGAGCAAAAGAGTGG |
| 2728 | Right | TCATTCAGTAATGATTTTTCAGCAA |
| 2729 | Left | AATTCATGTAAGAGCAAAAGAGTGG |
| 2730 | Right | TCAGTAATGATTTTTCAGCAAACAA |
| 2731 | Left | AATTCATGTAAGAGCAAAAGAGTGG |
| 2732 | Right | TTCAGTAATGATTTTTCAGCAAACA |
| 2733 | Left | TTAGAGCATTTCTGCTGTTACAGTG |
| 2734 | Right | ATAAGCAGTGCCAAAAATAATCATC |
| 2735 | Left | GGAGAAAATTCATGTAAGAGCAAAA |
| 2736 | Right | AAATTGCATGATAAATCCAGAAAGA |
| 2737 | Left | GGAGAAAATTCATGTAAGAGCAAAA |
| 2738 | Right | GAAATTGCATGATAAATCCAGAAAG |
| 2739 | Left | ATACCAAATTAGAGCATTTCTGCTG |
| 2740 | Right | ATAAGCAGTGCCAAAAATAATCATC |
| 2741 | Left | AGGCTGGTTTTCTTTTCTAGTTTTC |
| 2742 | Right | TCATTCAGTAATGATTTTTCAGCAA |
| 2743 | Left | GGCTGGTTTTCTTTTCTAGTTTTCT |
| 2744 | Right | TCATTCAGTAATGATTTTTCAGCAA |
| 2745 | Left | AATTCATGTAAGAGCAAAAGAGTGG |
| 2746 | Right | AAATTGCATGATAAATCCAGAAAGA |
| 2747 | Left | AATTCATGTAAGAGCAAAAGAGTGG |
| 2748 | Right | GAAATTGCATGATAAATCCAGAAAG |
| 2749 | Left | AGATTTTTACCTGTGGAACACTTTG |
| 2750 | Right | TGTTGTAATTGTGCGATCATTACTT |
| 2751 | Left | AGGCTGGTTTTCTTTTCTAGTTTTC |
| 2752 | Right | TTCAGTAATGATTTTTCAGCAAACA |
| 2753 | Left | AGATTTTTACCTGTGGAACACTTTG |
| 2754 | Right | CGATCATTACTTTTTGGTAACTTGG |
| 2755 | Left | TTAGAGAGGGAGTGAAGTGAATGTT |
| 2756 | Right | GAACCCTACTTAGTATTCCCCAAAA |
| 2757 | Left | AGGAGAAAATTCATGTAAGAGCAAA |
| 2758 | Right | TCATTCAGTAATGATTTTTCAGCAA |
| 2759 | Left | AAGGAGAAAATTCATGTAAGAGCAA |
| 2760 | Right | TCATTCAGTAATGATTTTTCAGCAA |

KIT Exon8 2 kb

| | | |
|---|---|---|
| 2761 | Left | GGAGAAAATTCATGTAAGAGCAAAA |
| 2762 | Right | GGACTGAAGTTTGAGTTCTAAGCAG |
| 2763 | Left | GGAGAAAATTCATGTAAGAGCAAAA |
| 2764 | Right | AGGACTGAAGTTTGAGTTCTAAGCA |
| 2765 | Left | TTAGAGCATTTCTGCTGTTACAGTG |
| 2766 | Right | CCTGTTTCCTTTCTTAACACCTACA |
| 2767 | Left | GGAGAAAATTCATGTAAGAGCAAAA |
| 2768 | Right | CCTGTTTCCTTTCTTAACACCTACA |
| 2769 | Left | AATTCATGTAAGAGCAAAAGAGTGG |
| 2770 | Right | ACTGAAGTTTGAGTTCTAAGCAGGA |
| 2771 | Left | AATTCATGTAAGAGCAAAAGAGTGG |
| 2772 | Right | GGACTGAAGTTTGAGTTCTAAGCAG |
| 2773 | Left | AATTCATGTAAGAGCAAAAGAGTGG |
| 2774 | Right | AGGACTGAAGTTTGAGTTCTAAGCA |
| 2775 | Left | GGAGAAAATTCATGTAAGAGCAAAA |
| 2776 | Right | GACCAGAAAATAGTCAAAGTGAGGA |
| 2777 | Left | TTCTGATCTGTCAGTCTTTCCTTCT |
| 2778 | Right | TCATTCAGTAATGATTTTTCAGCAA |
| 2779 | Left | ATACCAAATTAGAGCATTTCTGCTG |
| 2780 | Right | CCTGTTTCCTTTCTTAACACCTACA |
| 2781 | Left | TTAGAGCATTTCTGCTGTTACAGTG |
| 2782 | Right | GCTATTCCCTGTTTCCTTTCTTAAC |
| 2783 | Left | ATAAGTGCATCTTCCTTTCACTTTG |
| 2784 | Right | GAACCCTACTTAGTATTCCCCAAAA |
| 2785 | Left | GGAGAAAATTCATGTAAGAGCAAAA |
| 2786 | Right | GCTATTCCCTGTTTCCTTTCTTAAC |
| 2787 | Left | TTCTGATCTGTCAGTCTTTCCTTCT |
| 2788 | Right | TTCAGTAATGATTTTTCAGCAAACA |
| 2789 | Left | TTCTGATCTGTCAGTCTTTCCTTCT |
| 2790 | Right | TCAGTAATGATTTTTCAGCAAACAA |
| 2791 | Left | AATTCATGTAAGAGCAAAAGAGTGG |
| 2792 | Right | GACCAGAAAATAGTCAAAGTGAGGA |
| 2793 | Left | TTCCTTCTCACTGCATATATTTTCC |
| 2794 | Right | TCATTCAGTAATGATTTTTCAGCAA |
| 2795 | Left | ATACCAAATTAGAGCATTTCTGCTG |
| 2796 | Right | GCTATTCCCTGTTTCCTTTCTTAAC |
| 2797 | Left | TGAAATTGGTTATCCAAGAAAGGTA |
| 2798 | Right | TCATTCAGTAATGATTTTTCAGCAA |
| 2799 | Left | AGATTTTTACCTGTGGAACACTTTG |
| 2800 | Right | ACTGAAGTTTGAGTTCTAAGCAGGA |

KIT Exon8-9 5 kb

| | | |
|---|---|---|
| 2801 | Left | AGTTGCCCATGATAATTAAATGAAA |
| 2802 | Right | AGGCAGTGTTAACTTTTGGATACAG |
| 2803 | Left | CCCATGATAATTAAATGAAACTTGC |
| 2804 | Right | AGGCAGTGTTAACTTTTGGATACAG |
| 2805 | Left | GCCCATGATAATTAAATGAAACTTG |
| 2806 | Right | AGGCAGTGTTAACTTTTGGATACAG |
| 2807 | Left | TGCCCATGATAATTAAATGAAACTT |
| 2808 | Right | AGGCAGTGTTAACTTTTGGATACAG |
| 2809 | Left | TTGCCCATGATAATTAAATGAAACT |
| 2810 | Right | AGGCAGTGTTAACTTTTGGATACAG |
| 2811 | Left | AGTTTAGGCTTGCTTAGAAAAGGAG |
| 2812 | Right | AGGCAGTGTTAACTTTTGGATACAG |
| 2813 | Left | AGAGTTTAGGCTTGCTTAGAAAAGG |
| 2814 | Right | AGGCAGTGTTAACTTTTGGATACAG |
| 2815 | Left | GATTTCTTGTGTCGTGTCCTACTTT |
| 2816 | Right | AGGCAGTGTTAACTTTTGGATACAG |
| 2817 | Left | GCCCATGATAATTAAATGAAACTTG |
| 2818 | Right | GCTTCCTTTATGGACGGTTTATATT |
| 2819 | Left | TTGCCCATGATAATTAAATGAAACT |
| 2820 | Right | GCTTCCTTTATGGACGGTTTATATT |
| 2821 | Left | AGTTGCCCATGATAATTAAATGAAA |
| 2822 | Right | GCTTCCTTTATGGACGGTTTATATT |
| 2823 | Left | CCCATGATAATTAAATGAAACTTGC |
| 2824 | Right | GCTTCCTTTATGGACGGTTTATATT |
| 2825 | Left | TGCCCATGATAATTAAATGAAACTT |
| 2826 | Right | GCTTCCTTTATGGACGGTTTATATT |
| 2827 | Left | TTGCCCATGATAATTAAATGAAACT |
| 2828 | Right | CCCCTTAAATTGGATTAAAAAGAAA |
| 2829 | Left | AGTTGCCCATGATAATTAAATGAAA |
| 2830 | Right | CCCCTTAAATTGGATTAAAAAGAAA |
| 2831 | Left | GCCCATGATAATTAAATGAAACTTG |
| 2832 | Right | CCCCTTAAATTGGATTAAAAAGAAA |
| 2833 | Left | CCCATGATAATTAAATGAAACTTGC |
| 2834 | Right | CCCCTTAAATTGGATTAAAAAGAAA |
| 2835 | Left | TGCCCATGATAATTAAATGAAACTT |
| 2836 | Right | CCCCTTAAATTGGATTAAAAAGAAA |
| 2837 | Left | AGTTTAGGCTTGCTTAGAAAAGGAG |
| 2838 | Right | CCCCTTAAATTGGATTAAAAAGAAA |
| 2839 | Left | AGAGTTTAGGCTTGCTTAGAAAAGG |
| 2840 | Right | CCCCTTAAATTGGATTAAAAAGAAA |

KIT Exon9 200-250 bases

| | | |
|---|---|---|
| 2841 | Left | GGCTTTTGTTTTCTTCCCTTTAG |
| 2842 | Right | CATCCCCTTAAATTGGATTAAAAAG |
| 2843 | Left | GGCTTTTGTTTTCTTCCCTTTAG |
| 2844 | Right | ATCCCCTTAAATTGGATTAAAAAG |
| 2845 | Left | GGGCTTTTGTTTTCTTCCCTTTAG |
| 2846 | Right | ATCCCCTTAAATTGGATTAAAAAG |
| 2847 | Left | GGCTTTTGTTTTCTTCCCTTTAG |
| 2848 | Right | TCCCCTTAAATTGGATTAAAAAG |
| 2849 | Left | GCTTTTGTTTTCTTCCCTTTAG |
| 2850 | Right | CATCCCCTTAAATTGGATTAAAAAG |
| 2851 | Left | AGGGCTTTTGTTTTCTTCCCTTTAG |
| 2852 | Right | TCCCCTTAAATTGGATTAAAAAG |
| 2853 | Left | GGGCTTTTGTTTTCTTCCCTTTAG |
| 2854 | Right | TCCCCTTAAATTGGATTAAAAAG |
| 2855 | Left | GCTTTTGTTTTCTTCCCTTTAG |
| 2856 | Right | ACATCCCCTTAAATTGGATTAAAAAG |
| 2857 | Left | GGCTTTTGTTTTCTTCCCTTTAG |
| 2858 | Right | CCCCTTAAATTGGATTAAAAAG |
| 2859 | Left | GCTTTTGTTTTCTTCCCTTTAG |
| 2860 | Right | ATCCCCTTAAATTGGATTAAAAAG |
| 2861 | Left | AGGGCTTTTGTTTTCTTCCCTTTAG |
| 2862 | Right | CCCCTTAAATTGGATTAAAAAG |
| 2863 | Left | GGGCTTTTGTTTTCTTCCCTTTAG |
| 2864 | Right | CCCCTTAAATTGGATTAAAAAG |
| 2865 | Left | GCTTTTGTTTTCTTCCCTTTAG |
| 2866 | Right | TCCCCTTAAATTGGATTAAAAAG |
| 2867 | Left | CTTTTGTTTTCTTCCCTTTAG |
| 2868 | Right | CATCCCCTTAAATTGGATTAAAAAG |
| 2869 | Left | CAGGGCTTTTGTTTTCTTCC |
| 2870 | Right | CCCCTTAAATTGGATTAAAAAGAAAT |
| | | ATAC |
| 2871 | Left | CTTTTGTTTTCTTCCCTTTAG |
| 2872 | Right | ACATCCCCTTAAATTGGATTAAAAAG |
| 2873 | Left | CAGGGCTTTTGTTTTCTTCCCTTTAG |
| 2874 | Right | CCCCTTAAATTGGATTAAAAAG |
| 2875 | Left | CAGGGCTTTTGTTTTCTTCC |
| 2876 | Right | CCCCTTAAATTGGATTAAAAAG |
| 2877 | Left | GGCTTTTGTTTTCTTCCCTTTAG |
| 2878 | Right | CATCCCCTTAAATTGGATTA |
| 2879 | Left | GCTTTTGTTTTCTTCCCTTTAG |
| 2880 | Right | CCCCTTAAATTGGATTAAAAAG |

KIT Exon9 251-300 bases

| | | |
|---|---|---|
| 2881 | Left | CCACATCCCAAGTGTTTTATGTATT |
| 2882 | Right | CCCCTTAAATTGGATTAAAAAGAAA |
| 2883 | Left | CCACATCCCAAGTGTTTTATGTATT |
| 2884 | Right | CATCCCCTTAAATTGGATTAAAAAG |
| 2885 | Left | CCACATCCCAAGTGTTTTATGTATT |
| 2886 | Right | TCCCCTTAAATTGGATTAAAAAGAA |
| 2887 | Left | CCACATCCCAAGTGTTTTATGTATT |
| 2888 | Right | ATCCCCTTAAATTGGATTAAAAAGA |
| 2889 | Left | CCACATCCCAAGTGTTTTATGTATT |
| 2890 | Right | CCCCTTAAATTGGATTAAAAAGAAAT |
| 2891 | Left | CCACATCCCAAGTGTTTTATGTATT |
| 2892 | Right | ATCCCCTTAAATTGGATTAAAAAGAA |
| 2893 | Left | CCACATCCCAAGTGTTTTATGTAT |
| 2894 | Right | CCCCTTAAATTGGATTAAAAAGAAA |
| 2895 | Left | CCACATCCCAAGTGTTTTATGTATT |
| 2896 | Right | TCCCCTTAAATTGGATTAAAAAGA |
| 2897 | Left | CCACATCCCAAGTGTTTTATGTAT |
| 2898 | Right | CATCCCCTTAAATTGGATTAAAAAG |
| 2899 | Left | CCACATCCCAAGTGTTTTATGTAT |
| 2900 | Right | TCCCCTTAAATTGGATTAAAAAGAA |
| 2901 | Left | GCCACATCCCAAGTGTTTTATGTAT |
| 2902 | Right | CCCCTTAAATTGGATTAAAAAGAAA |
| 2903 | Left | CCACATCCCAAGTGTTTTATGTAT |
| 2904 | Right | ATCCCCTTAAATTGGATTAAAAAGA |
| 2905 | Left | CCACATCCCAAGTGTTTTATGTATT |
| 2906 | Right | CCCCTTAAATTGGATTAAAAAGAA |
| 2907 | Left | GGCTTTTGTTTTCTTCCCTTTAG |
| 2908 | Right | TATGGTAGACAGAGCCTAAACATCC |
| 2909 | Left | CTAGAGTAAGCCAGGGCTTTTGTTT |
| 2910 | Right | TATGGTAGACAGAGCCTAAACATCC |
| 2911 | Left | CTAGAGTAAGCCAGGGCTTTTGTT |
| 2912 | Right | TATGGTAGACAGAGCCTAAACATCC |
| 2913 | Left | CTAGAGTAAGCCAGGGCTTTTGTTT |
| 2914 | Right | AACATCCCCTTAAATTGGATTAAAA |
| 2915 | Left | CTAGAGTAAGCCAGGGCTTTTGTT |
| 2916 | Right | AACATCCCCTTAAATTGGATTAAAA |
| 2917 | Left | CCACATCCCAAGTGTTTTATGTATT |
| 2918 | Right | TCCCCTTAAATTGGATTAAAAAGAAA |
| 2919 | Left | GGCTTTTGTTTTCTTCCCTTTAG |
| 2920 | Right | TCATGACTGATATGGTAGACAGAGC |

KIT Exon9 301-400 bases

| | | |
|---|---|---|
| 2921 | Left | CTCACTAGGTCACCAAAGTGCTTAT |
| 2922 | Right | CCCCTTAAATTGGATTAAAAAGAAA |
| 2923 | Left | CTCACTAGGTCACCAAAGTGCTTAT |
| 2924 | Right | AACATCCCCTTAAATTGGATTAAAA |
| 2925 | Left | CCACATCCCAAGTGTTTTATGTATT |
| 2926 | Right | TATGGTAGACAGAGCCTAAACATCC |
| 2927 | Left | CTCACTAGGTCACCAAAGTGCTTAT |
| 2928 | Right | CATCCCCTTAAATTGGATTAAAAAG |
| 2929 | Left | CCACATCCCAAGTGTTTTATGTATT |
| 2930 | Right | GTGATGCATGTATTACCAGAAATGA |
| 2931 | Left | CCACATCCCAAGTGTTTTATGTATT |
| 2932 | Right | AACATCCCCTTAAATTGGATTAAAA |
| 2933 | Left | CACTAGGTCACCAAAGTGCTTATTC |
| 2934 | Right | CCCCTTAAATTGGATTAAAAAGAAA |
| 2935 | Left | TCACTAGGTCACCAAAGTGCTTATT |
| 2936 | Right | CCCCTTAAATTGGATTAAAAAGAAA |
| 2937 | Left | CTCACTAGGTCACCAAAGTGCTTAT |
| 2938 | Right | TCCCCTTAAATTGGATTAAAAAGAA |
| 2939 | Left | TTTGTTTTAAAAGTATGCCACATCC |
| 2940 | Right | CCCCTTAAATTGGATTAAAAAGAAA |
| 2941 | Left | TTTGTTTTAAAAGTATGCCACATCC |
| 2942 | Right | TATGGTAGACAGAGCCTAAACATCC |
| 2943 | Left | CTCACTAGGTCACCAAAGTGCTTAT |
| 2944 | Right | ATCCCCTTAAATTGGATTAAAAAGA |
| 2945 | Left | CCACATCCCAAGTGTTTTATGTATT |
| 2946 | Right | TCATGACTGATATGGTAGACAGAGC |
| 2947 | Left | CACTAGGTCACCAAAGTGCTTATTC |
| 2948 | Right | AACATCCCCTTAAATTGGATTAAAA |
| 2949 | Left | TCACTAGGTCACCAAAGTGCTTATT |
| 2950 | Right | AACATCCCCTTAAATTGGATTAAAA |
| 2951 | Left | TTTGTTTTAAAAGTATGCCACATCC |
| 2952 | Right | GTGATGCATGTATTACCAGAAATGA |
| 2953 | Left | TCACCAAAGTGCTTATTCTTAGACA |
| 2954 | Right | CCCCTTAAATTGGATTAAAAAGAAA |
| 2955 | Left | ACTCACTAGGTCACCAAAGTGCTTA |
| 2956 | Right | CCCCTTAAATTGGATTAAAAAGAAA |
| 2957 | Left | TTTGTTTTAAAAGTATGCCACATCC |
| 2958 | Right | AACATCCCCTTAAATTGGATTAAAA |
| 2959 | Left | AGGTCACCAAAGTGCTTATTCTTAG |
| 2960 | Right | CCCCTTAAATTGGATTAAAAAGAAA |

KIT Exon9 401-500 bases

| | | |
|---|---|---|
| 2961 | Left | CTCACTAGGTCACCAAAGTGCTTAT |
| 2962 | Right | TATGGTAGACAGAGCCTAAACATCC |
| 2963 | Left | TACAGTCGTAGAAACTCAGTGTTGG |
| 2964 | Right | CCCCTTAAATTGGATTAAAAAGAAA |
| 2965 | Left | CTCACTAGGTCACCAAAGTGCTTAT |
| 2966 | Right | GTGATGCATGTATTACCAGAAATGA |
| 2967 | Left | CTCACTAGGTCACCAAAGTGCTTAT |
| 2968 | Right | TCATGACTGATATGGTAGACAGAGC |
| 2969 | Left | TACAGTCGTAGAAACTCAGTGTTGG |
| 2970 | Right | CATCCCCTTAAATTGGATTAAAAAG |
| 2971 | Left | CACTAGGTCACCAAAGTGCTTATTC |
| 2972 | Right | TATGGTAGACAGAGCCTAAACATCC |
| 2973 | Left | TCACTAGGTCACCAAAGTGCTTATT |
| 2974 | Right | TATGGTAGACAGAGCCTAAACATCC |
| 2975 | Left | ACAGTCGTAGAAACTCAGTGTTGGT |
| 2976 | Right | CCCCTTAAATTGGATTAAAAAGAAA |
| 2977 | Left | TACAGTCGTAGAAACTCAGTGTTGG |
| 2978 | Right | TCCCCTTAAATTGGATTAAAAAGAA |
| 2979 | Left | CTCACTAGGTCACCAAAGTGCTTAT |
| 2980 | Right | GGTCAATGTTGGAATGAACTTAAAA |
| 2981 | Left | CACTAGGTCACCAAAGTGCTTATTC |
| 2982 | Right | GTGATGCATGTATTACCAGAAATGA |
| 2983 | Left | TCACTAGGTCACCAAAGTGCTTATT |
| 2984 | Right | GTGATGCATGTATTACCAGAAATGA |
| 2985 | Left | TACAGTCGTAGAAACTCAGTGTTGG |
| 2986 | Right | ATCCCCTTAAATTGGATTAAAAAGA |
| 2987 | Left | TCACCAAAGTGCTTATTCTTAGACA |
| 2988 | Right | TATGGTAGACAGAGCCTAAACATCC |
| 2989 | Left | ACTCACTAGGTCACCAAAGTGCTTA |
| 2990 | Right | TATGGTAGACAGAGCCTAAACATCC |
| 2991 | Left | ACAGTCGTAGAAACTCAGTGTTGGT |
| 2992 | Right | AACATCCCCTTAAATTGGATTAAAA |
| 2993 | Left | CTAGGTCACCAAAGTGCTTATTCTT |
| 2994 | Right | TATGGTAGACAGAGCCTAAACATCC |
| 2995 | Left | AGGTCACCAAAGTGCTTATTCTTAG |
| 2996 | Right | TATGGTAGACAGAGCCTAAACATCC |
| 2997 | Left | TCACCAAAGTGCTTATTCTTAGACA |
| 2998 | Right | GTGATGCATGTATTACCAGAAATGA |
| 2999 | Left | CACTAGGTCACCAAAGTGCTTATTC |
| 3000 | Right | TCATGACTGATATGGTAGACAGAGC |

KIT Exon9 501-600 bases

| | | |
|---|---|---|
| 3001 | Left | CCTCTATGCTATTTCTTTTCAACCA |
| 3002 | Right | TATGGTAGACAGAGCCTAAACATCC |
| 3003 | Left | CTCACTAGGTCACCAAAGTGCTTAT |
| 3004 | Right | AGGCAGTGTTAACTTTTGGATACAG |
| 3005 | Left | TTTTATGCTTTCCTCCTCTATGCTA |
| 3006 | Right | CCCCTTAAATTGGATTAAAAAGAAA |
| 3007 | Left | CTCACTAGGTCACCAAAGTGCTTAT |
| 3008 | Right | GGCAGTGTTAACTTTTGGATACAGT |
| 3009 | Left | GCTTTCCTCCTCTATGCTATTTCTT |
| 3010 | Right | TATGGTAGACAGAGCCTAAACATCC |
| 3011 | Left | TTTTATGCTTTCCTCCTCTATGCTA |
| 3012 | Right | TATGGTAGACAGAGCCTAAACATCC |
| 3013 | Left | CCACATCCCAAGTGTTTTATGTATT |
| 3014 | Right | AGGCAGTGTTAACTTTTGGATACAG |
| 3015 | Left | TCCTCCTCTATGCTATTTCTTTTCA |
| 3016 | Right | TATGGTAGACAGAGCCTAAACATCC |
| 3017 | Left | ATTTTATTGAATTCCTTTCCAATCC |
| 3018 | Right | GTGATGCATGTATTACCAGAAATGA |
| 3019 | Left | CTCACTAGGTCACCAAAGTGCTTAT |
| 3020 | Right | GTAAATATATTCCCCCATTTGCTTT |
| 3021 | Left | GCTTTCCTCCTCTATGCTATTTCTT |
| 3022 | Right | AACATCCCCTTAAATTGGATTAAAA |
| 3023 | Left | TTTTATGCTTTCCTCCTCTATGCTA |
| 3024 | Right | AACATCCCCTTAAATTGGATTAAAA |
| 3025 | Left | CCACATCCCAAGTGTTTTATGTATT |
| 3026 | Right | GGCAGTGTTAACTTTTGGATACAGT |
| 3027 | Left | TTTAGTAGAGACGAGGTTTCACCAT |
| 3028 | Right | CCCCTTAAATTGGATTAAAAAGAAA |
| 3029 | Left | GCTGAGATTACAGGTGTGAGCTACT |
| 3030 | Right | CCCCTTAAATTGGATTAAAAAGAAA |
| 3031 | Left | CTCACTAGGTCACCAAAGTGCTTAT |
| 3032 | Right | GATTGTTCTAATTCTGTTTGGGTGT |
| 3033 | Left | TACAGTCGTAGAAACTCAGTGTTGG |
| 3034 | Right | GTAAATATATTCCCCCATTTGCTTT |
| 3035 | Left | GCTGAGATTACAGGTGTGAGCTACT |
| 3036 | Right | TATGGTAGACAGAGCCTAAACATCC |
| 3037 | Left | CACTAGGTCACCAAAGTGCTTATTC |
| 3038 | Right | AGGCAGTGTTAACTTTTGGATACAG |
| 3039 | Left | TCACTAGGTCACCAAAGTGCTTATT |
| 3040 | Right | AGGCAGTGTTAACTTTTGGATACAG |

KIT Exon9 801-1000 bases

| | | |
|---|---|---|
| 3041 | Left | ATTCCTTTCCAATCCTTTCAGTAAC |
| 3042 | Right | AGGCAGTGTTAACTTTTGGATACAG |
| 3043 | Left | ATTTTATTGAATTCCTTTCCAATCC |
| 3044 | Right | AGGCAGTGTTAACTTTTGGATACAG |
| 3045 | Left | TACAGTCGTAGAAACTCAGTGTTGG |
| 3046 | Right | AGGCAGTGTTAACTTTTGGATACAG |
| 3047 | Left | TACATCCTTGATTTTGTTGTTGTTG |
| 3048 | Right | CCCCTTAAATTGGATTAAAAAGAAA |
| 3049 | Left | ATTCCTTTCCAATCCTTTCAGTAAC |
| 3050 | Right | GGCAGTGTTAACTTTTGGATACAGT |
| 3051 | Left | CCTCTATGCTATTTCTTTTCAACCA |
| 3052 | Right | GTAAATATATTCCCCCATTTGCTTT |
| 3053 | Left | ATTTTATTGAATTCCTTTCCAATCC |
| 3054 | Right | GGCAGTGTTAACTTTTGGATACAGT |
| 3055 | Left | TTCTGGTCTACATCCTTGATTTTGT |
| 3056 | Right | CCCCTTAAATTGGATTAAAAAGAAA |
| 3057 | Left | TACAGTCGTAGAAACTCAGTGTTGG |
| 3058 | Right | GGCAGTGTTAACTTTTGGATACAGT |
| 3059 | Left | CCTCTATGCTATTTCTTTTCAACCA |
| 3060 | Right | GATTGTTCTAATTCTGTTTGGGTGT |
| 3061 | Left | CCCTGTTTTACAGTCGTAGAAACTC |
| 3062 | Right | AGGCAGTGTTAACTTTTGGATACAG |
| 3063 | Left | ATTCCTTTCCAATCCTTTCAGTAAC |
| 3064 | Right | GTAAATATATTCCCCCATTTGCTTT |
| 3065 | Left | TACATCCTTGATTTTGTTGTTGTTG |
| 3066 | Right | AACATCCCCTTAAATTGGATTAAAA |
| 3067 | Left | AACCCTCTGCAATGGGTATTACTAT |
| 3068 | Right | AGGCAGTGTTAACTTTTGGATACAG |
| 3069 | Left | TTATTGAATTCCTTTCCAATCCTTT |
| 3070 | Right | AGGCAGTGTTAACTTTTGGATACAG |
| 3071 | Left | TTTTATTGAATTCCTTTCCAATCCT |
| 3072 | Right | AGGCAGTGTTAACTTTTGGATACAG |
| 3073 | Left | TTTATTGAATTCCTTTCCAATCCTT |
| 3074 | Right | AGGCAGTGTTAACTTTTGGATACAG |
| 3075 | Left | ATTTTATTGAATTCCTTTCCAATCC |
| 3076 | Right | GTAAATATATTCCCCCATTTGCTTT |
| 3077 | Left | CCTCTATGCTATTTCTTTTCAACCA |
| 3078 | Right | TGCTTTCTCTAGCTCTTTTTAATGG |
| 3079 | Left | CCACATCCCAAGTGTTTTATGTATT |
| 3080 | Right | ACTACTCAAAACCTGAGAAAACACG |

KIT Exon9 2 kb

| | | |
|---|---|---|
| 3081 | Left | TGTAGGTGTTAAGAAAGGAAACAGG |
| 3082 | Right | GCTTCCTTTATGGACGGTTTATATT |
| 3083 | Left | TCCTCACTTTGACTATTTTCTGGTC |
| 3084 | Right | GCTTCCTTTATGGACGGTTTATATT |
| 3085 | Left | GTTAAGAAAGGAAACAGGGAATAGC |
| 3086 | Right | GCTTCCTTTATGGACGGTTTATATT |
| 3087 | Left | TGTAGGTGTTAAGAAAGGAAACAGG |
| 3088 | Right | ACTACTCAAAACCTGAGAAAACACG |
| 3089 | Left | AAGTAATGATCGCACAATTACAACA |
| 3090 | Right | CCCCTTAAATTGGATTAAAAAGAAA |
| 3091 | Left | CCAAGTTACCAAAAAGTAATGATCG |
| 3092 | Right | CCCCTTAAATTGGATTAAAAAGAAA |
| 3093 | Left | TGGATAAGCTTGTTCTAGTGGGTAG |
| 3094 | Right | ACTACTCAAAACCTGAGAAAACACG |
| 3095 | Left | TGTAGGTGTTAAGAAAGGAAACAGG |
| 3096 | Right | CCTCACTACTCAAAACCTGAGAAAA |
| 3097 | Left | ATAACTAGGCCTTCCTGCTTAGAAC |
| 3098 | Right | GCTTCCTTTATGGACGGTTTATATT |
| 3099 | Left | GTTAAGAAAGGAAACAGGGAATAGC |
| 3100 | Right | CCTCACTACTCAAAACCTGAGAAAA |
| 3101 | Left | TGGATAAGCTTGTTCTAGTGGGTAG |
| 3102 | Right | CCTCACTACTCAAAACCTGAGAAAA |
| 3103 | Left | TCTGGTCTACATCCTTGATTTTGTT |
| 3104 | Right | GCTTCCTTTATGGACGGTTTATATT |
| 3105 | Left | TTCTGGTCTACATCCTTGATTTTGT |
| 3106 | Right | GCTTCCTTTATGGACGGTTTATATT |
| 3107 | Left | GGTCTACATCCTTGATTTTGTTGTT |
| 3108 | Right | GCTTCCTTTATGGACGGTTTATATT |
| 3109 | Left | GGAATAAGCCTCTTTATCACAACAA |
| 3110 | Right | GTAAATATATTCCCCCATTTGCTTT |
| 3111 | Left | TGTAGGTGTTAAGAAAGGAAACAGG |
| 3112 | Right | TCTTTAAGCTTTCCTGTATTTTCCA |
| 3113 | Left | AAGTAATGATCGCACAATTACAACA |
| 3114 | Right | AACATCCCCTTAAATTGGATTAAAA |
| 3115 | Left | TAACTAGGCCTTCCTGCTTAGAACT |
| 3116 | Right | GCTTCCTTTATGGACGGTTTATATT |
| 3117 | Left | TTCATGGAATAAGCCTCTTTATCAC |
| 3118 | Right | GTAAATATATTCCCCCATTTGCTTT |
| 3119 | Left | TTCTTTTGGGGAATACTAAGTAGGG |
| 3120 | Right | GTAAATATATTCCCCCATTTGCTTT |

KIT Exon9-10 2 kb

| | | |
|---|---|---|
| 3121 | Left | CCTCTATGCTATTTCTTTTCAACCA |
| 3122 | Right | ATTAGAGCACTCTGGAGAGAGAACA |
| 3123 | Left | ATTCCTTTCCAATCCTTTCAGTAAC |
| 3124 | Right | AGCACTCTGGAGAGAGAACAAATAA |
| 3125 | Left | ATTCCTTTCCAATCCTTTCAGTAAC |
| 3126 | Right | ATTAGAGCACTCTGGAGAGAGAACA |
| 3127 | Left | ATTTTATTGAATTCCTTTCCAATCC |
| 3128 | Right | ATTAGAGCACTCTGGAGAGAGAACA |
| 3129 | Left | GCTTTCCTCCTCTATGCTATTTCTT |
| 3130 | Right | ATTAGAGCACTCTGGAGAGAGAACA |
| 3131 | Left | AACCCTCTGCAATGGGTATTACTAT |
| 3132 | Right | AGCACTCTGGAGAGAGAACAAATAA |
| 3133 | Left | GCTGAGATTACAGGTGTGAGCTACT |
| 3134 | Right | AGCACTCTGGAGAGAGAACAAATAA |
| 3135 | Left | ATTCCTTTCCAATCCTTTCAGTAAC |
| 3136 | Right | GCACTCTGGAGAGAGAACAAATAAA |
| 3137 | Left | ATTCCTTTCCAATCCTTTCAGTAAC |
| 3138 | Right | CTCTGGAGAGAGAACAAATAAATGG |
| 3139 | Left | AACCCTCTGCAATGGGTATTACTAT |
| 3140 | Right | ATTAGAGCACTCTGGAGAGAGAACA |
| 3141 | Left | GCTGAGATTACAGGTGTGAGCTACT |
| 3142 | Right | ATTAGAGCACTCTGGAGAGAGAACA |
| 3143 | Left | ATTTTATTGAATTCCTTTCCAATCC |
| 3144 | Right | CTCTGGAGAGAGAACAAATAAATGG |
| 3145 | Left | TTATTGAATTCCTTTCCAATCCTTT |
| 3146 | Right | ATTAGAGCACTCTGGAGAGAGAACA |
| 3147 | Left | TTTATTGAATTCCTTTCCAATCCTT |
| 3148 | Right | ATTAGAGCACTCTGGAGAGAGAACA |
| 3149 | Left | TTTTATTGAATTCCTTTCCAATCCT |
| 3150 | Right | ATTAGAGCACTCTGGAGAGAGAACA |
| 3151 | Left | ATGCTTTCCTCCTCTATGCTATTTC |
| 3152 | Right | AGCACTCTGGAGAGAGAACAAATAA |
| 3153 | Left | TTTTATGCTTTCCTCCTCTATGCTA |
| 3154 | Right | CTCTGGAGAGAGAACAAATAAATGG |
| 3155 | Left | ATGCTTTCCTCCTCTATGCTATTTC |
| 3156 | Right | ATTAGAGCACTCTGGAGAGAGAACA |
| 3157 | Left | ATTCCTTTCCAATCCTTTCAGTAAC |
| 3158 | Right | GAGCACTCTGGAGAGAGAACAAATA |
| 3159 | Left | ATTCCTTTCCAATCCTTTCAGTAAC |
| 3160 | Right | TCTGGAGAGAGAACAAATAAATGGT |

KIT Exon9-11 2 kb

| | | |
|---|---|---|
| 3161 | Left | CCACATCCCAAGTGTTTTATGTATT |
| 3162 | Right | TTCTCTATGGCAAACCTATCAAAAG |
| 3163 | Left | CCACATCCCAAGTGTTTTATGTATT |
| 3164 | Right | GTTCTCTATGGCAAACCTATCAAAA |
| 3165 | Left | CCACATCCCAAGTGTTTTATGTATT |
| 3166 | Right | ATGTTGTCCAGAGACATTTTCCTAC |
| 3167 | Left | CACTAGGTCACCAAAGTGCTTATTC |
| 3168 | Right | TTCTCTATGGCAAACCTATCAAAAG |
| 3169 | Left | CCACATCCCAAGTGTTTTATGTATT |
| 3170 | Right | CATTTTCCTACGATGTTCTCTATGG |
| 3171 | Left | CCACATCCCAAGTGTTTTATGTATT |
| 3172 | Right | ATGTTCTCTATGGCAAACCTATCAA |
| 3173 | Left | CCACATCCCAAGTGTTTTATGTATT |
| 3174 | Right | AATGTTGTCCAGAGACATTTTCCTA |
| 3175 | Left | CCACATCCCAAGTGTTTTATGTATT |
| 3176 | Right | AGGAATTAAAAACAATGTTGTCCAG |
| 3177 | Left | TCACCAAAGTGCTTATTCTTAGACA |
| 3178 | Right | TTCTCTATGGCAAACCTATCAAAAG |
| 3179 | Left | TTTGTTTTAAAAGTATGCCACATCC |
| 3180 | Right | ATGTTGTCCAGAGACATTTTCCTAC |
| 3181 | Left | TCACCAAAGTGCTTATTCTTAGACA |
| 3182 | Right | GTTCTCTATGGCAAACCTATCAAAA |
| 3183 | Left | CTAGGTCACCAAAGTGCTTATTCTT |
| 3184 | Right | TTCTCTATGGCAAACCTATCAAAAG |
| 3185 | Left | TTTGTTTTAAAAGTATGCCACATCC |
| 3186 | Right | CATTTTCCTACGATGTTCTCTATGG |
| 3187 | Left | CACATCCCAAGTGTTTTATGTATT |
| 3188 | Right | GGAATTAAAAACAATGTTGTCCAGA |
| 3189 | Left | TTTGTTTTAAAAGTATGCCACATCC |
| 3190 | Right | AATGTTGTCCAGAGACATTTTCCTA |
| 3191 | Left | CTAGGTCACCAAAGTGCTTATTCTT |
| 3192 | Right | GTTCTCTATGGCAAACCTATCAAAA |
| 3193 | Left | AGGTCACCAAAGTGCTTATTCTTAG |
| 3194 | Right | GTTCTCTATGGCAAACCTATCAAAA |
| 3195 | Left | TTTGTTTTAAAAGTATGCCACATCC |
| 3196 | Right | AGGAATTAAAAACAATGTTGTCCAG |
| 3197 | Left | TTTGTTTTAAAAGTATGCCACATCC |
| 3198 | Right | TTGTGCAGTTTCAAAATCAATAAAG |
| 3199 | Left | TCACCAAAGTGCTTATTCTTAGACA |
| 3200 | Right | ATGTTCTCTATGGCAAACCTATCAA |

KIT Exon10 130-150 bases

| | | |
|---|---|---|
| 3201 | Left | TCCACATTTCTCTTCCATTGTA |
| 3202 | Right | GAGAACAAATAAATGGTTAC |
| 3203 | Left | CCACATTTCTCTTCCATTGTA |
| 3204 | Right | AGAGAACAAATAAATGGTTAC |
| 3205 | Left | TCCACATTTCTCTTCCATTGT |
| 3206 | Right | GAGAACAAATAAATGGTTAC |
| 3207 | Left | CCACATTTCTCTTCCATTGT |
| 3208 | Right | AGAGAACAAATAAATGGTTAC |
| 3209 | Left | TCCACATTTCTCTTCCATTGTA |
| 3210 | Right | GAGAACAAATAAATGGTTA |
| 3211 | Left | CACATTTCTCTTCCATTGTA |
| 3212 | Right | GAGAGAACAAATAAATGGTTAC |
| 3213 | Left | CCACATTTCTCTTCCATTGTA |
| 3214 | Right | AGAGAACAAATAAATGGTTA |
| 3215 | Left | CCACATTTCTCTTCCATTGTA |
| 3216 | Right | GAGAACAAATAAATGGTTAC |
| 3217 | Left | TCCACATTTCTCTTCCATTG |
| 3218 | Right | GAGAACAAATAAATGGTTAC |
| 3219 | Left | TCCACATTTCTCTTCCATTGT |
| 3220 | Right | GAGAACAAATAAATGGTTA |
| 3221 | Left | CCACATTTCTCTTCCATTG |
| 3222 | Right | AGAGAACAAATAAATGGTTAC |
| 3223 | Left | CACATTTCTCTTCCATTGT |
| 3224 | Right | GAGAGAACAAATAAATGGTTAC |
| 3225 | Left | CCACATTTCTCTTCCATTGT |
| 3226 | Right | AGAGAACAAATAAATGGTTA |
| 3227 | Left | CCACATTTCTCTTCCATTGT |
| 3228 | Right | GAGAACAAATAAATGGTTAC |
| 3229 | Left | CATTTCTCTTCCATTGTA |
| 3230 | Right | GAGAGAGAACAAATAAATGGTTAC |
| 3231 | Left | ACATTTCTCTTCCATTGTA |
| 3232 | Right | AGAGAGAACAAATAAATGGTTAC |
| 3233 | Left | CACATTTCTCTTCCATTGTA |
| 3234 | Right | GAGAGAACAAATAAATGGTTA |
| 3235 | Left | CCACATTTCTCTTCCATTGTA |
| 3236 | Right | GAGAACAAATAAATGGTTA |
| 3237 | Left | CACATTTCTCTTCCATTGTA |
| 3238 | Right | AGAGAACAAATAAATGGTTAC |
| 2239 | Left | TCCACATTTCTCTTCCATTG |
| 2240 | Right | GAGAACAAATAAATGGTTA |

KIT Exon10 151-200 bases

| | | |
|---|---|---|
| 3241 | Left | AGTTTGTGATTCCACATTTCTCTTC |
| 3242 | Right | AGCACTCTGGAGAGAGAACAAATAA |
| 3243 | Left | AGTTTGTGATTCCACATTTCTCTTC |
| 3244 | Right | GCACTCTGGAGAGAGAACAAATAAA |
| 3245 | Left | AGTTTGTGATTCCACATTTCTCTTC |
| 3246 | Right | GAGCACTCTGGAGAGAGAACAAATA |
| 3247 | Left | AGTTTGTGATTCCACATTTCTCTTC |
| 3248 | Right | TCTGGAGAGAGAACAAATAAATGGT |
| 3249 | Left | AAGTTTGTGATTCCACATTTCTCTT |
| 3250 | Right | AGCACTCTGGAGAGAGAACAAATAA |
| 3251 | Left | AAAGTTTGTGATTCCACATTTCTCT |
| 3252 | Right | AGCACTCTGGAGAGAGAACAAATAA |
| 3253 | Left | GATTCCACATTTCTCTTCCATTGTA |
| 3254 | Right | AGCACTCTGGAGAGAGAACAAATAA |
| 3255 | Left | AAGTTTGTGATTCCACATTTCTCTT |
| 3256 | Right | GCACTCTGGAGAGAGAACAAATAAA |
| 3257 | Left | AAAGTTTGTGATTCCACATTTCTCT |
| 3258 | Right | GCACTCTGGAGAGAGAACAAATAAA |
| 3259 | Left | AAGTTTGTGATTCCACATTTCTCTT |
| 3260 | Right | GAGCACTCTGGAGAGAGAACAAATA |
| 3261 | Left | AAAGTTTGTGATTCCACATTTCTCT |
| 3262 | Right | GAGCACTCTGGAGAGAGAACAAATA |
| 3263 | Left | CAAAGTTTGTGATTCCACATTTCTC |
| 3264 | Right | AGCACTCTGGAGAGAGAACAAATAA |
| 3265 | Left | CAAAGTTTGTGATTCCACATTTCTC |
| 3266 | Right | GCACTCTGGAGAGAGAACAAATAAA |
| 3267 | Left | CAAAGTTTGTGATTCCACATTTCTC |
| 3268 | Right | CTCTGGAGAGAGAACAAATAAATGG |
| 3269 | Left | AGTTTGTGATTCCACATTTCTCTTC |
| 3270 | Right | CTCTGGAGAGAGAACAAATAAATGGT |
| 3271 | Left | CAAAGTTTGTGATTCCACATTTCTC |
| 3272 | Right | GAGCACTCTGGAGAGAGAACAAATA |
| 3273 | Left | CAAAGTTTGTGATTCCACATTTCTC |
| 3274 | Right | TCTGGAGAGAGAACAAATAAATGGT |
| 3275 | Left | CAAAGTTTGTGATTCCACATTTCT |
| 3276 | Right | AGCACTCTGGAGAGAGAACAAATAA |
| 3277 | Left | GTGATTCCACATTTCTCTTCCATT |
| 3278 | Right | AGCACTCTGGAGAGAGAACAAATAA |
| 3279 | Left | CAAAGTTTGTGATTCCACATTTCT |
| 3280 | Right | ATTAGAGCACTCTGGAGAGAGAACA |

KIT Exon10 201-300 bases

| | | |
|---|---|---|
| 3281 | Left | GTACAATGTAACCAAGGTGAAGCTC |
| 3282 | Right | AGCACTCTGGAGAGAGAACAAATAA |
| 3283 | Left | GAGTACAATGTAACCAAGGTGAAGC |
| 3284 | Right | AGCACTCTGGAGAGAGAACAAATAA |
| 3285 | Left | TACAATGTAACCAAGGTGAAGCTCT |
| 3286 | Right | AGCACTCTGGAGAGAGAACAAATAA |
| 3287 | Left | TACAATGTAACCAAGGTGAAGCTCT |
| 3288 | Right | ATTAGAGCACTCTGGAGAGAGAACA |
| 3289 | Left | GTACAATGTAACCAAGGTGAAGCTC |
| 3290 | Right | GCACTCTGGAGAGAGAACAAATAAA |
| 3291 | Left | GAGTACAATGTAACCAAGGTGAAGC |
| 3292 | Right | GCACTCTGGAGAGAGAACAAATAAA |
| 3293 | Left | GTACAATGTAACCAAGGTGAAGCTC |
| 3294 | Right | CTCTGGAGAGAGAACAAATAAATGG |
| 3295 | Left | GAGTACAATGTAACCAAGGTGAAGC |
| 3296 | Right | CTCTGGAGAGAGAACAAATAAATGG |
| 3297 | Left | TACAATGTAACCAAGGTGAAGCTCT |
| 3298 | Right | GCACTCTGGAGAGAGAACAAATAAA |
| 3299 | Left | TACAATGTAACCAAGGTGAAGCTCT |
| 3300 | Right | CTCTGGAGAGAGAACAAATAAATGG |
| 3301 | Left | GTACAATGTAACCAAGGTGAAGCTC |
| 3302 | Right | GAGCACTCTGGAGAGAGAACAAATA |
| 3303 | Left | GAGTACAATGTAACCAAGGTGAAGC |
| 3304 | Right | GAGCACTCTGGAGAGAGAACAAATA |
| 3305 | Left | GTACAATGTAACCAAGGTGAAGCTC |
| 3306 | Right | TCTGGAGAGAGAACAAATAAATGGT |
| 3307 | Left | GAGTACAATGTAACCAAGGTGAAGC |
| 3308 | Right | TCTGGAGAGAGAACAAATAAATGGT |
| 3309 | Left | TACAATGTAACCAAGGTGAAGCTCT |
| 3310 | Right | GAGCACTCTGGAGAGAGAACAAATA |
| 3311 | Left | TACAATGTAACCAAGGTGAAGCTCT |
| 3312 | Right | TCTGGAGAGAGAACAAATAAATGGT |
| 3313 | Left | CTCTGAGACTCACATAGCTTTGCAT |
| 3314 | Right | AGCACTCTGGAGAGAGAACAAATAA |
| 3315 | Left | GTACAATGTAACCAAGGTGAAGCTC |
| 3316 | Right | TAGAGCACTCTGGAGAGAGAACAAA |
| 3317 | Left | CTCTGAGACTCACATAGCTTTGCAT |
| 3318 | Right | ATTAGAGCACTCTGGAGAGAGAACA |
| 3319 | Left | TACAATGTAACCAAGGTGAAGCTC |
| 3320 | Right | AGCACTCTGGAGAGAGAACAAATAA |

KIT Exon10 301-400 bases

| | | |
|---|---|---|
| 3321 | Left | TCTATTCTGCAGTATTGTGGTTTCA |
| 3322 | Right | AGCACTCTGGAGAGAGAACAAATAA |
| 3323 | Left | TCTGCAGTATTGTGGTTTCAAGTTA |
| 3324 | Right | AGCACTCTGGAGAGAGAACAAATAA |
| 3325 | Left | TCTATTCTGCAGTATTGTGGTTTCA |
| 3326 | Right | ATTAGAGCACTCTGGAGAGAGAACA |
| 3327 | Left | TCTGCAGTATTGTGGTTTCAAGTTA |
| 3328 | Right | ATTAGAGCACTCTGGAGAGAGAACA |
| 3329 | Left | TCTATTCTGCAGTATTGTGGTTTCA |
| 3330 | Right | GCACTCTGGAGAGAGAACAAATAAA |
| 3331 | Left | ATTCTGCAGTATTGTGGTTTCAAGT |
| 3332 | Right | AGCACTCTGGAGAGAGAACAAATAA |
| 3333 | Left | TCTATTCTGCAGTATTGTGGTTTCA |
| 3334 | Right | CTCTGGAGAGAGAACAAATAAATGG |
| 3335 | Left | TCTGCAGTATTGTGGTTTCAAGTTA |
| 3336 | Right | GCACTCTGGAGAGAGAACAAATAAA |
| 3337 | Left | TATTCTGCAGTATTGTGGTTTCAAG |
| 3338 | Right | AGCACTCTGGAGAGAGAACAAATAA |
| 3339 | Left | CTATTCTGCAGTATTGTGGTTTCAA |
| 3340 | Right | AGCACTCTGGAGAGAGAACAAATAA |
| 3341 | Left | TCTGCAGTATTGTGGTTTCAAGTTA |
| 3342 | Right | CTCTGGAGAGAGAACAAATAAATGG |
| 3343 | Left | GAGTACAATGTAACCAAGGTGAAGC |
| 3344 | Right | ATTAGAGCACTCTGGAGAGAGAACA |
| 3345 | Left | GTACAATGTAACCAAGGTGAAGCTC |
| 3346 | Right | ATTAGAGCACTCTGGAGAGAGAACA |
| 3347 | Left | ATTCTGCAGTATTGTGGTTTCAAGT |
| 3348 | Right | ATTAGAGCACTCTGGAGAGAGAACA |
| 3349 | Left | TATTCTGCAGTATTGTGGTTTCAAG |
| 3350 | Right | ATTAGAGCACTCTGGAGAGAGAACA |
| 3351 | Left | CTATTCTGCAGTATTGTGGTTTCAA |
| 3352 | Right | ATTAGAGCACTCTGGAGAGAGAACA |
| 3353 | Left | TCTATTCTGCAGTATTGTGGTTTCA |
| 3354 | Right | TCTGGAGAGAGAACAAATAAATGGT |
| 3355 | Left | TCTGCAGTATTGTGGTTTCAAGTTA |
| 3356 | Right | GAGCACTCTGGAGAGAGAACAAATA |
| 3357 | Left | TCTGCAGTATTGTGGTTTCAAGTTA |
| 3358 | Right | TCTGGAGAGAGAACAAATAAATGGT |
| 3359 | Left | ATTCTGCAGTATTGTGGTTTCAAGT |
| 3360 | Right | GCACTCTGGAGAGAGAACAAATAAA |

KIT Exon11 151-200 bases

| | | |
|---|---|---|
| 3361 | Left | AAGGTGATCTATTTTTCCCTTTCTC |
| 3362 | Right | GAAAGCCCCTGTTTCATACTGAC |
| 3363 | Left | AAGGTGATCTATTTTTCCCTTTCTC |
| 3364 | Right | AAAGCCCCTGTTTCATACTGAC |
| 3365 | Left | AAAGGTGATCTATTTTTCCCTTTCT |
| 3366 | Right | GAAAGCCCCTGTTTCATACTGAC |
| 3367 | Left | AAAGGTGATCTATTTTTCCCTTTCT |
| 3368 | Right | AAAGCCCCTGTTTCATACTGAC |
| 3369 | Left | AGGTGATCTATTTTTCCCTTTCTCC |
| 3370 | Right | GAAAGCCCCTGTTTCATACTGAC |
| 3371 | Left | AAGGTGATCTATTTTTCCCTTTCTC |
| 3372 | Right | ATGGAAAGCCCCTGTTTCATACT |
| 3373 | Left | GGTGATCTATTTTTCCCTTTCTCC |
| 3374 | Right | GAAAGCCCCTGTTTCATACTGAC |
| 3375 | Left | AAAGGTGATCTATTTTTCCCTTTCTC |
| 3376 | Right | GAAAGCCCCTGTTTCATACTGAC |
| 3377 | Left | AGGTGATCTATTTTTCCCTTTCTCC |
| 3378 | Right | AAAGCCCCTGTTTCATACTGAC |
| 3379 | Left | AAGGTGATCTATTTTTCCCTTTCTC |
| 3380 | Right | ATGGAAAGCCCCTGTTTCATAC |
| 3381 | Left | GGTGATCTATTTTTCCCTTTCTCC |
| 3382 | Right | AAAGCCCCTGTTTCATACTGAC |
| 3383 | Left | AAAGGTGATCTATTTTTCCCTTTCTC |
| 3384 | Right | AAAGCCCCTGTTTCATACTGAC |
| 3385 | Left | CTATTTTTCCCTTTCTCCCCACAG |
| 3386 | Right | TTATGTGTACCCAAAAAGGTGACAT |
| 3387 | Left | AAGGTGATCTATTTTTCCCTTTCTC |
| 3388 | Right | GGAAAGCCCCTGTTTCATACT |
| 3389 | Left | AAAGGTGATCTATTTTTCCCTTTCT |
| 3390 | Right | ATGGAAAGCCCCTGTTTCATACT |
| 3391 | Left | AAGGTGATCTATTTTTCCCTTTCTC |
| 3392 | Right | ATGGAAAGCCCCTGTTTCATA |
| 3393 | Left | TATTTTTCCCTTTCTCCCCACAG |
| 3394 | Right | TTATGTGTACCCAAAAAGGTGACAT |
| 3395 | Left | CTATTTTTCCCTTTCTCCCCACA |
| 3396 | Right | TTATGTGTACCCAAAAAGGTGACAT |
| 3397 | Left | AAAGGTGATCTATTTTTCCCTTTCT |
| 3398 | Right | CAAAAAGGTGACATGGAAAGC |
| 3399 | Left | AAAGGTGATCTATTTTTCCCTTTCT |
| 3400 | Right | ATGGAAAGCCCCTGTTTCATAC |

KIT Exon11 201-300 bases

| | | |
|---|---|---|
| 3401 | Left | TTATTTGTTCTCTCTCCAGAGTGCT |
| 3402 | Right | TTATGTGTACCCAAAAAGGTGACAT |
| 3403 | Left | TGTTCTCTCTCCAGAGTGCTCTAAT |
| 3404 | Right | TTATGTGTACCCAAAAAGGTGACAT |
| 3405 | Left | GTTCTCTCTCCAGAGTGCTCTAATG |
| 3406 | Right | TTATGTGTACCCAAAAAGGTGACAT |
| 3407 | Left | CAGAGTGCTCTAATGACTGAGACAA |
| 3408 | Right | TTATGTGTACCCAAAAAGGTGACAT |
| 3409 | Left | CTCTCTCCAGAGTGCTCTAATGACT |
| 3410 | Right | TTATGTGTACCCAAAAAGGTGACAT |
| 3411 | Left | TTATTTGTTCTCTCTCCAGAGTGCT |
| 3412 | Right | TGTTATGTGTACCCAAAAAGGTGAC |
| 3413 | Left | TTATTTGTTCTCTCTCCAGAGTGCT |
| 3414 | Right | GTTATGTGTACCCAAAAAGGTGACA |
| 3415 | Left | AAGGTGATCTATTTTTCCCTTTCTC |
| 3416 | Right | TTATGTGTACCCAAAAAGGTGACAT |
| 3417 | Left | AAGGTGATCTATTTTTCCCTTTCTC |
| 3418 | Right | GCAATTTCACAGAAAACTCATTGTT |
| 3419 | Left | TTATTTGTTCTCTCTCCAGAGTGCT |
| 3420 | Right | CTGTTATGTGTACCCAAAAAGGTG |
| 3421 | Left | TCTCTCTCCAGAGTGCTCTAATGAC |
| 3422 | Right | TTATGTGTACCCAAAAAGGTGACAT |
| 3423 | Left | TGTTCTCTCTCCAGAGTGCTCTAAT |
| 3424 | Right | TGTTATGTGTACCCAAAAAGGTGAC |
| 3425 | Left | GTTCTCTCTCCAGAGTGCTCTAATG |
| 3426 | Right | TGTTATGTGTACCCAAAAAGGTGAC |
| 3427 | Left | TGTTCTCTCTCCAGAGTGCTCTAAT |
| 3428 | Right | GTTATGTGTACCCAAAAAGGTGACA |
| 3429 | Left | GTTCTCTCTCCAGAGTGCTCTAATG |
| 3430 | Right | GTTATGTGTACCCAAAAAGGTGACA |
| 3431 | Left | GTTCTCTCTCCAGAGTGCTCTAATG |
| 3432 | Right | CTGTTATGTGTACCCAAAAAGGTG |
| 3433 | Left | TGTTCTCTCTCCAGAGTGCTCTAAT |
| 3434 | Right | CTGTTATGTGTACCCAAAAAGGTG |
| 3435 | Left | CAGAGTGCTCTAATGACTGAGACAA |
| 3436 | Right | GTTATGTGTACCCAAAAAGGTGACA |
| 3437 | Left | CAGAGTGCTCTAATGACTGAGACAA |
| 3438 | Right | TGTTATGTGTACCCAAAAAGGTGAC |
| 3439 | Left | TTATTTGTTCTCTCTCCAGAGTGCT |
| 3440 | Right | CTGTTATGTGTACCCAAAAAGGTGA |

KIT Exon11 301-400 bases

| | | |
|---|---|---|
| 3441 | Left | TTATTTGTTCTCTCTCCAGAGTGCT |
| 3442 | Right | TTCTCTATGGCAAACCTATCAAAAG |
| 3443 | Left | TTATTTGTTCTCTCTCCAGAGTGCT |
| 3444 | Right | GTTCTCTATGGCAAACCTATCAAAA |
| 3445 | Left | TGTTCTCTCTCCAGAGTGCTCTAAT |
| 3446 | Right | TTCTCTATGGCAAACCTATCAAAAG |
| 3447 | Left | GTTCTCTCTCCAGAGTGCTCTAATG |
| 3448 | Right | TTCTCTATGGCAAACCTATCAAAAG |
| 3449 | Left | CAGAGTGCTCTAATGACTGAGACAA |
| 3450 | Right | TTCTCTATGGCAAACCTATCAAAAG |
| 3451 | Left | GTTCTCTCTCCAGAGTGCTCTAATG |
| 3452 | Right | GTTCTCTATGGCAAACCTATCAAAA |
| 3453 | Left | TGTTCTCTCTCCAGAGTGCTCTAAT |
| 3454 | Right | GTTCTCTATGGCAAACCTATCAAAA |
| 3455 | Left | CAGAGTGCTCTAATGACTGAGACAA |
| 3456 | Right | GTTCTCTATGGCAAACCTATCAAAA |
| 3457 | Left | TTATTTGTTCTCTCTCCAGAGTGCT |
| 3458 | Right | ATGTTGTCCAGAGACATTTTCCTAC |
| 3459 | Left | TGTTCTCTCTCCAGAGTGCTCTAAT |
| 3460 | Right | ATGTTGTCCAGAGACATTTTCCTAC |
| 3461 | Left | GTTCTCTCTCCAGAGTGCTCTAATG |
| 3462 | Right | ATGTTGTCCAGAGACATTTTCCTAC |
| 3463 | Left | TTATTTGTTCTCTCTCCAGAGTGCT |
| 3464 | Right | CATTTTCCTACGATGTTCTCTATGG |
| 3465 | Left | CTCTCTCCAGAGTGCTCTAATGACT |
| 3466 | Right | TTCTCTATGGCAAACCTATCAAAAG |
| 3467 | Left | TTATTTGTTCTCTCTCCAGAGTGCT |
| 3468 | Right | ATGTTCTCTATGGCAAACCTATCAA |
| 3469 | Left | CAGAGTGCTCTAATGACTGAGACAA |
| 3470 | Right | ATGTTGTCCAGAGACATTTTCCTAC |
| 3471 | Left | TTATTTGTTCTCTCTCCAGAGTGCT |
| 3472 | Right | AATGTTGTCCAGAGACATTTTCCTA |
| 3473 | Left | CTCTCTCCAGAGTGCTCTAATGACT |
| 3474 | Right | GTTCTCTATGGCAAACCTATCAAAA |
| 3475 | Left | GTTCTCTCTCCAGAGTGCTCTAATG |
| 3476 | Right | CATTTTCCTACGATGTTCTCTATGG |
| 3477 | Left | TGTTCTCTCTCCAGAGTGCTCTAAT |
| 3478 | Right | CATTTTCCTACGATGTTCTCTATGG |
| 3479 | Left | GTTCTCTCTCCAGAGTGCTCTAATG |
| 3480 | Right | ATGTTCTCTATGGCAAACCTATCAA |

KIT Exon12 130-150 bases

| | | |
|---|---|---|
| 3481 | Left | CCTTGTTGTCTTCCTTCCTACAG |
| 3482 | Right | GCAGTACCATACAGGAACTTAC |
| 3483 | Left | TGTTGTCTTCCTTCCTACAG |
| 3484 | Right | CATGCAGTACCATACAGGAACTTAC |
| 3485 | Left | CTTGTTGTCTTCCTTCCTACAG |
| 3486 | Right | TGCAGTACCATACAGGAACTTAC |
| 3487 | Left | TTGTTGTCTTCCTTCCTACAG |
| 3488 | Right | ATGCAGTACCATACAGGAACTTAC |
| 3489 | Left | CCTTGTTGTCTTCCTTCCTACA |
| 3490 | Right | GCAGTACCATACAGGAACTTAC |
| 3491 | Left | CCTTGTTGTCTTCCTTCCTACAG |
| 3492 | Right | GCAGTACCATACAGGAACTTA |
| 3493 | Left | TGTTGTCTTCCTTCCTACAG |
| 3494 | Right | CATGCAGTACCATACAGGAACTTA |
| 3495 | Left | CTTGTTGTCTTCCTTCCTACAG |
| 3496 | Right | TGCAGTACCATACAGGAACTTA |
| 3497 | Left | CTTGTTGTCTTCCTTCCTACA |
| 3498 | Right | TGCAGTACCATACAGGAACTTAC |
| 3499 | Left | TTGTTGTCTTCCTTCCTACAG |
| 3500 | Right | TGCAGTACCATACAGGAACTTAC |
| 3501 | Left | TGTTGTCTTCCTTCCTACA |
| 3502 | Right | CATGCAGTACCATACAGGAACTTAC |
| 3503 | Left | TTGTTGTCTTCCTTCCTACAG |
| 3504 | Right | ATGCAGTACCATACAGGAACTTA |
| 3505 | Left | CCTTGTTGTCTTCCTTCCTACAG |
| 3506 | Right | GCAGTACCATACAGGAACTT |
| 3507 | Left | TGTTGTCTTCCTTCCTACAG |
| 3508 | Right | CATGCAGTACCATACAGGAACTT |
| 3509 | Left | ACCTTGTTGTCTTCCTTCCTACAG |
| 3510 | Right | CAGTACCATACAGGAACTTAC |
| 3511 | Left | TTGTTGTCTTCCTTCCTACA |
| 3512 | Right | ATGCAGTACCATACAGGAACTTAC |
| 3513 | Left | CTTGTTGTCTTCCTTCCTACAG |
| 3514 | Right | TGCAGTACCATACAGGAACTT |
| 3515 | Left | CCTTGTTGTCTTCCTTCCTACA |
| 3516 | Right | GCAGTACCATACAGGAACTTA |
| 3517 | Left | TGTTGTCTTCCTTCCTACAG |
| 3518 | Right | ATGCAGTACCATACAGGAACTTAC |
| 3519 | Left | TTGTTGTCTTCCTTCCTACAG |
| 3520 | Right | ATGCAGTACCATACAGGAACTT |

KIT Exon12 151-200 bases

| | | |
|---|---|---|
| 3521 | Left | TACCTTGTTGTCTTCCTTCCTACAG |
| 3522 | Right | CATGCAGTACCATACAGGAACTTAC |
| 3523 | Left | TTACCTTGTTGTCTTCCTTCCTACA |
| 3524 | Right | CATGCAGTACCATACAGGAACTTAC |
| 3525 | Left | TACCTTGTTGTCTTCCTTCCTACAG |
| 3526 | Right | GCATGCAGTACCATACAGGAACTTA |
| 3527 | Left | TTACCTTGTTGTCTTCCTTCCTACA |
| 3528 | Right | GCATGCAGTACCATACAGGAACTTA |
| 3529 | Left | ACCTTGTTGTCTTCCTTCCTACAG |
| 3530 | Right | CATGCAGTACCATACAGGAACTTAC |
| 3531 | Left | ACCACTTACCTTGTTGTCTTCCTTC |
| 3532 | Right | CATGCAGTACCATACAGGAACTTAC |
| 3533 | Left | CTTACCTTGTTGTCTTCCTTCCTAC |
| 3534 | Right | CATGCAGTACCATACAGGAACTTAC |
| 3535 | Left | ACTTACCTTGTTGTCTTCCTTCCTA |
| 3536 | Right | CATGCAGTACCATACAGGAACTTAC |
| 3537 | Left | CATCACCACTTACCTTGTTGTCTTC |
| 3538 | Right | CATGCAGTACCATACAGGAACTTAC |
| 3539 | Left | CACTTACCTTGTTGTCTTCCTTCCT |
| 3540 | Right | CATGCAGTACCATACAGGAACTTAC |
| 3541 | Left | CCACTTACCTTGTTGTCTTCCTTC |
| 3542 | Right | CATGCAGTACCATACAGGAACTTAC |
| 3543 | Left | CACTTACCTTGTTGTCTTCCTTCC |
| 3544 | Right | CATGCAGTACCATACAGGAACTTAC |
| 3545 | Left | ACTTACCTTGTTGTCTTCCTTCCTAC |
| 3546 | Right | CATGCAGTACCATACAGGAACTTAC |
| 3547 | Left | ACCACTTACCTTGTTGTCTTCCTT |
| 3548 | Right | CATGCAGTACCATACAGGAACTTAC |
| 3549 | Left | CATCACCACTTACCTTGTTGTCTT |
| 3550 | Right | CATGCAGTACCATACAGGAACTTAC |
| 3551 | Left | ACCTTGTTGTCTTCCTTCCTACAG |
| 3552 | Right | GCATGCAGTACCATACAGGAACTTA |
| 3553 | Left | ACCACTTACCTTGTTGTCTTCCTTC |
| 3554 | Right | GCATGCAGTACCATACAGGAACTTA |
| 3555 | Left | TACCTTGTTGTCTTCCTTCCTACA |
| 3556 | Right | CATGCAGTACCATACAGGAACTTAC |
| 3557 | Left | CTTACCTTGTTGTCTTCCTTCCTAC |
| 3558 | Right | GCATGCAGTACCATACAGGAACTTA |
| 3559 | Left | ACTTACCTTGTTGTCTTCCTTCCTA |
| 3560 | Right | GCATGCAGTACCATACAGGAACTTA |

KIT Exon12 201-300 bases

| | | |
|---|---|---|
| 3561 | Left | CTTTTGATAGGTTTGCCATAGAGAA |
| 3562 | Right | CATGCAGTACCATACAGGAACTTAC |
| 3563 | Left | AGAACATCGTAGGAAAATGTCTCTG |
| 3564 | Right | CATGCAGTACCATACAGGAACTTAC |
| 3565 | Left | CCATAGAGAACATCGTAGGAAAATG |
| 3566 | Right | CATGCAGTACCATACAGGAACTTAC |
| 3567 | Left | CTTTTGATAGGTTTGCCATAGAGAA |
| 3568 | Right | GCATGCAGTACCATACAGGAACTTA |
| 3569 | Left | TCCTTTATTGATTTTGAAACTGCAC |
| 3570 | Right | CATGCAGTACCATACAGGAACTTAC |
| 3571 | Left | TGATAGGTTTGCCATAGAGAACATC |
| 3572 | Right | CATGCAGTACCATACAGGAACTTAC |
| 3573 | Left | AGAACATCGTAGGAAAATGTCTCTG |
| 3574 | Right | GCATGCAGTACCATACAGGAACTTA |
| 3575 | Left | CCATAGAGAACATCGTAGGAAAATG |
| 3576 | Right | GCATGCAGTACCATACAGGAACTTA |
| 3577 | Left | ATTCCTTTATTGATTTTGAAACTGC |
| 3578 | Right | CATGCAGTACCATACAGGAACTTAC |
| 3579 | Left | ATGTCTCTGGACAACATTGTTTTTA |
| 3580 | Right | CATGCAGTACCATACAGGAACTTAC |
| 3581 | Left | GCCATAGAGAACATCGTAGGAAAAT |
| 3582 | Right | CATGCAGTACCATACAGGAACTTAC |
| 3583 | Left | TTGATAGGTTTGCCATAGAGAACA |
| 3584 | Right | CATGCAGTACCATACAGGAACTTAC |
| 3585 | Left | AATTCCTTTATTGATTTTGAAACTGC |
| 3586 | Right | CATGCAGTACCATACAGGAACTTAC |
| 3587 | Left | TTTGATAGGTTTGCCATAGAGAACA |
| 3588 | Right | CATGCAGTACCATACAGGAACTTAC |
| 3589 | Left | TCTCTGGACAACATTGTTTTTAATTC |
| 3590 | Right | CATGCAGTACCATACAGGAACTTAC |
| 3591 | Left | TAGGTTTGCCATAGAGAACATCGTA |
| 3592 | Right | CATGCAGTACCATACAGGAACTTAC |
| 3593 | Left | TGTCTCTGGACAACATTGTTTTTAAT |
| 3594 | Right | CATGCAGTACCATACAGGAACTTAC |
| 3595 | Left | AATGTCTCTGGACAACATTGTTTTTA |
| 3596 | Right | CATGCAGTACCATACAGGAACTTAC |
| 3597 | Left | TGGACAACATTGTTTTTAATTCCTT |
| 3598 | Right | GCATTTTAGCAAAAAGCACAACT |
| 3599 | Left | TCCTTTATTGATTTTGAAACTGCAC |
| 3600 | Right | GCATGCAGTACCATACAGGAACTTA |

KIT Exon12 301-400 bases

| | | |
|---|---|---|
| 3601 | Left | TGAAACAATGAGTTTTCTGTGAAAT |
| 3602 | Right | CATGCAGTACCATACAGGAACTTAC |
| 3603 | Left | CTGAAACAATGAGTTTTCTGTGAAAT |
| 3604 | Right | CATGCAGTACCATACAGGAACTTAC |
| 3605 | Left | CTTTTTGGGTACACATAACAGTGAC |
| 3606 | Right | CATGCAGTACCATACAGGAACTTAC |
| 3607 | Left | TTTTGGGTACACATAACAGTGACTT |
| 3608 | Right | CATGCAGTACCATACAGGAACTTAC |
| 3609 | Left | TTTTTGGGTACACATAACAGTGACTT |
| 3610 | Right | CATGCAGTACCATACAGGAACTTAC |
| 3611 | Left | CTTTTGATAGGTTTGCCATAGAGAA |
| 3612 | Right | GCATTTTAGCAAAAAGCACAACT |
| 3613 | Left | ACCTGAAACAATGAGTTTTCTGTGA |
| 3614 | Right | CATGCAGTACCATACAGGAACTTAC |
| 3615 | Left | AGAACATCGTAGGAAAATGTCTCTG |
| 3616 | Right | GCATTTTAGCAAAAAGCACAACT |
| 3617 | Left | AAACAATGAGTTTTCTGTGAAATTG |
| 3618 | Right | CATGCAGTACCATACAGGAACTTAC |
| 3619 | Left | GAAACAATGAGTTTTCTGTGAAATTG |
| 3620 | Right | CATGCAGTACCATACAGGAACTTAC |
| 3621 | Left | CCTTTTTGGGTACACATAACAGTGA |
| 3622 | Right | CATGCAGTACCATACAGGAACTTAC |
| 3623 | Left | CTTTTGATAGGTTTGCCATAGAGAA |
| 3624 | Right | ATTTTAGCAAAAAGCACAACTGG |
| 3625 | Left | ACCTGAAACAATGAGTTTTCTGTG |
| 3626 | Right | CATGCAGTACCATACAGGAACTTAC |
| 3627 | Left | AGAACATCGTAGGAAAATGTCTCTG |
| 3628 | Right | ATTTTAGCAAAAAGCACAACTGG |
| 3629 | Left | CCATAGAGAACATCGTAGGAAAATG |
| 3630 | Right | GCATTTTAGCAAAAAGCACAACT |
| 3631 | Left | TGAAACAATGAGTTTTCTGTGAAAT |
| 3632 | Right | GCATGCAGTACCATACAGGAACTTA |
| 3633 | Left | CCTGAAACAATGAGTTTTCTGTGA |
| 3634 | Right | CATGCAGTACCATACAGGAACTTAC |
| 3635 | Left | CCATAGAGAACATCGTAGGAAAATG |
| 3636 | Right | ATTTTAGCAAAAAGCACAACTGG |
| 3637 | Left | GTTCCACCTGAAACAATGAGTTTT |
| 3638 | Right | CATGCAGTACCATACAGGAACTTAC |
| 3639 | Left | CTGAAACAATGAGTTTTCTGTGAAAT |
| 3640 | Right | GCATGCAGTACCATACAGGAACTTA |

KIT Exon13 131-150 bases

| | | |
|---|---|---|
| 3641 | Left | TGCATGTTTCCAATTTTAG |
| 3642 | Right | CAGCTTGGACACGGCTTTAC |
| 3643 | Left | AATGCATGTTTCCAATTTTAG |
| 3644 | Right | GCTTGGACACGGCTTTAC |
| 3645 | Left | TGCATGTTTCCAATTTTAG |
| 3646 | Right | CAGCTTGGACACGGCTTTA |
| 3647 | Left | ATGCATGTTTCCAATTTTAG |
| 3648 | Right | AGCTTGGACACGGCTTTAC |
| 3649 | Left | TGCATGTTTCCAATTTTAG |
| 3650 | Right | CAGCTTGGACACGGCTTT |
| 3651 | Left | TGCATGTTTCCAATTTTAG |
| 3652 | Right | AGCTTGGACACGGCTTTAC |
| 3653 | Left | AATGCATGTTTCCAATTTTA |
| 3654 | Right | GCTTGGACACGGCTTTAC |
| 3655 | Left | ATGCATGTTTCCAATTTTAG |
| 3656 | Right | AGCTTGGACACGGCTTTA |
| 3657 | Left | TGCATGTTTCCAATTTTA |
| 3658 | Right | CAGCTTGGACACGGCTTTAC |
| 3659 | Left | ATGCATGTTTCCAATTTTAG |
| 3660 | Right | GCTTGGACACGGCTTTAC |
| 3661 | Left | TGCATGTTTCCAATTTTA |
| 3662 | Right | CAGCTTGGACACGGCTTTA |
| 3663 | Left | ATGCATGTTTCCAATTTTA |
| 3664 | Right | AGCTTGGACACGGCTTTAC |
| 3665 | Left | AATGCATGTTTCCAATTTT |
| 3666 | Right | GCTTGGACACGGCTTTAC |
| 3667 | Left | TGCATGTTTCCAATTTTA |
| 3668 | Right | CAGCTTGGACACGGCTTT |
| 3669 | Left | TGCATGTTTCCAATTTTAG |
| 3670 | Right | AGCTTGGACACGGCTTTA |
| 3671 | Left | ATGCATGTTTCCAATTTT |
| 3672 | Right | AGCTTGGACACGGCTTTAC |
| 3673 | Left | GCATGTTTCCAATTTTAG |
| 3674 | Right | CAGCTTGGACACGGCTTTAC |
| 3675 | Left | TGCATGTTTCCAATTTTAG |
| 3676 | Right | GCTTGGACACGGCTTTAC |
| 3677 | Left | GCATGTTTCCAATTTTAG |
| 3678 | Right | CAGCTTGGACACGGCTTTA |
| 3679 | Left | TGCATGTTTCCAATTTTA |
| 3680 | Right | AGCTTGGACACGGCTTTAC |

KIT Exon13 151-200 bases

| | | |
|---|---|---|
| 3681 | Left | TGCTAAAATGCATGTTTCCAAT |
| 3682 | Right | CATGTTTTGATAACCTGACAGACAA |
| 3683 | Left | TAAAATGCATGTTTCCAATTTTAG |
| 3684 | Right | CATGTTTTGATAACCTGACAGACAA |
| 3685 | Left | TGCTAAAATGCATGTTTCCAAT |
| 3686 | Right | TTGATAACCTGACAGACAATAAAAGG |
| 3687 | Left | TAAAATGCATGTTTCCAATTTTAG |
| 3688 | Right | TTGATAACCTGACAGACAATAAAAGG |
| 3689 | Left | TGCTAAAATGCATGTTTCCAAT |
| 3690 | Right | TGATAACCTGACAGACAATAAAAGG |
| 3691 | Left | TAAAATGCATGTTTCCAATTTTAG |
| 3692 | Right | TGATAACCTGACAGACAATAAAAGG |
| 3693 | Left | AAAATGCATGTTTCCAATTTTAG |
| 3694 | Right | CATGTTTTGATAACCTGACAGACAA |
| 3695 | Left | TGCTAAAATGCATGTTTCCAAT |
| 3696 | Right | CATGTTTTGATAACCTGACAGACAAT |
| 3697 | Left | TAAAATGCATGTTTCCAATTTTAG |
| 3698 | Right | CATGTTTTGATAACCTGACAGACAAT |
| 3699 | Left | TGCTAAAATGCATGTTTCCAAT |
| 3700 | Right | ATGTTTTGATAACCTGACAGACAAT |
| 3701 | Left | TGCTAAAATGCATGTTTCCAAT |
| 3702 | Right | TGTTTTGATAACCTGACAGACAATAA |
| 3703 | Left | TAAAATGCATGTTTCCAATTTTAG |
| 3704 | Right | ATGTTTTGATAACCTGACAGACAAT |
| 3705 | Left | TGCTAAAATGCATGTTTCCAAT |
| 3706 | Right | CATGTTTTGATAACCTGACAGACA |
| 3707 | Left | TAAAATGCATGTTTCCAATTTTAG |
| 3708 | Right | TGTTTTGATAACCTGACAGACAATAA |
| 3709 | Left | TAAAATGCATGTTTCCAATTTTAG |
| 3710 | Right | CATGTTTTGATAACCTGACAGACA |
| 3711 | Left | TGCTAAAATGCATGTTTCCAAT |
| 3712 | Right | CTGACAGACAATAAAAGGCAGCTT |
| 3713 | Left | TAAAATGCATGTTTCCAATTTTAG |
| 3714 | Right | CTGACAGACAATAAAAGGCAGCTT |
| 3715 | Left | TGCTAAAATGCATGTTTCCAAT |
| 3716 | Right | TGTTTTGATAACCTGACAGACAATA |
| 3717 | Left | TAAAATGCATGTTTCCAATTTTAG |
| 3718 | Right | TGTTTTGATAACCTGACAGACAATA |
| 3719 | Left | AAAATGCATGTTTCCAATTTTAG |
| 3720 | Right | TTGATAACCTGACAGACAATAAAAGG |

KIT Exon13 201-300 bases

| | | |
|---|---|---|
| 3721 | Left | CTTGACATCAGTTTGCCAGTTGT |
| 3722 | Right | GAGAGAACAACAGTCTGGGTAAAAA |
| 3723 | Left | TGCTAAAATGCATGTTTCCAAT |
| 3724 | Right | GAGAGAACAACAGTCTGGGTAAAAA |
| 3725 | Left | TAAAATGCATGTTTCCAATTTTAG |
| 3726 | Right | GAGAGAACAACAGTCTGGGTAAAAA |
| 3727 | Left | TTGACATCAGTTTGCCAGTTGT |
| 3728 | Right | GAGAGAACAACAGTCTGGGTAAAAA |
| 3729 | Left | CTTGACATCAGTTTGCCAGTTGT |
| 3730 | Right | AGAGAGAACAACAGTCTGGGTAAAA |
| 3731 | Left | TGCTAAAATGCATGTTTCCAAT |
| 3732 | Right | AGAGAGAACAACAGTCTGGGTAAAA |
| 3733 | Left | TGCTAAAATGCATGTTTCCAAT |
| 3734 | Right | AAGAGAGAACAACAGTCTGGGTAAA |
| 3735 | Left | TAAAATGCATGTTTCCAATTTTAG |
| 3736 | Right | AAGAGAGAACAACAGTCTGGGTAAA |
| 3737 | Left | CTTGACATCAGTTTGCCAGTTG |
| 3738 | Right | GAGAGAACAACAGTCTGGGTAAAAA |
| 3739 | Left | TTGACATCAGTTTGCCAGTTGT |
| 3740 | Right | AGAGAGAACAACAGTCTGGGTAAAA |
| 3741 | Left | TAAAATGCATGTTTCCAATTTTAG |
| 3742 | Right | ACAATGAGGAAAACAAAATCTAGCA |
| 3743 | Left | CTTGACATCAGTTTGCCAGTTG |
| 3744 | Right | AGAGAGAACAACAGTCTGGGTAAAA |
| 3745 | Left | CTTGACATCAGTTTGCCAGTTG |
| 3746 | Right | AAGAGAGAACAACAGTCTGGGTAAA |
| 3747 | Left | TGCTAAAATGCATGTTTCCAAT |
| 3748 | Right | GGAAAACAAAATCTAGCAAGAGAGA |
| 3749 | Left | TGCTAAAATGCATGTTTCCAAT |
| 3750 | Right | GAGGAAAACAAAATCTAGCAAGAGA |
| 3751 | Left | CTTGACATCAGTTTGCCAGTTG |
| 3752 | Right | CATGTTTTGATAACCTGACAGACAA |
| 3753 | Left | TAAAATGCATGTTTCCAATTTTAG |
| 3754 | Right | GGAAAACAAAATCTAGCAAGAGAGA |
| 3755 | Left | TAAAATGCATGTTTCCAATTTTAG |
| 3756 | Right | GAGGAAAACAAAATCTAGCAAGAGA |
| 3757 | Left | ATCAGTTTGCCAGTTGTGCTTT |
| 3758 | Right | GAGAGAACAACAGTCTGGGTAAAAA |
| 3759 | Left | ATCAGTTTGCCAGTTGTGCTT |
| 3760 | Right | GAGAGAACAACAGTCTGGGTAAAAA |

KIT Exon13 301-400 bases

| | | |
|---|---|---|
| 3761 | Left | CTTGACATCAGTTTGCCAGTTGT |
| 3762 | Right | ACGACAATAACTAGGGTATGTCCTG |
| 3763 | Left | TGCTAAAATGCATGTTTCCAAT |
| 3764 | Right | ACGACAATAACTAGGGTATGTCCTG |
| 3765 | Left | TGCTAAAATGCATGTTTCCAAT |
| 3766 | Right | TCGTTGATGTTACAAATACGACAAT |
| 3767 | Left | TAAAATGCATGTTTCCAATTTTAG |
| 3768 | Right | ACGACAATAACTAGGGTATGTCCTG |
| 3769 | Left | TAAAATGCATGTTTCCAATTTTAG |
| 3770 | Right | TCGTTGATGTTACAAATACGACAAT |
| 3771 | Left | CTTGACATCAGTTTGCCAGTTGT |
| 3772 | Right | GCTGTTCTACCCCATAATGATAAAA |
| 3773 | Left | TGCTAAAATGCATGTTTCCAAT |
| 3774 | Right | GCTGATGTCGTTGATGTTACAAATA |
| 3775 | Left | TGCTAAAATGCATGTTTCCAAT |
| 3776 | Right | GCTGTTCTACCCCATAATGATAAAA |
| 3777 | Left | TAAAATGCATGTTTCCAATTTTAG |
| 3778 | Right | GCTGATGTCGTTGATGTTACAAATA |
| 3779 | Left | TTGACATCAGTTTGCCAGTTGT |
| 3780 | Right | ACGACAATAACTAGGGTATGTCCTG |
| 3781 | Left | TTGACATCAGTTTGCCAGTTGT |
| 3782 | Right | GCTGTTCTACCCCATAATGATAAAA |
| 3783 | Left | CTTGACATCAGTTTGCCAGTTG |
| 3784 | Right | ACGACAATAACTAGGGTATGTCCTG |
| 3785 | Left | CTTGACATCAGTTTGCCAGTTGT |
| 3786 | Right | GAGGAAAACAAAATCTAGCAAGAGA |
| 3787 | Left | CTTGACATCAGTTTGCCAGTTG |
| 3788 | Right | GCTGTTCTACCCCATAATGATAAAA |
| 3789 | Left | TTGACATCAGTTTGCCAGTTGT |
| 3790 | Right | GAGGAAAACAAAATCTAGCAAGAGA |
| 3791 | Left | CTTGACATCAGTTTGCCAGTTG |
| 3792 | Right | ACAATGAGGAAAACAAAATCTAGCA |
| 3793 | Left | CTTGACATCAGTTTGCCAGTTG |
| 3794 | Right | GAGGAAAACAAAATCTAGCAAGAGA |
| 3795 | Left | CTTGACATCAGTTTGCCAGTTG |
| 3796 | Right | GGAAAACAAAATCTAGCAAGAGAGA |
| 3797 | Left | ATCAGTTTGCCAGTTGTGCTTT |
| 3798 | Right | ACGACAATAACTAGGGTATGTCCTG |
| 3799 | Left | ATCAGTTTGCCAGTTGTGCTTT |
| 3800 | Right | GCTGTTCTACCCCATAATGATAAAA |

KIT Exon10-11 301-400 bases

| | | |
|---|---|---|
| 3801 | Left | GATTCCACATTTCTCTTCCATTGTA |
| 3802 | Right | TTATGTGTACCCAAAAAGGTGACAT |
| 3803 | Left | GATTCCACATTTCTCTTCCATTGTA |
| 3804 | Right | TATGTGTACCCAAAAAGGTGACAT |
| 3805 | Left | GATTCCACATTTCTCTTCCATTGTA |
| 3806 | Right | TTATGTGTACCCAAAAAGGTGACA |
| 3807 | Left | GATTCCACATTTCTCTTCCATTGT |
| 3808 | Right | TTATGTGTACCCAAAAAGGTGACAT |
| 3809 | Left | ATTCCACATTTCTCTTCCATTGTA |
| 3810 | Right | TTATGTGTACCCAAAAAGGTGACAT |
| 3811 | Left | GATTCCACATTTCTCTTCCATTG |
| 3812 | Right | TTATGTGTACCCAAAAAGGTGACAT |
| 3813 | Left | GATTCCACATTTCTCTTCCATTGT |
| 3814 | Right | TATGTGTACCCAAAAAGGTGACAT |
| 3815 | Left | GATTCCACATTTCTCTTCCATTGTA |
| 3816 | Right | ATGTGTACCCAAAAAGGTGACAT |
| 3817 | Left | GATTCCACATTTCTCTTCCATTGT |
| 3818 | Right | TTATGTGTACCCAAAAAGGTGACA |
| 3819 | Left | GATTCCACATTTCTCTTCCATTGTA |
| 3820 | Right | TATGTGTACCCAAAAAGGTGACA |
| 3821 | Left | AGTTTGTGATTCCACATTTCTCTTC |
| 3822 | Right | GAAAGCCCCTGTTTCATACTGAC |
| 3823 | Left | ATTCCACATTTCTCTTCCATTGTA |
| 3824 | Right | GTTATGTGTACCCAAAAAGGTGACA |
| 3825 | Left | ATTCCACATTTCTCTTCCATTGTA |
| 3826 | Right | TATGTGTACCCAAAAAGGTGACAT |
| 3827 | Left | AGTTTGTGATTCCACATTTCTCTTC |
| 3828 | Right | AAAGCCCCTGTTTCATACTGAC |
| 3829 | Left | TGATTCCACATTTCTCTTCCATT |
| 3830 | Right | TATGTGTACCCAAAAAGGTGACAT |
| 3831 | Left | GATTCCACATTTCTCTTCCATTG |
| 3832 | Right | TATGTGTACCCAAAAAGGTGACAT |
| 3833 | Left | ATTCCACATTTCTCTTCCATTGTA |
| 3834 | Right | TTATGTGTACCCAAAAAGGTGACA |
| 3835 | Left | GATTCCACATTTCTCTTCCATTG |
| 3836 | Right | TTATGTGTACCCAAAAAGGTGACA |
| 3837 | Left | AAGTTTGTGATTCCACATTTCTCTT |
| 3838 | Right | GAAAGCCCCTGTTTCATACTGAC |
| 3839 | Left | AAAGTTTGTGATTCCACATTTCTCT |
| 3840 | Right | GAAAGCCCCTGTTTCATACTGAC |

KIT Exon10-11 401-500 bases

| | | |
|---|---|---|
| 3841 | Left | AGTTTGTGATTCCACATTTCTCTTC |
| 3842 | Right | TTATGTGTACCCAAAAAGGTGACAT |
| 3843 | Left | AGTTTGTGATTCCACATTTCTCTTC |
| 3844 | Right | GCAATTTCACAGAAAACTCATTGTT |
| 3845 | Left | AGTTTGTGATTCCACATTTCTCTTC |
| 3846 | Right | ATTTCACAGAAAACTCATTGTTTCA |
| 3847 | Left | AAGTTTGTGATTCCACATTTCTCTT |
| 3848 | Right | TTATGTGTACCCAAAAAGGTGACAT |
| 3849 | Left | AAAGTTTGTGATTCCACATTTCTCT |
| 3850 | Right | TTATGTGTACCCAAAAAGGTGACAT |
| 3851 | Left | AAGTTTGTGATTCCACATTTCTCTT |
| 3852 | Right | GCAATTTCACAGAAAACTCATTGTT |
| 3853 | Left | AAAGTTTGTGATTCCACATTTCTCT |
| 3854 | Right | GCAATTTCACAGAAAACTCATTGTT |
| 3855 | Left | AGTTTGTGATTCCACATTTCTCTTC |
| 3856 | Right | GTTATGTGTACCCAAAAAGGTGACA |
| 3857 | Left | AGTTTGTGATTCCACATTTCTCTTC |
| 3858 | Right | TGTTATGTGTACCCAAAAAGGTGAC |
| 3859 | Left | GATTCCACATTTCTCTTCCATTGTA |
| 3860 | Right | TTATGTGTACCCAAAAAGGTGACAT |
| 3861 | Left | GATTCCACATTTCTCTTCCATTGTA |
| 3862 | Right | GCAATTTCACAGAAAACTCATTGTT |
| 3863 | Left | AGTTTGTGATTCCACATTTCTCTTC |
| 3864 | Right | CTGTTATGTGTACCCAAAAAGGTG |
| 3865 | Left | AGTTTGTGATTCCACATTTCTCTTC |
| 3866 | Right | TCACAGAAAACTCATTGTTTCAGGT |
| 3867 | Left | AGTTTGTGATTCCACATTTCTCTTC |
| 3868 | Right | AATTTCACAGAAAACTCATTGTTTCA |
| 3869 | Left | AAGTTTGTGATTCCACATTTCTCTT |
| 3870 | Right | ATTTCACAGAAAACTCATTGTTTCA |
| 3871 | Left | AAAGTTTGTGATTCCACATTTCTCT |
| 3872 | Right | ATTTCACAGAAAACTCATTGTTTCA |
| 3873 | Left | AGTTTGTGATTCCACATTTCTCTTC |
| 3874 | Right | CTGTTATGTGTACCCAAAAAGGTGA |
| 3875 | Left | GATTCCACATTTCTCTTCCATTGTA |
| 3876 | Right | ATTTCACAGAAAACTCATTGTTTCA |
| 3877 | Left | AGTTTGTGATTCCACATTTCTCTTC |
| 3878 | Right | ACAGAAAACTCATTGTTTCAGGTG |
| 3879 | Left | AGTTTGTGATTCCACATTTCTCTTC |
| 3880 | Right | CACAGAAAACTCATTGTTTCAGGT |

KIT Exon10-11 501-600 bases

| | | |
|---|---|---|
| 3881 | Left | AGTTTGTGATTCCACATTTCTCTTC |
| 3882 | Right | TTCTCTATGGCAAACCTATCAAAAG |
| 3883 | Left | AGTTTGTGATTCCACATTTCTCTTC |
| 3884 | Right | GTTCTCTATGGCAAACCTATCAAAA |
| 3885 | Left | AGTTTGTGATTCCACATTTCTCTTC |
| 3886 | Right | ATGTTGTCCAGAGACATTTTCCTAC |
| 3887 | Left | AGTTTGTGATTCCACATTTCTCTTC |
| 3888 | Right | CATTTTCCTACGATGTTCTCTATGG |
| 3889 | Left | AGTTTGTGATTCCACATTTCTCTTC |
| 3890 | Right | ATGTTCTCTATGGCAAACCTATCAA |
| 3891 | Left | AGTTTGTGATTCCACATTTCTCTTC |
| 3892 | Right | CATTTGTGCAGTTTCAAAATCAATA |
| 3893 | Left | AGTTTGTGATTCCACATTTCTCTTC |
| 3894 | Right | AATGTTGTCCAGAGACATTTTCCTA |
| 3895 | Left | AGTTTGTGATTCCACATTTCTCTTC |
| 3896 | Right | AGGAATTAAAAACAATGTTGTCCAG |
| 3897 | Left | AGTTTGTGATTCCACATTTCTCTTC |
| 3898 | Right | TTGTGCAGTTTCAAAATCAATAAAG |
| 3899 | Left | AGTTTGTGATTCCACATTTCTCTTC |
| 3900 | Right | GGAATTAAAAACAATGTTGTCCAGA |
| 3901 | Left | AAGTTTGTGATTCCACATTTCTCTT |
| 3902 | Right | TTCTCTATGGCAAACCTATCAAAAG |
| 3903 | Left | AAAGTTTGTGATTCCACATTTCTCT |
| 3904 | Right | TTCTCTATGGCAAACCTATCAAAAG |
| 3905 | Left | AAGTTTGTGATTCCACATTTCTCTT |
| 3906 | Right | GTTCTCTATGGCAAACCTATCAAAA |
| 3907 | Left | AAAGTTTGTGATTCCACATTTCTCT |
| 3908 | Right | GTTCTCTATGGCAAACCTATCAAAA |
| 3909 | Left | GATTCCACATTTCTCTTCCATTGTA |
| 3910 | Right | TTCTCTATGGCAAACCTATCAAAAG |
| 3911 | Left | GATTCCACATTTCTCTTCCATTGTA |
| 3912 | Right | GTTCTCTATGGCAAACCTATCAAAA |
| 3913 | Left | AAGTTTGTGATTCCACATTTCTCTT |
| 3914 | Right | ATGTTGTCCAGAGACATTTTCCTAC |
| 3915 | Left | AAAGTTTGTGATTCCACATTTCTCT |
| 3916 | Right | ATGTTGTCCAGAGACATTTTCCTAC |
| 3917 | Left | GATTCCACATTTCTCTTCCATTGTA |
| 3918 | Right | ATGTTGTCCAGAGACATTTTCCTAC |
| 3919 | Left | AAGTTTGTGATTCCACATTTCTCTT |
| 3920 | Right | CATTTTCCTACGATGTTCTCTATGG |

KIT Exon10-11 601-800 bases

| | | |
|---|---|---|
| 3921 | Left | TCTATTCTGCAGTATTGTGGTTTCA |
| 3922 | Right | TTCTCTATGGCAAACCTATCAAAAG |
| 3923 | Left | TCTGCAGTATTGTGGTTTCAAGTTA |
| 3924 | Right | TTCTCTATGGCAAACCTATCAAAAG |
| 3925 | Left | TCTATTCTGCAGTATTGTGGTTTCA |
| 3926 | Right | GTTCTCTATGGCAAACCTATCAAAA |
| 3927 | Left | TCTGCAGTATTGTGGTTTCAAGTTA |
| 3928 | Right | GTTCTCTATGGCAAACCTATCAAAA |
| 3929 | Left | TCTATTCTGCAGTATTGTGGTTTCA |
| 3930 | Right | ATGTTGTCCAGAGACATTTTCCTAC |
| 3931 | Left | GGCAGGAATTTGATTGAAGTATAAA |
| 3932 | Right | TTCTCTATGGCAAACCTATCAAAAG |
| 3933 | Left | TCTGCAGTATTGTGGTTTCAAGTTA |
| 3934 | Right | ATGTTGTCCAGAGACATTTTCCTAC |
| 3935 | Left | GGCAGGAATTTGATTGAAGTATAAA |
| 3936 | Right | GTTCTCTATGGCAAACCTATCAAAA |
| 3937 | Left | TCTATTCTGCAGTATTGTGGTTTCA |
| 3938 | Right | CATTTTCCTACGATGTTCTCTATGG |
| 3939 | Left | TCTATTCTGCAGTATTGTGGTTTCA |
| 3940 | Right | ATGTTCTCTATGGCAAACCTATCAA |
| 3941 | Left | TCTATTCTGCAGTATTGTGGTTTCA |
| 3942 | Right | AATGTTGTCCAGAGACATTTTCCTA |
| 3943 | Left | TCTGCAGTATTGTGGTTTCAAGTTA |
| 3944 | Right | CATTTTCCTACGATGTTCTCTATGG |
| 3945 | Left | TCTGCAGTATTGTGGTTTCAAGTTA |
| 3946 | Right | ATGTTCTCTATGGCAAACCTATCAA |
| 3947 | Left | GTACAATGTAACCAAGGTGAAGCTC |
| 3948 | Right | TTCTCTATGGCAAACCTATCAAAAG |
| 3949 | Left | GAGTACAATGTAACCAAGGTGAAGC |
| 3950 | Right | TTCTCTATGGCAAACCTATCAAAAG |
| 3951 | Left | ATTCTGCAGTATTGTGGTTTCAAGT |
| 3952 | Right | TTCTCTATGGCAAACCTATCAAAAG |
| 3953 | Left | TCTGCAGTATTGTGGTTTCAAGTTA |
| 3954 | Right | CATTTGTGCAGTTTCAAAATCAATA |
| 3955 | Left | TCTGCAGTATTGTGGTTTCAAGTTA |
| 3956 | Right | AATGTTGTCCAGAGACATTTTCCTA |
| 3957 | Left | TCTGCAGTATTGTGGTTTCAAGTTA |
| 3958 | Right | AGGAATTAAAAACAATGTTGTCCAG |
| 3959 | Left | GTACAATGTAACCAAGGTGAAGCTC |
| 3960 | Right | GTTCTCTATGGCAAACCTATCAAAA |

KIT Exon12-13 301-400 bases

| | | |
|---|---|---|
| 3961 | Left | TACCTTGTTGTCTTCCTTCCTACAG |
| 3962 | Right | CATGTTTTGATAACCTGACAGACAA |
| 3963 | Left | TTACCTTGTTGTCTTCCTTCCTACA |
| 3964 | Right | CATGTTTTGATAACCTGACAGACAA |
| 3965 | Left | TACCTTGTTGTCTTCCTTCCTACAG |
| 3966 | Right | TTGATAACCTGACAGACAATAAAAGG |
| 3967 | Left | ACCTTGTTGTCTTCCTTCCTACAG |
| 3968 | Right | CATGTTTTGATAACCTGACAGACAA |
| 3969 | Left | TACCTTGTTGTCTTCCTTCCTACAG |
| 3970 | Right | TGATAACCTGACAGACAATAAAAGG |
| 3971 | Left | ACCACTTACCTTGTTGTCTTCCTTC |
| 3972 | Right | CATGTTTTGATAACCTGACAGACAA |
| 3973 | Left | CTTACCTTGTTGTCTTCCTTCCTAC |
| 3974 | Right | CATGTTTTGATAACCTGACAGACAA |
| 3975 | Left | ACTTACCTTGTTGTCTTCCTTCCTA |
| 3976 | Right | CATGTTTTGATAACCTGACAGACAA |
| 3977 | Left | CACTTACCTTGTTGTCTTCCTTCCT |
| 3978 | Right | CATGTTTTGATAACCTGACAGACAA |
| 3979 | Left | TTACCTTGTTGTCTTCCTTCCTACA |
| 3980 | Right | TTGATAACCTGACAGACAATAAAAGG |
| 3981 | Left | TACCTTGTTGTCTTCCTTCCTACAG |
| 3982 | Right | CATGTTTTGATAACCTGACAGACAAT |
| 3983 | Left | TTACCTTGTTGTCTTCCTTCCTACA |
| 3984 | Right | TGATAACCTGACAGACAATAAAAGG |
| 3985 | Left | CACTTACCTTGTTGTCTTCCTTCC |
| 3986 | Right | CATGTTTTGATAACCTGACAGACAA |
| 3987 | Left | CCACTTACCTTGTTGTCTTCCTTC |
| 3988 | Right | CATGTTTTGATAACCTGACAGACAA |
| 3989 | Left | ACTTACCTTGTTGTCTTCCTTCCTAC |
| 3990 | Right | CATGTTTTGATAACCTGACAGACAA |
| 3991 | Left | ACCACTTACCTTGTTGTCTTCCTT |
| 3992 | Right | CATGTTTTGATAACCTGACAGACAA |
| 3993 | Left | TTACCTTGTTGTCTTCCTTCCTACA |
| 3994 | Right | CATGTTTTGATAACCTGACAGACAAT |
| 3995 | Left | TACCTTGTTGTCTTCCTTCCTACAG |
| 3996 | Right | ATGTTTTGATAACCTGACAGACAAT |
| 3997 | Left | TACCTTGTTGTCTTCCTTCCTACAG |
| 3998 | Right | TGTTTTGATAACCTGACAGACAATAA |
| 3999 | Left | TACCTTGTTGTCTTCCTTCCTACAG |
| 4000 | Right | CATGTTTTGATAACCTGACAGACA |

KIT Exon12-13 401-500 bases

| | | |
|---|---|---|
| 4001 | Left | TACCTTGTTGTCTTCCTTCCTACAG |
| 4002 | Right | GAGAGAACAACAGTCTGGGTAAAAA |
| 4003 | Left | TACCTTGTTGTCTTCCTTCCTACAG |
| 4004 | Right | AGAGAGAACAACAGTCTGGGTAAAA |
| 4005 | Left | TACCTTGTTGTCTTCCTTCCTACAG |
| 4006 | Right | AAGAGAGAACAACAGTCTGGGTAAA |
| 4007 | Left | TGGACAACATTGTTTTTAATTCCTT |
| 4008 | Right | CATGTTTTGATAACCTGACAGACAA |
| 4009 | Left | AGAACATCGTAGGAAAATGTCTCTG |
| 4010 | Right | CATGTTTTGATAACCTGACAGACAA |
| 4011 | Left | TACCTTGTTGTCTTCCTTCCTACAG |
| 4012 | Right | ACAATGAGGAAAACAAAATCTAGCA |
| 4013 | Left | TTACCTTGTTGTCTTCCTTCCTACA |
| 4014 | Right | GAGAGAACAACAGTCTGGGTAAAAA |
| 4015 | Left | CCATAGAGAACATCGTAGGAAAATG |
| 4016 | Right | CATGTTTTGATAACCTGACAGACAA |
| 4017 | Left | CTGGACAACATTGTTTTTAATTCCT |
| 4018 | Right | CATGTTTTGATAACCTGACAGACAA |
| 4019 | Left | TTACCTTGTTGTCTTCCTTCCTACA |
| 4020 | Right | AGAGAGAACAACAGTCTGGGTAAAA |
| 4021 | Left | TTACCTTGTTGTCTTCCTTCCTACA |
| 4022 | Right | AAGAGAGAACAACAGTCTGGGTAAA |
| 4023 | Left | TCTGGACAACATTGTTTTTAATTCC |
| 4024 | Right | CATGTTTTGATAACCTGACAGACAA |
| 4025 | Left | TTACCTTGTTGTCTTCCTTCCTACA |
| 4026 | Right | ACAATGAGGAAAACAAAATCTAGCA |
| 4027 | Left | TGGACAACATTGTTTTTAATTCCTT |
| 4028 | Right | TTATAATCTAGCATTGCCAAAATCA |
| 4029 | Left | TACCTTGTTGTCTTCCTTCCTACAG |
| 4030 | Right | CAATGAGGAAAACAAAATCTAGCAA |
| 4031 | Left | TACCTTGTTGTCTTCCTTCCTACAG |
| 4032 | Right | AACAATGAGGAAAACAAAATCTAGC |
| 4033 | Left | TACCTTGTTGTCTTCCTTCCTACAG |
| 4034 | Right | TTATAATCTAGCATTGCCAAAATCA |
| 4035 | Left | TCCTTTATTGATTTTGAAACTGCAC |
| 4036 | Right | CATGTTTTGATAACCTGACAGACAA |
| 4037 | Left | TGATAGGTTTGCCATAGAGAACATC |
| 4038 | Right | CATGTTTTGATAACCTGACAGACAA |
| 4039 | Left | TGGACAACATTGTTTTTAATTCCTT |
| 4040 | Right | CAGTTTATAATCTAGCATTGCCAAAA |

KIT Exon12-13 501-600 bases

| | | |
|---|---|---|
| 4041 | Left | TACCTTGTTGTCTTCCTTCCTACAG |
| 4042 | Right | ATGAGATATTCAAGAGGCTGATGTC |
| 4043 | Left | TACCTTGTTGTCTTCCTTCCTACAG |
| 4044 | Right | GATGAGATATTCAAGAGGCTGATGT |
| 4045 | Left | CTTTTGATAGGTTTGCCATAGAGAA |
| 4046 | Right | GAGAGAACAACAGTCTGGGTAAAAA |
| 4047 | Left | TGGACAACATTGTTTTTAATTCCTT |
| 4048 | Right | GAGAGAACAACAGTCTGGGTAAAAA |
| 4049 | Left | AGAACATCGTAGGAAAATGTCTCTG |
| 4050 | Right | GAGAGAACAACAGTCTGGGTAAAAA |
| 4051 | Left | CTTTTGATAGGTTTGCCATAGAGAA |
| 4052 | Right | AGAGAGAACAACAGTCTGGGTAAAA |
| 4053 | Left | CTTTTGATAGGTTTGCCATAGAGAA |
| 4054 | Right | AAGAGAGAACAACAGTCTGGGTAAA |
| 4055 | Left | TGGACAACATTGTTTTTAATTCCTT |
| 4056 | Right | AGAGAGAACAACAGTCTGGGTAAAA |
| 4057 | Left | TGGACAACATTGTTTTTAATTCCTT |
| 4058 | Right | AAGAGAGAACAACAGTCTGGGTAAA |
| 4059 | Left | AGAACATCGTAGGAAAATGTCTCTG |
| 4060 | Right | AGAGAGAACAACAGTCTGGGTAAAA |
| 4061 | Left | AGAACATCGTAGGAAAATGTCTCTG |
| 4062 | Right | AAGAGAGAACAACAGTCTGGGTAAA |
| 4063 | Left | CCATAGAGAACATCGTAGGAAAATG |
| 4064 | Right | GAGAGAACAACAGTCTGGGTAAAAA |
| 4065 | Left | TACCTTGTTGTCTTCCTTCCTACAG |
| 4066 | Right | ACGACAATAACTAGGGTATGTCCTG |
| 4067 | Left | CTTTTGATAGGTTTGCCATAGAGAA |
| 4068 | Right | CATGTTTTGATAACCTGACAGACAA |
| 4069 | Left | CTGGACAACATTGTTTTTAATTCCT |
| 4070 | Right | GAGAGAACAACAGTCTGGGTAAAAA |
| 4071 | Left | TACCTTGTTGTCTTCCTTCCTACAG |
| 4072 | Right | TCGTTGATGTTACAAATACGACAAT |
| 4073 | Left | TACCTTGTTGTCTTCCTTCCTACAG |
| 4074 | Right | GCTGATGTCGTTGATGTTACAAATA |
| 4075 | Left | TACCTTGTTGTCTTCCTTCCTACAG |
| 4076 | Right | GCTGTTCTACCCCATAATGATAAAA |
| 4077 | Left | TGGACAACATTGTTTTTAATTCCTT |
| 4078 | Right | ACAATGAGGAAAACAAAATCTAGCA |
| 4079 | Left | TTACCTTGTTGTCTTCCTTCCTACA |
| 4080 | Right | ATGAGATATTCAAGAGGCTGATGTC |

KIT Exon12-13 601-800 bases

| | | |
|---|---|---|
| 4081 | Left | CTTTTGATAGGTTTGCCATAGAGAA |
| 4082 | Right | GATGAGATATTCAAGAGGCTGATGT |
| 4083 | Left | CTTTTGATAGGTTTGCCATAGAGAA |
| 4084 | Right | ATGAGATATTCAAGAGGCTGATGTC |
| 4085 | Left | TGGACAACATTGTTTTTAATTCCTT |
| 4086 | Right | TTTCAGTGGCTACATATGATCAAGA |
| 4087 | Left | TGGACAACATTGTTTTTAATTCCTT |
| 4088 | Right | TTCAGTGGCTACATATGATCAAGAA |
| 4089 | Left | AGAACATCGTAGGAAAATGTCTCTG |
| 4090 | Right | ATGAGATATTCAAGAGGCTGATGTC |
| 4091 | Left | AGAACATCGTAGGAAAATGTCTCTG |
| 4092 | Right | GATGAGATATTCAAGAGGCTGATGT |
| 4093 | Left | AGAACATCGTAGGAAAATGTCTCTG |
| 4094 | Right | TCAGTGGCTACATATGATCAAGAAA |
| 4095 | Left | AGAACATCGTAGGAAAATGTCTCTG |
| 4096 | Right | TTCAGTGGCTACATATGATCAAGAA |
| 4097 | Left | AGAACATCGTAGGAAAATGTCTCTG |
| 4098 | Right | TTTCAGTGGCTACATATGATCAAGA |
| 4099 | Left | TACCTTGTTGTCTTCCTTCCTACAG |
| 4100 | Right | TCAGTGGCTACATATGATCAAGAAA |
| 4101 | Left | TACCTTGTTGTCTTCCTTCCTACAG |
| 4102 | Right | TTCAGTGGCTACATATGATCAAGAA |
| 4103 | Left | TACCTTGTTGTCTTCCTTCCTACAG |
| 4104 | Right | TTTCAGTGGCTACATATGATCAAGA |
| 4105 | Left | ACAGTGACTTTAAGGAACTCCAGTG |
| 4106 | Right | GATGAGATATTCAAGAGGCTGATGT |
| 4107 | Left | ACAGTGACTTTAAGGAACTCCAGTG |
| 4108 | Right | ATGAGATATTCAAGAGGCTGATGTC |
| 4109 | Left | TACCTTGTTGTCTTCCTTCCTACAG |
| 4110 | Right | ATGTTTGCCTCATTTGGTGTATATT |
| 4111 | Left | ACAGTGACTTTAAGGAACTCCAGTG |
| 4112 | Right | GAGAGAACAACAGTCTGGGTAAAAA |
| 4113 | Left | CCATAGAGAACATCGTAGGAAAATG |
| 4114 | Right | GATGAGATATTCAAGAGGCTGATGT |
| 4115 | Left | CCATAGAGAACATCGTAGGAAAATG |
| 4116 | Right | ATGAGATATTCAAGAGGCTGATGTC |
| 4117 | Left | TACCTTGTTGTCTTCCTTCCTACAG |
| 4118 | Right | AAGGCTTCAAATTGGAAACTTATTT |
| 4119 | Left | CTTTTGATAGGTTTGCCATAGAGAA |
| 4120 | Right | ACGACAATAACTAGGGTATGTCCTG |

KIT Exon10-13 801-1000 bases

| | | |
|---|---|---|
| 4121 | Left | AAGTTTGTGATTCCACATTTCTCTT |
| 4122 | Right | CATGTTTTGATAACCTGACAGACAA |
| 4123 | Left | GATTCCACATTTCTCTTCCATTGTA |
| 4124 | Right | CATGTTTTGATAACCTGACAGACAA |
| 4125 | Left | AGTTTGTGATTCCACATTTCTCTTC |
| 4126 | Right | TTGATAACCTGACAGACAATAAAAGG |
| 4127 | Left | AGTTTGTGATTCCACATTTCTCTTC |
| 4128 | Right | TGATAACCTGACAGACAATAAAAGG |
| 4129 | Left | CAAAGTTTGTGATTCCACATTTCTC |
| 4130 | Right | CATGTTTTGATAACCTGACAGACAA |
| 4131 | Left | AGTTTGTGATTCCACATTTCTCTTC |
| 4132 | Right | CATGTTTTGATAACCTGACAGACAAT |
| 4133 | Left | AAGTTTGTGATTCCACATTTCTCTT |
| 4134 | Right | TTGATAACCTGACAGACAATAAAAGG |
| 4135 | Left | AAAGTTTGTGATTCCACATTTCTCT |
| 4136 | Right | TTGATAACCTGACAGACAATAAAAGG |
| 4137 | Left | AAGTTTGTGATTCCACATTTCTCTT |
| 4138 | Right | TGATAACCTGACAGACAATAAAAGG |
| 4139 | Left | AAAGTTTGTGATTCCACATTTCTCT |
| 4140 | Right | TGATAACCTGACAGACAATAAAAGG |
| 4141 | Left | CAAAGTTTGTGATTCCACATTTCT |
| 4142 | Right | CATGTTTTGATAACCTGACAGACAA |
| 4143 | Left | GTGATTCCACATTTCTCTTCCATT |
| 4144 | Right | CATGTTTTGATAACCTGACAGACAA |
| 4145 | Left | AAAGTTTGTGATTCCACATTTCTCTT |
| 4146 | Right | CATGTTTTGATAACCTGACAGACAA |
| 4147 | Left | AGTTTGTGATTCCACATTTCTCTTC |
| 4148 | Right | ATGTTTTGATAACCTGACAGACAAT |
| 4149 | Left | AGTTTGTGATTCCACATTTCTCTTC |
| 4150 | Right | TGTTTTGATAACCTGACAGACAATAA |
| 4151 | Left | AGTTTGTGATTCCACATTTCTCTTC |
| 4152 | Right | CATGTTTTGATAACCTGACAGACA |
| 4153 | Left | AAGTTTGTGATTCCACATTTCTCTT |
| 4154 | Right | CATGTTTTGATAACCTGACAGACAAT |
| 4155 | Left | AGTTTGTGATTCCACATTTCTCTTC |
| 4156 | Right | CTGACAGACAATAAAAGGCAGCTT |
| 4157 | Left | AGTTTGTGATTCCACATTTCTCTTC |
| 4158 | Right | TGTTTTGATAACCTGACAGACAATA |
| 4159 | Left | CAAAGTTTGTGATTCCACATTTCTC |
| 4160 | Right | TTGATAACCTGACAGACAATAAAAGG |

KIT Exon10-13 2 kb

| | | |
|---|---|---|
| 4161 | Left | AATATAAACCGTCCATAAAGGAAGC |
| 4162 | Right | ATTATGGAAATAATGAAGGCACAAA |
| 4163 | Left | AATATAAACCGTCCATAAAGGAAGC |
| 4164 | Right | TTCTGAGCGCTACTAGTTGAAAAAT |
| 4165 | Left | AATATAAACCGTCCATAAAGGAAGC |
| 4166 | Right | ATTTATGGGGATTATTGGAAGACAT |
| 4167 | Left | TCTATTCTGCAGTATTGTGGTTTCA |
| 4168 | Right | TTCTGAGCGCTACTAGTTGAAAAAT |
| 4169 | Left | AATATAAACCGTCCATAAAGGAAGC |
| 4170 | Right | CTGGAAAGTGAGTGAAAACCTAAAA |
| 4171 | Left | AATATAAACCGTCCATAAAGGAAGC |
| 4172 | Right | GACTGGAAAGTGAGTGAAAACCTAA |
| 4173 | Left | AGTTTGTGATTCCACATTTCTCTTC |
| 4174 | Right | GATTAATTCTGCCTCCCATAAAAAT |
| 4175 | Left | ACTGCCTTTATGAAGTCATGTTAGC |
| 4176 | Right | ATGAGATATTCAAGAGGCTGATGTC |
| 4177 | Left | TCTGCAGTATTGTGGTTTCAAGTTA |
| 4178 | Right | TTCTGAGCGCTACTAGTTGAAAAAT |
| 4179 | Left | AGTTTGTGATTCCACATTTCTCTTC |
| 4180 | Right | AATATTCAGCTTCAGTGGATCAGAC |
| 4181 | Left | AGTTTGTGATTCCACATTTCTCTTC |
| 4182 | Right | GAATCCTCTGGTTCACTTCTGTTTA |
| 4183 | Left | TCTATTCTGCAGTATTGTGGTTTCA |
| 4184 | Right | GAATCCTCTGGTTCACTTCTGTTTA |
| 4185 | Left | AGAATCTTCCATGTTTTTCAGACAG |
| 4186 | Right | GACTGGAAAGTGAGTGAAAACCTAA |
| 4187 | Left | AATATAAACCGTCCATAAAGGAAGC |
| 4188 | Right | ACAAGACTGGAAAGTGAGTGAAAAC |
| 4189 | Left | TTTTAACACTTTGCCAGACACTGTA |
| 4190 | Right | GATGAGATATTCAAGAGGCTGATGT |
| 4191 | Left | TTTTAACACTTTGCCAGACACTGTA |
| 4192 | Right | ATGAGATATTCAAGAGGCTGATGTC |
| 4193 | Left | GGATGTTTAGGCTCTGTCTACCATA |
| 4194 | Right | GAGAGAACAACAGTCTGGGTAAAAA |
| 4195 | Left | TCTGCAGTATTGTGGTTTCAAGTTA |
| 4196 | Right | GAATCCTCTGGTTCACTTCTGTTTA |
| 4197 | Left | AATATAAACCGTCCATAAAGGAAGC |
| 4198 | Right | TATGGAAATAATGAAGGCACAAACT |
| 4199 | Left | AATACATGCATCACACCATACTGTC |
| 4200 | Right | GAGAGAACAACAGTCTGGGTAAAAA |

KIT Exon10-13 5 kb

| | | |
|---|---|---|
| 4201 | Left | GGATGTTTAGGCTCTGTCTACCATA |
| 4202 | Right | CAGAAACTAGGTGTCCTTTTTGTGT |
| 4203 | Left | GGATGTTTAGGCTCTGTCTACCATA |
| 4204 | Right | ACCAATAATTTGGATGATTTTCTGA |
| 4205 | Left | GGATGTTTAGGCTCTGTCTACCATA |
| 4206 | Right | TAATTTGGATGATTTTCTGAACCAT |
| 4207 | Left | GGATGTTTAGGCTCTGTCTACCATA |
| 4208 | Right | TCTGATTAGCATAGAAACACCATGA |
| 4209 | Left | GGATGTTTAGGCTCTGTCTACCATA |
| 4210 | Right | GGTATCCCGTATTACCTCAAACTCT |
| 4211 | Left | GGATGTTTAGGCTCTGTCTACCATA |
| 4212 | Right | ACTTCCTGCTTCTGATTAGCATAGA |
| 4213 | Left | ACTGCCTTTATGAAGTCATGTTAGC |
| 4214 | Right | CAGAAACTAGGTGTCCTTTTTGTGT |
| 4215 | Left | AATACATGCATCACACCATACTGTC |
| 4216 | Right | TAATTTGGATGATTTTCTGAACCAT |
| 4217 | Left | AATACATGCATCACACCATACTGTC |
| 4218 | Right | ACCAATAATTTGGATGATTTTCTGA |
| 4219 | Left | AATACATGCATCACACCATACTGTC |
| 4220 | Right | TCTGATTAGCATAGAAACACCATGA |
| 4221 | Left | AATACATGCATCACACCATACTGTC |
| 4222 | Right | GGTATCCCGTATTACCTCAAACTCT |
| 4223 | Left | AATACATGCATCACACCATACTGTC |
| 4224 | Right | ACTTCCTGCTTCTGATTAGCATAGA |
| 4225 | Left | ACTGCCTTTATGAAGTCATGTTAGC |
| 4226 | Right | GGTATCCCGTATTACCTCAAACTCT |
| 4227 | Left | AGAATCTTCCATGTTTTTCAGACAG |
| 4228 | Right | CTCATCCCCTTTGCTACATAATAGA |
| 4229 | Left | TTTTAACACTTTGCCAGACACTGTA |
| 4230 | Right | CAGAAACTAGGTGTCCTTTTTGTGT |
| 4231 | Left | GGATGTTTAGGCTCTGTCTACCATA |
| 4232 | Right | CTCATCCCCTTTGCTACATAATAGA |
| 4233 | Left | TTTTAACACTTTGCCAGACACTGTA |
| 4234 | Right | GGTATCCCGTATTACCTCAAACTCT |
| 4235 | Left | AATACATGCATCACACCATACTGTC |
| 4236 | Right | CTCATCCCCTTTGCTACATAATAGA |
| 4237 | Left | AGAATCTTCCATGTTTTTCAGACAG |
| 4238 | Right | CATAATAGACTGGACCAATTTGAGG |
| 4239 | Left | TTTAATCCAATTTAAGGGGATGTTT |
| 4240 | Right | CAGAAACTAGGTGTCCTTTTTGTGT |

KIT Exon17 151-200 bases

| | | |
|---|---|---|
| 4241 | Left | GTTTTCTTTTCTCCTCCAACCTAAT |
| 4242 | Right | ACTGTCAAGCAGAGAATGGGTACT |
| 4243 | Left | GTTTTCTTTTCTCCTCCAACCTAAT |
| 4244 | Right | CTGTCAAGCAGAGAATGGGTACT |
| 4245 | Left | TGAATTTAAATGGTTTTCTTTTCTCC |
| 4246 | Right | ACTGTCAAGCAGAGAATGGGTACT |
| 4247 | Left | GTTTTCTTTTCTCCTCCAACCTAAT |
| 4248 | Right | GACTGTCAAGCAGAGAATGGGTACT |
| 4249 | Left | TTTTCTTTTCTCCTCCAACCTAATA |
| 4250 | Right | ACTGTCAAGCAGAGAATGGGTACT |
| 4251 | Left | TAAATGGTTTTCTTTTCTCCTCCA |
| 4252 | Right | ACTGTCAAGCAGAGAATGGGTACT |
| 4253 | Left | TAAATGGTTTTCTTTTCTCCTCCAA |
| 4254 | Right | ACTGTCAAGCAGAGAATGGGTACT |
| 4255 | Left | TTAAATGGTTTTCTTTTCTCCTCCA |
| 4256 | Right | ACTGTCAAGCAGAGAATGGGTACT |
| 4257 | Left | GTTTTCTTTTCTCCTCCAACCTAATA |
| 4258 | Right | ACTGTCAAGCAGAGAATGGGTACT |
| 4259 | Left | GTTTTCTTTTCTCCTCCAACCTAAT |
| 4260 | Right | GACTGTCAAGCAGAGAATGGGTA |
| 4261 | Left | TGAATTTAAATGGTTTTCTTTTCTCC |
| 4262 | Right | CTGTCAAGCAGAGAATGGGTACT |
| 4263 | Left | TGAATTTAAATGGTTTTCTTTTCTCC |
| 4264 | Right | GACTGTCAAGCAGAGAATGGGTACT |
| 4265 | Left | TTTTCTTTTCTCCTCCAACCTAATA |
| 4266 | Right | CTGTCAAGCAGAGAATGGGTACT |
| 4267 | Left | GTTTTCTTTTCTCCTCCAACCTAA |
| 4268 | Right | ACTGTCAAGCAGAGAATGGGTACT |
| 4269 | Left | TTTTCTTTTCTCCTCCAACCTAATA |
| 4270 | Right | GACTGTCAAGCAGAGAATGGGTACT |
| 4271 | Left | TAAATGGTTTTCTTTTCTCCTCCA |
| 4272 | Right | CTGTCAAGCAGAGAATGGGTACT |
| 4273 | Left | TAAATGGTTTTCTTTTCTCCTCCA |
| 4274 | Right | GACTGTCAAGCAGAGAATGGGTACT |
| 4275 | Left | GTTTTCTTTTCTCCTCCAACCTAAT |
| 4276 | Right | CAGGACTGTCAAGCAGAGAATG |
| 4277 | Left | TAAATGGTTTTCTTTTCTCCTCCAA |
| 4278 | Right | CTGTCAAGCAGAGAATGGGTACT |
| 4279 | Left | TTAAATGGTTTTCTTTTCTCCTCCA |
| 4280 | Right | CTGTCAAGCAGAGAATGGGTACT |

KIT Exon17 201-300

| | | |
|---|---|---|
| 4281 | Left | GTTTTCTTTTCTCCTCCAACCTAAT |
| 4282 | Right | ATCACAGGAAACAATTTTTATCGAA |
| 4283 | Left | GTTTTCTTTTCTCCTCCAACCTAAT |
| 4284 | Right | AAATGTGTGATATCCCTAGACAGGA |
| 4285 | Left | GTTTTCTTTTCTCCTCCAACCTAAT |
| 4286 | Right | TGTGTGATATCCCTAGACAGGATTT |
| 4287 | Left | GTTTTCTTTTCTCCTCCAACCTAAT |
| 4288 | Right | CACAGGAAACAATTTTTATCGAAAG |
| 4289 | Left | GTTTTCTTTTCTCCTCCAACCTAAT |
| 4290 | Right | AAAATGTGTGATATCCCTAGACAGG |
| 4291 | Left | TGAATTTAAATGGTTTTCTTTTCTCC |
| 4292 | Right | TGTGTGATATCCCTAGACAGGATTT |
| 4293 | Left | TGAATTTAAATGGTTTTCTTTTCTCC |
| 4294 | Right | AAATGTGTGATATCCCTAGACAGGA |
| 4295 | Left | TTTTCTTTTCTCCTCCAACCTAATA |
| 4296 | Right | ATCACAGGAAACAATTTTTATCGAA |
| 4297 | Left | GTTTTCTTTTCTCCTCCAACCTAAT |
| 4298 | Right | ATGTGTGATATCCCTAGACAGGATT |
| 4299 | Left | GTTTTCTTTTCTCCTCCAACCTAAT |
| 4300 | Right | AATGTGTGATATCCCTAGACAGGAT |
| 4301 | Left | TTTTCTTTTCTCCTCCAACCTAATA |
| 4302 | Right | AAATGTGTGATATCCCTAGACAGGA |
| 4303 | Left | TTTTCTTTTCTCCTCCAACCTAATA |
| 4304 | Right | TGTGTGATATCCCTAGACAGGATTT |
| 4305 | Left | TGAATTTAAATGGTTTTCTTTTCTCC |
| 4306 | Right | CACAGGAAACAATTTTTATCGAAAG |
| 4307 | Left | TGAATTTAAATGGTTTTCTTTTCTCC |
| 4308 | Right | AAAATGTGTGATATCCCTAGACAGG |
| 4309 | Left | TTTTCTTTTCTCCTCCAACCTAATA |
| 4310 | Right | CACAGGAAACAATTTTTATCGAAAG |
| 4311 | Left | TAAATGGTTTTCTTTTCTCCTCCA |
| 4312 | Right | ATCACAGGAAACAATTTTTATCGAA |
| 4313 | Left | TAAATGGTTTTCTTTTCTCCTCCA |
| 4314 | Right | AAATGTGTGATATCCCTAGACAGGA |
| 4315 | Left | TAAATGGTTTTCTTTTCTCCTCCA |
| 4316 | Right | TGTGTGATATCCCTAGACAGGATTT |
| 4317 | Left | TTTTCTTTTCTCCTCCAACCTAATA |
| 4318 | Right | AAAATGTGTGATATCCCTAGACAGG |
| 4319 | Left | TAAATGGTTTTCTTTTCTCCTCCAA |
| 4320 | Right | ATCACAGGAAACAATTTTTATCGAA |

KIT Exon17 301-400

| | | |
|---|---|---|
| 4321 | Left | ATTCAAGGCGTACTTTTGATTTTTA |
| 4322 | Right | ATCACAGGAAACAATTTTTATCGAA |
| 4323 | Left | TCAAGGCGTACTTTTGATTTTTATT |
| 4324 | Right | ATCACAGGAAACAATTTTTATCGAA |
| 4325 | Left | TTCAAGGCGTACTTTTGATTTTTAT |
| 4326 | Right | ATCACAGGAAACAATTTTTATCGAA |
| 4327 | Left | ATTCAAGGCGTACTTTTGATTTTTA |
| 4328 | Right | CACAGGAAACAATTTTTATCGAAAG |
| 4329 | Left | GTTTTCTTTTCTCCTCCAACCTAAT |
| 4330 | Right | TAGTAATGTTCAGCATACCATGCAA |
| 4331 | Left | ATTCAAGGCGTACTTTTGATTTTTAT |
| 4332 | Right | ATCACAGGAAACAATTTTTATCGAA |
| 4333 | Left | ATCATTCAAGGCGTACTTTTGATTT |
| 4334 | Right | ATCACAGGAAACAATTTTTATCGAA |
| 4335 | Left | TTCAAGGCGTACTTTTGATTTTTAT |
| 4336 | Right | TCGAAAGTTGAAACTAAAAATCCTTT |
| 4337 | Left | ATTCAAGGCGTACTTTTGATTTTTA |
| 4338 | Right | TCACAGGAAACAATTTTTATCGAA |
| 4339 | Left | TCAAGGCGTACTTTTGATTTTTATT |
| 4340 | Right | TCACAGGAAACAATTTTTATCGAA |
| 4341 | Left | TTCAAGGCGTACTTTTGATTTTTAT |
| 4342 | Right | TCACAGGAAACAATTTTTATCGAA |
| 4343 | Left | ATTCAAGGCGTACTTTTGATTTTT |
| 4344 | Right | ATCACAGGAAACAATTTTTATCGAA |
| 4345 | Left | TTCAAGGCGTACTTTTGATTTTTATT |
| 4346 | Right | ATCACAGGAAACAATTTTTATCGAA |
| 4347 | Left | AGTCCTGAGAAGAAAACAGCATTTA |
| 4348 | Right | ACTGTCAAGCAGAGAATGGGTACT |
| 4349 | Left | ATTCAAGGCGTACTTTTGATTTTT |
| 4350 | Right | CACAGGAAACAATTTTTATCGAAAG |
| 4351 | Left | GTTTTCTTTTCTCCTCCAACCTAAT |
| 4352 | Right | GTAATGTTCAGCATACCATGCAAAT |
| 4353 | Left | CATTCAAGGCGTACTTTTGATTTT |
| 4354 | Right | ATCACAGGAAACAATTTTTATCGAA |
| 4355 | Left | TTCAAGGCGTACTTTTGATTTTTA |
| 4356 | Right | ATCACAGGAAACAATTTTTATCGAA |
| 4357 | Left | GAACATCATTCAAGGCGTACTTTT |
| 4358 | Right | ATCACAGGAAACAATTTTTATCGAA |
| 4359 | Left | CATTCAAGGCGTACTTTTGATTTTT |
| 4360 | Right | ATCACAGGAAACAATTTTTATCGAA |

KIT Exon17 401-500 bases

| | | |
|---|---|---|
| 4361 | Left | ATGTATTTCCCTATGAATGAAAGCA |
| 4362 | Right | ATCACAGGAAACAATTTTTATCGAA |
| 4363 | Left | TATTTCCCTATGAATGAAAGCAGTC |
| 4364 | Right | ATCACAGGAAACAATTTTTATCGAA |
| 4365 | Left | AGTCCTGAGAAGAAAACAGCATTTA |
| 4366 | Right | ATCACAGGAAACAATTTTTATCGAA |
| 4367 | Left | AGTCCTGAGAAGAAAACAGCATTTA |
| 4368 | Right | TGTGTGATATCCCTAGACAGGATTT |
| 4369 | Left | ATGTATTTCCCTATGAATGAAAGCA |
| 4370 | Right | CACAGGAAACAATTTTTATCGAAAG |
| 4371 | Left | TATTTCCCTATGAATGAAAGCAGTC |
| 4372 | Right | CACAGGAAACAATTTTTATCGAAAG |
| 4373 | Left | AGTCCTGAGAAGAAAACAGCATTTA |
| 4374 | Right | CACAGGAAACAATTTTTATCGAAAG |
| 4375 | Left | TCCTGAGAAGAAAACAGCATTTATT |
| 4376 | Right | ATCACAGGAAACAATTTTTATCGAA |
| 4377 | Left | TCCTGAGAAGAAAACAGCATTTATT |
| 4378 | Right | TGTGTGATATCCCTAGACAGGATTT |
| 4379 | Left | AGTCCTGAGAAGAAAACAGCATTTA |
| 4380 | Right | AAAATGTGTGATATCCCTAGACAGG |
| 4381 | Left | GTTTTCTTTTCTCCTCCAACCTAAT |
| 4382 | Right | AATCAAGTTCATTGCTATTCTCAGG |
| 4383 | Left | TCCTGAGAAGAAAACAGCATTTATT |
| 4384 | Right | CACAGGAAACAATTTTTATCGAAAG |
| 4385 | Left | GTTTTCTTTTCTCCTCCAACCTAAT |
| 4386 | Right | GCAAAATCAAGTTCATTGCTATTCT |
| 4387 | Left | GTTTTCTTTTCTCCTCCAACCTAAT |
| 4388 | Right | AGCAAAATCAAGTTCATTGCTATTC |
| 4389 | Left | TCCTGAGAAGAAAACAGCATTTATT |
| 4390 | Right | AAAATGTGTGATATCCCTAGACAGG |
| 4391 | Left | TCAAGGCGTACTTTTGATTTTTATT |
| 4392 | Right | AAATGTGTGATATCCCTAGACAGGA |
| 4393 | Left | TCAAGGCGTACTTTTGATTTTTATT |
| 4394 | Right | TGTGTGATATCCCTAGACAGGATTT |
| 4395 | Left | TTCAAGGCGTACTTTTGATTTTTAT |
| 4396 | Right | AAATGTGTGATATCCCTAGACAGGA |
| 4397 | Left | ATTCAAGGCGTACTTTTGATTTTTA |
| 4398 | Right | AAATGTGTGATATCCCTAGACAGGA |
| 4399 | Left | TTCAAGGCGTACTTTTGATTTTTAT |
| 4400 | Right | TGTGTGATATCCCTAGACAGGATTT |

KIT Exon17 501-600

| | | |
|---|---|---|
| 4401 | Left | ATGTATTTCCCTATGAATGAAAGCA |
| 4402 | Right | AAATGTGTGATATCCCTAGACAGGA |
| 4403 | Left | ATGTATTTCCCTATGAATGAAAGCA |
| 4404 | Right | TGTGTGATATCCCTAGACAGGATTT |
| 4405 | Left | TATTTCCCTATGAATGAAAGCAGTC |
| 4406 | Right | AAATGTGTGATATCCCTAGACAGGA |
| 4407 | Left | TATTTCCCTATGAATGAAAGCAGTC |
| 4408 | Right | TGTGTGATATCCCTAGACAGGATTT |
| 4409 | Left | GATTGAATTTGCAAAGGCATATTAG |
| 4410 | Right | ATCACAGGAAACAATTTTTATCGAA |
| 4411 | Left | ATGTATTTCCCTATGAATGAAAGCA |
| 4412 | Right | AAAATGTGTGATATCCCTAGACAGG |
| 4413 | Left | TATTTCCCTATGAATGAAAGCAGTC |
| 4414 | Right | AAAATGTGTGATATCCCTAGACAGG |
| 4415 | Left | GATTGAATTTGCAAAGGCATATTAG |
| 4416 | Right | CACAGGAAACAATTTTTATCGAAAG |
| 4417 | Left | AAAGGCATATTAGGAACTCTGTGAA |
| 4418 | Right | ATCACAGGAAACAATTTTTATCGAA |
| 4419 | Left | AAGGCATATTAGGAACTCTGTGAAA |
| 4420 | Right | ATCACAGGAAACAATTTTTATCGAA |
| 4421 | Left | TCAAGGCGTACTTTTGATTTTTATT |
| 4422 | Right | AATCAAGTTCATTGCTATTCTCAGG |
| 4423 | Left | TTCAAGGCGTACTTTTGATTTTTAT |
| 4424 | Right | AATCAAGTTCATTGCTATTCTCAGG |
| 4425 | Left | ATTCAAGGCGTACTTTTGATTTTTA |
| 4426 | Right | AATCAAGTTCATTGCTATTCTCAGG |
| 4427 | Left | TCAAGGCGTACTTTTGATTTTTATT |
| 4428 | Right | GCAAAATCAAGTTCATTGCTATTCT |
| 4429 | Left | TTCAAGGCGTACTTTTGATTTTTAT |
| 4430 | Right | GCAAAATCAAGTTCATTGCTATTCT |
| 4431 | Left | ATTCAAGGCGTACTTTTGATTTTTA |
| 4432 | Right | GCAAAATCAAGTTCATTGCTATTCT |
| 4433 | Left | TCAAGGCGTACTTTTGATTTTTATT |
| 4434 | Right | AGCAAAATCAAGTTCATTGCTATTC |
| 4435 | Left | TTCAAGGCGTACTTTTGATTTTTAT |
| 4436 | Right | AGCAAAATCAAGTTCATTGCTATTC |
| 4437 | Left | ATTCAAGGCGTACTTTTGATTTTTA |
| 4438 | Right | AGCAAAATCAAGTTCATTGCTATTC |
| 4439 | Left | AAAGGCATATTAGGAACTCTGTGAA |
| 4440 | Right | TGTGTGATATCCCTAGACAGGATTT |

KIT Exon17 601-800

| | | |
|---|---|---|
| 4441 | Left | TATTTCCCTATGAATGAAAGCAGTC |
| 4442 | Right | AATCAAGTTCATTGCTATTCTCAGG |
| 4443 | Left | TATTTCCCTATGAATGAAAGCAGTC |
| 4444 | Right | GCAAAATCAAGTTCATTGCTATTCT |
| 4445 | Left | ACATTTCCCAACAATTACCAAACTA |
| 4446 | Right | ATCACAGGAAACAATTTTTATCGAA |
| 4447 | Left | ACATTTCCCAACAATTACCAAACTA |
| 4448 | Right | AAATGTGTGATATCCCTAGACAGGA |
| 4449 | Left | ACATTTCCCAACAATTACCAAACTA |
| 4450 | Right | TGTGTGATATCCCTAGACAGGATTT |
| 4451 | Left | GAAGGTTAGGAATGGAAAGAATGAT |
| 4452 | Right | ATCACAGGAAACAATTTTTATCGAA |
| 4453 | Left | GATTGAATTTGCAAAGGCATATTAG |
| 4454 | Right | AATCAAGTTCATTGCTATTCTCAGG |
| 4455 | Left | TTCCCAACAATTACCAAACTAAGAA |
| 4456 | Right | ATCACAGGAAACAATTTTTATCGAA |
| 4457 | Left | TCCCAACAATTACCAAACTAAGAAA |
| 4458 | Right | ATCACAGGAAACAATTTTTATCGAA |
| 4459 | Left | TTTCCCAACAATTACCAAACTAAGA |
| 4460 | Right | ATCACAGGAAACAATTTTTATCGAA |
| 4461 | Left | GATTGAATTTGCAAAGGCATATTAG |
| 4462 | Right | GCAAAATCAAGTTCATTGCTATTCT |
| 4463 | Left | GATTGAATTTGCAAAGGCATATTAG |
| 4464 | Right | AGCAAAATCAAGTTCATTGCTATTC |
| 4465 | Left | TTACCAGTCCTACCCTTAAATGTCA |
| 4466 | Right | ATCACAGGAAACAATTTTTATCGAA |
| 4467 | Left | TCCCAACAATTACCAAACTAAGAAA |
| 4468 | Right | AAATGTGTGATATCCCTAGACAGGA |
| 4469 | Left | TTCCCAACAATTACCAAACTAAGAA |
| 4470 | Right | AAATGTGTGATATCCCTAGACAGGA |
| 4471 | Left | TTTCCCAACAATTACCAAACTAAGA |
| 4472 | Right | AAATGTGTGATATCCCTAGACAGGA |
| 4473 | Left | TTCCCAACAATTACCAAACTAAGAA |
| 4474 | Right | TGTGTGATATCCCTAGACAGGATTT |
| 4475 | Left | TTTCCCAACAATTACCAAACTAAGA |
| 4476 | Right | TGTGTGATATCCCTAGACAGGATTT |
| 4477 | Left | TCCCAACAATTACCAAACTAAGAAA |
| 4478 | Right | TGTGTGATATCCCTAGACAGGATTT |
| 4479 | Left | ATTAACCGAACAGAATGAGTTACCA |
| 4480 | Right | ATCACAGGAAACAATTTTTATCGAA |

KIT Exon17 801-1000 bases

| | | |
|---|---|---|
| 4481 | Left | ACATTTCCCAACAATTACCAAACTA |
| 4482 | Right | AATCAAGTTCATTGCTATTCTCAGG |
| 4483 | Left | ACATTTCCCAACAATTACCAAACTA |
| 4484 | Right | GCAAAATCAAGTTCATTGCTATTCT |
| 4485 | Left | ACATTTCCCAACAATTACCAAACTA |
| 4486 | Right | AGCAAAATCAAGTTCATTGCTATTC |
| 4487 | Left | ATGTATTTCCCTATGAATGAAAGCA |
| 4488 | Right | CATGCCTTAGTTTCTCCAACTTTTA |
| 4489 | Left | TATTTCCCTATGAATGAAAGCAGTC |
| 4490 | Right | CATGCCTTAGTTTCTCCAACTTTTA |
| 4491 | Left | TCCCAACAATTACCAAACTAAGAAA |
| 4492 | Right | AATCAAGTTCATTGCTATTCTCAGG |
| 4493 | Left | TTTCCCAACAATTACCAAACTAAGA |
| 4494 | Right | AATCAAGTTCATTGCTATTCTCAGG |
| 4495 | Left | TCCCAACAATTACCAAACTAAGAAA |
| 4496 | Right | GCAAAATCAAGTTCATTGCTATTCT |
| 4497 | Left | TCCCAACAATTACCAAACTAAGAAA |
| 4498 | Right | AGCAAAATCAAGTTCATTGCTATTC |
| 4499 | Left | TTCCCAACAATTACCAAACTAAGAA |
| 4500 | Right | AGCAAAATCAAGTTCATTGCTATTC |
| 4501 | Left | TTTCCCAACAATTACCAAACTAAGA |
| 4502 | Right | GCAAAATCAAGTTCATTGCTATTCT |
| 4503 | Left | TTTCCCAACAATTACCAAACTAAGA |
| 4504 | Right | AGCAAAATCAAGTTCATTGCTATTC |
| 4505 | Left | GGGTGAAGCATAGACTTGAGTTTTA |
| 4506 | Right | ATCACAGGAAACAATTTTTATCGAA |
| 4507 | Left | ATTAACCGAACAGAATGAGTTACCA |
| 4508 | Right | AATCAAGTTCATTGCTATTCTCAGG |
| 4509 | Left | TTACCAGTCCTACCCTTAAATGTCA |
| 4510 | Right | GCAAAATCAAGTTCATTGCTATTCT |
| 4511 | Left | TTACCAGTCCTACCCTTAAATGTCA |
| 4512 | Right | AGCAAAATCAAGTTCATTGCTATTC |
| 4513 | Left | GGGTGAAGCATAGACTTGAGTTTTA |
| 4514 | Right | AAATGTGTGATATCCCTAGACAGGA |
| 4515 | Left | GGGTGAAGCATAGACTTGAGTTTTA |
| 4516 | Right | TGTGTGATATCCCTAGACAGGATTT |
| 4517 | Left | AGTCCTGAGAAGAAAACAGCATTTA |
| 4518 | Right | CATGCCTTAGTTTCTCCAACTTTTA |
| 4519 | Left | ATTAACCGAACAGAATGAGTTACCA |
| 4520 | Right | GCAAAATCAAGTTCATTGCTATTCT |

KIT Exon17 2 kb

| | | |
|---|---|---|
| 4521 | Left | AACCAAAAGCAGAGGAAATTTAGTT |
| 4522 | Right | AACACTCTTACAAAACCAAATCGAG |
| 4523 | Left | AGCCATAGTTAAAATGCAGAATGTC |
| 4524 | Right | AACACTCTTACAAAACCAAATCGAG |
| 4525 | Left | GAGCCATAGTTAAAATGCAGAATGT |
| 4526 | Right | AACACTCTTACAAAACCAAATCGAG |
| 4527 | Left | AGAGCCATAGTTAAAATGCAGAATG |
| 4528 | Right | AACACTCTTACAAAACCAAATCGAG |
| 4529 | Left | ACATTTCCCAACAATTACCAAACTA |
| 4530 | Right | AGTATGTAACCACCGTCACGATTAT |
| 4531 | Left | GGGTGAAGCATAGACTTGAGTTTTA |
| 4532 | Right | AGTATGTAACCACCGTCACGATTAT |
| 4533 | Left | GGGTGAAGCATAGACTTGAGTTTTA |
| 4534 | Right | TTGGACAACTCTTGACAAAATTACA |
| 4535 | Left | GAAGGTTAGGAATGGAAAGAATGAT |
| 4536 | Right | AGTATGTAACCACCGTCACGATTAT |
| 4537 | Left | GGGTGAAGCATAGACTTGAGTTTTA |
| 4538 | Right | ATTCAGAGGTATTGGACAACTCTTG |
| 4539 | Left | GAAGGTTAGGAATGGAAAGAATGAT |
| 4540 | Right | TTGGACAACTCTTGACAAAATTACA |
| 4541 | Left | ATGTATTTCCCTATGAATGAAAGCA |
| 4542 | Right | AAAACCCACAATTACTTTTACACCA |
| 4543 | Left | TATTTCCCTATGAATGAAAGCAGTC |
| 4544 | Right | AAAACCCACAATTACTTTTACACCA |
| 4545 | Left | AACCAAAAGCAGAGGAAATTTAGTT |
| 4546 | Right | ATTACATTATCATAAGGGGCACAAA |
| 4547 | Left | GAAGGTTAGGAATGGAAAGAATGAT |
| 4548 | Right | ATTCAGAGGTATTGGACAACTCTTG |
| 4549 | Left | AGCCATAGTTAAAATGCAGAATGTC |
| 4550 | Right | GGCAGAGAATATTATAAAGGGCAAT |
| 4551 | Left | GAGCCATAGTTAAAATGCAGAATGT |
| 4552 | Right | GGCAGAGAATATTATAAAGGGCAAT |
| 4553 | Left | AGAGCCATAGTTAAAATGCAGAATG |
| 4554 | Right | GGCAGAGAATATTATAAAGGGCAAT |
| 4555 | Left | AGCCATAGTTAAAATGCAGAATGTC |
| 4556 | Right | ATTACATTATCATAAGGGGCACAAA |
| 4557 | Left | GAGCCATAGTTAAAATGCAGAATGT |
| 4558 | Right | ATTACATTATCATAAGGGGCACAAA |
| 4559 | Left | AGAGCCATAGTTAAAATGCAGAATG |
| 4560 | Right | ATTACATTATCATAAGGGGCACAAA |

KIT Exon17 5 kb

| | | |
|---|---|---|
| 4561 | Left | ATGGTTCAGAAAATCATCCAAATTA |
| 4562 | Right | TCTGCCATAAAAAGCTAAATCAATC |
| 4563 | Left | TCAGAAAATCATCCAAATTATTGGT |
| 4564 | Right | TCTGCCATAAAAAGCTAAATCAATC |
| 4565 | Left | GTATATTGCTGCAGTTGTGTGGTAG |
| 4566 | Right | TAAGGGCTCCTAACCTGAGATCTAT |
| 4567 | Left | TCATGGTGTTTCTATGCTAATCAGA |
| 4568 | Right | TCTGCCATAAAAAGCTAAATCAATC |
| 4569 | Left | GTACCTACCTATCAAGCAACCAAGA |
| 4570 | Right | CTTGAGTACCATCTCACAAAAACCT |
| 4571 | Left | GAGTACCTACCTATCAAGCAACCAA |
| 4572 | Right | CTTGAGTACCATCTCACAAAAACCT |
| 4573 | Left | TCTATGCTAATCAGAAGCAGGAAGT |
| 4574 | Right | TCTGCCATAAAAAGCTAAATCAATC |
| 4575 | Left | ATGGTTCAGAAAATCATCCAAATTA |
| 4576 | Right | GGCCACTAAGTTGTAAGTGCTGTAT |
| 4577 | Left | TCAGAAAATCATCCAAATTATTGGT |
| 4578 | Right | GGCCACTAAGTTGTAAGTGCTGTAT |
| 4579 | Left | GTATATTGCTGCAGTTGTGTGGTAG |
| 4580 | Right | AAAACCCACAATTACTTTTACACCA |
| 4581 | Left | GTACCTACCTATCAAGCAACCAAGA |
| 4582 | Right | GGCCACTAAGTTGTAAGTGCTGTAT |
| 4583 | Left | GAGTACCTACCTATCAAGCAACCAA |
| 4584 | Right | GGCCACTAAGTTGTAAGTGCTGTAT |
| 4585 | Left | TCATGGTGTTTCTATGCTAATCAGA |
| 4586 | Right | TAAGGGCTCCTAACCTGAGATCTAT |
| 4587 | Left | GTAACCCAGCCTAGGATTGTTAAAT |
| 4588 | Right | CTTGAGTACCATCTCACAAAAACCT |
| 4589 | Left | TCATGGTGTTTCTATGCTAATCAGA |
| 4590 | Right | GGCCACTAAGTTGTAAGTGCTGTAT |
| 4591 | Left | AACCAAAAGCAGAGGAAATTTAGTT |
| 4592 | Right | CAGTGTGTCATAAAGAATCCAAGTG |
| 4593 | Left | TCTATGCTAATCAGAAGCAGGAAGT |
| 4594 | Right | TAAGGGCTCCTAACCTGAGATCTAT |
| 4595 | Left | ATGTTTTTGTGCCTGAGTATCTTTC |
| 4596 | Right | CAGTGTGTCATAAAGAATCCAAGTG |
| 4597 | Left | TCTATGCTAATCAGAAGCAGGAAGT |
| 4598 | Right | GGCCACTAAGTTGTAAGTGCTGTAT |
| 4599 | Left | AACCAAAAGCAGAGGAAATTTAGTT |
| 4600 | Right | CAATTTGCAACCTAAGATTAGGAGA |

TABLE 10
KRAS Capture Primer List for NGS Panel

| | | |
|---|---|---|
| Seq. | | |
| ID | Primer | Sequence |

KRAS Exon1 169-300 bases

| | | |
|---|---|---|
| 4601 | Left | CTCGGAGCTCGATTTTCCTA |
| 4602 | Right | GGGACCCCTAATTCATTCACTC |
| 4603 | Left | CTCGGAGCTCGATTTTCCTA |
| 4604 | Right | GGGACCCCTAATTCATTCACT |
| 4605 | Left | CTCGGAGCTCGATTTTCCTA |
| 4606 | Right | GGACCCCTAATTCATTCACTCG |
| 4607 | Left | CTCGGAGCTCGATTTTCCT |
| 4608 | Right | GGGACCCCTAATTCATTCACTC |
| 4609 | Left | GTGCTCGGAGCTCGATTTT |
| 4610 | Right | GGGACCCCTAATTCATTCACTC |
| 4611 | Left | CTCGGAGCTCGATTTTCCTA |
| 4612 | Right | GGGGACCCCTAATTCATTCACT |
| 4613 | Left | GCTCGGAGCTCGATTTTCCTA |
| 4614 | Right | GGGACCCCTAATTCATTCACTC |
| 4615 | Left | CTCGGAGCTCGATTTTCCTA |
| 4616 | Right | GACCCCTAATTCATTCACTCG |
| 4617 | Left | CTCGGAGCTCGATTTTCCTA |
| 4618 | Right | GGGGACCCCTAATTCATTCAC |
| 4619 | Left | GTGCTCGGAGCTCGATTTTC |
| 4620 | Right | GGGACCCCTAATTCATTCACTC |
| 4621 | Left | TGCTCGGAGCTCGATTTT |
| 4622 | Right | GGGACCCCTAATTCATTCACTC |
| 4623 | Left | CTCGGAGCTCGATTTTCCT |
| 4624 | Right | GGGACCCCTAATTCATTCACT |
| 4625 | Left | CTCGGAGCTCGATTTTCCTA |
| 4626 | Right | GGGGACCCCTAATTCATTCA |
| 4627 | Left | GTGCTCGGAGCTCGATTTT |
| 4628 | Right | GGGACCCCTAATTCATTCACT |
| 4629 | Left | TCGGAGCTCGATTTTCCTA |
| 4630 | Right | GGGACCCCTAATTCATTCACTC |
| 4631 | Left | CTCGGAGCTCGATTTTCCT |
| 4632 | Right | GGACCCCTAATTCATTCACTCG |
| 4633 | Left | GTGCTCGGAGCTCGATTTT |
| 4634 | Right | GGACCCCTAATTCATTCACTCG |
| 4635 | Left | GTGTGCTCGGAGCTCGATT |
| 4636 | Right | GGGACCCCTAATTCATTCACTC |
| 4637 | Left | CTCGGAGCTCGATTTTCCTA |
| 4638 | Right | GGGGACCCCTAATTCATTCACTC |
| 4639 | Left | TGCTCGGAGCTCGATTTTC |
| 4640 | Right | GGGACCCCTAATTCATTCACTC |

KRAS Exon1 301-400 bases

| | | |
|---|---|---|
| 4641 | Left | CCCGTCTGAAGAAGAATCGAG |
| 4642 | Right | GGGACCCCTAATTCATTCACTC |
| 4643 | Left | GTACGCCCGTCTGAAGAAGA |
| 4644 | Right | GGGACCCCTAATTCATTCACTC |
| 4645 | Left | CATCGATAGCTCTGCCCTCT |
| 4646 | Right | GGGACCCCTAATTCATTCACTC |
| 4647 | Left | ATCGATAGCTCTGCCCTCTG |
| 4648 | Right | GGGACCCCTAATTCATTCACTC |
| 4649 | Left | GTACGCCCGTCTGAAGAAGAATC |
| 4650 | Right | GGGACCCCTAATTCATTCACTC |
| 4651 | Left | GTACGCCCGTCTGAAGAAGAA |
| 4652 | Right | GGGACCCCTAATTCATTCACTC |
| 4653 | Left | GTACGCCCGTCTGAAGAAGAAT |
| 4654 | Right | GGGACCCCTAATTCATTCACT |
| 4655 | Left | TACGCCCGTCTGAAGAAGAATC |
| 4656 | Right | GGGACCCCTAATTCATTCACTC |
| 4657 | Left | GTACGCCCGTCTGAAGAAGAAT |
| 4658 | Right | GGACCCCTAATTCATTCACTCG |
| 4659 | Left | TACGCCCGTCTGAAGAAGAA |
| 4660 | Right | GGGACCCCTAATTCATTCACTC |
| 4661 | Left | GGAACGCATCGATAGCTCTG |
| 4662 | Right | GGGACCCCTAATTCATTCACTC |
| 4663 | Left | TACGCCCGTCTGAAGAAGAAT |
| 4664 | Right | GGGACCCCTAATTCATTCACT |
| 4665 | Left | TACGCCCGTCTGAAGAAGAAT |
| 4666 | Right | GGACCCCTAATTCATTCACTCG |
| 4667 | Left | CCCGTCTGAAGAAGAATCGAG |
| 4668 | Right | GGGACCCCTAATTCATTCACT |
| 4669 | Left | CATCGATAGCTCTGCCCTCTG |
| 4670 | Right | GGGACCCCTAATTCATTCACTC |
| 4671 | Left | GTACGCCCGTCTGAAGAAGA |
| 4672 | Right | GGGACCCCTAATTCATTCACT |
| 4673 | Left | GTACGCCCGTCTGAAGAAGAAT |
| 4674 | Right | GGGGACCCCTAATTCATTCACT |
| 4675 | Left | ATCGATAGCTCTGCCCTCTG |
| 4676 | Right | GGGACCCCTAATTCATTCACT |
| 4677 | Left | CATCGATAGCTCTGCCCTCT |
| 4678 | Right | GGGACCCCTAATTCATTCACT |
| 4679 | Left | GTACGCCCGTCTGAAGAAGAATC |
| 4680 | Right | GGGACCCCTAATTCATTCACT |

KRAS Exon1 401-600 bases

| | | |
|---|---|---|
| 4681 | Left | CTCTTCCCTCTTCCCACACC |
| 4682 | Right | GGGACCCCTAATTCATTCACTC |
| 4683 | Left | CCTCTTCCCTCTTCCCACAC |
| 4684 | Right | GGGACCCCTAATTCATTCACTC |
| 4685 | Left | TCTTCCCTCTTCCCACACC |
| 4686 | Right | GGGACCCCTAATTCATTCACTC |
| 4687 | Left | CCTCTTCCCTCTTCCCACA |
| 4688 | Right | GGGACCCCTAATTCATTCACTC |
| 4689 | Left | CTCTTCCCTCTTCCCACACC |
| 4690 | Right | GGGACCCCTAATTCATTCACT |
| 4691 | Left | CCTCTTCCCTCTTCCCACAC |
| 4692 | Right | GGGACCCCTAATTCATTCACT |
| 4693 | Left | TCTTCCCTCTTCCCACACC |
| 4694 | Right | GGGACCCCTAATTCATTCACT |
| 4695 | Left | CTCTTCCCTCTTCCCACACC |
| 4696 | Right | GGACCCCTAATTCATTCACTCG |
| 4697 | Left | CCTCTTCCCTCTTCCCACAC |
| 4698 | Right | GGACCCCTAATTCATTCACTCG |
| 4699 | Left | TCTTCCCTCTTCCCACACC |
| 4700 | Right | GGACCCCTAATTCATTCACTCG |
| 4701 | Left | CCTCTTCCCTCTTCCCACA |
| 4702 | Right | GGGACCCCTAATTCATTCACT |
| 4703 | Left | CCTCTTCCCTCTTCCCACA |
| 4704 | Right | GGACCCCTAATTCATTCACTCG |
| 4705 | Left | GTACGCCCGTCTGAAGAAGAAT |
| 4706 | Right | GAGGAGGAAGGAAGGGGTTC |
| 4707 | Left | GTACGCCCGTCTGAAGAAGAAT |
| 4708 | Right | AGGAGGAAGGAAGGGGTTC |
| 4709 | Left | TACGCCCGTCTGAAGAAGAAT |
| 4710 | Right | GAGGAGGAAGGAAGGGGTTC |
| 4711 | Left | CTCTTCCCTCTTCCCACACC |
| 4712 | Right | GGGGACCCCTAATTCATTCACT |
| 4713 | Left | CCTCTTCCCTCTTCCCACAC |
| 4714 | Right | GGGGACCCCTAATTCATTCACT |
| 4715 | Left | TCTTCCCTCTTCCCACACC |
| 4716 | Right | GGGGACCCCTAATTCATTCACT |
| 4717 | Left | CTCTTCCCTCTTCCCACACC |
| 4718 | Right | GACCCCTAATTCATTCACTCG |
| 4719 | Left | CCTCTTCCCTCTTCCCACAC |
| 4720 | Right | GACCCCTAATTCATTCACTCG |

KRAS Exon1 601-800 bases

| | | |
|---|---|---|
| 4721 | Left | GTACGCCCGTCTGAAGAAGAAT |
| 4722 | Right | GTTTATACCTTCGTCCTAGAGATGC |
| 4723 | Left | GTACGCCCGTCTGAAGAAGAAT |
| 4724 | Right | AGTTTATACCTTCGTCCTAGAGATGC |
| 4725 | Left | TACGCCCGTCTGAAGAAGAAT |
| 4726 | Right | GTTTATACCTTCGTCCTAGAGATGC |
| 4727 | Left | CCCGTCTGAAGAAGAATCGAG |
| 4728 | Right | GTTTATACCTTCGTCCTAGAGATGC |
| 4729 | Left | TACGCCCGTCTGAAGAAGAAT |
| 4730 | Right | AGTTTATACCTTCGTCCTAGAGATGC |
| 4731 | Left | GTACGCCCGTCTGAAGAAGA |
| 4732 | Right | GTTTATACCTTCGTCCTAGAGATGC |
| 4733 | Left | ATCGATAGCTCTGCCCTCTG |
| 4734 | Right | GTTTATACCTTCGTCCTAGAGATGC |
| 4735 | Left | CATCGATAGCTCTGCCCTCT |
| 4736 | Right | GTTTATACCTTCGTCCTAGAGATGC |
| 4737 | Left | GTACGCCCGTCTGAAGAAGAATC |
| 4738 | Right | GTTTATACCTTCGTCCTAGAGATGC |
| 4739 | Left | CCCGTCTGAAGAAGAATCGAG |
| 4740 | Right | AGTTTATACCTTCGTCCTAGAGATGC |
| 4741 | Left | CTCGGAGCTCGATTTTCCTA |
| 4742 | Right | CTTACTCACACATCCCCTACACAC |
| 4743 | Left | CTCGGAGCTCGATTTTCCTA |
| 4744 | Right | GTTTATACCTTCGTCCTAGAGATGC |
| 4745 | Left | CCCGTCTGAAGAAGAATCGAG |
| 4746 | Right | GGAGAAGTTTATACCTTCGTCCTAGA |
| 4747 | Left | GTACGCCCGTCTGAAGAAGA |
| 4748 | Right | AGTTTATACCTTCGTCCTAGAGATGC |
| 4749 | Left | ATCGATAGCTCTGCCCTCTG |
| 4750 | Right | AGTTTATACCTTCGTCCTAGAGATGC |
| 4751 | Left | CATCGATAGCTCTGCCCTCT |
| 4752 | Right | AGTTTATACCTTCGTCCTAGAGATGC |
| 4753 | Left | GTACGCCCGTCTGAAGAAGAATC |
| 4754 | Right | AGTTTATACCTTCGTCCTAGAGATGC |
| 4755 | Left | CTCGGAGCTCGATTTTCCTA |
| 4756 | Right | AGTTTATACCTTCGTCCTAGAGATGC |
| 4757 | Left | ATCGATAGCTCTGCCCTCTG |
| 4758 | Right | GGAGAAGTTTATACCTTCGTCCTAGA |
| 4759 | Left | CATCGATAGCTCTGCCCTCT |
| 4760 | Right | GGAGAAGTTTATACCTTCGTCCTAGA |

KRAS Exon1 801-1000 bases

| | | |
|---|---|---|
| 4761 | Left | ACTTTTGGTGACTGCTTGTTTATTT |
| 4762 | Right | GGGACCCCTAATTCATTCACTC |
| 4763 | Left | CACTTTTGGTGACTGCTTGTTTATT |
| 4764 | Right | GGGACCCCTAATTCATTCACTC |
| 4765 | Left | GTACGCCCGTCTGAAGAAGAAT |
| 4766 | Right | ACAGTGGTCTCTAAGCACTTTCCTA |
| 4767 | Left | GTACGCCCGTCTGAAGAAGAAT |
| 4768 | Right | AAACACAATAACCTCAAACAGTGGT |
| 4769 | Left | CTTTTGGTGACTGCTTGTTTATTTA |
| 4770 | Right | GGGACCCCTAATTCATTCACTC |
| 4771 | Left | GTACGCCCGTCTGAAGAAGAAT |
| 4772 | Right | TAACCTCAAACAGTGGTCTCTAAGC |
| 4773 | Left | GTACGCCCGTCTGAAGAAGAAT |
| 4774 | Right | ACACAATAACCTCAAACAGTGGTCT |
| 4775 | Left | TTTTGGTGACTGCTTGTTTATTTAC |
| 4776 | Right | GGGACCCCTAATTCATTCACTC |
| 4777 | Left | TACGCCCGTCTGAAGAAGAAT |
| 4778 | Right | ACAGTGGTCTCTAAGCACTTTCCTA |
| 4779 | Left | TACGCCCGTCTGAAGAAGAAT |
| 4780 | Right | AAACACAATAACCTCAAACAGTGGT |
| 4781 | Left | CACTTTTGGTGACTGCTTGTTTAT |
| 4782 | Right | GGGACCCCTAATTCATTCACTC |
| 4783 | Left | ACTTTTGGTGACTGCTTGTTTATTT |
| 4784 | Right | GGGACCCCTAATTCATTCACT |
| 4785 | Left | GTACGCCCGTCTGAAGAAGAAT |
| 4786 | Right | ACAATAACCTCAAACAGTGGTCTCT |
| 4787 | Left | CTTTTGGTGACTGCTTGTTTATTTAC |
| 4788 | Right | GGGACCCCTAATTCATTCACTC |
| 4789 | Left | ACTTTTGGTGACTGCTTGTTTATTTA |
| 4790 | Right | GGGACCCCTAATTCATTCACTC |
| 4791 | Left | ACTTTTGGTGACTGCTTGTTTATTT |
| 4792 | Right | GGACCCCTAATTCATTCACTCG |
| 4793 | Left | TACGCCCGTCTGAAGAAGAAT |
| 4794 | Right | TAACCTCAAACAGTGGTCTCTAAGC |
| 4795 | Left | CCCGTCTGAAGAAGAATCGAG |
| 4796 | Right | ACAGTGGTCTCTAAGCACTTTCCTA |
| 4797 | Left | TACGCCCGTCTGAAGAAGAAT |
| 4798 | Right | ACACAATAACCTCAAACAGTGGTCT |
| 4799 | Left | CCCGTCTGAAGAAGAATCGAG |
| 4800 | Right | AAACACAATAACCTCAAACAGTGGT |

KRAS Exon1 2 kb

| | | |
|---|---|---|
| 4801 | Left | GGGATTTAAATTCAGCTTTATTGGT |
| 4802 | Right | ACAGTGGTCTCTAAGCACTTTCCTA |
| 4803 | Left | GGGATTTAAATTCAGCTTTATTGGT |
| 4804 | Right | CATCTGGGATTAACTTTTTCCTTTT |
| 4805 | Left | TCAAGACTCTCCCAAGATACATTTC |
| 4806 | Right | TTTGCTATTGCTGTCTACACTCAAC |
| 4807 | Left | TCAAGACTCTCCCAAGATACATTTC |
| 4808 | Right | ACAGTGGTCTCTAAGCACTTTCCTA |
| 4809 | Left | GGGATTTAAATTCAGCTTTATTGGT |
| 4810 | Right | AAACACAATAACCTCAAACAGTGGT |
| 4811 | Left | TCAAGACTCTCCCAAGATACATTTC |
| 4812 | Right | CATCTGGGATTAACTTTTTCCTTTT |
| 4813 | Left | TCAAGACTCTCCCAAGATACATTTC |
| 4814 | Right | AAACACAATAACCTCAAACAGTGGT |
| 4815 | Left | GTAGAAAGGAAAGGATGACAGTTGA |
| 4816 | Right | AAACACAATAACCTCAAACAGTGGT |
| 4817 | Left | GATTACAGCCCGTGTAAGAGTAGAA |
| 4818 | Right | ACAGTGGTCTCTAAGCACTTTCCTA |
| 4819 | Left | GATTACAGCCCGTGTAAGAGTAGAA |
| 4820 | Right | AAACACAATAACCTCAAACAGTGGT |
| 4821 | Left | GGGATTTAAATTCAGCTTTATTGGT |
| 4822 | Right | TAACCTCAAACAGTGGTCTCTAAGC |
| 4823 | Left | TCAAGACTCTCCCAAGATACATTTC |
| 4824 | Right | ATAAGAAATAGGGGAAAGGACAAGA |
| 4825 | Left | TCAAGACTCTCCCAAGATACATTTC |
| 4826 | Right | TAACCTCAAACAGTGGTCTCTAAGC |
| 4827 | Left | TTCAGCTTTATTGGTGGTTTATGAT |
| 4828 | Right | ACAGTGGTCTCTAAGCACTTTCCTA |
| 4829 | Left | ATTCAGCTTTATTGGTGGTTTATGA |
| 4830 | Right | ACAGTGGTCTCTAAGCACTTTCCTA |
| 4831 | Left | GGGATTTAAATTCAGCTTTATTGGT |
| 4832 | Right | ACACAATAACCTCAAACAGTGGTCT |
| 4833 | Left | TTCAGCTTTATTGGTGGTTTATGAT |
| 4834 | Right | CATCTGGGATTAACTTTTTCCTTTT |
| 4835 | Left | ATTCAGCTTTATTGGTGGTTTATGA |
| 4836 | Right | CATCTGGGATTAACTTTTTCCTTTT |
| 4837 | Left | ATTCAGCTTTATTGGTGGTTTATGA |
| 4838 | Right | AAACACAATAACCTCAAACAGTGGT |
| 4839 | Left | TTCAGCTTTATTGGTGGTTTATGAT |
| 4840 | Right | AAACACAATAACCTCAAACAGTGGT |

KRAS Exon1 5 kb

| | | |
|---|---|---|
| 4841 | Left | GGGATTTAAATTCAGCTTTATTGGT |
| 4842 | Right | CAAAGCAATTAGGAATAGATGAGGA |
| 4843 | Left | ACGTAAGTAAGGAAGGGAGAACAGT |
| 4844 | Right | CAAAGCAATTAGGAATAGATGAGGA |
| 4845 | Left | AGCAGTAAATGAAACAGACCAAAAC |
| 4846 | Right | CAAAGCAATTAGGAATAGATGAGGA |
| 4847 | Left | GGGATTTAAATTCAGCTTTATTGGT |
| 4848 | Right | TCCTTTCCCTCATGTAACACATAAT |
| 4849 | Left | TGCTTTGAATGTTAGTCACAGAGAG |
| 4850 | Right | TGATGGATCTCAAGATTTAAGAAGG |
| 4851 | Left | ACGTAAGTAAGGAAGGGAGAACAGT |
| 4852 | Right | AACAGTTCTCAAAATGTGGTCTAGG |
| 4853 | Left | ACGTAAGTAAGGAAGGGAGAACAGT |
| 4854 | Right | TCCTTTCCCTCATGTAACACATAAT |
| 4855 | Left | CGAATCATGAGCCTAGATGATAACT |
| 4856 | Right | ATCCAACAATTTTGTAATGGAAGAA |
| 4857 | Left | CGAATCATGAGCCTAGATGATAACT |
| 4858 | Right | AATCCAACAATTTTGTAATGGAAGA |
| 4859 | Left | CGAATCATGAGCCTAGATGATAACT |
| 4860 | Right | GAATCCAACAATTTTGTAATGGAAG |
| 4861 | Left | AGCAGTAAATGAAACAGACCAAAAC |
| 4862 | Right | AACAGTTCTCAAAATGTGGTCTAGG |
| 4863 | Left | AGCTCTGGAGAAAAAGTAGGAAAAG |
| 4864 | Right | CAAAGCAATTAGGAATAGATGAGGA |
| 4865 | Left | TCAAGACTCTCCCAAGATACATTTC |
| 4866 | Right | TCCTTTCCCTCATGTAACACATAAT |
| 4867 | Left | TAGAGTGACTATGATCCGACATGAA |
| 4868 | Right | CAAAGCAATTAGGAATAGATGAGGA |
| 4869 | Left | CTCCTTTTAAAAACACTTTGGAACA |
| 4870 | Right | CAAAGCAATTAGGAATAGATGAGGA |
| 4871 | Left | ACGTAAGTAAGGAAGGGAGAACAGT |
| 4872 | Right | TCATGTTACCAAGTAATGGGCTTAT |
| 4873 | Left | GAAGAGGGTAGGGGATATCAAATAA |
| 4874 | Right | CAAAGCAATTAGGAATAGATGAGGA |
| 4875 | Left | GCTGGAAATTTAGCAGTAAATGAAA |
| 4876 | Right | CAAAGCAATTAGGAATAGATGAGGA |
| 4877 | Left | GGAAACAAGAACTTATCATGCACTT |
| 4878 | Right | ATCCAACAATTTTGTAATGGAAGAA |
| 4879 | Left | GGAAACAAGAACTTATCATGCACTT |
| 4880 | Right | GAATCCAACAATTTTGTAATGGAAG |

KRAS Exon2 169-210 bases

| | | |
|---|---|---|
| 4881 | Left | CATGTTCTAATATAGTCACATTTTCA |
| 4882 | Right | AAGAATGGTCCTGCACCAGTAATA |
| 4883 | Left | CATGTTCTAATATAGTCACATTTTCA |
| 4884 | Right | AGAATGGTCCTGCACCAGTAATA |
| 4885 | Left | ACATGTTCTAATATAGTCACATTTTCA |
| 4886 | Right | AGAATGGTCCTGCACCAGTAATA |
| 4887 | Left | GACATGTTCTAATATAGTCACATTTT |
| | | CA |
| 4888 | Right | GAATGGTCCTGCACCAGTAATATG |
| 4889 | Left | CATGTTCTAATATAGTCACATTTTCATT |
| 4890 | Right | AAGAATGGTCCTGCACCAGTAATA |
| 4891 | Left | CATGTTCTAATATAGTCACATTTTCATT |
| 4892 | Right | AGAATGGTCCTGCACCAGTAATA |
| 4893 | Left | CATGTTCTAATATAGTCACATTTTCAT |
| 4894 | Right | AAGAATGGTCCTGCACCAGTAATA |
| 4895 | Left | CATGTTCTAATATAGTCACATTTTCAT |
| 4896 | Right | AGAATGGTCCTGCACCAGTAATA |
| 4897 | Left | CATGTTCTAATATAGTCACATTTTCA |
| 4898 | Right | GAATGGTCCTGCACCAGTAATATG |
| 4899 | Left | ACATGTTCTAATATAGTCACATTTTC |
| | | AT |
| 4900 | Right | AGAATGGTCCTGCACCAGTAATA |
| 4901 | Left | GACATGTTCTAATATAGTCACATTTT |
| | | CATT |
| 4902 | Right | GAATGGTCCTGCACCAGTAATATG |
| 4901 | Left | GACATGTTCTAATATAGTCACATTTT |
| | | CATT |
| 4902 | Right | GAATGGTCCTGCACCAGTAATATG |
| 4903 | Left | ACATGTTCTAATATAGTCACATTTTCA |
| 4904 | Right | GAATGGTCCTGCACCAGTAATATG |
| 4905 | Left | GACATGTTCTAATATAGTCACATTTT |
| | | CAT |
| 4906 | Right | GAATGGTCCTGCACCAGTAATATG |
| 4907 | Left | CATGTTCTAATATAGTCACATTTTCATT |
| 4908 | Right | GAATGGTCCTGCACCAGTAATATG |
| 4909 | Left | CATGTTCTAATATAGTCACATTTTCA |
| 4910 | Right | AGAATGGTCCTGCACCAGTAAT |
| 4911 | Left | TGACATGTTCTAATATAGTCACATTT |
| | | TCAT |
| 4912 | Right | ATGGTCCTGCACCAGTAATATG |
| 4913 | Left | ACATGTTCTAATATAGTCACATTTTCA |
| 4914 | Right | AGAATGGTCCTGCACCAGTAAT |
| 4915 | Left | CATGTTCTAATATAGTCACATTTTCAT |
| 4916 | Right | GAATGGTCCTGCACCAGTAATATG |
| 4917 | Left | TGACATGTTCTAATATAGTCACATTT |
| | | TCAT |
| 4918 | Right | ATGGTCCTGCACCAGTAATATGC |
| 4919 | Left | TGACATGTTCTAATATAGTCACATTT |
| | | TCAT |
| 4920 | Right | AATGGTCCTGCACCAGTAATATGC |

KRAS Exon2 211-300 bases

| | | |
|---|---|---|
| 4921 | Left | AAAAGGTACTGGTGGAGTATTTGAT |
| 4922 | Right | ATGAAAATGGTCAGAGAAACCTTTA |
| 4923 | Left | TTAAAAGGTACTGGTGGAGTATTTGA |
| 4924 | Right | ATGAAAATGGTCAGAGAAACCTTTA |
| 4925 | Left | TAAAAGGTACTGGTGGAGTATTTGA |
| 4926 | Right | ATGAAAATGGTCAGAGAAACCTTTA |
| 4927 | Left | GGTACTGGTGGAGTATTTGATAGTG |
| 4928 | Right | ATGAAAATGGTCAGAGAAACCTTTA |
| 4929 | Left | AGGTACTGGTGGAGTATTTGATAGTG |
| 4930 | Right | ATGAAAATGGTCAGAGAAACCTTTA |
| 4931 | Left | TTTGTATTAAAAGGTACTGGTGGAG |
| 4932 | Right | ATGAAAATGGTCAGAGAAACCTTTA |
| 4933 | Left | AAGGTACTGGTGGAGTATTTGATAGTG |
| 4934 | Right | ATGAAAATGGTCAGAGAAACCTTTA |
| 4935 | Left | GGTACTGGTGGAGTATTTGATAGTGT |
| 4936 | Right | ATGAAAATGGTCAGAGAAACCTTTA |
| 4937 | Left | AAAAGGTACTGGTGGAGTATTTGAT |
| 4938 | Right | TACTCATGAAAATGGTCAGAGAAAC |
| 4939 | Left | GTTTGTATTAAAAGGTACTGGTGGA |
| 4940 | Right | ATGAAAATGGTCAGAGAAACCTTTA |
| 4941 | Left | TTAAAAGGTACTGGTGGAGTATTTGA |
| 4942 | Right | TACTCATGAAAATGGTCAGAGAAAC |
| 4943 | Left | AGGTACTGGTGGAGTATTTGATAGTGT |
| 4944 | Right | ATGAAAATGGTCAGAGAAACCTTTA |
| 4945 | Left | ATTAAAAGGTACTGGTGGAGTATTTGA |
| 4946 | Right | ATGAAAATGGTCAGAGAAACCTTTA |
| 4947 | Left | TTTGTATTAAAAGGTACTGGTGGAGT |
| 4948 | Right | ATGAAAATGGTCAGAGAAACCTTTA |
| 4949 | Left | GTTTGTATTAAAAGGTACTGGTGGAG |
| 4950 | Right | ATGAAAATGGTCAGAGAAACCTTTA |
| 4951 | Left | AAAAGGTACTGGTGGAGTATTTGATA |
| 4952 | Right | ATGAAAATGGTCAGAGAAACCTTTA |
| 4953 | Left | TAAAAGGTACTGGTGGAGTATTTGA |
| 4954 | Right | TACTCATGAAAATGGTCAGAGAAAC |
| 4955 | Left | TTGTATTAAAAGGTACTGGTGGAGT |
| 4956 | Right | ATGAAAATGGTCAGAGAAACCTTTA |
| 4957 | Left | GGTACTGGTGGAGTATTTGATAGTG |
| 4958 | Right | TACTCATGAAAATGGTCAGAGAAAC |
| 4959 | Left | AAAGGTACTGGTGGAGTATTTGATA |
| 4960 | Right | ATGAAAATGGTCAGAGAAACCTTTA |

KRAS Exon2 301-400 bases

| | | |
|---|---|---|
| 4961 | Left | AAAAGGTACTGGTGGAGTATTTGAT |
| 4962 | Right | CCAAGGAAAGTAAAGTTCCCATATT |
| 4963 | Left | TTAAAAGGTACTGGTGGAGTATTTGA |
| 4964 | Right | CCAAGGAAAGTAAAGTTCCCATATT |
| 4965 | Left | TAAAAGGTACTGGTGGAGTATTTGA |
| 4966 | Right | CCAAGGAAAGTAAAGTTCCCATATT |
| 4967 | Left | AAAAGGTACTGGTGGAGTATTTGAT |
| 4968 | Right | TAACTTGAAACCCAAGGTACATTTC |
| 4969 | Left | AAAAGGTACTGGTGGAGTATTTGAT |
| 4970 | Right | GAAACCCAAGGTACATTTCAGATAA |
| 4971 | Left | TTAAAAGGTACTGGTGGAGTATTTGA |
| 4972 | Right | TAACTTGAAACCCAAGGTACATTTC |
| 4973 | Left | GGTACTGGTGGAGTATTTGATAGTG |
| 4974 | Right | CCAAGGAAAGTAAAGTTCCCATATT |
| 4975 | Left | TGAAGTACAGTTCATTACGATACACG |
| 4976 | Right | ATGAAAATGGTCAGAGAAACCTTTA |
| 4977 | Left | TAAAAGGTACTGGTGGAGTATTTGA |
| 4978 | Right | TAACTTGAAACCCAAGGTACATTTC |
| 4979 | Left | AGGTACTGGTGGAGTATTTGATAGTG |
| 4980 | Right | CCAAGGAAAGTAAAGTTCCCATATT |
| 4981 | Left | CAGTCAACTGGAATTTTCATGATT |
| 4982 | Right | ATGAAAATGGTCAGAGAAACCTTTA |
| 4983 | Left | TTAAAAGGTACTGGTGGAGTATTTG |
| 4984 | Right | CCAAGGAAAGTAAAGTTCCCATATT |
| 4985 | Left | AAGGTACTGGTGGAGTATTTGATAGTG |
| 4986 | Right | CCAAGGAAAGTAAAGTTCCCATATT |
| 4987 | Left | GGTACTGGTGGAGTATTTGATAGTGT |
| 4988 | Right | CCAAGGAAAGTAAAGTTCCCATATT |
| 4989 | Left | GGTACTGGTGGAGTATTTGATAGTG |
| 4990 | Right | TAACTTGAAACCCAAGGTACATTTC |
| 4991 | Left | ATTACGATACACGTCTGCAGTCAAC |
| 4992 | Right | ATGAAAATGGTCAGAGAAACCTTTA |
| 4993 | Left | ACAGTTCATTACGATACACGTCTGC |
| 4994 | Right | ATGAAAATGGTCAGAGAAACCTTTA |
| 4995 | Left | TACAGTTCATTACGATACACGTCTG |
| 4996 | Right | ATGAAAATGGTCAGAGAAACCTTTA |
| 4997 | Left | TGGAGGAGTTTGTAAATGAAGTACAG |
| 4998 | Right | TTTATCTGTATCAAAGAATGGTCCTG |
| 4999 | Left | AGGTACTGGTGGAGTATTTGATAGTG |
| 5000 | Right | TAACTTGAAACCCAAGGTACATTTC |

KRAS Exon2 401-600 bases

| | | |
|---|---|---|
| 5001 | Left | AGTCATGATATGATCCTTTGAGAGC |
| 5002 | Right | ATGAAAATGGTCAGAGAAACCTTTA |
| 5003 | Left | ATATGATCCTTTGAGAGCCTTTAGC |
| 5004 | Right | GAAACCCAAGGTACATTTCAGATAA |
| 5005 | Left | TGATATGATCCTTTGAGAGCCTTTA |
| 5006 | Right | ATGAAAATGGTCAGAGAAACCTTTA |
| 5007 | Left | ATATGATCCTTTGAGAGCCTTTAGC |
| 5008 | Right | ATGAAAATGGTCAGAGAAACCTTTA |
| 5009 | Left | TGAAGTCATGATATGATCCTTTGAG |
| 5010 | Right | ATGAAAATGGTCAGAGAAACCTTTA |
| 5011 | Left | TGGAGGAGTTTGTAAATGAAGTACAG |
| 5012 | Right | CCAAGGAAAGTAAAGTTCCCATATT |
| 5013 | Left | TGGAGGAGTTTGTAAATGAAGTACAG |
| 5014 | Right | TGACATACTCCCAAGGAAAGTAAAG |
| 5015 | Left | TGGAGGAGTTTGTAAATGAAGTACAG |
| 5016 | Right | CTGACATACTCCCAAGGAAAGTAAA |
| 5017 | Left | ATGATATGATCCTTTGAGAGCCTTT |
| 5018 | Right | ATGAAAATGGTCAGAGAAACCTTTA |
| 5019 | Left | AGTCATGATATGATCCTTTGAGAGC |
| 5020 | Right | TTTATCTGTATCAAAGAATGGTCCTG |
| 5021 | Left | AGTCATGATATGATCCTTTGAGAGC |
| 5022 | Right | TACTCATGAAAATGGTCAGAGAAAC |
| 5023 | Left | TGGAGGAGTTTGTAAATGAAGTACAG |
| 5024 | Right | TAACTTGAAACCCAAGGTACATTTC |
| 5025 | Left | TGGAGGAGTTTGTAAATGAAGTACAG |
| 5026 | Right | GAAACCCAAGGTACATTTCAGATAA |
| 5027 | Left | AGTCATGATATGATCCTTTGAGAGC |
| 5028 | Right | TTATCTGTATCAAAGAATGGTCCTG |
| 5029 | Left | ATATGATCCTTTGAGAGCCTTTAGC |
| 5030 | Right | TGAAACCCAAGGTACATTTCAGATA |
| 5031 | Left | GATATGATCCTTTGAGAGCCTTTAG |
| 5032 | Right | GAAACCCAAGGTACATTTCAGATAA |
| 5033 | Left | TGAAGTACAGTTCATTACGATACACG |
| 5034 | Right | CCAAGGAAAGTAAAGTTCCCATATT |
| 5035 | Left | TGAAGTACAGTTCATTACGATACACG |
| 5036 | Right | CGAAACTCTGAAATACACTTCCAAT |
| 5037 | Left | TGAAGTACAGTTCATTACGATACACG |
| 5038 | Right | CTGACATACTCCCAAGGAAAGTAAA |
| 5039 | Left | TGAAGTACAGTTCATTACGATACACG |
| 5040 | Right | TGACATACTCCCAAGGAAAGTAAAG |

KRAS Exon2 601-800 bases

| | | |
|---|---|---|
| 5041 | Left | AGTCATGATATGATCCTTTGAGAGC |
| 5042 | Right | CCAAGGAAAGTAAAGTTCCCATATT |
| 5043 | Left | AGTCATGATATGATCCTTTGAGAGC |
| 5044 | Right | CGAAACTCTGAAATACACTTCCAAT |
| 5045 | Left | AGTCATGATATGATCCTTTGAGAGC |
| 5046 | Right | TGACATACTCCCAAGGAAAGTAAAG |
| 5047 | Left | AGTCATGATATGATCCTTTGAGAGC |
| 5048 | Right | CTGACATACTCCCAAGGAAAGTAAA |
| 5049 | Left | AGTCATGATATGATCCTTTGAGAGC |
| 5050 | Right | AATTTCTACCCTCTCACGAAACTCT |
| 5051 | Left | AGTCATGATATGATCCTTTGAGAGC |
| 5052 | Right | GATACAAATTTCTACCCTCTCACGA |
| 5053 | Left | TCTGTAGCTGTTGCATATTGACTTC |
| 5054 | Right | CCAAGGAAAGTAAAGTTCCCATATT |
| 5055 | Left | TTTTTCTGTAGCTGTTGCATATTGA |
| 5056 | Right | CCAAGGAAAGTAAAGTTCCCATATT |
| 5057 | Left | AGTCATGATATGATCCTTTGAGAGC |
| 5058 | Right | TAACTTGAAACCCAAGGTACATTTC |
| 5059 | Left | AGTCATGATATGATCCTTTGAGAGC |
| 5060 | Right | GAAACCCAAGGTACATTTCAGATAA |
| 5061 | Left | CTACTGCCATGATGCTTTAAAAGTT |
| 5062 | Right | TAACTTGAAACCCAAGGTACATTTC |
| 5063 | Left | AGAGCACTGTGAAGTCTCTACATGA |
| 5064 | Right | CCAAGGAAAGTAAAGTTCCCATATT |
| 5065 | Left | AGTCATGATATGATCCTTTGAGAGC |
| 5066 | Right | TACAAATTTCTACCCTCTCACGAAA |
| 5067 | Left | AGTCATGATATGATCCTTTGAGAGC |
| 5068 | Right | AAATTTCTACCCTCTCACGAAACTC |
| 5069 | Left | CTACTGCCATGATGCTTTAAAAGTT |
| 5070 | Right | GAAACCCAAGGTACATTTCAGATAA |
| 5071 | Left | AGAGCACTGTGAAGTCTCTACATGA |
| 5072 | Right | CTGACATACTCCCAAGGAAAGTAAA |
| 5073 | Left | AGAGCACTGTGAAGTCTCTACATGA |
| 5074 | Right | TGACATACTCCCAAGGAAAGTAAAG |
| 5075 | Left | TCTGTAGCTGTTGCATATTGACTTC |
| 5076 | Right | TAACTTGAAACCCAAGGTACATTTC |
| 5077 | Left | AGAGCACTGTGAAGTCTCTACATGA |
| 5078 | Right | AATTTCTACCCTCTCACGAAACTCT |
| 5079 | Left | AGTCATGATATGATCCTTTGAGAGC |
| 5080 | Right | ACGAAACTCTGAAATACACTTCCAA |

KRAS Exon2 801-1000 bases

| | | |
|---|---|---|
| 5081 | Left | ATCCAGCTTTATTTGACACTCATTC |
| 5082 | Right | CCAAGGAAAGTAAAGTTCCCATATT |
| 5083 | Left | CTACTGCCATGATGCTTTAAAAGTT |
| 5084 | Right | CCAAGGAAAGTAAAGTTCCCATATT |
| 5085 | Left | ATCCAGCTTTATTTGACACTCATTC |
| 5086 | Right | CGAAACTCTGAAATACACTTCCAAT |
| 5087 | Left | AATATTGTTCTTCTTTGCCTCAGTG |
| 5088 | Right | TGACATACTCCCAAGGAAAGTAAAG |
| 5089 | Left | AATATTGTTCTTCTTTGCCTCAGTG |
| 5090 | Right | CTGACATACTCCCAAGGAAAGTAAA |
| 5091 | Left | ATCCAGCTTTATTTGACACTCATTC |
| 5092 | Right | TGACATACTCCCAAGGAAAGTAAAG |
| 5093 | Left | ATCCAGCTTTATTTGACACTCATTC |
| 5094 | Right | CTGACATACTCCCAAGGAAAGTAAA |
| 5095 | Left | CTACTGCCATGATGCTTTAAAAGTT |
| 5096 | Right | CGAAACTCTGAAATACACTTCCAAT |
| 5097 | Left | CTACTGCCATGATGCTTTAAAAGTT |
| 5098 | Right | TGACATACTCCCAAGGAAAGTAAAG |
| 5099 | Left | CTACTGCCATGATGCTTTAAAAGTT |
| 5100 | Right | CTGACATACTCCCAAGGAAAGTAAA |
| 5101 | Left | ATCCAGCTTTATTTGACACTCATTC |
| 5102 | Right | AATTTCTACCCTCTCACGAAACTCT |
| 5103 | Left | ATCCAGCTTTATTTGACACTCATTC |
| 5104 | Right | GATACAAATTTCTACCCTCTCACGA |
| 5105 | Left | CTACTGCCATGATGCTTTAAAAGTT |
| 5106 | Right | GATACAAATTTCTACCCTCTCACGA |
| 5107 | Left | TCTGTAGCTGTTGCATATTGACTTC |
| 5108 | Right | CGAAACTCTGAAATACACTTCCAAT |
| 5109 | Left | TCTGTAGCTGTTGCATATTGACTTC |
| 5110 | Right | TGACATACTCCCAAGGAAAGTAAAG |
| 5111 | Left | TCTGTAGCTGTTGCATATTGACTTC |
| 5112 | Right | AATTTCTACCCTCTCACGAAACTCT |
| 5113 | Left | TCTGTAGCTGTTGCATATTGACTTC |
| 5114 | Right | GATACAAATTTCTACCCTCTCACGA |
| 5115 | Left | AAAAGTTTTTCTGTAGCTGTTGCAT |
| 5116 | Right | CCAAGGAAAGTAAAGTTCCCATATT |
| 5117 | Left | TTTTTCTGTAGCTGTTGCATATTGA |
| 5118 | Right | CGAAACTCTGAAATACACTTCCAAT |
| 5119 | Left | TTTTTCTGTAGCTGTTGCATATTGA |
| 5120 | Right | TGACATACTCCCAAGGAAAGTAAAG |

KRAS Exon2 2 kb

| | | |
|---|---|---|
| 5121 | Left | AGTCATGATATGATCCTTTGAGAGC |
| 5122 | Right | TCCTAACCCACTTTATCACATTCAT |
| 5123 | Left | AGTCATGATATGATCCTTTGAGAGC |
| 5124 | Right | AATTAGACTGTTCCCCTTTACTGCT |
| 5125 | Left | ATTATGTGTTACATGAGGGAAAGGA |
| 5126 | Right | CCAAGGAAAGTAAAGTTCCCATATT |
| 5127 | Left | AGTCATGATATGATCCTTTGAGAGC |
| 5128 | Right | TTGGAAACAAAGTGTAATGGAATTT |
| 5129 | Left | AGTCATGATATGATCCTTTGAGAGC |
| 5130 | Right | TCAATTAGACTGTTCCCCTTTACTG |
| 5131 | Left | ATTATGTGTTACATGAGGGAAAGGA |
| 5132 | Right | CGAAACTCTGAAATACACTTCCAAT |
| 5133 | Left | ATTATGTGTTACATGAGGGAAAGGA |
| 5134 | Right | TGACATACTCCCAAGGAAAGTAAAG |
| 5135 | Left | ATTATGTGTTACATGAGGGAAAGGA |
| 5136 | Right | CTGACATACTCCCAAGGAAAGTAAA |
| 5137 | Left | AATATTGTTCTTCTTTGCCTCAGTG |
| 5138 | Right | TCTTTGCAAATAGGCATTATTTCTC |
| 5139 | Left | ATCCAGCTTTATTTGACACTCATTC |
| 5140 | Right | TCTTTGCAAATAGGCATTATTTCTC |
| 5141 | Left | TCTTCAGTCAATTATGATGCTGTGT |
| 5142 | Right | CCAAGGAAAGTAAAGTTCCCATATT |
| 5143 | Left | GTCTTCAGTCAATTATGATGCTGTG |
| 5144 | Right | CCAAGGAAAGTAAAGTTCCCATATT |
| 5145 | Left | AATATTGTTCTTCTTTGCCTCAGTG |
| 5146 | Right | AATTAGACTGTTCCCCTTTACTGCT |
| 5147 | Left | ATCCAGCTTTATTTGACACTCATTC |
| 5148 | Right | AATTAGACTGTTCCCCTTTACTGCT |
| 5149 | Left | CTACTGCCATGATGCTTTAAAAGTT |
| 5150 | Right | TCTTTGCAAATAGGCATTATTTCTC |
| 5151 | Left | ATTATGTGTTACATGAGGGAAAGGA |
| 5152 | Right | GATACAAATTTCTACCCTCTCACGA |
| 5153 | Left | GCACATTCATTAATTTGGAGCTACT |
| 5154 | Right | CTGACATACTCCCAAGGAAAGTAAA |
| 5155 | Left | CTACTGCCATGATGCTTTAAAAGTT |
| 5156 | Right | AATTAGACTGTTCCCCTTTACTGCT |
| 5157 | Left | AATATTGTTCTTCTTTGCCTCAGTG |
| 5158 | Right | ACCAAAAATATGTGACGTTTCCTTA |
| 5159 | Left | AATATTGTTCTTCTTTGCCTCAGTG |
| 5160 | Right | TACCAAAAATATGTGACGTTTCCTT |

KRAS Exon2 5 kb

| | | |
|---|---|---|
| 5161 | Left | CAGCCAATAAGTCTAGGTAGAGCAG |
| 5162 | Right | TAAAGATGAAACAAACCAATCCAAT |
| 5163 | Left | AGCCTTCTTAAATCTTGAGATCCAT |
| 5164 | Right | TCTTTGCAAATAGGCATTATTTCTC |
| 5165 | Left | ATTATGTGTTACATGAGGGAAAGGA |
| 5166 | Right | TAAAGATGAAACAAACCAATCCAAT |
| 5167 | Left | GGCTAGTAAACTTTTTGGCCTTAAC |
| 5168 | Right | TCTTTGCAAATAGGCATTATTTCTC |
| 5169 | Left | AGCCTTCTTAAATCTTGAGATCCAT |
| 5170 | Right | AATTAGACTGTTCCCCTTTACTGCT |
| 5171 | Left | GGCTAGTAAACTTTTTGGCCTTAAC |
| 5172 | Right | TCCTAACCCACTTTATCACATTCAT |
| 5173 | Left | GGCTAGTAAACTTTTTGGCCTTAAC |
| 5174 | Right | AATTAGACTGTTCCCCTTTACTGCT |
| 5175 | Left | TTTGCTTTTAAGAGATGGTAGATGG |
| 5176 | Right | TCTTTGCAAATAGGCATTATTTCTC |
| 5177 | Left | CAGCCAATAAGTCTAGGTAGAGCAG |
| 5178 | Right | TTGAACTGAATTATAAGTGCCACAA |
| 5179 | Left | GGCTAGTAAACTTTTTGGCCTTAAC |
| 5180 | Right | TTGGAAACAAAGTGTAATGGAATTT |
| 5181 | Left | TCCTCATCTATTCCTAATTGCTTTG |
| 5182 | Right | TTGAACTGAATTATAAGTGCCACAA |
| 5183 | Left | TTTGCTTTTAAGAGATGGTAGATGG |
| 5184 | Right | TCCTAACCCACTTTATCACATTCAT |
| 5185 | Left | GCACATTCATTAATTTGGAGCTACT |
| 5186 | Right | TAAAGATGAAACAAACCAATCCAAT |
| 5187 | Left | GACTTAAACATGTGCATCTCCTTTT |
| 5188 | Right | AAATGACAACAAAGCAAAGGTAAAG |
| 5189 | Left | TCCTCATCTATTCCTAATTGCTTTG |
| 5190 | Right | CCTTACTGAATAGGAAACTGTTCCA |
| 5191 | Left | TTTGCTTTTAAGAGATGGTAGATGG |
| 5192 | Right | AATTAGACTGTTCCCCTTTACTGCT |
| 5193 | Left | AGTCATGATATGATCCTTTGAGAGC |
| 5194 | Right | AATTAGCATGATTGCCTAGAAACAC |
| 5195 | Left | AGCCTTCTTAAATCTTGAGATCCAT |
| 5196 | Right | ACCAAAAATATGTGACGTTTCCTTA |
| 5197 | Left | AGCCTTCTTAAATCTTGAGATCCAT |
| 5198 | Right | TACCAAAAATATGTGACGTTTCCTT |
| 5199 | Left | AGCCTTCTTAAATCTTGAGATCCAT |
| 5200 | Right | TCAATTAGACTGTTCCCCTTTACTG |

TABLE 11
ALK cDNA Capture Primer List for NGS Panel

| | | |
|---|---|---|
| Seq. | | |
| ID | Primer | Sequence |

ALK Region1 75-125 bases

| | | |
|---|---|---|
| 5201 | Left | ctgcacactggccgtct |
| 5202 | Right | aaccatgcttccctggagtg |
| 5203 | Left | actgcacactggccg |
| 5204 | Right | accatgcttccctgga |
| 5205 | Left | actgcacactgg |
| 5206 | Right | accatgcttccct |

ALK Region1 126-175 bases

| | | |
|---|---|---|
| 5207 | Left | acaccatcctgagtccgtg |
| 5208 | Right | cactgtccaaccatgcttcc |
| 5209 | Left | gactccaagcacaccatcct |
| 5210 | Right | aaccatgcttccctggagtg |
| 5211 | Left | cctgagtccgtggatgagg |
| 5212 | Right | ctggagcactgtccaaccat |
| 5213 | Left | tccaagcacaccatcctgag |
| 5214 | Right | ccaaccatgcttccctgga |
| 5215 | Left | ctgcacactggccgtct |
| 5216 | Right | ctcgaaatgggttgtctggac |
| 5217 | Left | ccgtggatgaggagcagc |
| 5218 | Right | gattcttccctggagcactgtc |
| 5219 | Left | gagcactgcacactggc |
| 5220 | Right | cgattcttccctggagcact |
| 5221 | Left | atgaggagcagcagtgag |
| 5222 | Right | gattcttccctggagcactgtccaa |
| 5223 | Left | catcctgagtccgtggat |
| 5224 | Right | agcactgtccaaccatgct |
| 5225 | Left | tgagcactgcacact |
| 5226 | Right | cccgattcttccctggagc |
| 5227 | Left | atgaggagcagcagt |
| 5228 | Right | gacgcccgattcttccct |
| 5229 | Left | gtgagcactgcac |
| 5230 | Right | aatgggttgtctggacgcc |

ALK Region1 176-225 bases

| | | |
|---|---|---|
| 5231 | Left | ctcctttctccttctcaacacct |
| 5232 | Right | attcttccctggagcactgtc |
| 5233 | Left | cttctcaacacctcagctgact |
| 5234 | Right | cgattcttccctggagcact |
| 5235 | Left | tttctccttctcaacacctcagct |
| 5236 | Right | gcactgtccaaccatgcttc |
| 5237 | Left | ctgactccaagcacaccatc |
| 5238 | Right | ctcgaaatgggttgtctggac |
| 5239 | Left | tccaagcacaccatcctgag |
| 5240 | Right | ccactcgaaatgggttgtctg |
| 5241 | Left | cctgagtccgtggatgagg |
| 5242 | Right | gagatgtattccagggccactc |
| 5243 | Left | ttctcaacacctcagctgactccaa |
| 5244 | Right | gattcttccctggagcactgtccaa |
| 5245 | Left | gaggctcctttctccttctca |
| 5246 | Right | ctggagcactgtccaacca |
| 5247 | Left | ctcagctgactccaagcaca |
| 5248 | Right | gagcactgtccaaccatgc |
| 5249 | Left | acaccatcctgagtccgtg |
| 5250 | Right | ttccagggccactcgaaat |
| 5251 | Left | accatcctgagtccgtggat |
| 5252 | Right | ggccactcgaaatgggttg |
| 5253 | Left | ccgtggatgaggagcagc |
| 5254 | Right | ggagatgtattccagggcca |
| 5255 | Left | gagcactgcacactggc |
| 5256 | Right | gacaagctgcggtttccac |
| 5257 | Left | gcacaccatcctgagtcc |
| 5258 | Right | gaaatgggttgtctggacgcc |
| 5259 | Left | atgaggagcagcagtgag |
| 5260 | Right | agggccactcgaaatggg |
| 5261 | Left | ccgtggatgaggagc |
| 5262 | Right | cccgattcttccctggagc |
| 5263 | Left | tgagcactgcacact |
| 5264 | Right | aagctgcggtttccactgg |
| 5265 | Left | atgaggagcagcagt |
| 5266 | Right | ggacgcccgattcttccc |
| 5267 | Left | gtgagcactgcac |
| 5268 | Right | tccactggagatgtattcca |
| 5269 | Left | ttctccttctcaaca |
| 5270 | Right | gcccgattcttccctgg |

ALK Region1 226-275 bases

| | | |
|---|---|---|
| 5271 | Left | ctcctttctccttctcaacacct |
| 5272 | Right | ctcgaaatgggttgtctggac |
| 5273 | Left | gaggctcctttctccttctcaa |
| 5274 | Right | ccactcgaaatgggttgtctg |
| 5275 | Left | cttctcaacacctcagctgactc |
| 5276 | Right | gagatgtattccagggccactc |
| 5277 | Left | acaccatcctgagtccgtg |
| 5278 | Right | caaagaagtccactgcagacaag |
| 5279 | Left | ctccttctcaacacctcagct |
| 5280 | Right | ggagatgtattccagggcca |
| 5281 | Left | ccagaggctcctttctccttc |
| 5282 | Right | ttccagggccactcgaaat |
| 5283 | Left | cctgagtccgtggatgagg |
| 5284 | Right | gcaaagaagtccactgcagac |
| 5285 | Left | ttctcaacacctcagctgactccaa |
| 5286 | Right | ggccactcgaaatgggttg |
| 5287 | Left | gagcactgcacactggc |
| 5288 | Right | gttccttcactgcagttcttcag |
| 5289 | Left | ctgactccaagcacaccatc |
| 5290 | Right | caagctgcggtttccactg |
| 5291 | Left | tccaagcacaccatcctgag |
| 5292 | Right | agacaagctgcggtttcca |
| 5293 | Left | ctcagctgactccaagcaca |
| 5294 | Right | gaaatgggttgtctggacgcc |
| 5295 | Left | ccgtggatgaggagcagc |
| 5296 | Right | ctgcagttcttcagggcaaag |
| 5297 | Left | accatcctgagtccgtggat |
| 5298 | Right | ggcaaagaagtccactgca |
| 5299 | Left | atgaggagcagcagtgag |
| 5300 | Right | cttcactgcagttcttcagggc |
| 5301 | Left | cccagaggctcctttctcc |
| 5302 | Right | gggccactcgaaatggg |
| 5303 | Left | gcacaccatcctgagtcc |
| 5304 | Right | tccactggagatgtattcca |
| 5305 | Left | gtgagcactgcaca |
| 5306 | Right | gatgttccttcactgcagttctt |
| 5307 | Left | ccgtggatgaggagc |
| 5308 | Right | ttcagggcaaagaagtcc |
| 5309 | Left | tgaggagcagcagt |
| 5310 | Right | tgttccttcactgcagtt |

ALK Region2 75-125 bases

| | | |
|---|---|---|
| 5311 | Left | cactccagggaagcatggt |
| 5312 | Right | caaagaagtccactgcagacaag |
| 5313 | Left | aacgaggctgcaagagagat |
| 5314 | Right | gagatgtattccagggccact |
| 5315 | Left | gaggctgcaagagagatcct |
| 5316 | Right | tggagatgtattccagggcc |
| 5317 | Left | ctcctgatgcccactccag |
| 5318 | Right | caagctgcggtttccactg |
| 5319 | Left | tcctcctgatgcccactc |
| 5320 | Right | agacaagctgcggtttcca |
| 5321 | Left | acgaggctgcaagaga |
| 5322 | Right | tccactggagatgtattcca |
| 5323 | Left | cactccagggaagcat |
| 5324 | Right | caaagaagtccactgcagac |
| 5325 | Left | tcctcctgatgccca |
| 5326 | Right | agacaagctgcggttt |
| 5327 | Left | ccactccagggaag |
| 5328 | Right | aaagaagtccactgca |
| 5329 | Left | acgaggctgcaag |
| 5330 | Right | tccactggagatgtatt |
| 5331 | Left | agagatcctcctga |
| 5332 | Right | tccactggagatgt |

ALK Region2 126-175 bases

| | | |
|---|---|---|
| 5333 | Left | aacgaggctgcaagagagat |
| 5334 | Right | caaagaagtccactgcagacaag |
| 5335 | Left | ctcctgatgcccactccag |
| 5336 | Right | gatgttccttcactgcagttctt |
| 5337 | Left | gaggctgcaagagagatcct |
| 5338 | Right | cttcactgcagttcttcaggg |
| 5339 | Left | cacaacgaggctgcaagag |
| 5340 | Right | gcaaagaagtccactgcagac |
| 5341 | Left | ctctggaaggtacattgcccag |
| 5342 | Right | caagctgcggtttccactg |
| 5343 | Left | gaaggtacattgcccagctg |
| 5344 | Right | agacaagctgcggtttcca |
| 5345 | Left | tcctcctgatgcccactc |
| 5346 | Right | ctgcagttcttcagggcaaag |
| 5347 | Left | cccacaacgaggctgcaa |
| 5348 | Right | ggcaaagaagtccactgca |
| 5349 | Left | tgctgccccacaacgag |
| 5350 | Right | ttcagggcaaagaagtcc |
| 5351 | Left | gtacattgcccagctgctg |
| 5352 | Right | agacaagctgcggttt |
| 5353 | Left | tcctcctgatgccca |
| 5354 | Right | gatgttccttcactgcagtt |
| 5355 | Left | gccccacaacgaggct |
| 5356 | Right | tccactggagatgtattc |
| 5357 | Left | gctgctgccccacaac |
| 5358 | Right | ggcaaagaagtccact |
| 5359 | Left | ctctggaaggtacattgcc |
| 5360 | Right | agacaagctgcgg |
| 5361 | Left | cacctgcagccctct |
| 5362 | Right | tccactggagatgta |
| 5363 | Left | agagatcctcctga |
| 5364 | Right | gatgttccttcactgca |
| 5365 | Left | ctgctgccccac |
| 5366 | Right | tcagggcaaagaag |

ALK Region2 176-225 bases

| | | |
|---|---|---|
| 5367 | Left | gaaggtacattgcccagctg |
| 5368 | Right | gatgttccttcactgcagttat |
| 5369 | Left | gaggctgcaagagagatcct |
| 5370 | Right | gttccttcactgcagttcttcag |
| 5371 | Left | gtacattgcccagctgctg |
| 5372 | Right | caaagaagtccactgcagacaag |
| 5373 | Left | ctcctgatgcccactccag |
| 5374 | Right | cattccaacaagtgaaggagctc |
| 5375 | Left | ctctggaaggtacattgccca |
| 5376 | Right | actgcagttcttcagggcaa |
| 5377 | Left | cacaacgaggctgcaagagagat |
| 5378 | Right | ctgcagttcttcagggcaaaga |
| 5379 | Left | tcctcctgatgcccactc |
| 5380 | Right | cccattccaacaagtgaaggag |
| 5381 | Left | acaacgaggctgcaagaga |
| 5382 | Right | atcttggagcctggggatgttc |
| 5383 | Left | ccacaacgaggctgcaag |
| 5384 | Right | atcttggagcctggggatg |
| 5385 | Left | gagcactgcacactggc |
| 5386 | Right | gacaagctgcggtttccac |
| 5387 | Left | actgcacactggccgtc |
| 5388 | Right | aagctgcggtttccactgg |
| 5389 | Left | tgctgccccacaacgag |
| 5390 | Right | gctctgcagggccatct |
| 5391 | Left | gccccacaacgaggct |
| 5392 | Right | gatgttccttcactgcagtt |
| 5393 | Left | cacctgcagccctct |
| 5394 | Right | gcaaagaagtccactgcagac |
| 5395 | Left | tcctcctgatgccca |
| 5396 | Right | gactgtcccattccaacaagtg |
| 5397 | Left | ctctggaaggtacattgc |
| 5398 | Right | ggcaaagaagtccactgca |
| 5399 | Left | gctgctgccccacaac |
| 5400 | Right | ttcagggcaaagaagtcc |
| 5401 | Left | cgtctcggtgcacagg |
| 5402 | Right | agacaagctgcggtttc |
| 5403 | Left | ccgtggatgaggagcagc |
| 5404 | Right | tccactggagatgtattc |
| 5405 | Left | agagatcctcctga |
| 5406 | Right | caacaagtgaaggagctctgc |

ALK Region2 226-275 bases

| | | |
|---|---|---|
| 5407 | Left | ctctggaaggtacattgcccag |
| 5408 | Right | cattccaacaagtgaaggagctc |
| 5409 | Left | aacgaggctgcaagagagatc |
| 5410 | Right | cccattccaacaagtgaaggag |
| 5411 | Left | acaccatcctgagtccgtg |
| 5412 | Right | caaagaagtccactgcagacaag |
| 5413 | Left | gaaggtacattgcccagctg |
| 5414 | Right | caacaagtgaaggagctctgc |
| 5415 | Left | gtacattgcccagctgctg |
| 5416 | Right | gactgtcccattccaacaagtg |
| 5417 | Left | ctgcacactggccgtct |
| 5418 | Right | gatgttccttcactgcagttctt |
| 5419 | Left | cctgagtccgtggatgagg |
| 5420 | Right | gcaaagaagtccactgcagac |
| 5421 | Left | gagcactgcacactggc |
| 5422 | Right | gttccttcactgcagttcttcag |
| 5423 | Left | ctgactccaagcacaccatc |
| 5424 | Right | caagctgcggtttccactg |
| 5425 | Left | tccaagcacaccatcctgag |
| 5426 | Right | agacaagctgcggtttcca |
| 5427 | Left | ccgtggatgaggagcagc |
| 5428 | Right | ctgcagttcttcagggcaaag |
| 5429 | Left | accatcctgagtccgtggat |
| 5430 | Right | ggcaaagaagtccactgca |
| 5431 | Left | tcctgatgcccactccag |
| 5432 | Right | ctcatcttctccctgggca |
| 5433 | Left | cacaacgaggctgcaagag |
| 5434 | Right | ggaagtcacaggcctgcc |
| 5435 | Left | atgaggagcagcagtgag |
| 5436 | Right | cttcactgcagttcttcagggc |
| 5437 | Left | ctctggaaggtacattgcc |
| 5438 | Right | atcttggagcctggggatgttc |
| 5439 | Left | ctgctgccccacaacga |
| 5440 | Right | tgaaggagctctgcaggg |
| 5441 | Left | cgtctcggtgcacagg |
| 5442 | Right | atcttggagcctggggatg |
| 5443 | Left | gccccacaacgaggct |
| 5444 | Right | gaaggagctctgcagggccatc |
| 5445 | Left | cccacaacgaggctgcaa |
| 5446 | Right | gactgtcccattccaacaa |

ALK Region3 76-125 bases

| | | |
|---|---|---|
| 5447 | Left | ggaatacatctccagtggaaacc |
| 5448 | Right | cattccaacaagtgaaggagctc |
| 5449 | Left | gtccagacaacccatttcgag |
| 5450 | Right | atcttggagcctggggatg |
| 5451 | Left | tggccctggaatacatctcc |
| 5452 | Right | acaagtgaaggagctctgca |
| 5453 | Left | cgagtggccctggaatacatc |
| 5454 | Right | tgaaggagctctgcaggg |
| 5455 | Left | tcgagtggccctggaatac |
| 5456 | Right | gctctgcagggccatct |
| 5457 | Left | catttcgagtggccctgga |
| 5458 | Right | gagctctgcagggcca |
| 5459 | Left | gcgtccagacaacccatttc |
| 5460 | Right | atcttggagcctgggg |
| 5461 | Left | tggaatacatctccagtggaa |
| 5462 | Right | attccaacaagtgaaggag |
| 5463 | Left | ggcgtccagacaacccat |
| 5464 | Right | catcttggagcctg |
| 5465 | Left | ggaatacatctccagtg |
| 5466 | Right | cattccaacaagtgaag |

ALK Region3 126-175 bases

| | | |
|---|---|---|
| 5467 | Left | gtccagacaacccatttcgag |
| 5468 | Right | cattccaacaagtgaaggagctc |
| 5469 | Left | gagtggccctggaatacatctc |
| 5470 | Right | cccattccaacaagtgaaggag |
| 5471 | Left | cgagtggccctggaatacat |
| 5472 | Right | gactgtcccattccaacaagtg |
| 5473 | Left | gacagtgctccagggaagaat |
| 5474 | Right | caacaagtgaaggagctctgc |
| 5475 | Left | ggaagcatggttggacagtg |
| 5476 | Right | gctctgcagggccatct |
| 5477 | Left | tgctccagggaagaatcgg |
| 5478 | Right | tgaaggagctctgcaggg |
| 5479 | Left | gcgtccagacaacccatttc |
| 5480 | Right | gagctctgcagggcca |
| 5481 | Left | gaagaatcgggcgtccaga |
| 5482 | Right | gactgtcccattccaacaa |
| 5483 | Left | tcgagtggccctggaata |
| 5484 | Right | ggaagtcacaggcctgcc |
| 5485 | Left | catttcgagtggccctgga |
| 5486 | Right | cccattccaacaagtgaag |
| 5487 | Left | ggcgtccagacaacccat |
| 5488 | Right | caagctggaggactgtc |
| 5489 | Left | gaagaatcgggcgtccagacaa |
| 5490 | Right | gactgtcccattccaa |
| 5491 | Left | ccatttcgagtggccct |
| 5492 | Right | caagctggaggact |
| 5493 | Left | ggaagaatcgggcgtcc |
| 5494 | Right | ggactgtcccattc |
| 5495 | Left | ccatttcgagtggc |
| 5496 | Right | ggcctgcccaag |

ALK Region3 176-225 bases

| | | |
|---|---|---|
| 5497 | Left | gagtggccctggaatacatctc |
| 5498 | Right | acatctggctctcatcttctcc |
| 5499 | Left | gacagtgctccagggaagaat |
| 5500 | Right | cattccaacaagtgaaggagctc |
| 5501 | Left | ggaagcatggttggacagtg |
| 5502 | Right | cccattccaacaagtgaaggag |
| 5503 | Left | ccagacaacccatttcgagtg |
| 5504 | Right | atctggctctcatcttctccctg |
| 5505 | Left | cactccagggaagcatggtt |
| 5506 | Right | gactgtcccattccaacaagtg |
| 5507 | Left | gaggctgcaagagagatcct |
| 5508 | Right | caacaagtgaaggagctctgc |
| 5509 | Left | cgagtggccctggaataca |
| 5510 | Right | gcacatctggctctcatcttc |
| 5511 | Left | cgtccagacaacccatttcg |
| 5512 | Right | ctctcatcttctccctgggc |
| 5513 | Left | aacgaggctgcaagagagat |
| 5514 | Right | gctctgcagggccatct |
| 5515 | Left | gcatggttggacagtgctc |
| 5516 | Right | tgaaggagctctgcaggg |
| 5517 | Left | atggttggacagtgctccag |
| 5518 | Right | ggaagtcacaggcctgcc |
| 5519 | Left | agggaagcatggttggaca |
| 5520 | Right | gagctctgcagggcca |
| 5521 | Left | tgctccagggaagaatcgg |
| 5522 | Right | gactgtcccattccaacaa |
| 5523 | Left | tcgagtggccctggaat |
| 5524 | Right | gcagtttccggcacatctg |
| 5525 | Left | ctcctgatgcccactccag |
| 5526 | Right | cccattccaacaagtgaag |
| 5527 | Left | ccatttcgagtggccctgg |
| 5528 | Right | gcacatctggctctcatc |
| 5529 | Left | gaagaatcgggcgtccaga |
| 5530 | Right | caagctggaggactgtc |
| 5531 | Left | gaagaatcgggcgtccagacaac |
| 5532 | Right | ttctccctgggcaca |
| 5533 | Left | ggacagtgctccagggaag |
| 5534 | Right | tggaagtcacaggcc |
| 5535 | Left | agggaagaatcgggcgtc |
| 5536 | Right | gactgtcccattccaa |

ALK Region3 226-275 bases

| | | |
|---|---|---|
| 5537 | Left | gagtggccctggaatacatctc |
| 5538 | Right | agccatcttcaaagttgcagtaaaa |
| 5539 | Left | gtccagacaacccatttcgag |
| 5540 | Right | aagccatcttcaaagttgcagta |
| 5541 | Left | ctctggaaggtacattgcccag |
| 5542 | Right | cattccaacaagtgaaggagctc |
| 5543 | Left | gacagtgctccagggaagaatc |
| 5544 | Right | acatctggctctcatcttctcc |
| 5545 | Left | aacgaggctgcaagagagatc |
| 5546 | Right | cccattccaacaagtgaaggag |
| 5547 | Left | ggaagcatggttggacagtg |
| 5548 | Right | gcacatctggctctcatcttc |
| 5549 | Left | gaaggtacattgcccagctg |
| 5550 | Right | caacaagtgaaggagctctgc |
| 5551 | Left | gtacattgcccagctgctg |
| 5552 | Right | gactgtcccattccaacaagtg |
| 5553 | Left | gcgtccagacaacccatttc |
| 5554 | Right | atctggctctcatcttctccctg |
| 5555 | Left | cactccagggaagcatggtt |
| 5556 | Right | ctctcatcttctccctgggc |
| 5557 | Left | cgagtggccctggaatacat |
| 5558 | Right | tccagccacagaagccatc |
| 5559 | Left | atggttggacagtgctccag |
| 5560 | Right | gcagtttccggcacatctg |
| 5561 | Left | cacaacgaggctgcaagag |
| 5562 | Right | ggaagtcacaggcctgcc |
| 5563 | Left | catttcgagtggccctgga |
| 5564 | Right | aagccatcttcaaagttgca |
| 5565 | Left | gacagtgctccagggaaga |
| 5566 | Right | cacaggcagtttccggc |
| 5567 | Left | cccacaacgaggctgcaa |
| 5568 | Right | tgaaggagctctgcaggg |
| 5569 | Left | gcatggttggacagtgctc |
| 5570 | Right | gcacatctggctctcatc |
| 5571 | Left | tgctgccccacaacgag |
| 5572 | Right | agctctgcagggccatc |
| 5573 | Left | tcgagtggccctggaata |
| 5574 | Right | gtccagccacagaagcc |
| 5575 | Left | gccccacaacgaggct |
| 5576 | Right | gactgtcccattccaacaa |

ALK Region4 226-275 bases

| | | |
|---|---|---|
| 5577 | Left | gagcactgcacactggc |
| 5578 | Right | ttggagcctggggatgttc |
| 5579 | Left | ctgcacactggccgtct |
| 5580 | Right | atcttggagcctggggatg |
| 5581 | Left | cgtctcggtgcacagg |
| 5582 | Right | gctctgcagggccatct |
| 5583 | Left | cgtctcggtgcac |
| 5584 | Right | agctctgcagggcca |
| 5585 | Left | tgagcactgcacact |
| 5586 | Right | tggagcctgggg |
| 5587 | Left | aggagcagcagtgag |
| 5588 | Right | gatgttccttcact |

ALK Region4 276-325 bases

| | | |
|---|---|---|
| 5589 | Left | ctgcacactggccgtct |
| 5590 | Right | cattccaacaagtgaaggagctc |
| 5591 | Left | ctgactccaagcacaccatc |
| 5592 | Right | atcttggagcctggggatgtt |
| 5593 | Left | cactgcacactggccg |
| 5594 | Right | cccattccaacaagtgaaggag |
| 5595 | Left | atgaggagcagcagtgag |
| 5596 | Right | caacaagtgaaggagctctgc |
| 5597 | Left | cctgagtccgtggatgagg |
| 5598 | Right | gctctgcagggccatct |
| 5599 | Left | tccaagcacaccatcctgag |
| 5600 | Right | atcttggagcctggggat |
| 5601 | Left | ccatcctgagtccgtggat |
| 5602 | Right | gagctctgcagggcca |
| 5603 | Left | tgagcactgcacactgg |
| 5604 | Right | ctgtcccattccaacaagtg |
| 5605 | Left | cgtctcggtgcacagg |
| 5606 | Right | tgaaggagctctgcaggg |
| 5607 | Left | acaccatcctgagtccgtg |
| 5608 | Right | atcttggagcctggg |
| 5609 | Left | tgagcactgcacac |
| 5610 | Right | gactgtcccattccaacaa |
| 5611 | Left | cgtctcggtgcac |
| 5612 | Right | cccattccaacaagtgaag |
| 5613 | Left | ttctcaacacctcagctgactc |
| 5614 | Right | gatgttccttcact |
| 5615 | Left | ccgtggatgaggagcagc |
| 5616 | Right | catcttggagcct |
| 5617 | Left | gtgagcactgca |
| 5618 | Right | gactgtcccattccaa |

ALK Region4 326-375 bases

| | | |
|---|---|---|
| 5619 | Left | cttctcaacacctcagctgactc |
| 5620 | Right | cattccaacaagtgaaggagctc |
| 5621 | Left | aacacctcagctgactccaag |
| 5622 | Right | cccattccaacaagtgaaggag |
| 5623 | Left | tttctccttctcaacacctcagct |
| 5624 | Right | caacaagtgaaggagctctgc |
| 5625 | Left | ctcagctgactccaagcaca |
| 5626 | Right | gactgtcccattccaacaagtg |
| 5627 | Left | cctttctccttctcaacacctca |
| 5628 | Right | tcttggagcctggggatg |
| 5629 | Left | gctcctttctccttctcaacac |
| 5630 | Right | agctctgcagggccatc |
| 5631 | Left | tgcacactggccgtctc |
| 5632 | Right | ctggctctcatcttctccctg |
| 5633 | Left | acaccatcctgagtccgtg |
| 5634 | Right | tgaaggagctctgcaggg |
| 5635 | Left | cactgcacactggccgt |
| 5636 | Right | ctctcatcttctccctgggc |
| 5637 | Left | cctgagtccgtggatgagg |
| 5638 | Right | ggaagtcacaggcctgcc |
| 5639 | Left | gactccaagcacaccatcct |
| 5640 | Right | gactgtcccattccaacaa |
| 5641 | Left | gaggctcctttctccttctcaa |
| 5642 | Right | atcttggagcctgggg |
| 5643 | Left | accatcctgagtccgtggat |
| 5644 | Right | cccattccaacaagtgaag |
| 5645 | Left | tccaagcacaccatcctgag |
| 5646 | Right | caagctggaggactgtc |
| 5647 | Left | gagcactgcacactggc |
| 5648 | Right | ttctccctgggcaca |
| 5649 | Left | ccagaggctcctttctccttc |
| 5650 | Right | gcagggccatcttg |
| 5651 | Left | gctgactccaagcacaccat |
| 5652 | Right | gactgtcccattccaa |
| 5653 | Left | ccgtggatgaggagcagc |
| 5654 | Right | tggaagtcacaggcc |
| 5655 | Left | cccagaggctcctttctcc |
| 5656 | Right | catcttggagcctg |
| 5657 | Left | gcacaccatcctgagtcc |
| 5658 | Right | caagctggaggact |

ALK Region4 376-425 bases

| | | |
|---|---|---|
| 5659 | Left | ctcctttctccttctcaacacct |
| 5660 | Right | gactgtcccattccaacaagtg |
| 5661 | Left | gaggctcctttctccttctcaac |
| 5662 | Right | cattccaacaagtgaaggagctc |
| 5663 | Left | ctccttctcaacacctcagct |
| 5664 | Right | cccattccaacaagtgaaggag |
| 5665 | Left | ccagaggctcctttctccttc |
| 5666 | Right | caacaagtgaaggagctctgc |
| 5667 | Left | acaccatcctgagtccgtg |
| 5668 | Right | acatctggctctcatcttctcc |
| 5669 | Left | ctccaagcacaccatcctga |
| 5670 | Right | ctggctctcatcttctccctg |
| 5671 | Left | accatcctgagtccgtggat |
| 5672 | Right | gcacatctggctctcatcttc |
| 5673 | Left | ctgactccaagcacaccatc |
| 5674 | Right | ctctcatcttctccctgggc |
| 5675 | Left | cttctcaacacctcagctgactc |
| 5676 | Right | ggaagtcacaggcctgcc |
| 5677 | Left | cctgagtccgtggatgagg |
| 5678 | Right | gcagtttccggcacatctg |
| 5679 | Left | cccagaggctcctttctcc |
| 5680 | Right | aaggagctctgcagggc |
| 5681 | Left | ttctcaacacctcagctgactccaa |
| 5682 | Right | gactgtcccattccaacaa |
| 5683 | Left | ccgtggatgaggagcagc |
| 5684 | Right | cacaggcagtttccggc |
| 5685 | Left | ctcagctgactccaagcaca |
| 5686 | Right | caagctggaggactgtc |
| 5687 | Left | tggggcagagcgttct |
| 5688 | Right | cccattccaacaagtgaag |
| 5689 | Left | gcacaccatcctgagtcc |
| 5690 | Right | ttctccctgggcaca |
| 5691 | Left | ccgtggatgaggagc |
| 5692 | Right | gcacatctggctctcatc |
| 5693 | Left | agagcgttctaaggagatg |
| 5694 | Right | gactgtcccattccaa |
| 5695 | Left | atgaggagcagcagt |
| 5696 | Right | gcagtttccggcacat |
| 5697 | Left | cccagaggctcctttc |
| 5698 | Right | caagctggaggact |

ALK Region4 426-475 bases

| | | |
|---|---|---|
| 5699 | Left | ctcctttctccttctcaacacctc |
| 5700 | Right | acatctggctctcatcttctcc |
| 5701 | Left | gactccaagcacaccatcct |
| 5702 | Right | agccatcttcaaagttgcagtaaaa |
| 5703 | Left | gctcctttctccttctcaacac |
| 5704 | Right | atctggctctcatcttctccctg |
| 5705 | Left | cttctcaacacctcagctgactc |
| 5706 | Right | gcacatctggctctcatcttc |
| 5707 | Left | acaccatcctgagtccgtg |
| 5708 | Right | aagccatcttcaaagttgcagta |
| 5709 | Left | agatggacttgctggatggg |
| 5710 | Right | cattccaacaagtgaaggagctc |
| 5711 | Left | gaggctcctttctccttctcaa |
| 5712 | Right | ctctcatcttctccctgggc |
| 5713 | Left | tttctccttctcaacacctcagct |
| 5714 | Right | gcagtttccggcacatctg |
| 5715 | Left | ccgcatcccctccgag |
| 5716 | Right | cccattccaacaagtgaaggag |
| 5717 | Left | cctgagtccgtggatgagg |
| 5718 | Right | tccagccacagaagccatc |
| 5719 | Left | ccagaggctcctttctccttc |
| 5720 | Right | ggaagtcacaggcctgcc |
| 5721 | Left | ttctcaacacctcagctgactccaa |
| 5722 | Right | cacaggcagtttccggc |
| 5723 | Left | tccaagcacaccatcctgag |
| 5724 | Right | aagccatcttcaaagttgca |
| 5725 | Left | cagagctggtcctggcg |
| 5726 | Right | aacaagtgaaggagctctgca |
| 5727 | Left | cagatggacttgctggat |
| 5728 | Right | gactgtcccattccaacaagtg |
| 5729 | Left | ccatcctgagtccgtggat |
| 5730 | Right | gtccagccacagaagcc |
| 5731 | Left | ccgtggatgaggagcagc |
| 5732 | Right | tgtgccttgggtccagc |
| 5733 | Left | ctcagctgactccaagcaca |
| 5734 | Right | gcacatctggctctcatc |
| 5735 | Left | tcctggcgccgcatc |
| 5736 | Right | tgaaggagctctgcaggg |
| 5737 | Left | gctgactccaagcacaccat |
| 5738 | Right | gcagtttccggcacat |

ALK Region4 476-525 bases

| | | |
|---|---|---|
| 5739 | Left | ctcctttctccttctcaacacct |
| 5740 | Right | agccatcttcaaagttgcagtaaaa |
| 5741 | Left | gaggctcctttctccttctcaac |
| 5742 | Right | aagccatcttcaaagttgcagta |
| 5743 | Left | gagtattcccctccactgcat |
| 5744 | Right | cattccaacaagtgaaggagctc |
| 5745 | Left | tattcccctccactgcatgac |
| 5746 | Right | cccattccaacaagtgaaggag |
| 5747 | Left | agatggacttgctggatggg |
| 5748 | Right | acatctggctctcatcttctcc |
| 5749 | Left | cactgcatgacctcaggaac |
| 5750 | Right | gactgtcccattccaacaagtg |
| 5751 | Left | gcatgacctcaggaaccaga |
| 5752 | Right | caacaagtgaaggagctctgc |
| 5753 | Left | cttctcaacacctcagctgactc |
| 5754 | Right | tccagccacagaagccatc |
| 5755 | Left | ctgactccaagcacaccatc |
| 5756 | Right | attgaggagtgtggggtgac |
| 5757 | Left | ctccttctcaacacctcagct |
| 5758 | Right | tgacagtgtgccttgggtc |
| 5759 | Left | ccagaggctcctttctccttc |
| 5760 | Right | gcagtttccggcacatctg |
| 5761 | Left | aacacctcagctgactccaag |
| 5762 | Right | agtgtgccttgggtccag |
| 5763 | Left | ctccaagcacaccatcctga |
| 5764 | Right | agtgtggggtgacagtgtg |
| 5765 | Left | gtattcccctccactgcatgacctc |
| 5766 | Right | tgaaggagctctgcaggg |
| 5767 | Left | acaccatcctgagtccgtg |
| 5768 | Right | gtcctgacctgccattgag |
| 5769 | Left | ctcagctgactccaagcaca |
| 5770 | Right | ggggtgacagtgtgcctt |
| 5771 | Left | cccagaggctcctttctcc |
| 5772 | Right | aagccatcttcaaagttgca |
| 5773 | Left | agagcgttctaaggagatg |
| 5774 | Right | gcacatctggctctcatcttc |
| 5775 | Left | accatcctgagtccgtggat |
| 5776 | Right | gtccagccacagaagcc |
| 5777 | Left | tccactgcatgacctcagg |
| 5778 | Right | gactgtcccattccaacaa |

ALK Region4 750-1250 bases

| | | |
|---|---|---|
| 5779 | Left | tggaatctcacctggataatgaaag |
| 5780 | Right | ttttgttctccactagcaccaag |
| 5781 | Left | gaatcaccaacaaacatgccttc |
| 5782 | Right | agccatcttcaaagttgcagtaaaa |
| 5783 | Left | cacctggataatgaaagactccttc |
| 5784 | Right | tggtcactgtagcactttcagaa |
| 5785 | Left | tggataatgaaagactccttccctt |
| 5786 | Right | caatagagcatggtcttggtgg |
| 5787 | Left | attttacatggaatctcacctggat |
| 5788 | Right | gaaacgtagcactggtcactgtag |
| 5789 | Left | atctcacctggataatgaaagactc |
| 5790 | Right | cacccggttttgttctccactag |
| 5791 | Left | tccttctcctgattattttacatgga |
| 5792 | Right | aagccatcttcaaagttgcagta |
| 5793 | Left | acatggaatctcacctggataatga |
| 5794 | Right | gaaacgtagcactggtcactg |
| 5795 | Left | gactggtcatagctccttggaatc |
| 5796 | Right | ggttttgttctccactagcacc |
| 5797 | Left | cagatcttcgggactggtcatag |
| 5798 | Right | acatctggctctcatcttctcc |
| 5799 | Left | cctgattattttacatggaatctcacc |
| 5800 | Right | tgtcagacacatcgaggagag |
| 5801 | Left | ctcctttctccttctcaacacctc |
| 5802 | Right | ctccttcccggttttgttctc |
| 5803 | Left | gctcctttctccttctcaacac |
| 5804 | Right | tttgttctccactagcaccaaggac |
| 5805 | Left | aaagactccttccctttcctgt |
| 5806 | Right | ctcaagactccacgaatgagc |
| 5807 | Left | aatcaccaacaaacatgccttctcc |
| 5808 | Right | ctagcaccaaggacacgtttc |
| 5809 | Left | cttctccactcctgattattttaca |
| 5810 | Right | cttcccggttttgttctccac |
| 5811 | Left | tctcctgattattttacatggaatctc |
| 5812 | Right | gtagcactggtcactgtagcacttt |
| 5813 | Left | gactggtcatagctccttgga |
| 5814 | Right | gcacatctggctctcatcttc |
| 5815 | Left | gagaagaaggcgtcggaagt |
| 5816 | Right | cattccaacaagtgaaggagctc |
| 5817 | Left | gatcttcgggactggtcatagctc |
| 5818 | Right | atctggctctcatcttctccctg |

TABLE 12
EGFR cDNA Capture Primer List for NGS Panel

| | | |
|---|---|---|
| Seq. | | |
| ID | Primer | Sequence |

EGFR Region1 75-125 bases

| | | |
|---|---|---|
| 5819 | Left | CCCAGTGGAGAAGCTC |
| 5820 | Right | GGGATCCAGAGTCCCTTATACA |
| 5821 | Left | GAGAAGCTCCCAACCA |
| 5822 | Right | GGGATCCAGAGTCCCTTAT |
| 5823 | Left | CCCAGTGGAGAAG |
| 5824 | Right | TGGGATCCAGAGTCCC |
| 5825 | Left | GAGAAGCTCCCAA |
| 5826 | Right | TGGGATCCAGAGT |

EGFR Region1 126-175 bases

| | | |
|---|---|---|
| 5827 | Left | TTGTGGAGCCTCTTACACCC |
| 5828 | Right | ACGGGAATTTTAACTTTCTCACCTT |
| 5829 | Left | CTTGTGGAGCCTCTTACACCCAGT |
| 5830 | Right | TGATAGCGACGGGAATTTTAACT |
| 5831 | Left | GGAGAGGGAGCTTGTGGAG |
| 5832 | Right | GGGATCCAGAGTCCCTTATACA |
| 5833 | Left | CTTGTGGAGCCTCTTACA |
| 5834 | Right | CGACGGGAATTTTAACTTTCTCAC |
| 5835 | Left | CTGCAGGAGAGGGAGCTTG |
| 5836 | Right | AATTTTAACTTTCTCACCTTCTG |
| 5837 | Left | CCCAGTGGAGAAGCTC |
| 5838 | Right | TTCTCTTAATTCCTTGATAGCGACG |
| 5839 | Left | GAGAAGCTCCCAACCA |
| 5840 | Right | CTTAATTCCTTGATAGCGACGGGAA |
| 5841 | Left | TGCTGCAGGAGAGGGA |
| 5842 | Right | GGGATCCAGAGTCCCTTAT |
| 5843 | Left | CTTGTGGAGCCTCTT |
| 5844 | Right | CCTTGATAGCGACGGGAATTTTA |
| 5845 | Left | GAGAAGCTCCCAA |
| 5846 | Right | GCGACGGGAATTTTAACTTTCT |
| 5847 | Left | GCACGCTGCGGA |
| 5848 | Right | TGGGATCCAGAGTCCC |
| 5849 | Left | CCCAGTGGAGAAG |
| 5850 | Right | TCTCTTAATTCCTTGATAGCG |
| 5851 | Left | CGGAGGCTGCTG |
| 5852 | Right | TGGGATCCAGAGT |
| 5853 | Left | TGCTGCAGGAGAG |
| 5854 | Right | CTTCTGGGATCCA |

EGFR Region1 176-225 bases

| | | |
|---|---|---|
| 5855 | Left | TTGTGGAGCCTCTTACACCC |
| 5856 | Right | TTCTCTTAATTCCTTGATAGCGACG |
| 5857 | Left | CAGGAGAGGGAGCTTGTGG |
| 5858 | Right | ACGGGAATTTTAACTTTCTCACCTT |
| 5859 | Left | CTGCAGGAGAGGGAGCTTG |
| 5860 | Right | TGATAGCGACGGGAATTTTAACTT |
| 5861 | Left | CTTGTGGAGCCTCTTACACCCAGT |
| 5862 | Right | CTTAATTCCTTGATAGCGACGGGAA |
| 5863 | Left | CCACATCGTTCGGAAGCG |
| 5864 | Right | CGACGGGAATTTTAACTTTCTCAC |
| 5865 | Left | CATCGTTCGGAAGCGCAC |
| 5866 | Right | CCTTGATAGCGACGGGAATTTTAA |
| 5867 | Left | ATCGGCCTCTTCATGCGAA |
| 5868 | Right | GGGATCCAGAGTCCCTTATACA |
| 5869 | Left | GAAGGCGCCACATCGTTC |
| 5870 | Right | GCGACGGGAATTTTAACTTTCT |
| 5871 | Left | TGCTGCAGGAGAGGGAG |
| 5872 | Right | TCCTTGATAGCGACGGGAATTT |
| 5873 | Left | GCGCCACATCGTTCGGAA |
| 5874 | Right | AATTTTAACTTTCTCACCTTCTG |
| 5875 | Left | GGATCGGCCTCTTCATGC |
| 5876 | Right | GGGATCCAGAGTCCCTTAT |
| 5877 | Left | CCCAGTGGAGAAGCTC |
| 5878 | Right | ACGTAGGCTTCATCGAGGATTT |
| 5879 | Left | CTTGTGGAGCCTCTTACA |
| 5880 | Right | CCTTGTTGGCTTTCGGAGA |
| 5881 | Left | GAGAAGCTCCCAACCA |
| 5882 | Right | CACGTAGGCTTCATCGAGGA |
| 5883 | Left | CGAAGGCGCCACATCG |
| 5884 | Right | TGGGATCCAGAGTCCC |
| 5885 | Left | TGCTGCAGGAGAGG |
| 5886 | Right | AGATGTTGCTTCTCTTAATTCC |
| 5887 | Left | GCACGCTGCGGA |
| 5888 | Right | TTCTCTTAATTCCTTGATAGCG |
| 5889 | Left | CCCAGTGGAGAAG |
| 5890 | Right | GGCCATCACGTAGGCTTCAT |
| 5891 | Left | CTTGTGGAGCCTCTT |
| 5892 | Right | TCGGAGATGTTGCTTCT |
| 5893 | Left | GAGAAGCTCCCAA |
| 5894 | Right | CTGGCCATCACGTAGGCTT |

EGFR Region1 226-275 bases

| | | |
|---|---|---|
| 5895 | Left | ATCGGCCTCTTCATGCGAA |
| 5896 | Right | TTCTCTTAATTCCTTGATAGCGACG |
| 5897 | Left | CCTCCTCTTGCTGCTGGT |
| 5898 | Right | ACGGGAATTTTAACTTTCTCACCTT |
| 5899 | Left | TTGTGGAGCCTCTTACACCC |
| 5900 | Right | CGTAGGCTTCATCGAGGATTTC |
| 5901 | Left | GAAGGCGCCACATCGTTC |
| 5902 | Right | TGATAGCGACGGGAATTTTAACTT |
| 5903 | Left | CCTCTTGCTGCTGGTGGT |
| 5904 | Right | CGACGGGAATTTTAACTTTCTCAC |
| 5905 | Left | CCACATCGTTCGGAAGCG |
| 5906 | Right | CTTAATTCCTTGATAGCGACGGGAA |
| 5907 | Left | GGATCGGCCTCTTCATGC |
| 5908 | Right | CCTTGATAGCGACGGGAATTTTAA |
| 5909 | Left | GGAGAGGGAGCTTGTGGAG |
| 5910 | Right | CACGTAGGCTTCATCGAGGAT |
| 5911 | Left | GCCCTCCTCTTGCTGCT |
| 5912 | Right | GGGATCCAGAGTCCCTTATACA |
| 5913 | Left | CTTGTGGAGCCTCTTACACCCAGT |
| 5914 | Right | GGCCATCACGTAGGCTTCAT |
| 5915 | Left | CTGGTGGTGGCCCTGG |
| 5916 | Right | TTCCTTGATAGCGACGGGAATTT |
| 5917 | Left | CTGCAGGAGAGGGAGCTTG |
| 5918 | Right | CTGGCCATCACGTAGGCTT |
| 5919 | Left | CTGCTGGTGGTGGCCC |
| 5920 | Right | GCGACGGGAATTTTAACTTTCT |
| 5921 | Left | CATCGTTCGGAAGCGCAC |
| 5922 | Right | CCTTGTTGGCTTTCGGAGA |
| 5923 | Left | GCGCCACATCGTTCGGA |
| 5924 | Right | AGATGTTGCTTCTCTTAATTCC |
| 5925 | Left | TGCTGCAGGAGAGGGAG |
| 5926 | Right | TCACGTAGGCTTCATCGAG |
| 5927 | Left | CGAAGGCGCCACATCG |
| 5928 | Right | TTCTCTTAATTCCTTGATAGCG |
| 5929 | Left | GGGCCCTCCTCTTGCT |
| 5930 | Right | GGGATCCAGAGTCCCTTAT |
| 5931 | Left | CTTGTGGAGCCTCTTACA |
| 5932 | Right | CGCTGGCCATCACGTAGG |
| 5933 | Left | GAGAAGCTCCCAACCA |
| 5934 | Right | GGTGGAGGTGAGGCAGATG |

EGFR Region2 75-125 bases

| | | |
|---|---|---|
| 5935 | Left | AATCCTCGATGAAGCCTACGT |
| 5936 | Right | CCAGGAGGCAGCCGAAG |
| 5937 | Left | AATCCTCGATGAAGCCTACGTGATG |
| 5938 | Right | AGGAGGCAGCCG |
| 5939 | Left | GGAAATCCTCGATGAAGCCTA |
| 5940 | Right | CGAAGGGCATGAG |

EGFR Region2 126-175 bases

| | | |
|---|---|---|
| 5941 | Left | GAAATCCTCGATGAAGCCTACG |
| 5942 | Right | GAGCCAATATTGTCTTTGTGTTCC |
| 5943 | Left | GAAATCCTCGATGAAGCCTACGTGA |
| 5944 | Right | AATATTGTCTTTGTGTTCCCGGAC |
| 5945 | Left | AACATCTCCGAAAGCCAACAAG |
| 5946 | Right | CTTTGTGTTCCCGGACATAGTC |
| 5947 | Left | CTCGATGAAGCCTACGTGATGG |
| 5948 | Right | ATTGTCTTTGTGTTCCCGGACATA |
| 5949 | Left | TCGCTATCAAGGAATTAAGAGAAGC |
| 5950 | Right | CCAGGAGGCAGCCGAAG |
| 5951 | Left | GGAAATCCTCGATGAAGCC |
| 5952 | Right | GGGAGCCAATATTGTCTTTGTGT |
| 5953 | Left | CAAGGAATTAAGAGAAGCAACATCT |
| 5954 | Right | GACATAGTCCAGGAGG |
| 5955 | Left | CCGTCGCTATCAAGGAATTAAGAG |
| 5956 | Right | AGGAGGCAGCCG |
| 5957 | Left | TTAAAATTCCCGTCGCTATCAAGG |
| 5958 | Right | CGAAGGGCATGAG |
| 5959 | Left | GGAAATCCTCGATGAA |
| 5960 | Right | TGGGAGCCAATATTGTCTTTG |
| 5961 | Left | CAACAAGGAAATCC |
| 5962 | Right | TGGGAGCCAATATTGTCT |

EGFR Region2 176-225 bases

| | | |
|---|---|---|
| 5963 | Left | GAAAGTTAAAATTCCCGTCGCTATC |
| 5964 | Right | GAGCCAATATTGTCTTTGTGTTCC |
| 5965 | Left | GAAGGTGAGAAAGTTAAAATTCCCG |
| 5966 | Right | ATTGTCTTTGTGTTCCCGGACATAG |
| 5967 | Left | GTGAGAAAGTTAAAATTCCCGTCG |
| 5968 | Right | AATATTGTCTTTGTGTTCCCGGAC |
| 5969 | Left | CCGTCGCTATCAAGGAATTAAGAG |
| 5970 | Right | GGGAGCCAATATTGTCTTTGTGT |
| 5971 | Left | GAAATCCTCGATGAAGCCTACG |
| 5972 | Right | GACGGTCCTCCAAGTAGTTCAT |
| 5973 | Left | TCGCTATCAAGGAATTAAGAGAAGC |
| 5974 | Right | TGGGAGCCAATATTGTCTTTG |
| 5975 | Left | AGAAGCAACATCTCCGAAAGC |
| 5976 | Right | GATCTGCACACACCAGTTGAG |
| 5977 | Left | GAAATCCTCGATGAAGCCTACGTGA |
| 5978 | Right | CGACGGTCCTCCAAGTAGTT |
| 5979 | Left | CTCGATGAAGCCTACGTGATGG |
| 5980 | Right | TCCTCCAAGTAGTTCATGCCC |
| 5981 | Left | CAACATCTCCGAAAGCCAACAA |
| 5982 | Right | CACACACCAGTTGAGCAGG |
| 5983 | Left | TTAAAATTCCCGTCGCTATCAAGG |
| 5984 | Right | CCAGGAGGCAGCCGAAG |
| 5985 | Left | GAAGCAACATCTCCGAAAGCCAA |
| 5986 | Right | CGATCTGCACACACCAGTT |
| 5987 | Left | AATTCCCGTCGCTATCAAGGAATTA |
| 5988 | Right | TGGGAGCCAATATTGTCT |
| 5989 | Left | AAATCCTCGATGAAGCCT |
| 5990 | Right | CCTCCAAGTAGTTCATGCCCTTT |
| 5991 | Left | TCAAGGAATTAAGAGAAGCAACATCT |
| 5992 | Right | GACATAGTCCAGGAGG |
| 5993 | Left | TATCAAGGAATTAAGAGAAGCAACA |
| 5994 | Right | GGTACTGGGAGCCA |
| 5995 | Left | CCCAGAAGGTGAGAAAGTTAAAAT |
| 5996 | Right | AGGAGGCAGCCG |
| 5997 | Left | AGAAGCAACATCTCCGAA |
| 5998 | Right | CGATCTGCACACACCA |
| 5999 | Left | GAAATCCTCGATGAAG |
| 6000 | Right | GCGACGGTCCTCCAAGTA |
| 6001 | Left | GGCACGGTGTATAAGGGAC |
| 6002 | Right | CGAAGGGCATGAG |

EGFR Region2 226-275 bases

| | | |
|---|---|---|
| 6003 | Left | AAGGTGAGAAAGTTAAAATTCCCGT |
| 6004 | Right | GGAGCCAATATTGTCTTTGTGTTC |
| 6005 | Left | GAAAGTTAAAATTCCCGTCGCTATC |
| 6006 | Right | TGGGAGCCAATATTGTCTTTGTG |
| 6007 | Left | TCGCTATCAAGGAATTAAGAGAAGC |
| 6008 | Right | GACGGTCCTCCAAGTAGTTCAT |
| 6009 | Left | GTGAGAAAGTTAAAATTCCCGTCGC |
| 6010 | Right | CCAATATTGTCTTTGTGTTCCCG |
| 6011 | Left | CCGTCGCTATCAAGGAATTAAGAG |
| 6012 | Right | CCTCCAAGTAGTTCATGCCCTTT |
| 6013 | Left | TCAAGGAATTAAGAGAAGCAACATCT |
| 6014 | Right | TCCTCCAAGTAGTTCATGCCC |
| 6015 | Left | TTAAAATTCCCGTCGCTATCAAGG |
| 6016 | Right | GATCTGCACACACCAGTTGAG |
| 6017 | Left | GAAATCCTCGATGAAGCCTACG |
| 6018 | Right | TGTTTTCACCAGTACGTTCCTG |
| 6019 | Left | AGAAGGTGAGAAAGTTAAAATTCC |
| 6020 | Right | ATATTGTCTTTGTGTTCCCGGAC |
| 6021 | Left | TATCAAGGAATTAAGAGAAGCAACA |
| 6022 | Right | CGACGGTCCTCCAAGTAGTT |
| 6023 | Left | ATTCCCGTCGCTATCAAGGAATTAA |
| 6024 | Right | CACACACCAGTTGAGCAGG |
| 6025 | Left | CCCAGAAGGTGAGAAAGTTAAAAT |
| 6026 | Right | GTCTTTGTGTTCCCGGACATA |
| 6027 | Left | AATTCCCGTCGCTATCAAGGAAT |
| 6028 | Right | CGATCTGCACACACCAGTT |
| 6029 | Left | AACATCTCCGAAAGCCAACAAG |
| 6030 | Right | AAGCGACGGTCCTCCAAGTA |
| 6031 | Left | GGCACGGTGTATAAGGGAC |
| 6032 | Right | CTTTGTGTTCCCGGACATAGTC |
| 6033 | Left | TCGATGAAGCCTACGTGATGG |
| 6034 | Right | GACATGCTGCGGTGTTTTCA |
| 6035 | Left | GAAATCCTCGATGAAGCCTACGTGA |
| 6036 | Right | AGTACGTTCCTGGCTGCC |
| 6037 | Left | AGAAGCAACATCTCCGAAAGC |
| 6038 | Right | CAAGCGACGGTCCTCCAA |
| 6039 | Left | AAAAGATCAAAGTGCTGGGCTC |
| 6040 | Right | CCAGGAGGCAGCCGAAG |
| 6041 | Left | GAAGCAACATCTCCGAAAGCCAAC |
| 6042 | Right | CTGCCAGGTCGCGGT |

EGFR Region3 75-125 bases

| | | |
|---|---|---|
| 6043 | Left | CAAAGGGCATGAACTACTTGGAG |
| 6044 | Right | CCAGCAGTTTGGCC |
| 6045 | Left | AAAGGGCATGAACTACTTGGAGGAC |
| 6046 | Right | CACCCAGCAGTTTG |
| 6047 | Left | GCATGAACTACTTG |
| 6048 | Right | TTCCGCACCCAG |

EGFR Region3 126-175 bases

| | | |
|---|---|---|
| 6049 | Left | CAAAGGGCATGAACTACTTGGAG |
| 6050 | Right | CCTTCTGCATGGTATTCTTTCTCTT |
| 6051 | Left | AAAGGGCATGAACTACTTGGAGGAC |
| 6052 | Right | CCTCCTTCTGCATGGTATTCTTTC |
| 6053 | Left | CAACTGGTGTGTGCAGATCG |
| 6054 | Right | CTGCATGGTATTCTTTCTCTTCCG |
| 6055 | Left | CTCCCAGTACCTGCTCAACT |
| 6056 | Right | CTTTCTCTTCCGCACCCAG |
| 6057 | Left | GCTCAACTGGTGTGTGC |
| 6058 | Right | CATGGTATTCTTTCTCTTCCGCAC |
| 6059 | Left | AAAGGGCATGAACTACTTG |
| 6060 | Right | GCCTCCTTCTGCATGGTATTCT |
| 6061 | Left | GGCTCCCAGTACCTGCTC |
| 6062 | Right | CCAGCAGTTTGGCC |
| 6063 | Left | CAAAGGGCATGAACTAC |
| 6064 | Right | CACTTTGCCTCCTTCTGC |
| 6065 | Left | GCTCAACTGGTGTGTGCAGA |
| 6066 | Right | CACCCAGCAGTTTG |
| 6067 | Left | CAGATCGCAAAGGG |
| 6068 | Right | GCCTCCTTCTGCATGGTAT |
| 6069 | Left | CAAAGGGCATGAA |
| 6070 | Right | GCCTCCTTCTGCATGG |

EGFR Region3 176-225 bases

| | | |
|---|---|---|
| 6071 | Left | CAAAGGGCATGAACTACTTGGAG |
| 6072 | Right | CTCTGGTGGGTATAGATTCTGTGTA |
| 6073 | Left | CTATGTCCGGGAACACAAAGAC |
| 6074 | Right | CCTTCTGCATGGTATTCTTTCTCTT |
| 6075 | Left | GACTATGTCCGGGAACACAAA |
| 6076 | Right | CCTCCTTCTGCATGGTATTCTTTC |
| 6077 | Left | AAAGGGCATGAACTACTTGGAGGAC |
| 6078 | Right | ACTCTGGTGGGTATAGATTCTGT |
| 6079 | Left | CTCCCAGTACCTGCTCAACT |
| 6080 | Right | CTGCATGGTATTCTTTCTCTTCCG |
| 6081 | Left | CTCAACTGGTGTGTGCAGATC |
| 6082 | Right | GCCTCCTTCTGCATGGTATTCT |
| 6083 | Left | GGCTCCCAGTACCTGCTC |
| 6084 | Right | CATGGTATTCTTTCTCTTCCGCAC |
| 6085 | Left | GTCCGGGAACACAAAGACAATATT |
| 6086 | Right | GCCTCCTTCTGCATGGTAT |
| 6087 | Left | CTGCTCAACTGGTGTGTGC |
| 6088 | Right | TTCCAATGCCATCCACTTGAT |
| 6089 | Left | GTCCGGGAACACAAAGACAAT |
| 6090 | Right | TTTCTCTTCCGCACCCAG |
| 6091 | Left | CAAAGGGCATGAACTACTTG |
| 6092 | Right | AGATTCTGTGTAAAATTGATTCCA |
| 6093 | Left | TGGCTCCCAGTACCTG |
| 6094 | Right | CACTTTGCCTCCTTCTGC |
| 6095 | Left | GACTATGTCCGGGAACAC |
| 6096 | Right | GCCTCCTTCTGCATGG |
| 6097 | Left | CTTCGGCTGCCTCCTGG |
| 6098 | Right | CCAGCAGTTTGGCC |
| 6099 | Left | AAAGGGCATGAACTAC |
| 6100 | Right | ACTCTGGTGGGTATAGATTC |
| 6101 | Left | CTGCTCAACTGGTGT |
| 6102 | Right | TTCCAATGCCATCCACTT |
| 6103 | Left | CCCTTCGGCTGCCTCC |
| 6104 | Right | CACCCAGCAGTTTG |
| 6105 | Left | ACAATATTGGCTCCCA |
| 6106 | Right | ATCCACTTGATAGGCAC |
| 6107 | Left | CAGATCGCAAAGGG |
| 6108 | Right | AGATTCTGTGTAAAATTGAT |
| 6109 | Left | CAAAGGGCATGAAC |
| 6110 | Right | CTCTGGTGGGTATAGA |

EGFR Region3 226-275 bases

| | | |
|---|---|---|
| 6111 | Left | GTCCGGGAACACAAAGACAATATT |
| 6112 | Right | ATTCCAATGCCATCCACTTGAT |
| 6113 | Left | CTCCCAGTACCTGCTCAACT |
| 6114 | Right | CTCTGGTGGGTATAGATTCTGTGTA |
| 6115 | Left | GCTCATCACGCAGCTCATG |
| 6116 | Right | CCTTCTGCATGGTATTCTTTCTCTT |
| 6117 | Left | CAACTGGTGTGTGCAGATCG |
| 6118 | Right | ACTCTGGTGGGTATAGATTCTGT |
| 6119 | Left | CAGCTCATCACGCAGCTC |
| 6120 | Right | CCTCCTTCTGCATGGTATTCTTTC |
| 6121 | Left | CTCCACCGTGCAGCTCAT |
| 6122 | Right | CTGCATGGTATTCTTTCTCTTCCG |
| 6123 | Left | CGTGCAGCTCATCACGC |
| 6124 | Right | CATGGTATTCTTTCTCTTCCGCAC |
| 6125 | Left | CTATGTCCGGGAACACAAAGAC |
| 6126 | Right | GATTCTGTGTAAAATTGATTCCA |
| 6127 | Left | GCTCAACTGGTGTGTGCAG |
| 6128 | Right | CCGTAGCTCCAGACATCACT |
| 6129 | Left | CTATGTCCGGGAACACAAAGACAAT |
| 6130 | Right | CACTTTGCCTCCTTCTGC |
| 6131 | Left | CTTCGGCTGCCTCCTGG |
| 6132 | Right | GCCTCCTTCTGCATGGTATTCT |
| 6133 | Left | CTGCCTCACCTCCACCG |
| 6134 | Right | CTTTCTCTTCCGCACCCAG |
| 6135 | Left | GACTATGTCCGGGAACACAAA |
| 6136 | Right | ATTCCAATGCCATCCACTT |
| 6137 | Left | CTGCTCAACTGGTGTGTG |
| 6138 | Right | AAACAGTCACCCCGTAGCTC |
| 6139 | Left | GCTCCCAGTACCTGCTCA |
| 6140 | Right | CCCGTAGCTCCAGACATC |
| 6141 | Left | CCTCCACCGTGCAGCT |
| 6142 | Right | GCCTCCTTCTGCATGGTAT |
| 6143 | Left | TGGCTCCCAGTACCTGC |
| 6144 | Right | ACTCTGGTGGGTATAGATTC |
| 6145 | Left | CCCTTCGGCTGCCTCC |
| 6146 | Right | GCCTCCTTCTGCATGG |
| 6147 | Left | CAGATCGCAAAGGG |
| 6148 | Right | AAAGGTCATCAACTCCCAAACAG |
| 6149 | Left | CTGCTCAACTGGTGT |
| 6150 | Right | CACCCCGTAGCTCCAGAC |

EGFR Region4 276-325 bases

| | | |
|---|---|---|
| 6151 | Left | CTCCCAACCAAGCTCTCTT |
| 6152 | Right | TTGTGTTCCCGGACATAGTC |
| 6153 | Left | GAGAAGCTCCCAACCAAG |
| 6154 | Right | CCAGGAGGCAGCCGAAG |
| 6155 | Left | TCTCTTGAGGATCTTGA |
| 6156 | Right | GCCAATATTGTCTTTGTGTTCCC |
| 6157 | Left | CTCCCAACCAAGCTCT |
| 6158 | Right | TTGTGTTCCCGGACATA |
| 6159 | Left | TTGTGGAGCCTCTTACACCC |
| 6160 | Right | CGAAGGGCATGAG |
| 6161 | Left | CCCAGTGGAGAAGCTC |
| 6162 | Right | AGGAGGCAGCCG |
| 6163 | Left | GAGAAGCTCCCAACC |
| 6164 | Right | GACATAGTCCAGGAGG |

EGFR Region4 326-375 bases

| | | |
|---|---|---|
| 6165 | Left | CTTGTGGAGCCTCTTACACCCAG |
| 6166 | Right | GAGCCAATATTGTCTTTGTGTTCC |
| 6167 | Left | AGGAGAGGGAGCTTGTGGA |
| 6168 | Right | ATTGTCTTTGTGTTCCCGGACATAG |
| 6169 | Left | CTCCCAACCAAGCTCTCTT |
| 6170 | Right | AATATTGTCTTTGTGTTCCCGGAC |
| 6171 | Left | CTTGTGGAGCCTCTTACACC |
| 6172 | Right | TGGGAGCCAATATTGTCTTTGT |
| 6173 | Left | CTGCAGGAGAGGGAGCTTG |
| 6174 | Right | CCAGGAGGCAGCCGAAG |
| 6175 | Left | GAGAAGCTCCCAACCAAG |
| 6176 | Right | CACACACCAGTTGAGCAGG |
| 6177 | Left | CTCCCAACCAAGCTCT |
| 6178 | Right | GATCTGCACACACCAGTTGAG |
| 6179 | Left | TCTCTTGAGGATCTTGA |
| 6180 | Right | CGATCTGCACACACCAGTT |
| 6181 | Left | CTTGTGGAGCCTCTTAC |
| 6182 | Right | TGGGAGCCAATATTGTCTT |
| 6183 | Left | TGCTGCAGGAGAGGGAG |
| 6184 | Right | GACATAGTCCAGGAGG |
| 6185 | Left | CCACATCGTTCGGAAGCG |
| 6186 | Right | CGAAGGGCATGAG |
| 6187 | Left | CCCAGTGGAGAAGCT |
| 6188 | Right | CCAGTTGAGCAGGTAC |
| 6189 | Left | GAGAAGCTCCCAACC |
| 6190 | Right | TGGGAGCCAATATTGT |
| 6191 | Left | TCGGAAGCGCACG |
| 6192 | Right | AGGAGGCAGCCG |
| 6193 | Left | TGTGGAGCCTCT |
| 6194 | Right | TACTGGGAGCCA |

EGFR Region4 376-425 bases

| | | |
|---|---|---|
| 6195 | Left | AGGAGAGGGAGCTTGTGGA |
| 6196 | Right | GAGCCAATATTGTCTTTGTGTTCC |
| 6197 | Left | GAAGGCGCCACATCGTTC |
| 6198 | Right | AATATTGTCTTTGTGTTCCCGGAC |
| 6199 | Left | TTGTGGAGCCTCTTACACCC |
| 6200 | Right | CTCCAAGTAGTTCATGCCCTTT |
| 6201 | Left | CCACATCGTTCGGAAGCG |
| 6202 | Right | GGGAGCCAATATTGTCTTTGTGT |
| 6203 | Left | CTGCAGGAGAGGGAGCTTG |
| 6204 | Right | TTGTCTTTGTGTTCCCGGACATAG |
| 6205 | Left | TGGAGCCTCTTACACCCAGT |
| 6206 | Right | TCCTCCAAGTAGTTCATGCCC |
| 6207 | Left | CTCCCAACCAAGCTCTCTT |
| 6208 | Right | GACGGTCCTCCAAGTAGTTCAT |
| 6209 | Left | TGCTGCAGGAGAGGGAG |
| 6210 | Right | GATCTGCACACACCAGTTGAG |
| 6211 | Left | CATCGTTCGGAAGCGCAC |
| 6212 | Right | TGGGAGCCAATATTGTCTTTG |
| 6213 | Left | ATCGGCCTCTTCATGCGAA |
| 6214 | Right | CCAGGAGGCAGCCGAAG |
| 6215 | Left | GAGAAGCTCCCAACCAAG |
| 6216 | Right | CGACGGTCCTCCAAGTAGTT |
| 6217 | Left | CTTGTGGAGCCTCTTACA |
| 6218 | Right | CACACACCAGTTGAGCAGG |
| 6219 | Left | CCCAGTGGAGAAGCTC |
| 6220 | Right | AAGCGACGGTCCTCCAAGTA |
| 6221 | Left | CGCCACATCGTTCGGAA |
| 6222 | Right | TGGGAGCCAATATTGTCT |
| 6223 | Left | CTCCCAACCAAGCTCT |
| 6224 | Right | CAAGCGACGGTCCTCCAA |
| 6225 | Left | GAGAAGCTCCCAACC |
| 6226 | Right | CGATCTGCACACACCAGTT |
| 6227 | Left | TCTCTTGAGGATCTTGA |
| 6228 | Right | GTACGTTCCTGGCTGCCA |
| 6229 | Left | GGATCGGCCTCTTCATGC |
| 6230 | Right | GACATAGTCCAGGAGG |
| 6231 | Left | CTGGTGGTGGCCCTGG |
| 6232 | Right | AGGAGGCAGCCG |
| 6233 | Left | CCTCTTGCTGCTGGTGGT |
| 6234 | Right | CGAAGGGCATGAG |

EGFR Region4 426-475 bases

| | | |
|---|---|---|
| 6235 | Left | ATCGGCCTCTTCATGCGAA |
| 6236 | Right | GAGCCAATATTGTCTTTGTGTTCC |
| 6237 | Left | CCTCCTCTTGCTGCTGGT |
| 6238 | Right | AATATTGTCTTTGTGTTCCCGGAC |
| 6239 | Left | TTGTGGAGCCTCTTACACCC |
| 6240 | Right | GACGGTCCTCCAAGTAGTTCAT |
| 6241 | Left | GAAGGCGCCACATCGTTC |
| 6242 | Right | GGGAGCCAATATTGTCTTTGTGT |
| 6243 | Left | CTTGTGGAGCCTCTTACACCCAGT |
| 6244 | Right | CCTCCAAGTAGTTCATGCCCTTT |
| 6245 | Left | CCTCTTGCTGCTGGTGGT |
| 6246 | Right | ATTGTCTTTGTGTTCCCGGACATAG |
| 6247 | Left | GGAGAGGGAGCTTGTGGAG |
| 6248 | Right | TCCTCCAAGTAGTTCATGCCC |
| 6249 | Left | CCACATCGTTCGGAAGCG |
| 6250 | Right | GATCTGCACACACCAGTTGAG |
| 6251 | Left | CTCCCAACCAAGCTCTCTT |
| 6252 | Right | TGTTTTCACCAGTACGTTCCTG |
| 6253 | Left | CTGCAGGAGAGGGAGCTTG |
| 6254 | Right | CGACGGTCCTCCAAGTAGTT |
| 6255 | Left | CATCGTTCGGAAGCGCAC |
| 6256 | Right | CACACACCAGTTGAGCAGG |
| 6257 | Left | GAGAAGCTCCCAACCAAG |
| 6258 | Right | GATCTTGACATGCTGCGGTGTTTTC |
| 6259 | Left | GCGCCACATCGTTCGGAA |
| 6260 | Right | TGGGAGCCAATATTGTCTTTG |
| 6261 | Left | TGCTGCAGGAGAGGGAG |
| 6262 | Right | AAGCGACGGTCCTCCAAGTA |
| 6263 | Left | GCCCTCCTCTTGCTGCT |
| 6264 | Right | CCAGGAGGCAGCCGAAG |
| 6265 | Left | CTTGTGGAGCCTCTTACA |
| 6266 | Right | AGTACGTTCCTGGCTGCC |
| 6267 | Left | TCTCTTGAGGATCTTGA |
| 6268 | Right | CCAAAATCTGTGATCTTGACATGC |
| 6269 | Left | GAAGGCGCCACATCG |
| 6270 | Right | CGATCTGCACACACCAGTT |
| 6271 | Left | CTCCCAACCAAGCTCT |
| 6272 | Right | GATCTTGACATGCTGCGGTG |
| 6273 | Left | CTGGTGGTGGCCCTGG |
| 6274 | Right | TGGGAGCCAATATTGTCT |

EGFR Region5 276-325 bases

| | | |
|---|---|---|
| 6275 | Left | ATCCTCGATGAAGCCTACGTG |
| 6276 | Right | TCTCTTCCGCACCCAG |
| 6277 | Left | CGATGAAGCCTACGTGATGG |
| 6278 | Right | TTCTTTCTCTTCCGCAC |
| 6279 | Left | GGAAATCCTCGATGAAGCCTAC |
| 6280 | Right | CCAGCAGTTTGGCC |
| 6281 | Left | GGAAATCCTCGATGAAGCC |
| 6282 | Right | CACCCAGCAGTTTG |

EGFR Region5 326-375 bases

| | | |
|---|---|---|
| 6283 | Left | GAAATCCTCGATGAAGCCTACG |
| 6284 | Right | CCTTCTGCATGGTATTCTTTCTCTT |
| 6285 | Left | GAAATCCTCGATGAAGCCTACGTGA |
| 6286 | Right | CTGCATGGTATTCTTTCTCTTCCG |
| 6287 | Left | AAGCAACATCTCCGAAAGCCAA |
| 6288 | Right | CTCCTTCTGCATGGTATTCTTTCT |
| 6289 | Left | AACATCTCCGAAAGCCAACAAG |
| 6290 | Right | GCCTCCTTCTGCATGGTATT |
| 6291 | Left | AGAAGCAACATCTCCGAAAG |
| 6292 | Right | CATGGTATTCTTTCTCTTCCGCAC |
| 6293 | Left | GGAAATCCTCGATGAAGCCT |
| 6294 | Right | GCCTCCTTCTGCATGGTATTCTT |
| 6295 | Left | TCGCTATCAAGGAATTAAGAGAAGC |
| 6296 | Right | TCTCTTCCGCACCCAG |
| 6297 | Left | CTCGATGAAGCCTACGTGATGG |
| 6298 | Right | CACTTTGCCTCCTTCTGC |
| 6299 | Left | TCAAGGAATTAAGAGAAGCAACATCT |
| 6300 | Right | CCAGCAGTTTGGCC |
| 6301 | Left | CCGTCGCTATCAAGGAATTAAGAG |
| 6302 | Right | CACCCAGCAGTTTG |
| 6303 | Left | GGAAATCCTCGATGAAG |
| 6304 | Right | GCCTCCTTCTGCATGGT |
| 6305 | Left | GGAAATCCTCGATG |
| 6306 | Right | ATCCACTTGATAGGCAC |
| 6307 | Left | CAACAAGGAAATCC |
| 6308 | Right | CACTTTGCCTCCTTC |

EGFR Region5 376-425 bases

| | | |
|---|---|---|
| 6309 | Left | AAGGTGAGAAAGTTAAAATTCCCGT |
| 6310 | Right | CCTTCTGCATGGTATTCTTTCTCTT |
| 6311 | Left | GAAAGTTAAAATTCCCGTCGCTATC |
| 6312 | Right | CTCCTTCTGCATGGTATTCTTTCT |
| 6313 | Left | GAAATCCTCGATGAAGCCTACG |
| 6314 | Right | CTCTGGTGGGTATAGATTCTGTGTA |
| 6315 | Left | CCGTCGCTATCAAGGAATTAAGAG |
| 6316 | Right | CATGGTATTCTTTCTCTTCCGCAC |
| 6317 | Left | GTTAAAATTCCCGTCGCTATCAAG |
| 6318 | Right | TGCATGGTATTCTTTCTCTTCCG |
| 6319 | Left | ATTCCCGTCGCTATCAAGGAATTA |
| 6320 | Right | GCCTCCTTCTGCATGGTATTCTT |
| 6321 | Left | TCGCTATCAAGGAATTAAGAGAAGC |
| 6322 | Right | GCCTCCTTCTGCATGGTATT |
| 6323 | Left | AACATCTCCGAAAGCCAACAAG |
| 6324 | Right | ATTCCAATGCCATCCACTTGAT |
| 6325 | Left | CTCGATGAAGCCTACGTGATGG |
| 6326 | Right | ACTCTGGTGGGTATAGATTCTGT |
| 6327 | Left | TGAGAAAGTTAAAATTCCCGTCGC |
| 6328 | Right | CTTTCTCTTCCGCACCCAG |
| 6329 | Left | GCAACATCTCCGAAAGCCAA |
| 6330 | Right | AGATTCTGTGTAAAATTGATTCCA |
| 6331 | Left | TCAAGGAATTAAGAGAAGCAACATCT |
| 6332 | Right | CACTTTGCCTCCTTCTGC |
| 6333 | Left | AGAAGCAACATCTCCGAAAGC |
| 6334 | Right | ATTCCAATGCCATCCACTT |
| 6335 | Left | ATTCCCGTCGCTATCAAGGAA |
| 6336 | Right | GCCTCCTTCTGCATGGT |
| 6337 | Left | TCGATGAAGCCTACGTGA |
| 6338 | Right | CCCGTAGCTCCAGACATCA |
| 6339 | Left | GGAAATCCTCGATGAAGCCT |
| 6340 | Right | ACTCTGGTGGGTATAGATTC |
| 6341 | Left | CAGAAGGTGAGAAAGTTAAAATTCC |
| 6342 | Right | CCAGCAGTTTGGCC |
| 6343 | Left | CCCAGAAGGTGAGAAAGTTAAAAT |
| 6344 | Right | CACCCAGCAGTTTG |
| 6345 | Left | TATCAAGGAATTAAGAGAAGCAACA |
| 6346 | Right | TCCAATGCCATCCA |
| 6347 | Left | AGAAGCAACATCTCCGAA |
| 6348 | Right | ATCCACTTGATAGGCAC |

EGFR Region5 426-475 bases

| | | |
|---|---|---|
| 6349 | Left | AAGTTAAAATTCCCGTCGCTATCA |
| 6350 | Right | CTCTGGTGGGTATAGATTCTGTGTA |
| 6351 | Left | TCGCTATCAAGGAATTAAGAGAAGC |
| 6352 | Right | ATTCCAATGCCATCCACTTGAT |
| 6353 | Left | CCGTCGCTATCAAGGAATTAAGAG |
| 6354 | Right | ACTCTGGTGGGTATAGATTCTGT |
| 6355 | Left | AAGGTGAGAAAGTTAAAATTCCCGT |
| 6356 | Right | AGATTCTGTGTAAAATTGATTCCA |
| 6357 | Left | GAAATCCTCGATGAAGCCTACG |
| 6358 | Right | GTCATCAACTCCCAAACAGTCAC |
| 6359 | Left | AAAGATCAAAGTGCTGGGCTC |
| 6360 | Right | CATGGTATTCTTTCTCTTCCGCAC |
| 6361 | Left | TCAAGGAATTAAGAGAAGCAACATCT |
| 6362 | Right | CCCCGTAGCTCCAGACATC |
| 6363 | Left | GGCACGGTGTATAAGGGAC |
| 6364 | Right | CCTTCTGCATGGTATTCTTTCTCTT |
| 6365 | Left | CCCAGAAGGTGAGAAAGTTAAA |
| 6366 | Right | CCTCCTTCTGCATGGTATTCTTTC |
| 6367 | Left | AGAAGCAACATCTCCGAAAGC |
| 6368 | Right | CGTAGCTCCAGACATCACTCT |
| 6369 | Left | CTCGATGAAGCCTACGTGATGG |
| 6370 | Right | TGGATCCAAAGGTCATCAACTC |
| 6371 | Left | AACATCTCCGAAAGCCAACAAG |
| 6372 | Right | CATCAACTCCCAAACAGTCACCCC |
| 6373 | Left | AAATCCTCGATGAAGCCTACGTGA |
| 6374 | Right | AAACAGTCACCCCGTAGCTC |
| 6375 | Left | AAAGATCAAAGTGCTGGGCTCCGG |
| 6376 | Right | TGCATGGTATTCTTTCTCTTCCG |
| 6377 | Left | GGAAATCCTCGATGAAGCCT |
| 6378 | Right | AAGGTCATCAACTCCCAAACAG |
| 6379 | Left | GAGAAAGTTAAAATTCCCGTCGCTA |
| 6380 | Right | ATTCCAATGCCATCCACTT |
| 6381 | Left | GAAGCAACATCTCCGAAAGCCAAC |
| 6382 | Right | CACCCCGTAGCTCCAGAC |
| 6383 | Left | TTAAAATTCCCGTCGCTATCAAGG |
| 6384 | Right | ACTCTGGTGGGTATAGATTC |
| 6385 | Left | CAGAAGGTGAGAAAGTTAAAATTCC |
| 6386 | Right | CACTTTGCCTCCTTCTGC |
| 6387 | Left | GGGCTCCGGTGCGTT |
| 6388 | Right | GCCTCCTTCTGCATGGTATTCT |

EGFR Region6 476-525 bases

| | | |
|---|---|---|
| 6389 | Left | GGAGCCTCTTACACCCAGT |
| 6390 | Right | TTCTGCATGGTATTCTTTCTCTTCC |
| 6391 | Left | CTCCCAACCAAGCTCTCTT |
| 6392 | Right | CTCCTTCTGCATGGTATTCTTTCTC |
| 6393 | Left | CTTGTGGAGCCTCTTACAC |
| 6394 | Right | CATGGTATTCTTTCTCTTCCGCAC |
| 6395 | Left | GAGAAGCTCCCAACCAAG |
| 6396 | Right | CCTCCTTCTGCATGGTATTCTTT |
| 6397 | Left | GGAGAGGGAGCTTGTGGA |
| 6398 | Right | TTTCTCTTCCGCACCCAG |
| 6399 | Left | CCCAGTGGAGAAGCTC |
| 6400 | Right | GCCTCCTTCTGCATGGTATTC |
| 6401 | Left | TCTCTTGAGGATCTTGA |
| 6402 | Right | ATTCCAATGCCATCCACTTGAT |
| 6403 | Left | CTCCCAACCAAGCTCT |
| 6404 | Right | GCCTCCTTCTGCATGGTA |
| 6405 | Left | CTGCAGGAGAGGGAGCTTG |
| 6406 | Right | CCAGCAGTTTGGCC |
| 6407 | Left | GAGAAGCTCCCAACC |
| 6408 | Right | CACTTTGCCTCCTTCTGC |
| 6409 | Left | TGCTGCAGGAGAGGGA |
| 6410 | Right | CACCCAGCAGTTTG |
| 6411 | Left | CCCAGTGGAGAAG |
| 6412 | Right | GCCTCCTTCTGCATG |

EGFR Region6 526-575 bases

| | | |
|---|---|---|
| 6413 | Left | CTTGTGGAGCCTCTTACACCCAG |
| 6414 | Right | CCTTCTGCATGGTATTCTTTCTCTT |
| 6415 | Left | CAGGAGAGGGAGCTTGTGG |
| 6416 | Right | CTGCATGGTATTCTTTCTCTTCCG |
| 6417 | Left | CTGCAGGAGAGGGAGCTTG |
| 6418 | Right | CCTCCTTCTGCATGGTATTCTTTC |
| 6419 | Left | CTTGTGGAGCCTCTTACAC |
| 6420 | Right | CATGGTATTCTTTCTCTTCCGCAC |
| 6421 | Left | CTCCCAACCAAGCTCTCTT |
| 6422 | Right | ATTCCAATGCCATCCACTTGAT |
| 6423 | Left | GAGAAGCTCCCAACCAAG |
| 6424 | Right | GCCTCCTTCTGCATGGTATTCT |
| 6425 | Left | TGCAGGAGAGGGAGC |
| 6426 | Right | CTTTCTCTTCCGCACCCAG |
| 6427 | Left | CCCAGTGGAGAAGCTC |
| 6428 | Right | GCCTCCTTCTGCATGGTAT |
| 6429 | Left | TCTCTTGAGGATCTTGA |
| 6430 | Right | AGATTCTGTGTAAAATTGATTCCA |
| 6431 | Left | CTCCCAACCAAGCTCT |
| 6432 | Right | CACTTTGCCTCCTTCTGC |
| 6433 | Left | CCACATCGTTCGGAAGCG |
| 6434 | Right | CCAGCAGTTTGGCC |
| 6435 | Left | GAGAAGCTCCCAACC |
| 6436 | Right | ATTCCAATGCCATCCACTT |
| 6437 | Left | CATCGTTCGGAAGCGCAC |
| 6438 | Right | CACCCAGCAGTTTG |
| 6439 | Left | CTTGTGGAGCCTCTTA |
| 6440 | Right | GCCTCCTTCTGCATGG |
| 6441 | Left | CCCAGTGGAGAAG |
| 6442 | Right | ATCCACTTGATAGGCAC |
| 6443 | Left | CTTGTGGAGCCTC |
| 6444 | Right | CACTTTGCCTCCTTC |

EGFR Region6 576-625 bases

| | | |
|---|---|---|
| 6445 | Left | CTTGTGGAGCCTCTTACACCCAG |
| 6446 | Right | CTCTGGTGGGTATAGATTCTGTGTA |
| 6447 | Left | CCACATCGTTCGGAAGCG |
| 6448 | Right | CCTTCTGCATGGTATTCTTTCTCTT |
| 6449 | Left | ATCGGCCTCTTCATGCGAA |
| 6450 | Right | CATGGTATTCTTTCTCTTCCGCAC |
| 6451 | Left | GAAGGCGCCACATCGTTC |
| 6452 | Right | CCTCCTTCTGCATGGTATTCTTTC |
| 6453 | Left | CTTGTGGAGCCTCTTACACC |
| 6454 | Right | ACTCTGGTGGGTATAGATTCTGT |
| 6455 | Left | CAGGAGAGGGAGCTTGTGG |
| 6456 | Right | ATTCCAATGCCATCCACTTGAT |
| 6457 | Left | CGTTCGGAAGCGCACG |
| 6458 | Right | CTGCATGGTATTCTTTCTCTTCCG |
| 6459 | Left | CTGCAGGAGAGGGAGCTTG |
| 6460 | Right | GCCTCCTTCTGCATGGTATTCT |
| 6461 | Left | CTCCCAACCAAGCTCTCTT |
| 6462 | Right | CGTAGCTCCAGACATCACTCT |
| 6463 | Left | GGATCGGCCTCTTCATGC |
| 6464 | Right | CTTTCTCTTCCGCACCCAG |
| 6465 | Left | GAGAAGCTCCCAACCAAG |
| 6466 | Right | AGATTCTGTGTAAAATTGATTCCA |
| 6467 | Left | GCGCCACATCGTTCGGAA |
| 6468 | Right | GCCTCCTTCTGCATGGTAT |
| 6469 | Left | CGAAGGCGCCACATCG |
| 6470 | Right | CACTTTGCCTCCTTCTGC |
| 6471 | Left | TGCTGCAGGAGAGGGAG |
| 6472 | Right | ATTCCAATGCCATCCACTT |
| 6473 | Left | CCCAGTGGAGAAGCTC |
| 6474 | Right | CCCGTAGCTCCAGACATCAC |
| 6475 | Left | TCTCTTGAGGATCTTGA |
| 6476 | Right | AAAGGTCATCAACTCCCAAACAG |
| 6477 | Left | CTCCCAACCAAGCTCT |
| 6478 | Right | AAACAGTCACCCCGTAGCTC |
| 6479 | Left | GAGAAGCTCCCAACC |
| 6480 | Right | ACCCCGTAGCTCCAGACAT |
| 6481 | Left | CTTGTGGAGCCTCTTAC |
| 6482 | Right | ACTCTGGTGGGTATAGATTC |
| 6483 | Left | CTCTTGCTGCTGGTGGTG |
| 6484 | Right | CCAGCAGTTTGGCC |

EGFR Region6 750-1250 bases

| | | |
|---|---|---|
| 6485 | Left | TTTGTGGAGAACTCTGAGTGCATA |
| 6486 | Right | CTCTGGTGGGTATAGATTCTGTGTA |
| 6487 | Left | CAGGAGTCATGGGAGAAAACAAC |
| 6488 | Right | CCTTCTGCATGGTATTCTTTCTCTT |
| 6489 | Left | ACCAAAATTATAAGCAACAGAGGTG |
| 6490 | Right | CCTCCTTCTGCATGGTATTCTTT |
| 6491 | Left | TATAAGCAACAGAGGTGAAAACAGC |
| 6492 | Right | ACTCTGGTGGGTATAGATTCTGT |
| 6493 | Left | GAGTCATGGGAGAAAACAACACC |
| 6494 | Right | TTTTGGAGAATTCGATGATCAACTC |
| 6495 | Left | GAGTTTGTGGAGAACTCTGAGTG |
| 6496 | Right | CATGGTATTCTTTCTCTTCCGCAC |
| 6497 | Left | GAGAAAACAACACCCTGGTCTG |
| 6498 | Right | TGCATGGTATTCTTTCTCTTCCG |
| 6499 | Left | AAAACAACACCCTGGTCTGGAAGTA |
| 6500 | Right | GATTCCGTCATATGGCTTGGATC |
| 6501 | Left | GCAGGAGTCATGGGAGAAAAC |
| 6502 | Right | CTATCATCCAGCACTTGACCATG |
| 6503 | Left | CCAAGGGAGTTTGTGGAGAAC |
| 6504 | Right | AAAGGTCATCAACTCCCAAACAG |
| 6505 | Left | GAGAAAACAACACCCTGGTCTGGAA |
| 6506 | Right | CGACTATCTGCGTCTATCATCCAG |
| 6507 | Left | AACGAATGGGCCTAAGATCCC |
| 6508 | Right | CATTTCTATCAATGCAAGCCACG |
| 6509 | Left | GTCAGAAAACCAAAATTATAAGCAA |
| | | CAG |
| 6510 | Right | ATTCCAATGCCATCCACTTGAT |
| 6511 | Left | ATCCAAACTGCACCTACGGAT |
| 6512 | Right | GATCCAAAGGTCATCAACTCCCAA |
| 6513 | Left | CCATGAACATCACCTGCACAG |
| 6514 | Right | CTATCTGCGTCTATCATCCAGCAC |
| 6515 | Left | AATTATAAGCAACAGAGGTGAAAAC |
| 6516 | Right | CCGTAGCTCCAGACATCACT |
| 6517 | Left | AGGTGAAAACAGCTGCAAGG |
| 6518 | Right | GTCATCAACTCCCAAACAGTCAC |
| 6519 | Left | GGGAGTTTGTGGAGAACTCTG |
| 6520 | Right | CATCCAGCACTTGACCATGATC |
| 6521 | Left | CCATCCAAACTGCACCTACG |
| 6522 | Right | TGATCAACTCACGGAACTTTGG |
| 6523 | Left | AATGGGCCTAAGATCCCGTC |
| 6524 | Right | CGTCTATCATCCAGCACTTGAC |

TABLE 13
BRAF cDNA Capture Primer List for NGS Panel

| | | |
|---|---|---|
| Seq. | | |
| ID | Primer | Sequence |

BRAF Region1 75-125 bases

| | | |
|---|---|---|
| 6525 | Left | gacaggaatcgaatgaaaacacttg |
| 6526 | Right | tttcccttgtagactgttccaaat |
| 6527 | Left | ggaatcgaatgaaaacacttggtag |
| 6528 | Right | cactttcccttgtagactgttcc |
| 6529 | Left | ggaatcgaatgaaaacac |
| 6530 | Right | ccactttcccttgtagactgt |
| 6531 | Left | ggaatcgaatgaaaa |
| 6532 | Right | ccactttcccttgtagac |

BRAF Region1 126-175 bases

| | | |
|---|---|---|
| 6533 | Left | gaagacaggaatcgaatgaaaacac |
| 6534 | Right | gctgtcacattcaacattttcactg |
| 6535 | Left | aggaatcgaatgaaaacacttggta |
| 6536 | Right | cacattcaacattttcactgccac |
| 6537 | Left | tcttcatcctcagaagacaggaatc |
| 6538 | Right | caacattttcactgccacatcac |
| 6539 | Left | aagtcatcttcatcctcagaagaca |
| 6540 | Right | cattttcactgccacatcaccat |
| 6541 | Left | agacaggaatcgaatgaaaacacttg |
| 6542 | Right | tttcccttgtagactgttccaaat |
| 6543 | Left | ctcagaagacaggaatcgaatgaaa |
| 6544 | Right | catcaccatgccactttccc |
| 6545 | Left | aatcgaatgaaaacacttggtagac |
| 6546 | Right | gctgtcacattcaacattttca |
| 6547 | Left | aaatctccaggacctcagcgagaaa |
| 6548 | Right | actttcccttgtagactgttcca |
| 6549 | Left | atcctcagaagacaggaatcgaa |
| 6550 | Right | atcaccatgccactttcccttg |
| 6551 | Left | cagcgagaaaggaagtcatct |
| 6552 | Right | gccactttcccttgtagactgtt |
| 6553 | Left | aagtcatcttcatcctcagaag |
| 6554 | Right | atcaccatgccactttcccttgtag |
| 6555 | Left | ctccaggacctcagcgag |
| 6556 | Right | tgccactttcccttgtagact |
| 6557 | Left | catcttcatcctcagaagacagga |
| 6558 | Right | catcaccatgccacttt |
| 6559 | Left | ctcagcgagaaaggaagtca |
| 6560 | Right | catcaccatgccac |

BRAF Region1 176-225 bases

| | | |
|---|---|---|
| 6561 | Left | aagtcatcttcatcctcagaagaca |
| 6562 | Right | gctgtcacattcaacattttcactg |
| 6563 | Left | actaactaacgtgaaagccttacag |
| 6564 | Right | attttcactgccacatcaccat |
| 6565 | Left | ccttacagaaatctccaggacct |
| 6566 | Right | tcacattcaacattttcactgcca |
| 6567 | Left | aagccttacagaaatctccagga |
| 6568 | Right | caacattttcactgccacatcac |
| 6569 | Left | ctcagcgagaaaggaagtcatct |
| 6570 | Right | cttgtaactgctgaggtgtaggt |
| 6571 | Left | ttacagaaatctccaggacctcagc |
| 6572 | Right | tgctgtcacattcaacattttca |
| 6573 | Left | ccaggacctcagcgagaaa |
| 6574 | Right | ctgaggtgtaggtgctgtca |
| 6575 | Left | ctaacgtgaaagccttacagaaat |
| 6576 | Right | caacattttcactgccacat |
| 6577 | Left | aagtcatcttcatcctcagaag |
| 6578 | Right | ctgctgaggtgtaggtgct |
| 6579 | Left | tcagcgagaaaggaagtca |
| 6580 | Right | gcttgtaactgctgaggtgta |
| 6581 | Left | gaaatctccaggacctcagcgag |
| 6582 | Right | tgctgtcacattcaacattt |
| 6583 | Left | aagtcatcttcatcctcag |
| 6584 | Right | ttttgaaggcttgtaactgctg |
| 6585 | Left | ctcactaactaacgtgaaagcctta |
| 6586 | Right | catcaccatgcca |
| 6587 | Left | aagtcatcttcatcct |
| 6588 | Right | aggcttgtaactgctgaggt |
| 6589 | Left | ctcagcgagaaaggaag |
| 6590 | Right | tgctgtcacattcaaca |
| 6591 | Left | agccttacagaaatctcca |
| 6592 | Right | gctgtcacattca |

BRAF Region1 226-275 bases

| | | |
|---|---|---|
| 6593 | Left | agtcatcttcatcctcagaagaca |
| 6594 | Right | tgaagagtaggatattcacatgtcg |
| 6595 | Left | ctcactaactaacgtgaaagcctta |
| 6596 | Right | cacattcaacattttcactgccac |
| 6597 | Left | actaactaacgtgaaagccttacag |
| 6598 | Right | ttttgaaggcttgtaactgctgag |
| 6599 | Left | cctcattacctggctcactaacta |
| 6600 | Right | gctgtcacattcaacattttcactg |
| 6601 | Left | actaacgtgaaagccttacagaaat |
| 6602 | Right | cttgtaactgctgaggtgtaggt |
| 6603 | Left | attacctggctcactaactaacgt |
| 6604 | Right | caacattttcactgccacatcac |
| 6605 | Left | ctggctcactaactaacgtgaaag |
| 6606 | Right | tgctgtcacattcaacattttca |
| 6607 | Left | aacgtgaaagccttacagaaatctc |
| 6608 | Right | ggcttgtaactgctgaggtgta |
| 6609 | Left | cttacagaaatctccaggacctca |
| 6610 | Right | taactgctgaggtgtaggtgct |
| 6611 | Left | aagccttacagaaatctccagga |
| 6612 | Right | ttttgaaggcttgtaactgct |
| 6613 | Left | cctcattacctggctcactaa |
| 6614 | Right | cattttcactgccacatcaccat |
| 6615 | Left | gaaatctccaggacctcagcgagaa |
| 6616 | Right | ctgaggtgtaggtgctgtca |
| 6617 | Left | agtcatcttcatcctcagaag |
| 6618 | Right | gcccatgaagagtaggatattc |
| 6619 | Left | ctcagcgagaaaggaagtcatct |
| 6620 | Right | catgtcgtgttttcctga |
| 6621 | Left | aaatctccaggacctcagcg |
| 6622 | Right | gttttcctgagtactcctac |
| 6623 | Left | cccctgcctcattacctg |
| 6624 | Right | tgctgtcacattcaacattt |
| 6625 | Left | aagtcatcttcatcctcag |
| 6626 | Right | tgaagagtaggatattcacatg |
| 6627 | Left | ctcagcgagaaaggaagtca |
| 6628 | Right | tttgaaggcttgtaact |
| 6629 | Left | cctcattacctggctcac |
| 6630 | Right | tgctgtcacattcaaca |
| 6631 | Left | caggtttgtctgctacccc |
| 6632 | Right | catcaccatgcca |

BRAF Region1 276-325 bases

| | | |
|---|---|---|
| 6633 | Left | aagtcatcttcatcctcagaagaca |
| 6634 | Right | ggaatagcccatgaagagtaggata |
| 6635 | Left | actaactaacgtgaaagccttacag |
| 6636 | Right | tgaagagtaggatattcacatgtcg |
| 6637 | Left | ctcattacctggctcactaactaac |
| 6638 | Right | ttttgaaggcttgtaactgctgag |
| 6639 | Left | aacgtgaaagccttacagaaatctc |
| 6640 | Right | atagcccatgaagagtaggatattc |
| 6641 | Left | ttgatgacttgattagagaccaagg |
| 6642 | Right | attttcactgccacatcaccat |
| 6643 | Left | gatttcgtggtgatggaggatc |
| 6644 | Right | gctgtcacattcaacattttcactg |
| 6645 | Left | attacctggctcactaactaacgtg |
| 6646 | Right | gcttgtaactgctgaggtgtag |
| 6647 | Left | caaggatttcgtggtgatggag |
| 6648 | Right | tcacattcaacattttcactgcca |
| 6649 | Left | cctcattacctggctcactaact |
| 6650 | Right | cttgtaactgctgaggtgtaggtg |
| 6651 | Left | ctggctcactaactaacgtgaaag |
| 6652 | Right | ttgaaggcttgtaactgctgaggtg |
| 6653 | Left | aagccttacagaaatctccagga |
| 6654 | Right | ggaatagcccatgaagagtagg |
| 6655 | Left | tattgatgacttgattagagacca |
| 6656 | Right | caacattttcactgccacatcac |
| 6657 | Left | ttgattagagaccaaggatttcgtg |
| 6658 | Right | tgctgtcacattcaacatttt |
| 6659 | Left | gctcactaactaacgtgaaagcctt |
| 6660 | Right | ttttgaaggcttgtaactgct |
| 6661 | Left | cttacagaaatctccaggacctca |
| 6662 | Right | tgaagagtaggatattcacatg |
| 6663 | Left | cccctgcctcattacctgg |
| 6664 | Right | gtaactgctgaggtgtaggtgctg |
| 6665 | Left | aaatctccaggacctcagcgagaaa |
| 6666 | Right | cagttgtggctttgtggaat |
| 6667 | Left | ctcagcgagaaaggaagtcatct |
| 6668 | Right | ggtaacaatagccagttgtg |
| 6669 | Left | atgacttgattagagaccaaggatt |
| 6670 | Right | aacattttcactgccacat |
| 6671 | Left | ctaacgtgaaagccttacagaaat |
| 6672 | Right | catgtcgtgttttcctgag |

BRAF Region1 326-375 bases

| | | |
|---|---|---|
| 6673 | Left | ctcattacctggctcactaactaac |
| 6674 | Right | ggaatagcccatgaagagtaggata |
| 6675 | Left | ctcactaactaacgtgaaagcctta |
| 6676 | Right | tgaagagtaggatattcacatgtcg |
| 6677 | Left | agtcatcttcatcctcagaagaca |
| 6678 | Right | gatatggagatggtgatacaagctg |
| 6679 | Left | acttgattagagaccaaggatttcg |
| 6680 | Right | ttttgaaggcttgtaactgctgag |
| 6681 | Left | aacgtgaaagccttacagaaatctc |
| 6682 | Right | gaatagcccatgaagagtaggatattc |
| 6683 | Left | tgatgacttgattagagaccaagga |
| 6684 | Right | cttgtaactgctgaggtgtaggt |
| 6685 | Left | actaacgtgaaagccttacagaaat |
| 6686 | Right | ggaatagcccatgaagagtagg |
| 6687 | Left | tataaacacaatagaacctgtcaat |
| 6688 | Right | gctgtcacattcaacattttcactg |
| 6689 | Left | ctcagcgagaaaggaagtcatct |
| 6690 | Right | atggagatggtgatacaagctggag |
| 6691 | Left | gcatataaacacaatagaacctgtc |
| 6692 | Right | cattttcactgccacatcacca |
| 6693 | Left | tattgatgacttgattagagacca |
| 6694 | Right | cacattcaacattttcactgccac |
| 6695 | Left | gatttcgtggtgatggaggatc |
| 6696 | Right | gaaggcttgtaactgctgaggtgta |
| 6697 | Left | aaacacaatagaacctgtcaatatt |
| 6698 | Right | tgctgtcacattcaacattttca |
| 6699 | Left | caaggatttcgtggtgatggag |
| 6700 | Right | taactgctgaggtgtaggtgct |
| 6701 | Left | attacctggctcactaactaacgtg |
| 6702 | Right | cagttgtggctttgtggaat |
| 6703 | Left | actaactaacgtgaaagccttacag |
| 6704 | Right | ggtaacaatagccagttgtg |
| 6705 | Left | cttacagaaatctccaggacctca |
| 6706 | Right | agccctcacaccactgg |
| 6707 | Left | ccgatcctcatcagctccc |
| 6708 | Right | caacattttcactgccacatca |
| 6709 | Left | ctggctcactaactaacgtgaaag |
| 6710 | Right | tgaagagtaggatattcacatg |
| 6711 | Left | ccaggacctcagcgagaaa |
| 6712 | Right | tgatatggagatggtgatacaag |

BRAF Region1 376-425 bases

| | | |
|---|---|---|
| 6713 | Left | actaactaacgtgaaagccttacag |
| 6714 | Right | gatatggagatggtgatacaagctg |
| 6715 | Left | cttgattagagaccaaggatttcgt |
| 6716 | Right | ggaatagcccatgaagagtaggata |
| 6717 | Left | aagtcatcttcatcctcagaagaca |
| 6718 | Right | gcagtctgtcgtgcaatatctataa |
| 6719 | Left | gaagatcatcgaaatcaatttgggc |
| 6720 | Right | gctgtcacattcaacattttcactg |
| 6721 | Left | ttgatgacttgattagagaccaagg |
| 6722 | Right | tgaagagtaggatattcacatgtcg |
| 6723 | Left | tgacttgattagagaccaaggattt |
| 6724 | Right | atagcccatgaagagtaggatattc |
| 6725 | Left | gatcatcgaaatcaatttgggcaac |
| 6726 | Right | cacattcaacattttcactgccac |
| 6727 | Left | ctcactaactaacgtgaaagcctta |
| 6728 | Right | atggagatggtgatacaagctggag |
| 6729 | Left | gcatataaacacaatagaacctgtc |
| 6730 | Right | ttttgaaggcttgtaactgctgag |
| 6731 | Left | cttccgaccagcagatgaagatc |
| 6732 | Right | caacattttcactgccacatcac |
| 6733 | Left | ctcagcgagaaaggaagtcatct |
| 6734 | Right | gcagtctgtcgtgcaatatcta |
| 6735 | Left | cctcattacctggctcactaacta |
| 6736 | Right | tgatatggagatggtgatacaag |
| 6737 | Left | ttacctggctcactaactaacgt |
| 6738 | Right | ttggtctcaatgatatggagatg |
| 6739 | Left | gatttcgtggtgatggaggatc |
| 6740 | Right | ggaatagcccatgaagagtagg |
| 6741 | Left | ataaacacaatagaacctgtcaata |
| 6742 | Right | cttgtaactgctgaggtgtaggt |
| 6743 | Left | agaaatctccaggacctcagc |
| 6744 | Right | cgtgcaatatctataagtttgatca |
| 6745 | Left | actaacgtgaaagccttacagaaat |
| 6746 | Right | ctggagccctcacaccac |
| 6747 | Left | aatctccaggacctcagcgagaaag |
| 6748 | Right | ctgtcgtgcaatatctataagtttg |
| 6749 | Left | gagaccgatcctcatcagctc |
| 6750 | Right | aacattttcactgccacatcaccat |
| 6751 | Left | gaaatctccaggacctcagcgaga |
| 6752 | Right | tgatcatctcaaatttggtctcaa |

BRAF Region1 426-475 bases

| | | |
|---|---|---|
| 6753 | Left | actaacgtgaaagccttacagaaat |
| 6754 | Right | gcagtctgtcgtgcaatatctataa |
| 6755 | Left | ccttcaaaatccattccaattccac |
| 6756 | Right | gctgtcacattcaacattttcactg |
| 6757 | Left | attgatgacttgattagagaccaagg |
| 6758 | Right | ggaatagcccatgaagagtaggata |
| 6759 | Left | actaactaacgtgaaagccttacag |
| 6760 | Right | agtctgtcgtgcaatatctataagtt |
| 6761 | Left | gaagatcatcgaaatcaatttgggc |
| 6762 | Right | ttttgaaggcttgtaactgctga |
| 6763 | Left | tgcatataaacacaatagaacctgtc |
| 6764 | Right | tgaagagtaggatattcacatgtcg |
| 6765 | Left | caaaatccattccaattccacagc |
| 6766 | Right | cacattcaacattttcactgccac |
| 6767 | Left | aacgtgaaagccttacagaaatctc |
| 6768 | Right | gtgtaagtaatccatgccctgtg |
| 6769 | Left | gatttcgtggtgatggaggatc |
| 6770 | Right | gatatggagatggtgatacaagctg |
| 6771 | Left | ctcattacctggctcactaactaac |
| 6772 | Right | cgtgcaatatctataagtttgatca |
| 6773 | Left | aagtcatcttcatcctcagaagaca |
| 6774 | Right | ggatgattgacttggcgtgtaag |
| 6775 | Left | gatcatcgaaatcaatttgggcaac |
| 6776 | Right | cttgtaactgctgaggtgtaggt |
| 6777 | Left | ctcactaactaacgtgaaagcctta |
| 6778 | Right | gcagtctgtcgtgcaatatcta |
| 6779 | Left | gtccgtctccttcaaaatccatt |
| 6780 | Right | caacattttcactgccacatcac |
| 6781 | Left | attacctggctcactaactaacgtg |
| 6782 | Right | tgatcatctcaaatttggtctcaa |
| 6783 | Left | atattgatgacttgattagagacca |
| 6784 | Right | aatagcccatgaagagtaggatattc |
| 6785 | Left | ctcagcgagaaaggaagtcatc |
| 6786 | Right | gaggtctctgtggatgattgactt |
| 6787 | Left | ttacagaaatctccaggacctca |
| 6788 | Right | cttggcgtgtaagtaatccatgc |
| 6789 | Left | caaattctcaccagtccgtctc |
| 6790 | Right | aacattttcactgccacatcaccat |
| 6791 | Left | ctggctcactaactaacgtgaaa |
| 6792 | Right | cgtgcaatatctataagtttgatcatct |

BRAF Region1 476-525 bases

| | | |
|---|---|---|
| 6793 | Left | cttgattagagaccaaggatttcgt |
| 6794 | Right | gatatggagatggtgatacaagctg |
| 6795 | Left | tcactaactaacgtgaaagccttac |
| 6796 | Right | ttattactcttgaggtctctgtgga |
| 6797 | Left | gaagatcatcgaaatcaatttgggc |
| 6798 | Right | ggaatagcccatgaagagtaggata |
| 6799 | Left | aagtcatcttcatcctcagaagaca |
| 6800 | Right | actgtagctagaccaaaatcaccta |
| 6801 | Left | gatcatcgaaatcaatttgggcaac |
| 6802 | Right | tgaagagtaggatattcacatgtcg |
| 6803 | Left | ctcattacctggctcactaactaac |
| 6804 | Right | ctcttgaggtctctgtggatgatt |
| 6805 | Left | actaacgtgaaagccttacagaaat |
| 6806 | Right | gtctctgtggatgattgacttgg |
| 6807 | Left | cttcaaaatccattccaattccaca |
| 6808 | Right | ttttgaaggcttgtaactgctgag |
| 6809 | Left | ctaactaacgtgaaagccttacaga |
| 6810 | Right | gaggtctctgtggatgattgact |
| 6811 | Left | gatttcgtggtgatggaggatc |
| 6812 | Right | gcagtctgtcgtgcaatatctataa |
| 6813 | Left | tgatgacttgattagagaccaagga |
| 6814 | Right | atggagatggtgatacaagctggag |
| 6815 | Left | caaattctcaccagtccgtctc |
| 6816 | Right | gctgtcacattcaacattttcactg |
| 6817 | Left | ctggctcactaactaacgtgaaag |
| 6818 | Right | gtgtaagtaatccatgccctgtg |
| 6819 | Left | cttacagaaatctccaggacctca |
| 6820 | Right | ggatgattgacttggcgtgtaag |
| 6821 | Left | gtccgtctccttcaaaatccattc |
| 6822 | Right | cttgtaactgctgaggtgtaggt |
| 6823 | Left | aaattctcaccagtccgtctccttc |
| 6824 | Right | cacattcaacattttcactgccac |
| 6825 | Left | cttccgaccagcagatgaagatc |
| 6826 | Right | atagcccatgaagagtaggatattc |
| 6827 | Left | ttacctggctcactaactaacgtga |
| 6828 | Right | cttggcgtgtaagtaatccatgc |
| 6829 | Left | caaggatttcgtggtgatggag |
| 6830 | Right | ctgtcgtgcaatatctataagtttg |
| 6831 | Left | gactgccctaacatctggatcat |
| 6832 | Right | attttcactgccacatcaccat |

BRAF Region2 75-125 bases

| | | |
|---|---|---|
| 6833 | Left | aatcatccacagagacctcaagag |
| 6834 | Right | tgttcaaactgatgggaccc |
| 6835 | Left | tccacagagacctcaagagtaa |
| 6836 | Right | agacaactgttcaaactga |
| 6837 | Left | tcaatcatccacagagacctcaa |
| 6838 | Right | tgttcaaactgatggga |
| 6839 | Left | caagtcaatcatccacagagacc |
| 6840 | Right | tgggacccactc |

BRAF Region2 126-175 bases

| | | |
|---|---|---|
| 6841 | Left | aatcatccacagagacctcaagag |
| 6842 | Right | attctgatgacttctggtgccat |
| 6843 | Left | caagtcaatcatccacagagacc |
| 6844 | Right | aactgttcaaactgatgggacc |
| 6845 | Left | catccacagagacctcaagagtaa |
| 6846 | Right | cacaaaatggatccagacaact |
| 6847 | Left | tcaatcatccacagagacctcaa |
| 6848 | Right | attctgatgacttctggtgc |
| 6849 | Left | cacagggcatggattacttacac |
| 6850 | Right | agacaactgttcaaactgat |
| 6851 | Left | cttacacgccaagtcaatcatcc |
| 6852 | Right | aactgttcaaactgatggg |
| 6853 | Left | gcatggattacttacacgccaag |
| 6854 | Right | cacaaaatggatccagaca |
| 6855 | Left | gccaagtcaatcatccacagag |
| 6856 | Right | tgccatccacaaaatg |
| 6857 | Left | ttacacgccaagtcaatcatccaca |
| 6858 | Right | agacaactgttcaaact |
| 6859 | Left | ggcatggattacttacacgcc |
| 6860 | Right | cacaaaatggatccag |
| 6861 | Left | ttatagatattgcacgacagactgc |
| 6862 | Right | tgggacccactc |
| 6863 | Left | cttacacgccaagtcaatca |
| 6864 | Right | tgccatccacaaa |
| 6865 | Left | gcacgacagactgcacag |
| 6866 | Right | agacaactgttcaa |
| 6867 | Left | acgccaagtcaa |
| 6868 | Right | ctgatgacttctgg |

BRAF Region2 176-225 bases

| | | |
|---|---|---|
| 6869 | Left | ttatagatattgcacgacagactgc |
| 6870 | Right | attctgatgacttctggtgccat |
| 6871 | Left | cttacacgccaagtcaatcatcc |
| 6872 | Right | tctgactgaaagctgtatggatttt |
| 6873 | Left | tacacgccaagtcaatcatccacag |
| 6874 | Right | atgcatatacatctgactgaaagct |
| 6875 | Left | aacttatagatattgcacgacagac |
| 6876 | Right | cacaaaatggatccagacaact |
| 6877 | Left | catggattacttacacgccaag |
| 6878 | Right | tctgactgaaagctgtatggat |
| 6879 | Left | cacagggcatggattacttacac |
| 6880 | Right | attctgatgacttctggtgc |
| 6881 | Left | aaacttatagatattgcacgaca |
| 6882 | Right | cacaaaatggatccagaca |
| 6883 | Left | acacgccaagtcaatca |
| 6884 | Right | aatgcatatacatctgactgaaa |
| 6885 | Left | tgatcaaacttatagatattgcacg |
| 6886 | Right | tgccatccacaaaatg |
| 6887 | Left | gcatggattacttacacgcc |
| 6888 | Right | ctgactgaaagctgtatg |
| 6889 | Left | gcacgacagactgcacag |
| 6890 | Right | ttttatcttgcattctgat |
| 6891 | Left | gagatgatcaaacttatagatattgc |
| 6892 | Right | agacaactgttcaaact |
| 6893 | Left | tgagaccaaatttgagatgatc |
| 6894 | Right | cacaaaatggatccag |
| 6895 | Left | cacagggcatggattactt |
| 6896 | Right | attctgatgacttctgg |
| 6897 | Left | acacgccaagtcaa |
| 6898 | Right | aatgcatatacatctgactg |
| 6899 | Left | catctccatatcattgagacca |
| 6900 | Right | agacaactgttcaa |
| 6901 | Left | gagatgatcaaacttatagatat |
| 6902 | Right | tgccatccacaaa |
| 6903 | Left | cacagggcatggatta |
| 6904 | Right | ttttatcttgcattct |

BRAF Region2 226-275 bases

| | | |
|---|---|---|
| 6905 | Left | ttatagatattgcacgacagactgc |
| 6906 | Right | tccaaatgcatatacatctgactga |
| 6907 | Left | aacttatagatattgcacgacagac |
| 6908 | Right | tctgactgaaagctgtatggatttt |
| 6909 | Left | tcaaacttatagatattgcacgaca |
| 6910 | Right | atgcatatacatctgactgaaagct |
| 6911 | Left | atcaccatctccatatcattgagac |
| 6912 | Right | attctgatgacttctggtgccat |
| 6913 | Left | cacagggcatggattacttacac |
| 6914 | Right | acagaacaattccaaatgcatataca |
| 6915 | Left | gcatggattacttacacgccaa |
| 6916 | Right | acaattccaaatgcatatacatctgac |
| 6917 | Left | tgatcaaacttatagatattgcacg |
| 6918 | Right | tctgactgaaagctgtatggat |
| 6919 | Left | cagcttgtatcaccatctccatatc |
| 6920 | Right | ttctgatgacttctggtgc |
| 6921 | Left | gcacgacagactgcacag |
| 6922 | Right | acagaacaattccaaatgcatat |
| 6923 | Left | cacagggcatggattactta |
| 6924 | Right | tccagtcatcaattcatacaga |
| 6925 | Left | gagatgatcaaacttatagatattgc |
| 6926 | Right | tctgactgaaagctgtatg |
| 6927 | Left | tgtatcaccatctccatatcattga |
| 6928 | Right | tgccatccacaaaatg |
| 6929 | Left | cacagggcatggattac |
| 6930 | Right | caattccaaatgcatatacatct |
| 6931 | Left | ccagcttgtatcaccatctccat |
| 6932 | Right | cacaaaatggatccag |
| 6933 | Left | ttgagaccaaatttgagatgatc |
| 6934 | Right | attctgatgacttctgg |
| 6935 | Left | ctccagcttgtatcaccatctc |
| 6936 | Right | tgccatccacaaa |
| 6937 | Left | cacagggcatggat |
| 6938 | Right | acagaacaattccaaatgca |
| 6939 | Left | gagatgatcaaacttatagatat |
| 6940 | Right | tttatcttgcattctga |

BRAF Region2 276-325 bases

| | | |
|---|---|---|
| 6941 | Left | cagcttgtatcaccatctccatatc |
| 6942 | Right | tctgactgaaagctgtatggatttt |
| 6943 | Left | atcaccatctccatatcattgagac |
| 6944 | Right | atgcatatacatctgactgaaagct |
| 6945 | Left | acttatagatattgcacgacagactg |
| 6946 | Right | ctgtccagtcatcaattcatacaga |
| 6947 | Left | cacagggcatggattacttacac |
| 6948 | Right | aaaattatctggtccctgttgttga |
| 6949 | Left | caaacttatagatattgcacgacaga |
| 6950 | Right | ccaaatgcatatacatctgactgaa |
| 6951 | Left | gcatggattacttacacgccaa |
| 6952 | Right | attatctggtccctgttgttgatgt |
| 6953 | Left | cacaactggctattgttacccag |
| 6954 | Right | attctgatgacttctggtgccat |
| 6955 | Left | gcttgtatcaccatctccatatcatt |
| 6956 | Right | tctgactgaaagctgtatggat |
| 6957 | Left | gcacgacagactgcacag |
| 6958 | Right | cctgttgttgatgtttgaataaggt |
| 6959 | Left | gagatgatcaaacttatagatattgc |
| 6960 | Right | attccaaatgcatatacatctgact |
| 6961 | Left | ttgagaccaaatttgagatgatc |
| 6962 | Right | acagaacaattccaaatgcatataca |
| 6963 | Left | cacagggcatggattactta |
| 6964 | Right | ggtccctgttgttgatgtttgaa |
| 6965 | Left | tgatcaaacttatagatattgcacgac |
| 6966 | Right | ctgtccagtcatcaattcata |
| 6967 | Left | ccacaactggctattgttacc |
| 6968 | Right | attctgatgacttctggtgc |
| 6969 | Left | ttgagaccaaatttgagatg |
| 6970 | Right | acaattccaaatgcatatacatctg |
| 6971 | Left | ctccagcttgtatcaccatctcc |
| 6972 | Right | tctgactgaaagctgtatg |
| 6973 | Left | gagatgatcaaacttatagat |
| 6974 | Right | acagaacaattccaaatgcatat |
| 6975 | Left | cacagggcatggattac |
| 6976 | Right | aaaattatctggtccctgttgt |
| 6977 | Left | tatcctactatcatgggctattcc |
| 6978 | Right | cacaaaatggatccag |
| 6979 | Left | attccacaaagccacaactg |
| 6980 | Right | tgccatccacaaaatg |

BRAF Region2 326-375 bases

| | | |
|---|---|---|
| 6981 | Left | ttatagatattgcacgacagactgc |
| 6982 | Right | aaaattatctggtccctgttgttga |
| 6983 | Left | atcaccatctccatatcattgagac |
| 6984 | Right | ctgtccagtcatcaattcatacaga |
| 6985 | Left | ccagcttgtatcaccatctccatat |
| 6986 | Right | tccaaatgcatatacatctgactga |
| 6987 | Left | cacaactggctattgttacccag |
| 6988 | Right | tctgactgaaagctgtatggatttt |
| 6989 | Left | tatcctactcttcatgggctattcc |
| 6990 | Right | attctgatgacttctggtgccat |
| 6991 | Left | aacttatagatattgcacgacagac |
| 6992 | Right | tatctggtccctgttgttgatgttt |
| 6993 | Left | gcatggattacttacacgccaa |
| 6994 | Right | ttttggacagttactccgtacctta |
| 6995 | Left | cacagggcatggattacttacac |
| 6996 | Right | ccgtaccttactgagatctggag |
| 6997 | Left | tcaaacttatagatattgcacgaca |
| 6998 | Right | ggtatcctcgtcccaccataaaa |
| 6999 | Left | ctccagcttgtatcaccatctcc |
| 7000 | Right | acagaacaattccaaatgcatataca |
| 7001 | Left | gctattccacaaagccacaac |
| 7002 | Right | aatgcatatacatctgactgaaagc |
| 7003 | Left | agatgatcaaacttatagatattgcac |
| 7004 | Right | ggtccctgttgttgatgtttgaa |
| 7005 | Left | gcttgtatcaccatctccatatcatt |
| 7006 | Right | ctgtccagtcatcaattcatac |
| 7007 | Left | ccacaactggctattgttacc |
| 7008 | Right | acaattccaaatgcatatacatctgac |
| 7009 | Left | tgaatatcctactcttcatgggcta |
| 7010 | Right | attctgatgacttctggtgc |
| 7011 | Left | agatgatcaaacttatagatattg |
| 7012 | Right | cctgttgttgatgtttgaataaggt |
| 7013 | Left | gcacgacagactgcacag |
| 7014 | Right | aggtatcctcgtcccaccata |
| 7015 | Left | ttgagaccaaatttgagatgatc |
| 7016 | Right | aaaattatctggtccctgttgt |
| 7017 | Left | ccagtggtgtgagggctc |
| 7018 | Right | tctgactgaaagctgtatggat |
| 7019 | Left | cacagggcatggattactta |
| 7020 | Right | caggtatcctcgtcccacc |

BRAF Region2 376-425 bases

| | | |
|---|---|---|
| 7021 | Left | cagcttgtatcaccatctccatatc |
| 7022 | Right | aaaattatctggtccctgttgttga |
| 7023 | Left | tgaatatcctactcttcatgggcta |
| 7024 | Right | tctgactgaaagctgtatggatttt |
| 7025 | Left | tatcctactcttcatgggctattcc |
| 7026 | Right | ctgtccagtcatcaattcatacaga |
| 7027 | Left | cgacatgtgaatatcctactcttca |
| 7028 | Right | atgcatatacatctgactgaaagct |
| 7029 | Left | acttatagatattgcacgacagact |
| 7030 | Right | ttttggacagttactccgtacctta |
| 7031 | Left | atcaccatctccatatcattgagac |
| 7032 | Right | ggtccctgttgttgatgtttgaa |
| 7033 | Left | ccacaactggctattgttaccc |
| 7034 | Right | cctgttgttgatgtttgaataaggt |
| 7035 | Left | tcaaacttatagatattgcacgaca |
| 7036 | Right | cttttggacagttactccgtacc |
| 7037 | Left | ctccagcttgtatcaccatctcc |
| 7038 | Right | attatctggtccctgttgttgatgt |
| 7039 | Left | acgacatgtgaatatcctactct |
| 7040 | Right | tccaaatgcatatacatctgactga |
| 7041 | Left | tcagcagttacaagccttcaaaa |
| 7042 | Right | ttctgatgacttctggtgccat |
| 7043 | Left | tgatcaaacttatagatattgcacg |
| 7044 | Right | ccgtaccttactgagatctggag |
| 7045 | Left | cacagggcatggattacttacac |
| 7046 | Right | ggcttttggacagttactccg |
| 7047 | Left | gcttgtatcaccatctccatatcatt |
| 7048 | Right | aaaattatctggtccctgttgt |
| 7049 | Left | gagatgatcaaacttatagatattgc |
| 7050 | Right | ggtatcctcgtcccaccataaaa |
| 7051 | Left | tgaatatcctactcttcatggg |
| 7052 | Right | acagaacaattccaaatgcatataca |
| 7053 | Left | ttgagaccaaatttgagatgatc |
| 7054 | Right | aggtatcctcgtcccaccata |
| 7055 | Left | acctacacctcagcagttacaag |
| 7056 | Right | attctgatgacttctggtgc |
| 7057 | Left | attccacaaagccacaactg |
| 7058 | Right | attccaaatgcatatacatctgac |
| 7059 | Left | gcacgacagactgcacag |
| 7060 | Right | cactctgccattaatctcttcat |

BRAF Region2 426-475 bases

| | | |
|---|---|---|
| 7061 | Left | tatcctactcttcatgggctattcc |
| 7062 | Right | aaaattatctggtccctgttgttga |
| 7063 | Left | tgaatatcctactcttcatgggcta |
| 7064 | Right | ctgtccagtcatcaattcatacaga |
| 7065 | Left | agcttgtatcaccatctccatatca |
| 7066 | Right | ttttggacagttactccgtacctta |
| 7067 | Left | cgacatgtgaatatcctactcttca |
| 7068 | Right | attatctggtccctgttgttgatgt |
| 7069 | Left | atcaccatctccatatcattgagac |
| 7070 | Right | cttttggacagttactccgtacc |
| 7071 | Left | ctcagcagttacaagccttcaaaa |
| 7072 | Right | tccaaatgcatatacatctgactga |
| 7073 | Left | acctacacctcagcagttacaag |
| 7074 | Right | atgcatatacatctgactgaaagct |
| 7075 | Left | tatagatattgcacgacagactgc |
| 7076 | Right | gagaatttggggaaagagtggtc |
| 7077 | Left | gatgtggcagtgaaaatgttgaatg |
| 7078 | Right | attctgatgacttctggtgccat |
| 7079 | Left | cagggcatggattacttacacg |
| 7080 | Right | agtggtctctcatctcttttctttt |
| 7081 | Left | tgtatcaccatctccatatcattga |
| 7082 | Right | ccgtaccttactgagatctggag |
| 7083 | Left | tgacagcacctacacctcag |
| 7084 | Right | tctgactgaaagctgtatggatttt |
| 7085 | Left | cacaactggctattgttacccag |
| 7086 | Right | ggtatcctcgtcccaccataaaa |
| 7087 | Left | gcatggattacttacacgccaa |
| 7088 | Right | aatagaggcgagaatttggggaaag |
| 7089 | Left | tacacctcagcagttacaagcctt |
| 7090 | Right | acagaacaattccaaatgcatataca |
| 7091 | Left | aacttatagatattgcacgacagac |
| 7092 | Right | cactctgccattaatctcttcat |
| 7093 | Left | acgacatgtgaatatcctactct |
| 7094 | Right | ggtccctgttgttgatgtttgaa |
| 7095 | Left | taggagtactcaggaaaacacgac |
| 7096 | Right | acagaacaattccaaatgcatat |
| 7097 | Left | ctccagcttgtatcaccatctcc |
| 7098 | Right | ggcttttggacagttactccg |
| 7099 | Left | tcaaacttatagatattgcacgaca |
| 7100 | Right | agtggtctctcatctcttttct |

BRAF Region2 476-525 bases

| | | |
|---|---|---|
| 7101 | Left | cgacatgtgaatatcctactcttca |
| 7102 | Right | aaaattatctggtccctgttgttga |
| 7103 | Left | cagtgaaaatgttgaatgtgacagc |
| 7104 | Right | ctgtccagtcatcaattcatacaga |
| 7105 | Left | tatcctactcttcatgggctattcc |
| 7106 | Right | tttggacagttactccgtacctta |
| 7107 | Left | gcttgtatcaccatctccatatcatt |
| 7108 | Right | agtggtctctcatctcttttctttt |
| 7109 | Left | ttatagatattgcacgacagactgc |
| 7110 | Right | aatagaggcgagaatttggggaaag |
| 7111 | Left | gatgtggcagtgaaaatgttgaatg |
| 7112 | Right | ccaaatgcatatacatctgactgaa |
| 7113 | Left | atcaccatctccatatcattgagac |
| 7114 | Right | gagaatttggggaaagagtggtc |
| 7115 | Left | tgaatatcctactcttcatgggcta |
| 7116 | Right | ggtatcctcgtcccaccataaaa |
| 7117 | Left | catggtgatgtggcagtgaaaat |
| 7118 | Right | tctgactgaaagctgtatggatttt |
| 7119 | Left | tcagcagttacaagccttcaaaa |
| 7120 | Right | attatctggtccctgttgttgatgt |
| 7121 | Left | gtctacaagggaaagtggcatg |
| 7122 | Right | atgcatatacatctgactgaaagct |
| 7123 | Left | aacttatagatattgcacgacagac |
| 7124 | Right | aatagaggcgagaatttgggga |
| 7125 | Left | ccacaactggctattgttaccc |
| 7126 | Right | cttttggacagttactccgtacc |
| 7127 | Left | acctacacctcagcagttaca |
| 7128 | Right | cctgttgttgatgtttgaataaggt |
| 7129 | Left | ccagcttgtatcaccatctccatat |
| 7130 | Right | cactctgccattaatctcttcat |
| 7131 | Left | tggaacagtctacaagggaaagt |
| 7132 | Right | ttctgatgacttctggtgccat |
| 7133 | Left | aacagtctacaagggaaagtggc |
| 7134 | Right | attccaaatgcatatacatctgact |
| 7135 | Left | ctacacctcagcagttacaagcc |
| 7136 | Right | ggtccctgttgttgatgtttgaa |
| 7137 | Left | tgacagcacctacacctcag |
| 7138 | Right | acagaacaattccaaatgcatataca |
| 7139 | Left | cacagggcatggattacttacac |
| 7140 | Right | aaggagggttctgatgcactg |

BRAF Region2 750-1250 bases

| | | |
|---|---|---|
| 7141 | Left | taaatttcaccagcgttgtagtaca |
| 7142 | Right | aaaattatctggtccctgttgttga |
| 7143 | Left | catgtggttataaatttcaccagcg |
| 7144 | Right | ctgtccagtcatcaattcatacaga |
| 7145 | Left | cttgattagagaccaaggatttcgt |
| 7146 | Right | ttttggacagttactccgtacctta |
| 7147 | Left | tatgaccaacttgatttgctgtttg |
| 7148 | Right | cctgttgttgatgtttgaataaggt |
| 7149 | Left | gaagatcatcgaaatcaatttgggc |
| 7150 | Right | agtggtctctcatctcttttctttt |
| 7151 | Left | ccttcaaaatccattccaattccac |
| 7152 | Right | attatctggtccctgttgttgatgt |
| 7153 | Left | tgatgacttgattagagaccaagga |
| 7154 | Right | cttttggacagttactccgtacc |
| 7155 | Left | tggttataaatttcaccagcgttgt |
| 7156 | Right | acagaacaattccaaatgcatataca |
| 7157 | Left | ttgtagtacagaagttccactgatg |
| 7158 | Right | ccgtaccttactgagatctggag |
| 7159 | Left | gatcatcgaaatcaatttgggcaac |
| 7160 | Right | aatagaggcgagaatttggggaaag |
| 7161 | Left | tgacttgattagagaccaaggattt |
| 7162 | Right | gagaatttggggaaagagtggtc |
| 7163 | Left | aattatgaccaacttgatttgctgt |
| 7164 | Right | ggtccctgttgttgatgtttgaa |
| 7165 | Left | cttgatttgctgtttgtctccaag |
| 7166 | Right | ggtatcctcgtcccaccataaaa |
| 7167 | Left | cctcattacctggctcactaactaa |
| 7168 | Right | aatagaggcgagaatttgggga |
| 7169 | Left | caaaatccattccaattccacagc |
| 7170 | Right | gcatatagactaaaatcctctgtttgg |
| 7171 | Left | cgttgtagtacagaagttccactg |
| 7172 | Right | tgttgttgatgtttgaataaggtaac |
| 7173 | Left | gtctccttcaaaatccattccaatt |
| 7174 | Right | ggcttttggacagttactccg |
| 7175 | Left | cgtctccttcaaaatccattcca |
| 7176 | Right | agcatatagactaaaatcctctgtt |
| 7177 | Left | caacttgatttgctgtttgtctcc |
| 7178 | Right | aggtatcctcgtcccaccata |
| 7179 | Left | tgcatataaacacaatagaacctgt |
| 7180 | Right | gcgagaatttggggaaagagtg |

TABLE 14
KRAS cDNA Capture Primer List for NGS Panel

| | | |
|---|---|---|
| Seq. | | |
| ID | Primer | Sequence |

KRAS Region1 75-125 bases

| | | |
|---|---|---|
| 7181 | Left | GCCTGCTGAAAATGACTGAATATAA |
| 7182 | Right | ATTCGTCCACAAAATGATTCTGAAT |
| 7183 | Left | GAGAGAGGCCTGCTGAAAATG |
| 7184 | Right | ATTGTTGGATCATATTCGTCCACAA |
| 7185 | Left | GGCCTGCTGAAAATGACTGAA |
| 7186 | Right | TCTATTGTTGGATCATATTCGTCCA |
| 7187 | Left | GGAGAGAGGCCTGCTGAAA |
| 7188 | Right | TGGATCATATTCGTCCACAAAATGA |
| 7189 | Left | GAGAGGCCTGCTGAAAATGACT |
| 7190 | Right | ATCATATTCGTCCACAAAATGATTC |
| 7191 | Left | CTCAGCGGCTCCCAGG |
| 7192 | Right | AAATGATTCTGAATTAGCTGTAT |
| 7193 | Left | GTGCGGGAGAGAGGCC |
| 7194 | Right | AAATGATTCTGAATTAGCTG |
| 7195 | Left | GGGAGAGAGGCCTGCTG |
| 7196 | Right | TGTTGGATCATATTCGT |

KRAS Region1 126-175 bases

| | | |
|---|---|---|
| 7197 | Left | GCCTGCTGAAAATGACTGAATATAAA |
| 7198 | Right | GTTTCTCCATCAATTACTACTTGCT |
| 7199 | Left | GAGAGAGGCCTGCTGAAAATG |
| 7200 | Right | TATTGTTGGATCATATTCGTCCACA |
| 7201 | Left | GAGAGGCCTGCTGAAAATGACTGAA |
| 7202 | Right | TCCTCTATTGTTGGATCATATTCGT |
| 7203 | Left | GGAGAGAGGCCTGCTGAAA |
| 7204 | Right | TCCATCAATTACTACTTGCTTCCTG |
| 7205 | Left | GAGAGAGGCCTGCTGAAAATGACT |
| 7206 | Right | TATTGTTGGATCATATTCGTCCACAA |
| | | AA |
| 7207 | Left | CTCAGCGGCTCCCAGG |
| 7208 | Right | TGGATCATATTCGTCCACAAAATGA |
| 7209 | Left | GTGCGGGAGAGAGGCC |
| 7210 | Right | TCAATTACTACTTGCTTCCTGTAGG |
| 7211 | Left | CCAGGTGCGGGAGAGAG |
| 7212 | Right | ATCATATTCGTCCACAAAATGATTC |
| 7213 | Left | GCACTGAAGGCGGCG |
| 7214 | Right | ATTCGTCCACAAAATGATTCTGAAT |
| 7215 | Left | GGGAGAGAGGCCTGCTG |
| 7216 | Right | ATCCTCTATTGTTGGATCATATT |
| 7217 | Left | GGACTGGGAGCGAGCG |
| 7218 | Right | AAATGATTCTGAATTAGCTGTAT |
| 7219 | Left | GCTCCCAGGTGCGGGA |
| 7220 | Right | AGGAATCCTCTATTGTTGGA |
| 7221 | Left | AGCGGCTCCCAGGTGC |
| 7222 | Right | ATCCTCTATTGTTGGATCAT |
| 7223 | Left | CTGAAAATGACTGAATAT |
| 7224 | Right | GAATATCCAAGAGACAGGTTTCT |
| 7225 | Left | CCAGAGGCTCAGCGG |
| 7226 | Right | AAATGATTCTGAATTAGCTG |
| 7227 | Left | TCAGCGGCTCCC |
| 7228 | Right | AGGAATCCTCTATTGTT |

KRAS Region1 176-225 bases

| | | |
|---|---|---|
| 7229 | Left | ATTTCGGACTGGGAGCGAG |
| 7230 | Right | ATTGTTGGATCATATTCGTCCACAA |
| 7231 | Left | CTCGGCCAGTACTCCCG |
| 7232 | Right | TGGATCATATTCGTCCACAAAATGA |
| 7233 | Left | CAGGTGCGGGAGAGAGG |
| 7234 | Right | TCGAGAATATCCAAGAGACAGGTTT |
| 7235 | Left | CTCAGCGGCTCCCAGG |
| 7236 | Right | TCCATCAATTACTACTTGCTTCCTG |
| 7237 | Left | GCCAGTACTCCCGGCC |
| 7238 | Right | ATTCGTCCACAAAATGATTCTGAAT |
| 7239 | Left | TGCGGGAGAGAGGCCT |
| 7240 | Right | TGTGTCGAGAATATCCAAGAGACAG |
| 7241 | Left | GCACTGAAGGCGGCG |
| 7242 | Right | TCAATTACTACTTGCTTCCTGTAGG |
| 7243 | Left | GCTCCCAGGTGCGGGA |
| 7244 | Right | GTTTCTCCATCAATTACTACTTGCT |
| 7245 | Left | CATTTCGGACTGGGAGC |
| 7246 | Right | TCTATTGTTGGATCATATTCGTCCA |
| 7247 | Left | AGCGGCTCCCAGGTGC |
| 7248 | Right | GCTGTGTCGAGAATATCCAAGAG |
| 7249 | Left | TCCCAGGTGCGGGAGAG |
| 7250 | Right | CTCTTGACCTGCTGTGTCGA |
| 7251 | Left | CCAGAGGCTCAGCGG |
| 7252 | Right | CCTGCTGTGTCGAGAATATCCAA |
| 7253 | Left | GCTCGGCCAGTACTC |
| 7254 | Right | TCATATTCGTCCACAAAATGATTCT |
| 7255 | Left | CCCCGCCATTTCG |
| 7256 | Right | TCCTCTATTGTTGGATCATATTCGT |
| 7257 | Left | TCAGCGGCTCCC |
| 7258 | Right | TTGACCTGCTGTGTCGAGAATATC |
| 7259 | Left | GGCACTGAAGGCG |
| 7260 | Right | ATCCTCTATTGTTGGATCATATT |
| 7261 | Left | CATTTCGGACTGGG |
| 7262 | Right | AGGAATCCTCTATTGTTGGA |
| 7229 | Left | CCAGAGGCTCAG |
| 7230 | Right | GTTTCTCCATCAATTACTACTT |

KRAS Region2 75-125 bases

| | | |
|---|---|---|
| 7231 | Left | CTTGGATATTCTCGACACAGCAG |
| 7232 | Right | TTATGGCAAATACACAAAGAAAGCC |
| 7233 | Left | AACCTGTCTCTTGGATATTCTCGAC |
| 7234 | Right | AAATACACAAAGAAAGCCCTCCC |
| 7235 | Left | GGAAGCAAGTAGTAATTGATGGAGA |
| 7236 | Right | AATACACAAAGAAAGCCCTCCCCAG |
| 7237 | Left | AGAAACCTGTCTCTTGGATATTCTC |
| 7238 | Right | TCCCCAGTCCTCATGTACTG |
| 7239 | Left | TGTCTCTTGGATATTCTCGACACAG |
| 7240 | Right | AAAGAAAGCCCTCCCCAGTCC |
| 7241 | Left | AGCAAGTAGTAATTGATGGAGAAAC |
| 7242 | Right | TCCCCAGTCCTCATGTA |
| 7243 | Left | CTACAGGAAGCAAGTAGTAATTGA |
| 7244 | Right | TCCCCAGTCCTCAT |
| 7245 | Left | AGAAACCTGTCTCTTGGATATT |
| 7246 | Right | GATTTAGTATTATTTATGGC |
| 7247 | Left | AGAAACCTGTCTCTTGGA |
| 7248 | Right | GGCAAATACACAAAGAAA |
| 7249 | Left | AGAAACCTGTCTCTT |
| 7250 | Right | TTTATGGCAAATACACAAAG |
| 7251 | Left | CAAGTAGTAATTGATGG |
| 7252 | Right | ATGGCAAATACACA |

KRAS Region2 126-175 bases

| | | |
|---|---|---|
| 7253 | Left | TTTGTGGACGAATATGATCCAACAA |
| 7254 | Right | TTATGGCAAATACACAAAGAAAGCC |
| 7255 | Left | TGGACGAATATGATCCAACAATAGAG |
| 7256 | Right | AAATACACAAAGAAAGCCCTCCC |
| 7257 | Left | CTTGGATATTCTCGACACAGCAG |
| 7258 | Right | AGGTACATCTTCAGAGTCCTTAAC |
| 7259 | Left | TTCAGAATCATTTTGTGGACGAATA |
| 7260 | Right | AATACACAAAGAAAGCCCTCCCCAG |
| 7261 | Left | CCTTGACGATACAGCTAATTCAGAA |
| 7262 | Right | TCCCCAGTCCTCATGTACTG |
| 7263 | Left | CATTTTGTGGACGAATATGATCCAA |
| 7264 | Right | GAAAGCCCTCCCCAGTCC |
| 7265 | Left | TGTCTCTTGGATATTCTCGACACAG |
| 7266 | Right | GTCCTTAACTCTTTTAATTTGTTC |
| 7267 | Left | AACCTGTCTCTTGGATATTCTCGA |
| 7268 | Right | TAATTTGTTCTCTATAATGGTGAA |
| 7269 | Left | GCAAGTAGTAATTGATGGAGAAAC |
| 7270 | Right | TTTATGGCAAATACACAAAGAAA |
| 7271 | Left | TGACGATACAGCTAATTCAGAATCA |
| 7272 | Right | TCCCCAGTCCTCATGTA |
| 7273 | Left | CAGGAAGCAAGTAGTAATTGATGGA |
| 7274 | Right | GATTTAGTATTATTTATGGC |
| 7275 | Left | AGAAACCTGTCTCTTGGATATTCT |
| 7276 | Right | AATTTGTTCTCTATAATGGT |
| 7277 | Left | CTACAGGAAGCAAGTAGTAATTG |
| 7278 | Right | TTTATGGCAAATACACAAAG |
| 7279 | Left | AGAAACCTGTCTCTTGGATAT |
| 7280 | Right | GTCCTTAACTCTTTTAATTTG |
| 7281 | Left | ACAGCTAATTCAGAATCATTTTGTGG |
| 7282 | Right | TCCCCAGTCCTCAT |
| 7283 | Left | AGAAACCTGTCTCTTGG |
| 7284 | Right | ACTCTTTTAATTTGTTCTCT |
| 7285 | Left | CTACAGGAAGCAAGTAGTAA |
| 7286 | Right | TATAATGGTGAATATCTTC |
| 7287 | Left | TCCAACAATAGAGGATTCC |
| 7288 | Right | TTTATGGCAAATACACA |
| 7289 | Left | AGAAACCTGTCTCT |
| 7290 | Right | GTCCTTAACTCTTTTAAT |

KRAS Region2 176-225 bases

| | | |
|---|---|---|
| 7291 | Left | CCTTGACGATACAGCTAATTCAGAA |
| 7292 | Right | TTATGGCAAATACACAAAGAAAGCC |
| 7293 | Left | TTCAGAATCATTTTGTGGACGAATA |
| 7294 | Right | GCAAATACACAAAGAAAGCCCTC |
| 7295 | Left | CAGGAAGCAAGTAGTAATTGATGGA |
| 7296 | Right | CCATAGGTACATCTTCAGAGTC |
| 7297 | Left | TGACGATACAGCTAATTCAGAATCA |
| 7298 | Right | TTTATGGCAAATACACAAAGAAA |
| 7299 | Left | GGACGAATATGATCCAACAATAGAGG |
| 7300 | Right | TTTAATTTGTTCTCTATAATGGTG |
| 7301 | Left | TACAGGAAGCAAGTAGTAA |
| 7302 | Right | CCATAGGTACATCTTCAGAGTCCTT |
| 7303 | Left | GCTAATTCAGAATCATTTTGTGGACG |
| 7304 | Right | TTTATGGCAAATACACAAAG |
| 7305 | Left | GCAAGTAGTAATTGATGGAGAA |
| 7306 | Right | GTACATCTTCAGAGTCCTTAAC |
| 7307 | Left | GTGGACGAATATGATCCAACAATAG |
| 7308 | Right | AACTCTTTTAATTTGTTCTC |
| 7309 | Left | CTACAGGAAGCAAGTAGTAATTGA |
| 7310 | Right | CCATAGGTACATCTTCAGA |
| 7311 | Left | TCATTTTGTGGACGAATATGATCCA |
| 7312 | Right | GATTTAGTATTATTTATGGC |
| 7313 | Left | ACAGCTAATTCAGAATCATTTTGTGG |
| 7314 | Right | TTTATGGCAAATACACA |
| 7315 | Left | TCCAACAATAGAGGATTCC |
| 7316 | Right | GTCCTTAACTCTTTTAATTTGTT |
| 7317 | Left | GAATCATTTTGTGGACGAATATGA |
| 7318 | Right | ATAATGGTGAATATCTTC |
| 7319 | Left | AGAAACCTGTCTCTTG |
| 7320 | Right | CTAGAAGGCAAATCACATTTATTT |
| 7321 | Left | TACAGGAAGCAAGTAG |
| 7322 | Right | GTCCTTAACTCTTTTAATTT |
| 7323 | Left | GAATATGATCCAACAA |
| 7324 | Right | GTCCTTAACTCTTTTAA |
| 7325 | Left | CTACAGGAAGCAAG |
| 7326 | Right | CCATAGGTACATCTTC |

KRAS Region3 176-225 bases

| | | |
|---|---|---|
| 7327 | Left | gactgaatataaacttgtggtagt |
| 7328 | Right | tccccagtcctcatgtactg |
| 7329 | Left | gactgaatataaacttgtggt |
| 7330 | Right | tccccagtcctcatgta |
| 7331 | Left | tgactgaatataaacttgt |
| 7332 | Right | ccccagtcctcat |

KRAS Region3 226-275 bases

| | | |
|---|---|---|
| 7333 | Left | atgactgaatataaacttgtggtag |
| 7334 | Right | ttatggcaaatacacaaagaaagcc |
| 7335 | Left | GCCTGCTGAAAatgactgaatataaa |
| 7336 | Right | atacacaaagaaagccctcccc |
| 7337 | Left | GGAGAGAGGCCTGCTGAAAat |
| 7338 | Right | gcaaatacacaaagaaagccctc |
| 7339 | Left | CCTGCTGAAAatgactgaatataaactt |
| 7340 | Right | tccccagtcctcatgtactg |
| 7341 | Left | GAGAGGCCTGCTGAAAatgact |
| 7342 | Right | atacacaaagaaagccctccccagt |
| 7343 | Left | GGGAGAGAGGCCTGCTG |
| 7344 | Right | caaagaaagccctccccagtcct |
| 7345 | Left | GGCCTGCTGAAAatgactgaata |
| 7346 | Right | ccccagtcctcatgta |
| 7347 | Left | TGCGGGAGAGAGGCCT |
| 7348 | Right | tatggcaaatacacaaagaaa |
| 7349 | Left | Aatgactgaatataaacttgtgg |
| 7350 | Right | tccccagtcctcat |
| 7351 | Left | GGTGCGGGAGAGAGG |
| 7352 | Right | ggcaaatacacaaag |

KRAS Region3 276-325 bases

| | | |
|---|---|---|
| 7353 | Left | CTCAGCGGCTCCCAGG |
| 7354 | Right | ttatggcaaatacacaaagaaagcc |
| 7355 | Left | tgactgaatataaacttgtggtagt |
| 7356 | Right | gtccttaactcttttaatttgttc |
| 7357 | Left | TCCCAGGTGCGGGAGA |
| 7358 | Right | aaatacacaaagaaagccctccc |
| 7359 | Left | GGCCTGCTGAAAatgactgaat |
| 7360 | Right | tttaatttgttctctataatggtg |
| 7361 | Left | ATTTCGGACTGGGAGCGAG |
| 7362 | Right | tccccagtcctcatgtactg |
| 7363 | Left | GCACTGAAGGCGGCG |
| 7364 | Right | aatacacaaagaaagccctccccag |
| 7365 | Left | TGCGGGAGAGAGGCCT |
| 7366 | Right | tttatggcaaatacacaaagaaa |
| 7367 | Left | GGCCTGCTGAAAatgactgaatata |
| 7368 | Right | actcttttaatttgttctct |
| 7369 | Left | AGCGGCTCCCAGGTGC |
| 7370 | Right | cacaaagaaagccctccccagtcc |
| 7371 | Left | CCTGCTGAAAatgactgaatataaact |
| 7372 | Right | gatttagtattatttatggc |
| 7373 | Left | tgactgaatataaacttgtggt |
| 7374 | Right | acatcttcagagtccttaa |
| 7375 | Left | GAGAGAGGCCTGCTGAAAatg |
| 7376 | Right | tataatggtgaatatcttc |
| 7377 | Left | CATTTCGGACTGGGAGC |
| 7378 | Right | tccccagtcctcatgta |
| 7379 | Left | CCAGAGGCTCAGCGG |
| 7380 | Right | tttatggcaaatacacaaag |
| 7381 | Left | CCAGGTGCGGGAGAGAG |
| 7382 | Right | tttatggcaaatacaca |
| 7383 | Left | CTGAAAatgactgaatataaacttgt |
| 7384 | Right | gtccttaactcttttaa |
| 7385 | Left | GGCCTGCTGAAAatgact |
| 7386 | Right | ccttaactcttttaatttg |
| 7387 | Left | GGCACTGAAGGCG |
| 7388 | Right | tccccagtcctcat |

KRAS Region3 326-375 bases

| | | |
|---|---|---|
| 7389 | Left | ATTTCGGACTGGGAGCGAG |
| 7390 | Right | ttatggcaaatacacaaagaaagcc |
| 7391 | Left | CTCAGCGGCTCCCAGG |
| 7392 | Right | ccataggtacatcttcagagtcctt |
| 7393 | Left | CTCGGCCAGTACTCCCG |
| 7394 | Right | aaatacacaaagaaagccctccc |
| 7395 | Left | GTGCGGGAGAGAGGCC |
| 7396 | Right | taggtacatcttcagagtccttaac |
| 7397 | Left | GCCAGTACTCCCGGCC |
| 7398 | Right | aatacacaaagaaagccctccccag |
| 7399 | Left | CCAGGTGCGGGAGAGAG |
| 7400 | Right | ccataggtacatcttcagagtc |
| 7401 | Left | GCTCCCAGGTGCGGGA |
| 7402 | Right | gtccttaactcttttaatttgac |
| 7403 | Left | GCACTGAAGGCGGCG |
| 7404 | Right | taatttgttctctataatggtgaa |
| 7405 | Left | CATTTCGGACTGGGAGC |
| 7406 | Right | aaagaaagccctccccagtcc |
| 7407 | Left | AGCGGCTCCCAGGTGC |
| 7408 | Right | gtccttaactcttttaatttg |
| 7409 | Left | GCTCGGCCAGTACTC |
| 7410 | Right | tatggcaaatacacaaagaaa |
| 7411 | Left | CCAGAGGCTCAGCGG |
| 7412 | Right | actcttttaatttgttctct |
| 7413 | Left | CCCCGCCATTTCG |
| 7414 | Right | tttatggcaaatacacaaag |
| 7415 | Left | TCAGCGGCTCCC |
| 7416 | Right | ccataggtacatcttcaga |
| 7417 | Left | GGCACTGAAGGCG |
| 7418 | Right | aatttgttctctataatggt |

KRAS Region3 376-425 bases

| | | |
|---|---|---|
| 7419 | Left | CCAGGTGCGGGAGAGAG |
| 7420 | Right | actgttctagaaggcaaatcacatt |
| 7421 | Left | CTCAGCGGCTCCCAGG |
| 7422 | Right | tctactgttctagaaggcaaatcac |
| 7423 | Left | TTTCGGACTGGGAGCGAG |
| 7424 | Right | aggtacatcttcagagtccttaac |
| 7425 | Left | CTCGGCCAGTACTCCCG |
| 7426 | Right | tttatggcaaatacacaaagaaagcc |
| 7427 | Left | GTGCGGGAGAGAGGCC |
| 7428 | Right | gttttgtgtctactgactagaagg |
| 7429 | Left | GCACTGAAGGCGGCG |
| 7430 | Right | ccataggtacatcttcagagtcctt |
| 7431 | Left | TCCCAGGTGCGGGAGA |
| 7432 | Right | tgttctagaaggcaaatcacatttat |
| 7433 | Left | AGCGGCTCCCAGGTGC |
| 7434 | Right | gcctgttttgtgtctactgttc |
| 7435 | Left | GCCAGTACTCCCGGCC |
| 7436 | Right | taatttgttctctataatggtgaa |
| 7437 | Left | CATTTCGGACTGGGAGC |
| 7438 | Right | gtccttaactcttttaatttgttc |
| 7439 | Left | CCAGAGGCTCAGCGG |
| 7440 | Right | ccataggtacatcttcagagtc |
| 7441 | Left | GCTCGGCCAGTACTC |
| 7442 | Right | ttatggcaaatacacaaagaaagccctc |
| 7443 | Left | AAGGTGGCGGCG |
| 7444 | Right | aaatacacaaagaaagccctcccc |
| 7445 | Left | GGCGGCGAAGGT |
| 7446 | Right | atacacaaagaaagccctccccagt |
| 7447 | Left | GCGAAGGTGGCG |
| 7448 | Right | caaagaaagccctccccagtcct |
| 7449 | Left | TCAGCGGCTCCC |
| 7450 | Right | tctactgttctagaaggcaaat |
| 7419 | Left | CGGAGGCAGCAG |
| 7420 | Right | tttatggcaaatacacaaagaaa |
| 7421 | Left | CCCCGCCATTTCG |
| 7422 | Right | gtccttaactcttttaatttg |
| 7423 | Left | GGCACTGAAGGCG |
| 7424 | Right | ccataggtacatcttcaga |
| 7425 | Left | CATTTCGGACTGGG |
| 7426 | Right | actcttttaatttgttctct |

KRAS Region3 426-475 bases

| | | |
|---|---|---|
| 7427 | Left | ATTTCGGACTGGGAGCGAG |
| 7428 | Right | actgttctagaaggcaaatcacatt |
| 7429 | Left | GCTCGGCCAGTACTCCC |
| 7430 | Right | ccataggtacatcttcagagtcctt |
| 7431 | Left | GTGCGGGAGAGAGGCC |
| 7432 | Right | gtcttgtctttgctgatgtttcaat |
| 7433 | Left | GCCAGTACTCCCGGCC |
| 7434 | Right | taggtacatcttcagagtccttaac |
| 7435 | Left | CTCAGCGGCTCCCAGG |
| 7436 | Right | agcctgttttgtgtctactgttc |
| 7437 | Left | GCACTGAAGGCGGCG |
| 7438 | Right | tctactgttctagaaggcaaatcac |
| 7439 | Left | CCAGGTGCGGGAGAGAG |
| 7440 | Right | cttcttgctaagtcctgagcct |
| 7441 | Left | GCTCCCAGGTGCGGGA |
| 7442 | Right | tgctgatgtttcaataaaaggaattcc |
| 7443 | Left | CCAGAGGCTCAGCGG |
| 7444 | Right | gttttgtgtctactgttctagaagg |
| 7445 | Left | AGCGGCTCCCAGGTGC |
| 7446 | Right | ttgctgatgtttcaataaaaggaat |
| 7447 | Left | CATTTCGGACTGGGAGC |
| 7448 | Right | tgttctagaaggcaaatcacatttat |
| 7449 | Left | GGCACTGAAGGCG |
| 7450 | Right | ttcttgctaagtcctgagcctgttt |
| 7451 | Left | GCTCGGCCAGTACT |
| 7452 | Right | ccataggtacatcttcagagtc |
| 7453 | Left | AAGGTGGCGGCG |
| 7454 | Right | gtccttaactcttttaatttgttc |
| 7455 | Left | GGCGGCGAAGGT |
| 7456 | Right | taatttgttctctataatggtgaa |
| 7457 | Left | TCAGCGGCTCCC |
| 7458 | Right | gaattccataacttcttgctaag |
| 7459 | Left | CATTTCGGACTGGG |
| 7460 | Right | tctactgttctagaaggcaaat |
| 7461 | Left | CCCCGCCATTTCG |
| 1462 | Right | ccataggtacatcttcaga |
| 7463 | Left | GCGAAGGTGGCG |
| 7464 | Right | gtccttaactcttttaatttg |
| 7465 | Left | CCAGAGGCTCAG |
| 7466 | Right | agcctgttttgtgtctactg |

KRAS Region3 476-525 bases

| | | |
|---|---|---|
| 7467 | Left | GCTCGGCCAGTACTCCC |
| 7468 | Right | actgttctagaaggcaaatcacatt |
| 7469 | Left | CATTTCGGACTGGGAGCGA |
| 7470 | Right | gttttgtgtctactgttctagaagg |
| 7471 | Left | CAGGTGCGGGAGAGAGG |
| 7472 | Right | atgtatagaaggcatcatcaacacc |
| 7473 | Left | CTCAGCGGCTCCCAGG |
| 7474 | Right | gtcttgtctttgctgatgtttcaat |
| 7475 | Left | TCCCAGGTGCGGGAGA |
| 7476 | Right | tatagaaggcatcatcaacaccctg |
| 7477 | Left | TGCGGGAGAGAGGCCT |
| 7478 | Right | atcatcaacaccctgtcttgtcttt |
| 7479 | Left | GCCAGTACTCCCGGCC |
| 7480 | Right | agcctgttttgtgtctactgttc |
| 7481 | Left | GCACTGAAGGCGGCG |
| 7482 | Right | ctgtcttgtctttgctgatgtttc |
| 7483 | Left | GGACTGGGAGCGAGCG |
| 7484 | Right | ttcttgctaagtcctgagcctgttt |
| 7485 | Left | AGCGGCTCCCAGGTGC |
| 7486 | Right | catcatcaacaccctgtcttgtc |
| 7487 | Left | CCAGAGGCTCAGCGG |
| 7488 | Right | tgctgatgtttcaataaaaggaattcc |
| 7489 | Left | GCTCGGCCAGTACT |
| 7490 | Right | tctactgttctagaaggcaaatcac |
| 7491 | Left | CATTTCGGACTGGGAG |
| 7492 | Right | cttcttgctaagtcctgagcct |
| 7493 | Left | GGCGGCGAAGGT |
| 7494 | Right | ccataggtacatcttcagagtcctt |
| 7495 | Left | AAGGTGGCGGCG |
| 7496 | Right | tgttctagaaggcaaatcacatttat |
| 7497 | Left | GGCACTGAAGGCG |
| 7498 | Right | tcttgtctttgctgatgtttcaataaa |
| 7499 | Left | TCAGCGGCTCCC |
| 7500 | Right | ttgctgatgtttcaataaaaggaat |
| 7501 | Left | CGGAGGCAGCAG |
| 7502 | Right | taggtacatcttcagagtccttaac |
| 7503 | Left | CCCCGCCATTTCG |
| 7504 | Right | tctactgttctagaaggcaaat |
| 7505 | Left | GCGAAGGTGGCG |
| 7506 | Right | gttttgtgtctactgttctaga |

KRAS Region3 526-575 bases

| | | |
|---|---|---|
| 7507 | Left | ATTTCGGACTGGGAGCGAG |
| 7508 | Right | atgtatagaaggcatcatcaacacc |
| 7509 | Left | GCTCGGCCAGTACTCCC |
| 7510 | Right | gtcttgtctttgctgatgtttcaat |
| 7511 | Left | GCCAGTACTCCCGGCC |
| 7512 | Right | ctgtcttgtctttgctgatgtttc |
| 7513 | Left | GTGCGGGAGAGAGGCC |
| 7514 | Right | ttttaccatctttgctcatcttttc |
| 7515 | Left | GCACTGAAGGCGGCG |
| 7516 | Right | tatagaaggcatcatcaacaccctg |
| 7517 | Left | CATTTCGGACTGGGAGC |
| 7518 | Right | atcatcaacaccctgtcttgtcttt |
| 7519 | Left | CTCAGCGGCTCCCAGG |
| 7520 | Right | ttttaccatctttgctcatctt |
| 7521 | Left | GCTCGGCCAGTACT |
| 7522 | Right | tgctgatgtttcaataaaaggaattcc |
| 7523 | Left | AGCGGCTCCCAGGTGC |
| 7524 | Right | tgctcatcttttctttatgttt |
| 7525 | Left | GGCGGCGAAGGT |
| 7526 | Right | gttttgtgtctactgttctagaagg |
| 7527 | Left | CCCCGCCATTTCG |
| 7528 | Right | tcttgtctttgctgatgtttcaataaa |
| 7529 | Left | AAGGTGGCGGCG |
| 7530 | Right | ttgctgatgtttcaataaaaggaat |
| 7531 | Left | GGCACTGAAGGCG |
| 7532 | Right | catcatcaacaccctgtcttgtc |
| 7533 | Left | GCTCCCAGGTGCGGGA |
| 7534 | Right | ctttatgttttcgaatttctc |
| 7535 | Left | CGGAGGCAGCAG |
| 7536 | Right | actgttctagaaggcaaatcacatt |
| 7537 | Left | GCGAAGGTGGCG |
| 7538 | Right | agcctgttttgtgtctactgttc |
| 7539 | Left | CCAGGTGCGGGAGAGAG |
| 7540 | Right | tttaccatctttgctcat |
| 7541 | Left | CCAGAGGCTCAGCGG |
| 7542 | Right | gaatttctcgaactaatgtata |
| 7543 | Left | CATTTCGGACTGGG |
| 7544 | Right | cctgtcttgtctttgctgatgt |
| 7545 | Left | TCAGCGGCTCCC |
| 7546 | Right | ttgctcatcttttctttatg |

KRAS Region3 576-625 bases

| | | |
|---|---|---|
| 7547 | Left | GCTCGGCCAGTACTCCC |
| 7548 | Right | atgtatagaaggcatcatcaacacc |
| 7549 | Left | CATTTCGGACTGGGAGCGA |
| 7550 | Right | ttttaccatctttgctcatcttttc |
| 7551 | Left | GCCAGTACTCCCGGCC |
| 7552 | Right | tatagaaggcatcatcaacaccctg |
| 7553 | Left | GGACTGGGAGCGAGCG |
| 7554 | Right | ttttaccatctttgctcatctt |
| 7555 | Left | GCACTGAAGGCGGCGG |
| 7556 | Right | tgctcatcttttctttatgttt |
| 7557 | Left | GCTCGGCCAGTACT |
| 7558 | Right | atcatcaacaccctgtcttgtcttt |
| 7559 | Left | AAGGTGGCGGCG |
| 7560 | Right | gtcttgtctttgctgatgtttcaat |
| 7561 | Left | GGCGGCGAAGGT |
| 7562 | Right | ctgtcttgtctttgctgatgtttc |
| 7563 | Left | GCGAAGGTGGCG |
| 7564 | Right | tgctgatgtttcaataaaaggaattcc |
| 7565 | Left | CAGGTGCGGGAGAGAGG |
| 7566 | Right | ttacataattacacactttg |
| 7567 | Left | CCCCGCCATTTCG |
| 7568 | Right | taatgtatagaaggcatcatcaac |
| 7569 | Left | CTCAGCGGCTCCCAGG |
| 7570 | Right | cacactttgtctttgac |
| 7571 | Left | CGGAGGCAGCAG |
| 7572 | Right | cctgtcttgtctttgctgatgt |
| 7573 | Left | CATTTCGGACTGGGAG |
| 7574 | Right | gaatttctcgaactaatgtata |
| 7575 | Left | GGCACTGAAGGCGG |
| 7576 | Right | ttttaccatctttgctcat |
| 7577 | Left | CTCCCAGGTGCGGGA |
| 7578 | Right | gtctttgacttcttt |
| 7579 | Left | CATTTCGGACTGG |
| 7580 | Right | ctttatgttttcgaatttctc |

KRAS Region3 626-675 bases

| | | |
|---|---|---|
| 7581 | Left | GCTCGGCCAGTACTCCC |
| 7582 | Right | ttttaccatctttgctcatcttttc |
| 7583 | Left | GCCAGTACTCCCGGCC |
| 7584 | Right | ttttaccatctttgctcatctt |
| 7585 | Left | ATTTCGGACTGGGAGCGAG |
| 7586 | Right | ttacataattacacactttg |
| 7587 | Left | CGGAGGCAGCAG |
| 7588 | Right | atgtatagaaggcatcatcaacacc |
| 7589 | Left | GCTCGGCCAGTACT |
| 7590 | Right | tgctcatcttttctttatgttt |
| 7591 | Left | CATTTCGGACTGGGAGC |
| 7592 | Right | cacactttgtctttgac |
| 7593 | Left | GGCGGCGAAGGT |
| 7594 | Right | gaatttctcgaactaatgtata |
| 7595 | Left | AAGGTGGCGGCG |
| 7596 | Right | ctttatgttttcgaatttctc |
| 7597 | Left | CCCCGCCATTTCG |
| 7598 | Right | ttttaccatctttgctcat |
| 7599 | Left | GCGAAGGTGGCG |
| 7600 | Right | ttgctcatcttttctttatg |
| 7601 | Left | CATTTCGGACTGGG |
| 7602 | Right | gtctttgacttcttt |

KRAS Region3 676-725 bases

| | | |
|---|---|---|
| 7603 | Left | GCTCGGCCAGTACTCCC |
| 7604 | Right | ttacataattacacactttg |
| 7605 | Left | CGGAGGCAGCAG |
| 7606 | Right | ttttaccatctttgctcatctttt |
| 7607 | Left | GCTCGGCCAGTACT |
| 7608 | Right | cacactttgtctttgac |
| 7609 | Left | AAGGTGGCGGCG |
| 7610 | Right | gtctttgacttcttt |

TABLE 15
IRID Hybridization-based capture probes for NGS panel

| ID | Probe Sequence |
|---|---|
| 7611 | CGGTCCAGAGCCAAGCGGCGGCAGAGCGAGGGGCATCAGCTACCGCCAAGTCCAGAG |
| | CCATTTCCATCCTGCAGAAGAAGCCCCGCCACCAG |
| 7612 | AGAAATGGATACAGGTCAAGTCTAAGTCGAATCCATCCTCTTGATATCTCCTTTTGTTTC |
| | TGCTAACGATCTCTTTGATGATGGCTGTCATGTCTGGGAGCCTGTGGCTGAAGAAAAAG |
| | GAGGAGAGAGATGGCAGAAGCTGC |
| 7613 | AAGTTGACAGGGTACCAGGAGATGATGTAAGGGACAAGCAGCCACACCCCATTCTTGA |
| | GGGGCTGAGGTGGAAG |
| 7614 | GATTGGAGACTTCGGGATGGCCCGAGACATCTACAGGTGAGTAAAGACTGCCTCACCC |
| | CTCCGGGCCTGTCTCTTCCACCTCAGCCCCTCAAGAATGGGG |
| 7615 | CTTGGCCACTCTTCCAGGGCCTGGACAGGTCAAGAGGCAGTTTCTGGCAGCAATGTCTC |
| | TGGGAAGAAAGGAAATGCATTTCCTAATTTTATCCCTAGGAAGATGAGTGTACAACGG |
| 7616 | CACTTTGACTCACCGGTGGATGAAGTGGTTTTCCTCCAAATACTGACAGCCACAGGCAA |
| | TGTCCCGAGCCACGTGCAGAAGGTCCAGCATGGCCAGGGAGGAGGGCTGGCTCTGTGG |
| | GGAGACAGAAGCGGGC |
| 7617 | CCCTGGGTTCCATCGAGGAACTTGCTACCCAGGCTGCCCACTCTTGCTCCTTCCATCCTT |
| | GCTCCTGTCCTTGGC |
| 7618 | GTTCATCCTGCTGGAGCTCATGGCGGGGGGAGACCTCAAGTCCTTCCTCCGAGAGACCC |
| | GCCCTCGCCCGGTGAGTGAGAACCAGTCTTTGCTGCAGTTGTTGTGCC |
| 7619 | CCGGGGCAGGGATTGCAGGCTCACCCCAATGCAGCGAACAATGTTCTGGTGGTTGAAT |
| | TTGCTGCAGAGCAGAGAGGGATGTAACCAAAATTAACTGAGCTGAGTCTGGGCAAATC |
| | TTAAACTGGGAGGAACA |
| 7620 | GGGAGGGCTCTGCCGGCCTTTTGTGGCTAGAGGAGTCTGCGGTGCTGTGATAACATTCA |
| | GCCCCTACACTGCACCCCTCTCCTCCCAGGACGGC |
| 7621 | CCCAACTACTGCTTTGCTGGCAAGACCTCCTCCATCAGTGACCTGAAGGAGGTGCCGCG |
| | GAAAAACATCACCCTCATTCGGTGAGCGCCCTGCTGC |
| 7622 | GGTTGTAGTCGGTCATGATGGTCGAGGTGCGGAGCTTGCTCAGCTTGTACTCAGGGCTC |
| | TGCAGCTCCATCTGCATGGCTTGCAGCTCCTGGTGCTTCCGGCGGTACACTGCAGGTGG |
| | GTG |
| 7623 | TTTTAAATACCTGTTAAGTTTGTATGCAACATTTCTAAAGTTACCTACTTGTTAATTAAA |
| | AATTCAAGAGTTTTTTTTTCTTATTCTGAGGTTATCTTTTTACCACAG |
| 7624 | TTAGCCATTGGTCAAGATCTTCACAAAAGGGTTTGATAAGTTCTAGCTGTGGTGGGTTA |
| | TGGTCTTCAAAAGGATATTGTGCAACT |
| 7625 | AAGTGAAGATGACAATCATGTTGCAGCAATTCACTGTAAAGCTGGAAAGGGACGAACT |
| | GGTGTAATGATATGTGCATATTTATTACATCGGGGCAA |
| 7626 | GAAGAGGAAAGGAAAAACATCAAAAAATAACTTACCTTTTTGTCTCTGGTCCTTACTTC |
| | CCCATAGAAATCTAGGGCCTCTTGTGCCTTTAAAAATT |
| 7627 | CAGTTACCATAGCAATTTAGTGAAATAACTATAATGGAACATTTTTTTTCAATTTGGCTT |
| | CTCTTTTTTTTCTGTCCACCAGGGAGTAACTA |
| 7628 | ACATCATCTTGTGAAACAACAGTGCCACTGGTCTATAATCCAGATGATTCTTTAACAGG |
| | TAGCTATAATAATACACATAGCGCCTCTGACTGGGAAT |
| 7629 | TTTGAAACTATTCCAATGTTCAGTGGCGGAACTTGCAGTAAGTGCTTGAAATTCTCATC |
| | CTTCCATGTATTGGAACAGTTTTCTTAACCATATCTAGAAGTTTACATAAAAATTTAGA |
| | AAGAAATTT |
| 7630 | ATATTTCGTGTATATTGCTGATATTAATCATTAAAATCGTTTTTGACAGTTTGACAGTTA |
| | AAGGCATTTCCTGTGAAATAATAC |
| 7631 | CGTGTGGGTCCTGAATTGGAGGAATATATCTTCACCTTTAGCTGGCAGACCACAAACTG |
| | AGGATCTGCATGGTTAAATACATACCAGTATT |
| 7632 | GACGGGAAGACAAGTTCATGTACTTTGAGTTCCCTCAGCCGTTACCTGTGTGTGGTGAT |
| | ATCAAAGTAGAGTTCT |
| 7633 | TTATAGTTCCTTACATGTCATAAAATAAAATATAGCTTTTAATCTGTCCTTATTTTGGAT |
| | ATTTCTCCCAATGAAAGTAAAGTACAAACCTTTTTTAGCATCTTGTTCTGTTTGTGGAA |
| 7634 | TAACATAGGTGACAGATTTTCTTTTTTAAAAAAATAAAACATCATTAATTAAATATGTC |
| | ATTTCATTTCTTTTTCTTTTCTTTTTTTTTTTTTTTAGGACAAAATGTTTCACTTTTGGGTA |
| 7635 | ATCACATAGACTTCCATTTTCTACTTTTTCTGAGGTTTCCTCTGGTCCTGGTATGAAGAA |
| | TGTATTTA |
| 7636 | TATGTGATCAAGAAATCGATAGCATTTGCAGTATAGAGCGTGCAGATAATGACAAGGA |
| | ATATCTAGTACTTACTTTAACAAAAAATGATCTTGACAAAGCAAATAAAGACAA |
| 7637 | TACAAGTCAACAACCCCCACAAAATGTTTAATTTAACTGACCTTAAAATTTGGAGAAAA |
| | GTATCGGTTGGCTT |
| 7638 | AGAGAAACCTTTATCTGTATCAAAGAATGGTCCTGCACCAGTAATATGCATATTAAAAC |
| | AAGATTTACCTCTATTGTTGGATCA |
| 7639 | CTGGTGGCGTAGGCAAGAGTGCCTTGACGATACAGCTAATTCAGAATCATTTTGTGGAC |
| | GAATATG |
| 7640 | GCTCCAACTACCACAAGTTTATATTCAGTCATTTTCAGCAGGCCTTATAATAAAAATAA |
| | TGAAAATGTGACTATATTAGAACATGTCACACATAAGGTTAATACA |
| 7641 | GCCCATGCCGTGGCTGCTGGTCCCCCTGCTGGGCCATGTCTGGCACTGCTTTCCAGCAT |
| | GGTGAGGGCTGAGGTGACCCTTGTCTCTGTGTTCTTGTCCCCCCCAGCTTGTGGAGCCT |
| | CTTACACC |
| 7642 | CAGAGGCCTGTGCCAGGGACCTTACCTTATACACCGTGCCGAACGCACCGGAGCCCAG |
| | CACTTTGATCTTTTTGAATTCAGTTTCCTTCAAGATCCTCAAGAGAGCTTGGTTGGGAGC |
| | TTCTCCACTGG |
| 7643 | CCTTCGGGGTGCATCGCTGGTAACATCCACCCAGATCACTGGGCAGCATGTGGCACCAT |
| | CTCACAATTGCCAGTTAACGTCTTCCT |
| 7644 | TCTCTTAATTCCTTGATAGCGACGGGAATTTTAACTTTCTCACCTTCTGGGATCCAGAGT |
| | CCCTATGACAGAGAGAGAAG |
| 7645 | AAGCAACATCTCCGAAAGCCAACAAGGAAATCCTCGATGTGAGTTTCTGCTTTGCTGTG |
| | TGGGGGTCCATGGCTCTGAACCTCAGGCCCACCTTTTCTCATGTCTGGCAGC |
| 7646 | ATTTTGAAACTCAAGATCGCATTCATGCGTCTTCACCTGGAAGGGGTCCATGTGCCCCT |
| | CCTTCTGGCCACCATGCGAAG |
| 7647 | GAGGTGAGGCAGATGCCCAGCAGGCGGCACACGTGGGGGTTGTCCACGCTGGCCATCA |
| | CGTAGGCTTCCTGGAGGGAGGGAGAGGCACGTCAGTGTGGC |
| 7648 | CCACCGTGCAGCTCATCACGCAGCTCATGCCCTTCGGCTGCCTCCTGGACTATGTCCGG |
| | GAACACAAAGACAATATTGGCTCCCAGTACCTGCTCAACTGGTGTGTGCAGATCGCAA |
| | AGGTAATCAGGGAAGGGA |
| 7649 | CGTGGAGAGGCTCAGAGCCTGGCATGAACATGACCCTGAATTCGGATGCAGAGCTTCT |
| | TCCCATGATGATCTGTCCCTCACAGCAGGGT |
| 7650 | GCGGTGTTTTCACCAGTACGTTCCTGGCTGCCAGGTCGCGGTGCACCAAGCGACGGTCC |
| | TCCAAGTAGTTCATGCCCTGAAACAGAGAAGACCCTGC |
| 7651 | CAGCATGTCAAGATCACAGATTTTGGGCTGGCCAAACTGCTGGGTGCGGAAGAGAAAG |
| | AATACCATGCAGAAGGAGGCAAAGTAAGGAGGTGGCTTTAGGTCAGCCAGCATTT |
| 7652 | GATTATCTGTCTGGCCCCAGACCTGGAGCTTTCTTTCCATGATAGGAGTACTTCTTTGGG |
| | TTGACTTCTCTGGTGACAG |
| 7653 | CTCATCAAGCTCTAGCTCCTCCAGCTTCTTCTGCAAGGCCTCCAAGTTGGTCCTGTTCCA |
| | AAGTGGGGAGCACAAGTCAATACT |
| 7654 | GCAGCAGCGAAAGCGCCTTGAGGCCTTTCTTACCCAGAAGCAGAAGGTGGGAGAACTG |
| | AAGGATGACGACTTTGAGAAGATCAGTGAGCTGGGGGCTGGCAATGGCGGTGTGGTGT |
| | TCAAG |
| 7655 | CCCCAGGCTTCTAAGTACCCTGAGAAATAATCCAATTACCTGTTAATCAAGGCAAACTC |
| | ACCTTTCTGGCCATGACCAGGCCAGAAGGCTTGTGGGAGACC |
| 7656 | AAAATACTATAGTTGAGACCTTCAATGACTTTCTAGTAACTCAGCAGCATCTCAGGGCC |
| | AAAAATTTAATCAGTGGAAAAATAGCCTCA |
| 7657 | CTACAGTGAAATCTCGATGGAGTGGGTCCCATCAGTTTGAACAGTTGTCTGGATCCATT |
| | TTGTGGATGGTAAGAATT |
| 7658 | GCTAGACCAAAATCACCTATTTTTACTGTGAGGTCTTCATGAAGAAATATATCTGAGGT |
| | GTAGTAAGTAAAGGAAAACAGTAGAT |
| 7659 | ATATATACATAAGAGAGAAGGTTTGACTGCCATAAAAAATATCTAATTTATGACAATA |
| | AAAACCTTACTTTATTTGGATTTGAT |
| 7660 | ATATATAATAGCTTTTCTTCCATCTCTTAGGAAACTCCATGCTTAGAGTTGGAGTTTGAC |
| | TGGTTCAGCAGTGTGGTAAAGT |
| 7661 | TTATAATTGATACTTAATAAACTCAGTGATTTGCCTTACCAGTCCTGCGTGGGAATAGC |
| | TAAATCCTGCTTCTCGGGATACAGACCAATTGGCATGCTCTTCAATCACTGACATATCT |
| | GGGAA |
| 7662 | TTTCTCCTGCAGCTGTGTGGACCTGGATGACAAGGGCTGCCCCGCCGAGCAGAGAGCC |
| | AGGTTGGCCTGGACCC |
| 7663 | GCTAGGGACAACACGATTTCCCTTGGAGATATCGATCTGTTAGAAACCTCTCCAGGTTC |
| | TTTGGGGGCAGAG |
| 7664 | TCATGGAAGCCCTGATCATCAGGTAAAGCCACAGAGAGACACCCTCACCCCAACTCCC |
| | CTCTGCCCCCAAAGAAC |
| 7665 | GAGGAAATCCAGTTCGTCCTGTTCAGAGCACACTTCAGGCAGCGTCTGGGCAGAGAAG |
| | GGGAGGGTGGGGAGGAGGAGGAGGCTGTGA |
| 7666 | CAAGTGGCTGTGAAGGTAAGAAGTGGCTCACTCTTGAGCCTGCCCTTGGCTTGCGGACT |
| | CTGTAGGCTGCAGTTCTCAGCTC |
| 7667 | GCAGGGGGCTTGGGTCGTTGGGCATTCCGGACACCTGGCCTTCATACACCTCCCCAAAG |
| | GCGCCATGGCCCAGACCCCTGTGCAAAGGAGAAGACAAGAGG |
| 7668 | CCCTCTAGGGTTGTCAATGAAATGAATTCACCAACATAAAATGGTTTTGAAAAATCCTA |
| | AAGAGCTCTACCAATGTGAGTGACCATTATCACTCCT |
| 7669 | GCTGGGCTTTACACACAGAATCTACCCACTGAATCACAATTTTGTTCTGGCTTCCATGG |
| | AGTTTGCCTTCCAGAACATCCTCACATGTA |
| 7670 | CCCCCCAACACATGGGCCAGGGCAAATGAGTCACCCGCTATGTGCTCAGTTCCCTCCTC |
| | TATGCAATGGACCGACCGTGATCAGATTAGGGTTACCTGAGGATCGAATGAATTGAAA |
| | TG |
| 7671 | TAATATCACATTGTATAGACATACTGTGTTTTTAAAATCTATTCATCAGTTGAGGAACA |
| | GTTTTAGAAAATGAATAGTTGTTAAACCTGAATT |
| 7672 | AGAACCTTATTGAAATATGGCCAAAGTAAGTCAATAAACAGCCCTTAGTCCTCTTGAAA |
| | AAATTCAGGTTTAACAACTATTCATTTT |
| 7673 | TACCTTCCCATTTTGAACCTGGTAACTAATTCTGTAGGACATCATCTTCCAAAAAGCTTT |
| | ACTCTTGCTCTGCTAGCCTATCTAGCTAAAGAGCTTCTCTG |
| 7674 | GGCCACTTCCCAGCTGGCGCGGACACGGCAGGCTGGAGAGCCATGAGGCAGAGCATAC |
| | GCAGCCTGTACCCAGTGGTGCCGAGCCTCTGGCG |
| 7675 | CGATGAAGGAGAAGAGGACAGCGGCTGCGATCACCGTGCGGCACAGCTCGTCGCACA |
| | GTGGATCTGTGGGTGGGGGTGGTGTGAGGCTTGGCACCG |
| 7676 | CATCGTCTCGGTGCTGCTGTCTGCCTTCTGCATCCACTGCTACCACAAGTTTGCCCACAA |
| | GCCACCCATCTCCTCAGCTGAGATGAC |
| 7677 | GAGACCTGGTTCTCCATGGAGTCCAGCGAGGGCCGGCGGGCACCGGAAGAGGAGTAGC |
| | TGACCGGGAAGGCCTGGGCGGGCCTCCGGAAGG |
| 7678 | CCGTGGATGCCTTCAAGATCCTGGTGAGGGTCCCTGCGGGGCAGGGAAGATCCCCTGC |
| | CCTCCCCAGCTGCCTTCCAGGGAGGGAGGCCAGCTGG |
| 7679 | CCAGGAGTGTCTACAGCACTCCTCTGGTTACTGAAAGCTCAGGGATAGGGCCTGGCCTT |
| | CTCCTTTACCCCTCCTTCCTAGAGAGTTAGA |
| 7680 | AAAAGGGATTCAATTGCCATCCATTTAACTGGAATCCGACCCTAAAGAGAAGATGGAA |
| | TAAAGACATTGAAGTTACTC |
| 7681 | TTGATCATATCTACACCACGCAAAGTGATGTGTAAGTGTGGGTGTTGCTCTCTTGGGGT |
| | GGAGGTTACAGAAACACCCTTATACATGTAGTGGGGCCACGACGCCCGTCTGTGCAGC |
| | TTGGCCAG |
| 7682 | TCTTATTCCTTTAATACAGAATATGGGTAAAGATGATCCGACAAGTGAGAGACAGGAT |
| | CAGGTCAGCGGGCTACCACTGGGCC |
| 7683 | CAGGTGGTGTTGGGAAAAGCGCACTGACAATCCAGCTAATCCAGAACCACTTTGTAGA |
| | TGAATATGATCCCACCATAGAGGTGAGG |
| 7684 | GCTCCAACCACCACCAGTTTGTACTCAGTCATTTCACACCAGCAAGAACCTGTTGGAAA |
| | CCAGTAATCAGGGTTAATTGGCGAGCCACATCTACAGTACTTTAAAGCTTTCTATAATCA |
| 7685 | AAGAAGAGTACAGTGCCATGAGAGACCAATACATGAGGACAGGCGAAGGCTTCCTCTG |
| | TGTATTTGCCAT |
| 7686 | GTACTCTTCTTGTCCAGCTGTATCCAGTATGTCCAACAAACAGGTTTCACCATCTATAAC |
| | CACTTGTTTTCTGTAAGAATCCTGGGGGTGT |
| 7687 | CTTGGCAATAGCATTGCATTCCCTGTGGTTTTTAATAAAAATTGAACTTCCCTCCCTCCC |
| | TGCCCCCTTACCCTCCA |
| 7688 | GGTTAAATAAGCATCTAACTATTCAAGCCCATTTCTGCCTATCTGGTTTGTCCCTCAAAT |
| | TGCTAATATATAATCACAAACAAAAAGTATCCAATATCACCCTACATAAAAGAAAACCC |
| 7689 | GGGCTTTCTTTGTGTATTTGCCATAAATAATACTAAATCATTTGAAGATATTCACCATTA |
| | TAGGTGGGTTTAAATTGAATATAATAAGCTGACATTAAGGAGTAATTATAGTTTTTATT |
| | TTTTGAG |
| 7690 | CCTCCCCAGTCCTCATGTACTGGTCCCTCATTGCACTGTACTCCTCTTGACCTGCTGTGT |
| | CGAGAATATCCAAGAGACAGGT |
| 7691 | TTTTTAAATTGAATTTTTTGTTGTTGAGTTGTATATAACACCTTTTTTGAAGTAAAAGGT |
| | GCACTGTAATAATCCAGACTGTGTTTCTCCCTTCTCAGGATTCCTACAGGAAGCAAGTA |
| | GTAATTGATGGAGAAA |
| 7692 | GCTTGTTGCTTGCCAGCCCAGGACTTGGAGGCTCCAGGGGACCCCCATCGTGGGGCCTG |
| | GTGGGCAAAGAGGGCTCCAGCCAACCCCCCAAAT |
| 7693 | CTACAAGGAGCGGCCGCAGGATGTGGACCAACGTGAGGCTCCCCTCAACAACTTCTCT |
| | GTGGCGCGTAAGTATCCCCTTGGCCTCTCGGGATTCAGATTTGGGGGGTTGGCTGGAGC |
| | CC |
| 7694 | GCCAATGAAGGTGCCATCATTCTTGAGGAGGAAGTAGCGTGGCCGCCAGGTCTTGATG |
| | TACTCCCCTACAGACGTGCGGGTGGTGAGAGCCACGCACACTCTACCCGTCAGACCCTC |
| | GCCAGGCAGCCAGG |
| 7695 | AAACTAATTTTTGAGACAAGATAATTTTTTATAAATAAATATTTCAGAATTCTAAGGTC |
| | AAAATTAGAACAGTAGATGCTTAGTTTA |
| 7696 | TTTCATGCCTTTGGCTACTTGAAGACCAAAGCCAATAAGATCTTTTACAGTTGGATTCT |
| | GCAGTCAAAGAGAGTTAGAAAAGCAT |
| 7697 | ATATCTTGCAAGCAAAAAGTTTGTCCACAGAGACTTGGCTGCAAGAAACTGTATGTAA |
| | GTATCAGAATCTCTGTGCCACAATCCAAATTAAGTGACAAGGAGGAATCTG |
| 7698 | AAGTAGTAATAACAGTGCTGTTGATATTGAGACAACACCAGCAATCAATCCTGTGAAA |
| | TTCTGATCTGGTTGAA |
| 7699 | TTGGGTTTTTCCTGTGGCTGAAAAAGAGAAAGCAAATTAAAGGTGCATTTTTGTTACTG |
| | TTCATTTTTAGAAGTTA |
| 7700 | GGTTTTATGGACTACATATAAGACAGCACACAAGAATCGACGACAATCTTAAACTGTA |
| | ATGACTGTGTTCTTAAGGTA |
| 7701 | ATAAAACCCATGAGTTCTGGGCACTGGGTCAAAGTCTCCTGGGGCCCATGATAGCCGTC |
| | TTTAACAAGCTCTTT |
| 7702 | CTTCGGGCACTTACAAGCCTATCCAAATGAGGAGTGTGTACTCTTGCATCGTAGCGAAC |
| | TAATTCACTGCCCAGATCTTAAAACAGAGAGAAAGA |
| 7703 | GTGTAAGCCCAACTACAGAAATGGTTTCAAATGAATCTGTAGACTACCGAGCTACTTTT |
| | CCAGAAGGTATATTTCAGTTTATTGTTCTGAGAAATACCTATACAT |
| 7704 | GCAGCAGCGAAAGCACCTTGAGGCCTTTCTTACCCAGAGATAGAAGGTGGGAGAACTA |
| | AAGGATGACGACTTTGAGAAGATCAGTGAGCTGGGGGCTGGCAATGGCGGTGTGGTGT |
| | CCAAA |
| 7705 | TATGTGATCAAGAAATTGATAGCATTTGCAGTATAGAGCGTGCAGATAATGACAAGGA |
| | GTATCTAGTACTTACTTTAACAAAAAATGATCTTGACAAAGCAAATAAAGACAA |
| 7706 | ATCACATAGACTTCCATTTTCTACTTTTTCTGAGGTTTCCTCTGGTCCTGGTATGAAGAA |
| | TGTATTTACCCAAAAGTGAAACATTTTGTCCTTTTTTAGCATCTTGTTCTGTTTGTGGAA |
| 7707 | GATGGGAGGACAAGTTCATGTATTTTGAGTTCCCTCAGCCGTTACCTGTGTGTGGTGAT |
| | ATCAAAGTAGAGTTCTTCCACAAACAGAACAAGATGCTAAAAAAGGACAAAATGTTTC |
| | ACTTTTGGGTA |
| 7708 | TTTGAAACTATTCCAATGTTCAGTGGCGGAACTTGCAATCCTCAGTTTGTGGTCTGCCA |
| | GCTAAAGGTGAAGATGTATTCCTCCAATTCAGGACCCACACGATGGGAGGACAAGTTC |
| | ATGTATTTTGAGTTCCCTCAGCCGTTACCTGTGTGTGGTGATATCAAAGTAGAGTTCT |
| 7709 | ACATCATCTTGTGAAACAACAGTGCCACTGGTCTATAATCCACATGATTCTTTACCAGG |
| | TAGCTATAGTAATACACATAGCGCCTCTGACTGGGAATAGTTACTCCCTTTTTGTCTCTG |
| | GTCCTTACTTCCCCATAGAAATCTAGGGCCTCTTGTGCCTTTAAAAATT |
| 7710 | AAGTGAAGATGACAATCATGTTGCAGCAATTCACTGTAAAGCTGGAAAGGGACGAACT |
| | GGTATAATGATTTATGCATATTTATTACATCGGGGCAA |
| 7711 | CGGTCCAGAGCCAAGCAGCGGCTGAGCGAGGGGCATCAGCTACCGCCAAGTCCAGAGC |
| | CATTTCCATCCTGCAGAGCCCCGCCACCAG |
| 7712 | CTGGTGGCGTAAGCAAAAGTGTCTTGACGATACAGCTAATTCAGAATCATTTTGTGGAC |
| | CAATATGATCCAACAATAGAGAATTCCTACAGGAAGCAAGTAGTAATTGATGGAGAAA |
| 7713 | CCCCCCCCCCCCCCCGCCCAGTCCTCATGTACTGGTCCTCATTGCATTGTACTCTTCTTG |
| | ACCTGTTGTGTCAAGAATATCCAAGAGACAGGT |

### REFERENCES

1. Bang Y, Kwak E L, Shaw A T, Camidge D R, Iafrate A J, Maki R G, Solomon B J, Ou R, Salgia R, Clark J W. Activity of the oral ALK inhibitor PF-02341066 in ALK-positive patients with non-small cell lung cancer (NSCLC). J Clin Oncol 28: 18s (suppl; abstr 3), 2010.
2. Butrynski J E, D'Adamo D R, Hornick J L, Dal Cin P, Antonescu C R, Jhanwar S C, Ladanyi M, Capelletti M, Rodig S J, Ramaiya N, Kwak E L, Clark J W, Wilner K D, Christensen J G, Jinne P A, Maki R G, Demetri G D, Shapiro G I. Crizotinib in ALK-rearranged inflammatory myofibroblastic tumor. N Engl J Med. 2010 Oct. 28; 363(18):1727-33.
3. Camidge D R, Doebele R C. Treating ALK-positive lung cancer—early successes and future challenges. Nat Rev Clin Oncol. 2012 Apr. 3; 9(5):268-77.
4. Cheng M, Ott G R. Anaplastic lymphoma kinase as a therapeutic target in anaplastic large cell lymphoma, non-small cell lung cancer and neuroblastoma. Anticancer Agents Med Chem 10: 236-249, 2010.
5. Choi Y L, Soda M, Yamashita Y, Ueno T, Takashima J, Nakajima T, Yatabe Y, Takeuchi K, Hamada T, Haruta H, Ishikawa Y, Kimura H, Mitsudomi T, Tanio Y, Mano H; ALK Lung Cancer Study Group. EML4-ALK mutations in lung cancer that confer resistance to ALK inhibitors. N Engl J Med. 2010 Oct. 28; 363(18):1734-9.
6. Choi Y L, Soda M, Yamashita Y, Ueno T, Takashima J, Nakajima T, Yatabe Y, Takeuchi K, Hamada T, Haruta H, Ishikawa Y, Kimura H, Mitsudomi T, Tanio Y, Mano H. ALK Lung Cancer Study Group. EML4-ALK mutations in lung cancer that confer resistance to ALK inhibitors. N Engl J Med. 2010 Oct. 28; 363(18):1734-9.
7. Christensen J G, Zou H Y, Arango M E, Li Q, Lee J H, McDonnell S R, Yamazaki S, Alton G R, Mroczkowski B, Los G. Cytoreductive antitumor activity of PF-2341066, a novel inhibitor of anaplastic lymphoma kinase and c-Met, in experimental models of anaplastic large-cell lymphoma. Mol Cancer Ther 6 (12, Pt. 1): 3314-3322, 2007.
8. Cui J J et al. Structure based drug design for the discovery of clinical candidate PF-2341066 as potent and highly selective c-Met inhibitor. Abstracts of Papers, 235th ACS National Meeting, New Orleans, La., United States, Apr. 6-10, 2008, 2008: p. MEDI-177.
9. Doebele R C, Pilling A B, Aisner D L, Kutateladze T G, Le A T, Weickhardt A J, Kondo K L, Linderman D J, Heasley L E, Franklin W A, Varella-Garcia M, Camidge D R. Mechanisms of resistance to crizotinib in patients with ALK gene rearranged non-small cell lung cancer. Clin Cancer Res. 2012 Mar. 1; 18(5):1472-82.
10. Doebele R C, Pilling A B, Aisner D L, Kutateladze T G, Le A T, Weickhardt A J, Kondo K L, Linderman D J, Heasley L E, Franklin W A, Varella-Garcia M, Camidge DR. Mechanisms of resistance to crizotinib in patients with ALK gene rearranged non-small cell lung cancer. Clin Cancer Res. 2012 Mar. 1; 18(5):1472-82. Epub 2012 Jan. 10.
11. Engelman J A, Zejnullahu K, Mitsudomi T, Song Y, Hyland C, Park J O, Lindeman N, Gale C M, Zhao X, Christensen J, Kosaka T, Holmes A J, Rogers A M, Cappuzzo F, Mok T, Lee C, Johnson B E, Cantley L C, Jainne P A. Science. 2007 May 18; 316(5827):1039-43. Epub 2007 Apr. 26.
12. Hallberg B, Palmer R H. Crizotinib-latest champion in the cancer wars?N Engl J Med 363: 1760-1762, 2010.
13. Koivunen J P, Mermel C, Zejnullahu K, Murphy C, Lifshits E, Holmes A J, Choi H G, Kim J, Chiang D, Thomas R, Lee J, Richards W G, Sugarbaker D J, Ducko C, Lindeman N, Marcoux J P, Engelman J A, Gray N S, Lee C, Meyerson M, Janne P A. EML4-ALK fusion gene and efficacy of an ALK kinase inhibitor in lung cancer. Clin Cancer Res 14: 4275-4283, 2008.
14. Lovly C M, Pao. Escaping ALK inhibition: mechanisms of and strategies to overcome resistance. W. Sci Transl Med. 2012 Feb. 8; 4(120):120ps2.
15. McDermott U, Iafrate A J, Gray N S, Shioda T, Classon M, Maheswaran S, Zhou W, Choi H G, Smith S L, Dowell L, Ulkus L E, Kuhlmann G, Greninger P, Christensen J G, Haber D A, Settleman J. Genomic alterations of anaplastic lymphoma kinase may sensitize tumors to anaplastic lymphoma kinase inhibitors. Cancer Res 68: 3389-3395, 2008.
16. Sasaki T, Okuda K, Zheng W, Butrynski J, Capelletti M, Wang L, Gray N S, Wilner K, Christensen J G, Demetri G, Shapiro G I, Rodig S J, Eck M J, Jainne P A. The neuroblastoma associated F1174L ALK mutation causes resistance to an ALK kinase inhibitor in ALK translocated cancers. Cancer Res. 2010 Dec. 15; 70(24):10038-43.
17. Sasaki T, Rodig S J, Chirieac L R, Janne P A. The biology and treatment of EML4-ALK non-small cell lung cancer. Eur J Cancer 46: 1773-1780, 2010.
18. Shaw A T, Yeap B Y, Mino-Kenudson M, Digumarthy S R, Costa D B, Heist R S, Solomon B, Stubbs H, Admane S, McDermott U, Settleman J, Kobayashi S, Mark E J, Rodig S J, Chirieac L R, Kwak E L, Lynch T J, Iafrate A J. Clinical features and outcome of patients with non-small-cell lung cancer who harbor EML4-ALK. J Clin Oncol 27: 4247-4253, 2009.
19. Wood A C, Laudenslager M, Haglund E A, Attiyeh E F, Pawel B, Courtright J, Plegaria J, Christensen J G, Mosse Y P. Inhibition of ALK mutated neuroblastomas by the selective inhibitor PF-02341066. J Clin Oncol 27:15s, 2009 (suppl; abstr 10008b).
20. Zou H Y, Li Q, Lee J H, Arango M E, McDonnell S R, Yamazaki S, Koudrakova T B, Alton G, Cui J J, Kung P P, Nambu M D, Los G, Bender S L, Mroczkowski B, Christensen J G. An orally available small-molecule inhibitor of c-Met, PF-2341066, exhibits cytoreductive antitumor efficacy through antiproliferative and antiangiogenic mechanisms. Cancer Res 67: 4408-4417, 2007.

SEQUENCES

anaplastic lymphoma kinase (ALK) SEQ ID NO: 7714

| | |
|---|---|
| 1 | mgaigllwllplllstaavgsgmgtgqragspaagpplqpreplsysrlqrkslavdfvv |
| 61 | pslfrvyardlllppssselkagrpeargslaldcapllrllgpapgvswtagspapaea |
| 121 | rtlsrvlkggsvrklrrakqlvlelgeeailegcvgppgeaavgllqfnlselfswwirq |
| 181 | gegrlrirlmpekkasevgregrlsaairasqprllfqifgtghsslesptnmpspspdy |
| 241 | ftwnltwimkdsfpflshrsryglecsfdfpceleyspplhdlrnqswswrripseeasq |
| 301 | mdlldgpgaerskemprgsflllntsadskhtilspwmrsssehctlavsvhrhlqpsgr |
| 361 | yiaqllphneaareillmptpgkhgwtvlqgrigrpdnpfrvaleyissgnrslsavdff |
| 421 | alkncsegtspgskmalqssftcwngtvlqlgqacdfhqdcaqgedesqmcrklpvgfyc |
| 481 | nfedgfcgwtqgtlsphtpqwqvrtlkdarfqdhqdhalllsttdvpasesatvtsatfp |
| 541 | apiksspcelrmswlirgvlrgnvslvlvenktgkeqgrmvwhvaayeglslwqwmvlpl |
| 601 | ldvsdrfwlqmvawwgqgsraivafdnisisldcyltisgedkilqntapksrnlfernp |
| 661 | nkelkpgensprqtpifdptvhwlfttcgasgphgptqaqcnnayqnsnlsvevgsegpl |
| 721 | kgiqiwkvpatdtysisgygaaggkggkntmmrshgvsvlgifnlekddmlyilvgqqge |
| 781 | dacpstnqliqkvcigennvieeeirvnrsvhewaggggggggatyvfkmkdgvpvplii |
| 841 | aaggggraygaktdtfhperlennssvlglngnsgaagggggwndntsllwagkslqega |
| 901 | tgghscpqamkkwgwetrggfggggggcssggggggyiggnaasnndpemdgedgvsfis |
| 961 | plgilytpalkvmeghgevnikhylncshcevdechmdpeshkvicfcdhgtvlaedgvs |
| 1021 | civsptpephlplslilsvvtsalvaalvlafsgimivyrrkhgelqamqmelqspeykl |
| 1081 | sklrtstimtdynpnycfagktssisdlkevprknitlirglghgafgevyegqvsgmpn |
| 1141 | dpsplqvavktlpevcseqdeldflmealiiskfnhqnivrcigvslqslprfillelma |
| 1201 | ggdlksflretrprpsqpsslamldllhvardiacgcqyleenhfihrdiaarnclltcp |
| 1261 | gpgrvakigdfgmardiyrasyyrkggcamlpvkwmppeafmegiftsktdtwsfgvllw |
| 1321 | eifslgympypsksnqevlefvtsggrmdppkncpgpvyrimtqcwqhqpedrpnfaiil |
| 1381 | erieyctqdpdvintalpieygplveeeekvpvrpkdpegvppllvsqqakreeerspaa |
| 1441 | ppplpttssgkaakkptaaeisvrvprgpavegghvnmafsqsnppselhkvhgsrnkpt |
| 1501 | slwnptygswftekptkknnpiakkephdrgnlglegsctvppnvatgrlpgasllleps |
| 1561 | sltanmkevplfrlrhfpcgnvnygyqqqglpleaatapgaghyedtilksknsmnqpgp |

ALK cDNA Sequence Reference SEQ ID NO: 7715
atgggagccatcgggctcctgtggctcctgccgctgctgctttccacggcagctgtgggc
tccgggatggggaccggccagcgcgcgggctccccagctgcggggccgccgctgcagccc
cgggagccactcagctactcgcgcctgcagaggaagagtctggcagttgacttcgtggtg
ccctcgctcttccgtgtctacgcccgggacctactgctgccaccatcctcctcggagctg
aaggctggcaggcccgaggcccgcggctcgctagctctggactgcgccccgctgctcagg
ttgctggggccggcgccgggggtctcctggaccgccggttcaccagccccggcagaggcc
cggacgctgtccagggtgctgaagggcggctccgtgcgcaagctccggcgtgccaagcag
ttggtgctggagctgggcgaggaggcgatcttggagggttgcgtcgggccccccggggag
gcggctgtggggctgctccagttcaatctcagcgagctgttcagttggtggattcgccaa
ggcgaagggcgactgaggatccgcctgatgcccgagaagaaggcgtcggaagtgggcaga
gagggaaggctgtccgcggcaattcgcgcctcccagccccgccttctcttccagatcttc
gggactggtcatagctccttggaatcaccaacaaacatgccttctccttctcctgattat
tttacatggaatctcacctggataatgaaagactccttccctttcctgtctcatcgcagc
cgatatggtctggagtgcagctttgacttcccctgtgagctggagtattcccctccactg
catgacctcaggaaccagagctggtcctggcgccgcatcccctccgaggaggcctcccag
atggacttgctggatgggcctggggcagagcgttctaaggagatgcccagaggctccttt
ctccttctcaacacctcagctgactccaagcacaccatcctgagtccgtggatgaggagc
agcagtgagcactgcacactggccgtctcggtgcacaggcacctgcagccctctggaagg
tacattgcccagctgctgccccacaacgaggctgcaagagagatcctcctgatgcccact
ccagggaagcatggttggacagtgctccagggaagaatcgggcgtccagacaacccattt
cgagtggccctggaatacatctccagtggaaaccgcagcttgtctgcagtggacttcttt
gccctgaagaactgcagtgaaggaacatccccaggctccaagatggccctgcagagctcc
ttcacttgttggaatgggacagtcctccagcttgggcaggcctgtgacttccaccaggac
tgtgcccagggagaagatgagagccagatgtgccggaaactgcctgtgggtttttactgc
aactttgaagatggcttctgtggctggacccaaggcacactgtcaccccacactcctcaa
tggcaggtcaggaccctaaaggatgcccggttccaggaccaccaagaccatgctctattg
ctcagtaccactgatgtccccgcttctgaaagtgctacagtgaccagtgctacgtttcct
gcaccgatcaagagctctccatgtgagctccgaatgtcctggctcattcgtggagtcttg
aggggaaacgtgtccttggtgctagtggagaacaaaaccgggaaggagcaaggcaggatg
gtctggcatgtcgccgcctatgaaggcttgagcctgtggcagtggatggtgttgcctctc
ctcgatgtgtctgacaggttctggctgcagatggtcgcatggtggggacaaggatccaga
gccatcgtggcttttgacaatatctccatcagcctggactgctacctcaccattagcgga
gaggacaagatcctgcagaatacagcacccaaatcaagaaacctgtttgagagaaaccca
aacaaggagctgaaacccggggaaaattcaccaagacagacccccatctttgaccctaca
gttcattggctgttcaccacatgtggggccagcgggccccatggccccacccaggcacag
tgcaacaacgcctaccagaactccaacctgagcgtggaggtggggagcgagggccccctg
aaaggcatccagatctggaaggtgccagccaccgacacctacagcatctcgggctacgga
gctgctggcgggaaaggcgggaagaacaccatgatgcggtcccacggcgtgtctgtgctg
ggcatcttcaacctggagaaggatgacatgctgtacatcctggttgggcagcagggagag
gacgcctgccccagtacaaaccagttaatccagaaagtctgcattggagagaacaatgtg
atagaagaagaaatccgtgtgaacagaagcgtgcatgagtgggcaggaggcggaggagga
gggggtggagccacctacgtatttaagatgaaggatggagtgccggtgcccctgatcatt
gcagccggaggtggtggcagggcctacggggccaagacagacacgttccacccagagaga
ctggagaataactcctcggttctagggctaaacggcaattccggagccgcaggtggtgga
ggtggctggaatgataacacttccttgctctgggccggaaaatctttgcaggagggtgcc
accggaggacattcctgcccccaggccatgaagaagtgggggtgggagacaagagggggt
ttcggagggggtggaggggggtgctcctcaggtggaggaggcggaggatatataggcggc
aatgcagcctcaaacaatgaccccgaaatggatggggaagatggggtttccttcatcagt
ccactgggcatcctgtacaccccagctttaaaagtgatggaaggccacggggaagtgaat
attaagcattatctaaactgcagtcactgtgaggtagacgaatgtcacatggaccctgaa
agccacaaggtcatctgcttctgtgaccacgggacggtgctggctgaggatggcgtctcc
tgcattgtgtcacccaccccggagccacacctgccactctcgctgatcctctctgtggtg
acctctgccctcgtggccgccctggtcctggctttctccggcatcatgattgtgtaccgc
cggaagcaccaggagctgcaagccatgcagatggagctgcagagccctgagtacaagctg
agcaagctccgcacctcgaccatcatgaccgactacaaccccaactactgctttgctggc
aagacctcctccatcagtgacctgaaggaggtgccgcggaaaaacatcaccctcattcgg
ggtctgggccatggcgcctttggggaggtgtatgaaggccaggtgtccggaatgcccaac
gacccaagccccctgcaagtggctgtgaagacgctgcctgaagtgtgctctgaacaggac
gaactggatttcctcatggaagccctgatcatcagcaaattcaaccaccagaacattgtt
cgctgcattggggtgagcctgcaatccctgccccggttcatcctgctggagctcatggcg
gggggagacctcaagtccttcctccgagagacccgccctcgcccgagccagccctcctcc
ctggccatgctggaccttctgcacgtggctcgggacattgcctgtggctgtcagtatttg
gaggaaaaccacttcatccaccgagacattgctgccagaaactgcctcttgacctgtcca
ggccctggaagagtggccaagattggagacttcgggatggcccgagacatctacagggcg
agctactatagaaagggaggctgtgccatgctgccagttaagtggatgcccccagaggcc
ttcatggaaggaatattcacttctaaaacagacacatggtcctttggagtgctgctatgg
gaaatcttttctcttggatatatgccataccccagcaaaagcaaccaggaagttctggag
tttgtcaccagtggaggccggatggacccacccaagaactgccctgggcctgtataccgg
ataatgactcagtgctggcaacatcagcctgaagacaggcccaactttgccatcattttg
gagaggattgaatactgcacccaggacccggatgtaatcaacaccgctttgccgatagaa
tatggtccacttgtggaagaggaagagaaagtgcctgtgaggcccaaggaccctgagggg
gttcctcctctcctggtctctcaacaggcaaaacgggaggaggagcgcagcccagctgcc
ccaccacctctgcctaccacctcctctggcaaggctgcaaagaaacccacagctgcagag
atctctgttcgagtccctagagggccggccgtggaagggggacacgtgaatatggcattc
tctcagtccaaccctccttcggagttgcacaaggtccacggatccagaaacaagcccacc
agcttgtggaacccaacgtacggctcctggtttacagagaaacccaccaaaaagaataat
cctatagcaaagaaggagccacacgacaggggtaacctggggctggagggaagctgtact
gtcccacctaacgttgcaactgggagacttccgggggcctcactgctcctagagccctct
tcgctgactgccaatatgaaggaggtacctctgttcaggctacgtcacttcccttgtggg
aatgtcaattacggctaccagcaacagggcttgcccttagaagccgctactgcccctgga
gctggtcattacgaggataccattctgaaaagcaagaatagcatgaaccagcctgggccc
tga
EGFR cDNA Sequence Reference SEQ ID NO: 7716
ATGCGACCCTCCGGGACGGCCGGGGCAGCGCTCCTGGCGCTGCTGGCTGCGCTCTGCCCG
GCGAGTCGGCCTCTGGAGGAAAAGAAAGTTTGCCAAGGCACGAGTAACAACCTCACGCAG
TTGGGCACTTTTGAAGATCATTTTCTCAGCCTCCAGAGGATGTTCAATAACTGTGAGGTG
GTCCTTGGGAATTTGGAAATTACCTATGTGCAGAGGAATTATGATCTTTCCTTCTTAAAG
ACCATCCAGGAGGTGGCTGGTTATGTCCTCATTGCCCTCAACACAGTGGAGCGAATTCCT
TTGGAAAACCTGCAGATCATCAGAGGAAATATGTACTACGAAAATTCCTATGCCTTAGCA
GTCTTATCTAACTATGATGCAAATAAAACCGGACTGAAGGAGCTGCCCATGAGAAATTTA
CAGGAAATCCTGCATGGCGCCGTGCGGTTCAGCAACAACCCTGCCCTGTGCAACGTGGAG
AGCATCCAGTGGCGGGACATAGTCAGCAGTGACTTTCTCAGCAACATGTCGATGGACTTC
CAGAACCACCTGGGCAGCTGCCAAAAGTGTGATCCAAGCTGTCCCAATGGGAGCTGCTGG
GGTGCAGGAGAGGAGAACTGCCAGAAACTGACCAAAATCATCTGTGCCCAGCAGTGCTCC
GGGCGCTGCCGTGGCAAGTCCCCCAGTGACTGCTGCCACAACCAGTGTGCTGCAGGCTGC
ACAGGCCCCCGGGAGAGCGACTGCCTGGTCTGCCGCAAATTCCGAGACGAAGCCACGTGC
AAGGACACCTGCCCCCCACTCATGCTCTACAACCCCACCACGTACCAGATGGATGTGAAC
CCCGAGGGCAAATACAGCTTTGGTGCCACCTGCGTGAAGAAGTGTCCCCGTAATTATGTG
GTGACAGATCACGGCTCGTGCGTCCGAGCCTGTGGGGCCGACAGCTATGAGATGGAGGAA
GACGGCGTCCGCAAGTGTAAGAAGTGCGAAGGGCCTTGCCGCAAAGTGTGTAACGGAATA
GGTATTGGTGAATTTAAAGACTCACTCTCCATAAATGCTACGAATATTAAACACTTCAAA
AACTGCACCTCCATCAGTGGCGATCTCCACATCCTGCCGGTGGCATTTAGGGGTGACTCC
TTCACACATACTCCTCCTCTGGATCCACAGGAACTGGATATTCTGAAAACCGTAAAGGAA
ATCACAGGGTTTTTGCTGATTCAGGCTTGGCCTGAAAACAGGACGGACCTCCATGCCTTT
GAGAACCTAGAAATCATACGCGGCAGGACCAAGCAACATGGTCAGTTTTCTCTTGCAGTC
GTCAGCCTGAACATAACATCCTTGGGATTACGCTCCCTCAAGGAGATAAGTGATGGAGAT
GTGATAATTTCAGGAAACAAAAATTTGTGCTATGCAAATACAATAAACTGGAAAAAACTG
TTTGGGACCTCCGGTCAGAAAACCAAAATTATAAGCAACAGAGGTGAAAACAGCTGCAAG
GCCACAGGCCAGGTCTGCCATGCCTTGTGCTCCCCCGAGGGCTGCTGGGGCCCGGAGCCC
AGGGACTGCGTCTCTTGCCGGAATGTCAGCCGAGGCAGGGAATGCGTGGACAAGTGCAAC
CTTCTGGAGGGTGAGCCAAGGGAGTTTGTGGAGAACTCTGAGTGCATACAGTGCCACCCA
GAGTGCCTGCCTCAGGCCATGAACATCACCTGCACAGGACGGGGACCAGACAACTGTATC
CAGTGTGCCCACTACATTGACGGCCCCCACTGCGTCAAGACCTGCCCGGCAGGAGTCATG
GGAGAAAACAACACCCTGGTCTGGAAGTACGCAGACGCCGGCCATGTGTGCCACCTGTGC
CATCCAAACTGCACCTACGGATGCACTGGGCCAGGTCTTGAAGGCTGTCCAACGAATGGG
CCTAAGATCCCGTCCATCGCCACTGGGATGGTGGGGGCCCTCCTCTTGCTGCTGGTGGTG
GCCCTGGGGATCGGCCTCTTCATGCGAAGGCGCCACATCGTTCGGAAGCGCACGCTGCGG
AGGCTGCTGCAGGAGAGGGAGCTTGTGGAGCCTCTTACACCCAGTGGAGAACCTCCCAAC
CAAGCTCTCTTGAGGATCTTGAAGGAAACTGAATTCAAAAAGATCAAAGTGCTGGGCTCC
GGTGCGTTCGGCACGGTGTATAAGGGACTCTGGATCCCAGAAGGTGAGAAAGTTAAAATT
CCCGTCGCTATCAAGGAATTAAGAGAAGCAACATCTCCGAAAGCCAACAAGGAAATCCTC
GATGAAGCCTACGTGATGGCCAGCGTGGACAACCCCCACGTGTGCCGCCTGCTGGGCATC
TGCCTCACCTCCACCGTGCAGCTCATCACGCAGCTCATGCCCTTCGGCTGCCTCCTGGAC
TATGTCCGGGAACACAAAGACAATATTGGCTCCCAGTACCTGCTCAACTGGTGTGTGCAG
ATCGCAAAGGGCATGAACTACTTGGAGGACCGTCGCTTGGTGCACCGCGACCTGGCAGCC
AGGAACGTACTGGTGAAAACACCGCAGCATGTCAAGATCACAGATTTTGGGCTGGCCAAA
CTGCTGGGTGCGGAAGAGAAAGAATACCATGCAGAAGGAGGCAAAGTGCCTATCAAGTGG
ATGGCATTGGAATCAATTTTACACAGAATCTATACCCACCAGAGTGATGTCTGGAGCTAC
GGGGTGACTGTTTGGGAGTTGATGACCTTTGGATCCAAGCCATATGACGGAATCCCTGCC
AGCGAGATCTCCTCCATCCTGGAGAAAGGAGAACGCCTCCCTCAGCCACCCATATGTACC
ATCGATGTCTACATGATCATGGTCAAGTGCTGGATGATAGACGCAGATAGTCGCCCAAAG
TTCCGTGAGTTGATCATCGAATTCTCCAAAATGGCCCGAGACCCCCAGCGCTACCTTGTC
ATTCAGGGGGATGAAAGAATGCATTTGCCAAGTCCTACAGACTCCAACTTCTACCGTGCC
CTGATGGATGAAGAAGACATGGACGACGTGGTGGATGCCGACGAGTACCTCATCCCACAG
CAGGGCTTCTTCAGCAGCCCCTCCACGTCACGGACTCCCCTCCTGAGCTCTCTGAGTGCA
ACCAGCAACAATTCCACCGTGGCTTGCATTGATAGAAATGGGCTGCAAAGCTGTCCCATC
AAGGAAGACAGCTTCTTGCAGCGATACAGCTCAGACCCCACAGGCGCCTTGACTGAGGAC
AGCATAGACGACACCTTCCTCCCAGTGCCTGAATACATAAACCAGTCCGTTCCCAAAAGG
CCCGCTGGCTCTGTGCAGAATCCTGTCTATCACAATCAGCCTCTGAACCCCGCGCCCAGC
AGAGACCCACACTACCAGGACCCCCACAGCACTGCAGTGGGCAACCCCGAGTATCTCAAC
ACTGTCCAGCCCACCTGTGTCAACAGCACATTCGACAGCCCTGCCCACTGGGCCCAGAAA
GGCAGCCACCAAATTAGCCTGGACAACCCTGACTACCAGCAGGACTTCTTTCCCAAGGAA
GCCAAGCCAAATGGCATCTTTAAGGGCTCCACAGCTGAAAATGCAGAATACCTAAGGGTC
GCGCCACAAAGCAGTGAATTTATTGGAGCATGA
BRAF cDNA Sequence Reference SEQ ID NO: 7717
atggcggcgctgagcggtggcggtggtggcggcgcggagccgggccaggctctgttcaac
ggggacatggagcccgaggccggcgccggcgccggcgccgcggcctcttcggctgcggac
cctgccattccggaggaggtgtggaatatcaaacaaatgattaagttgacacaggaacat
atagaggccctattggacaaatttggtggggagcataatccaccatcaatatatctggag
gcctatgaagaatacaccagcaagctagatgcactccaacaaagagaacaacagttattg
gaatctctggggaacggaactgatttttctgtttctagctctgcatcaatggataccgtt
acatcttcttcctcttctagcctttcagtgctaccttcatctctttcagtttttcaaaat
cccacagatgtggcacggagcaaccccaagtcaccacaaaaacctatcgttagagtcttc
ctgcccaacaaacagaggacagtggtacctgcaaggtgtggagttacagtccgagacagt
ctaaagaaagcactgatgatgagaggtctaatcccagagtgctgtgctgtttacagaatt
caggatggagagaagaaaccaattggttgggacactgatatttcctggcttactggagaa
gaattgcatgtggaagtgttggagaatgttccacttacaacacacaactttgtacgaaaa
acgtttttcaccttagcattttgtgacttttgtcgaaagctgcttttccagggtttccgc
tgtcaaacatgtggttataaatttcaccagcgttgtagtacagaagttccactgatgtgt
gttaattatgaccaacttgatttgctgtttgtctccaagttctttgaacaccacccaata
ccacaggaagaggcgtccttagcagagactgccctaacatctggatcatccccttccgca
cccgcctcggactctattgggccccaaattctcaccagtccgtctccttcaaaatccatt
ccaattccacagcccttccgaccagcagatgaagatcatcgaaatcaatttgggcaacga
gaccgatcctcatcagctcccaatgtgcatataaacacaatagaacctgtcaatattgat
gacttgattagagaccaaggatttcgtggtgatggaggatcaaccacaggtttgtctgct
accccccctgcctcattacctggctcactaactaacgtgaaagccttacagaaatctcca
ggacctcagcgagaaaggaagtcatcttcatcctcagaagacaggaatcgaatgaaaaca
cttggtagacgggactcgagtgatgattgggagattcctgatgggcagattacagtggga
caaagaattggatctggatcatttggaacagtctacaagggaaagtggcatggtgatgtg
gcagtgaaaatgttgaatgtgacagcacctacacctcagcagttacaagccttcaaaaat
gaagtaggagtactcaggaaaacacgacatgtgaatatcctactcttcatgggctattcc
acaaagccacaactggctattgttacccagtggtgtgagggctccagcttgtatcaccat
ctccatatcattgagaccaaatttgagatgatcaaacttatagatattgcacgacagact
gcacagggcatggattacttacacgccaagtcaatcatccacagagacctcaagagtaat
aatatatttcttcatgaagacctcacagtaaaaataggtgattttggtctagctacagtg
aaatctcgatggagtgggtcccatcagtttgaacagttgtctggatccattttgtggatg
gcaccagaagtcatcagaatgcaagataaaaatccatacagctttcagtcagatgtatat
gcatttggaattgttctgtatgaattgatgactggacagttaccttattcaaacatcaac
aacagggaccagataatttttatggtgggacgaggatacctgtctccagatctcagtaag
gtacggagtaactgtccaaaagccatgaagagattaatggcagagtgcctcaaaaagaaa
agagatgagagaccactctttccccaaattctcgcctctattgagctgctggcccgctca
ttgccaaaaattcaccgcagtgcatcagaaccctccttgaatcgggctggtttccaaaca
gaggattttagtctatatgcttgtgcttctccaaaaacacccatccaggcagggggatat
ggtgcgtttcctgtccactga
KRAS cDNA Sequence Reference SEQ ID NO: 7718
atgactgaatataaacttgtggtagttggagctggtggcgtaggcaagagtgccttgacg
atacagctaattcagaatcattttgtggacgaatatgatccaacaatagaggattcctac
aggaagcaagtagtaattgatggagaaacctgtctcttggatattctcgacacagcaggt
caagaggagtacagtgcaatgagggaccagtacatgaggactggggagggctttctttgt
gtatttgccataaataatactaaatcatttgaagatattcaccattatagagaacaaatt
aaaagagttaaggactctgaagatgtacctatggtcctagtaggaaataaatgtgatttg
ccttctagaacagtagacacaaaacaggctcaggacttagcaagaagttatggaattcct
tttattgaaacatcagcaaagacaagacagggtgttgatgatgccttctatacattagtt
cgagaaattcgaaaacataaagaaaagatgagcaaagatggtaaaaagaagaaaaagaag
tcaaagacaaagtgtgtaattatgtaa

What is claimed is:

1. A kinase inhibitor resistance panel comprising one or more primer sets from one or more of the genes selected from group of genes comprising KRAS, BRAF, EGFR, ALK, and KIT.

2. The kinase inhibitor resistance panel of claim 1, wherein at least one primer sets hybridizes and amplifies nucleic acid from exon 1 or 2 of KRAS.

3. The kinase inhibitor panel of claim 2, wherein one or more KRAS hybridizing primers or primer sets comprise one or more of the primers of Tables 10 and/or 14.

4. The kinase inhibitor panel of claim 2, wherein one or more KRAS hybridizing primers or primer sets comprise one or more of the primers of SEQ ID NOs: 4601-5200 and 7181-7610.

5. The kinase inhibitor resistance panel of claim 1, wherein at least one primer sets hybridizes and amplifies nucleic acid from exon 18, 19, 20, 21 or 22 of EGFR.

6. The kinase inhibitor panel of claim 5, wherein one or more EGFR hybridizing primers or primer sets comprise one or more of the primers of Tables 8 and/or 12.

7. The kinase inhibitor panel of claim 6, wherein one or more EGFR hybridizing primers or primer sets comprise one or more of the primers of SEQ ID NOs: 1641-2440 and 5819-6524.

8. The kinase inhibitor resistance panel of claim 1, wherein at least one primer sets hybridizes and amplifies nucleic acid from exon 21, 22, 23, 24, or 25 of ALK.

9. The kinase inhibitor panel of claim 8, wherein one or more ALK hybridizing primers or primer sets comprise one or more of the primers of Tables 7 and/or 11.

10. The kinase inhibitor panel of claim 9, wherein one or more ALK hybridizing primers or primer sets comprise one or more of the primers of SEQ ID NOs: 1-1640 and 5201-5818.

11. The kinase inhibitor resistance panel of claim 1, wherein at least one primer sets hybridizes and amplifies nucleic acid from exon 8, 9, 10, 11, 12, 13, or 17 of KIT.

12. The kinase inhibitor panel of claim 11, wherein one or more KIT hybridizing primers or primer sets comprise one or more of the primers of Table 9.

13. The kinase inhibitor panel of claim 12, wherein one or more KIT hybridizing primers or primer sets comprise one or more of the primers of SEQ ID NOs: 2441-4600.

14. The kinase inhibitor resistance panel of claim 1, wherein at least one primer sets hybridizes and amplifies nucleic acid from exon 10, 11, 13, 14, or 15 of BRAF.

15. The kinase inhibitor panel of claim 14, wherein one or more BRAF hybridizing primers or primer sets comprise one or more of the primers of Table 13.

16. The kinase inhibitor panel of claim 15, wherein one or more BRAF hybridizing primers or primer sets comprise one or more of the primers of SEQ ID NOs: 6525-7180.

17. The kinase inhibitor panel of claim 1, wherein the panel comprises two or more primer sets for one or more of the genes selected from group of genes comprising KRAS, BRAF, EGFR, ALK, and KIT.

18. The kinase inhibitor panel of claim 1, wherein the panel comprises 3, 4, 5, 6, 7, 8, 9, 10, or more primer sets for one or more of the genes selected from group of genes comprising KRAS, BRAF, EGFR, ALK, and KIT.

19. The kinase inhibitor panel of claim 1, wherein the panel comprises one or more primer sets for 2, 3, 4, of all 5 of the genes selected from group of genes comprising KRAS, BRAF, EGFR, ALK, and KIT.

20. The kinase inhibitor of claim 1, wherein the kinase inhibitor resistance is resistance to an ALK kinase inhibitor.

21. The kinase inhibitor resistance panel of claim 20, wherein the kinase inhibitor is selected from the group consisting of crizotinib, afatinib, Axitinib, bevacizumab, Bosutinib, Cetuximab, Dasatinib, Erlotinib, Fostamati nib, Gefitinib, Imatinib, Lapatinib, Lenvatinib, Nilotinib, Panitumumab, Pazopanib, Pegaptanib, Ranibizumab, Ruxolitinib, Sorafenib, Sunitinib, Trastuzumab, and Vemurafenib.

22. The kinase inhibitor resistance panel of claim 21, wherein the kinase inhibitor comprises crizotinib.

23. The kinase inhibitor panel of claim 1, wherein the one or more primer sets amplify one or more mutations associated with kinase inhibitor resistance, wherein the mutation is identified in Table 2, 3, 4, 5, or 6.

24. A method for the detection of kinase inhibitor resistance comprising obtaining a tissue sample from a subject with a cancer and conducting a high throughput sequencing (also known as next generation sequencing) reaction on the sample using the kinase inhibitor resistant panel of claim 1, wherein the presence of a mutation in the nucleic acid sequence of a gene associated with kinase inhibitor resistance indicates that that the cancer is resistant or will become resistant to a kinase inhibitor.

25. A method for the detection of kinase inhibitor resistance comprising obtaining a tissue sample from a subject with a cancer and conducting a high throughput sequencing (also known as next generation sequencing) reaction on the sample using one or more primer sets or primer panels with primer sets that specifically hybridizes to one or more of the genes selected from the group consisting ofALK, KRAS, EGFR, KIT, and BRAF, wherein the presence of a mutation in the nucleic acid sequence of a gene associated with kinase inhibitor resistance indicates that that the cancer is resistant or will become resistant to a kinase inhibitor.

26. The method of claim 25, wherein at least one primer sets hybridizes and amplifies nucleic acid from exon 1 or 2 of KRAS.

27. The method of claim 26, wherein one or more KRAS hybridizing primers or primer sets comprise one or more of the primers of Tables 10 and/or 14.

28. The method of claim 26, wherein one or more KRAS hybridizing primers or primer sets comprise one or more of the primers of SEQ ID NOs: 4601-5200 and 7181-7610.

29. The method of claim 25, wherein at least one primer sets hybridizes and amplifies nucleic acid from exon 18, 19, 20, 21 or 22 of EGFR.

30. The method of claim 29, wherein one or more EGFR hybridizing primers or primer sets comprise one or more of the primers of Tables 8 and/or 12.

31. The method of claim 29, wherein one or more EGFR hybridizing primers or primer sets comprise one or more of the primers of SEQ ID NOs: 1641-2440 and 5819-6524.

32. The method of claim 25, wherein at least one primer sets hybridizes and amplifies nucleic acid from exon 21, 22, 23, 24, or 25 of ALK.

33. The method of claim 32, wherein one or more ALK hybridizing primers or primer sets comprise one or more of the primers of Tables 7 and/or 11.

34. The method of claim 32, wherein one or more ALK hybridizing primers or primer sets comprise one or more of the primers of SEQ ID NOs: 1-1640 and 5201-5818.

35. The method of claim 25, wherein at least one primer sets hybridizes and amplifies nucleic acid from exon 8, 9, 10, 11, 12, 13, or 17 of KIT.

36. The method of claim 35, wherein one or more KIT hybridizing primers or primer sets comprise one or more of the primers of Table 9.

37. The method of claim 35, wherein one or more KIT hybridizing primers or primer sets comprise one or more of the primers of SEQ ID NOs: 2441-4600.

38. The method of claim 25, wherein at least one primer sets hybridizes and amplifies nucleic acid from exon 10, 11, 13, 14, or 15 of BRAF.

39. The method of claim 38, wherein one or more BRAF hybridizing primers or primer sets comprise one or more of the primers of Table 13.

40. The method of claim 38, wherein one or more BRAF hybridizing primers or primer sets comprise one or more of the primers of SEQ ID NOs: 6525-7180.

41. The method of claim 25, wherein the panel comprises two or more primer sets for one or more of the genes selected from group of genes comprising KRAS, BRAF, EGFR, ALK, and KIT.

42. The method of claim 25, wherein the panel comprises 3, 4, 5, 6, 7, 8, 9, 10, or more primer sets for one or more of the genes selected from group of genes comprising KRAS, BRAF, EGFR, ALK, and KIT.

43. The method of claim 25, wherein the panel comprises one or more primer sets for 2, 3, 4, of all 5 of the genes selected from group of genes comprising KRAS, BRAF, EGFR, ALK, and KIT.

44. The method of claim 25, wherein the kinase inhibitor resistance is resistance to an ALK kinase inhibitor.

45. The method of claim 25, wherein the kinase inhibitor is selected from the group consisting of crizotinib, afatinib, Axitinib, bevacizumab, Bosutinib, Cetuximab, Dasatinib, Erlotinib, Fostamati nib, Gefitinib, Imatinib, Lapatinib, Lenvatinib, Nilotinib, Panitumumab, Pazopanib, Pegaptanib, Ranibizumab, Ruxolitinib, Sorafenib, Sunitinib, Trastuzumab, and Vemurafenib.

46. The method of claim 45, wherein the kinase inhibitor comprises crizotinib.

47. The method of claim 25, wherein the one or more primer sets amplify one or more mutations associated with kinase inhibitor resistance, wherein the mutation is identified in Table 2, 3, 4, 5, or 6.
